(19) United States
(12) Patent Application Publication
(10) Pub. No.: US 2026/0159543 A1
(43) Pub. Date: Jun. 11, 2026

(54) METHOD OF TREATING MELANOCORTIN-4 RECEPTOR-ASSOCIATED DISORDERS IN HETEROZYGOUS CARRIERS

(71) Applicant: RHYTHM PHARMACEUTICALS, INC., Boston, MA (US)

(72) Inventors: Louis Anthony Tartaglia, Newton, MA (US); Bart Henderson, Belmont, MA (US); Leonardus H.T. Van Der Ploeg, Newton, MA (US)

(21) Appl. No.: 19/532,060

(22) Filed: Feb. 6, 2026

Related U.S. Application Data
(63) Continuation of application No. 18/204,672, filed on Jun. 1, 2023.
Application No. 18/204,672 is a continuation of application No. 17/097,122, filed on Nov. 13, 2020, now Pat. No. 11,702,448.
Application No. 17/097,122 is a continuation of application No. 16/383,889, filed on Apr. 15, 2019, now Pat. No. 10,954,268.
Application No. 16/383,889 is a continuation of application No. 16/117,767, filed on Aug. 30, 2018, now abandoned.
Application No. 16/117,767 is a continuation of application No. 15/789,118, filed on Oct. 20, 2017, now Pat. No. 10,167,312.
Application No. 15/789,118 is a continuation of application No. 14/369,116, now Pat. No. 9,845,339.
(60) Provisional application No. 61/581,391, filed on Dec. 29, 2011.

(51) Int. Cl. C07K 5/08; A61K 38/08; C07K 7/06; C07K 14/68; C07K 14/72

(52) U.S. Cl. CPC C07K 5/0802; A61K 38/08; C07K 7/06; C07K 14/68; C07K 14/723

(57) ABSTRACT

A method of treating a disorder in a subject. The method comprises administering to said subject an effective amount of an agonist of the melanocortin-4 receptor (MC4R). The subject is a heterozygous carrier of an MC4R mutation, and the disorder results from an attenuated response of MC4R to α-melanocortin stimulating hormone (α-MSH).

### RELATED APPLICATIONS

The present application is a continuation of U.S. application Ser. No. 18/204,672, filed Jun. 1, 2023, which is a continuation of U.S. application Ser. No. 17/097,122, now U.S. Pat. No. 11,702,448, filed Nov. 13, 2020, which is a continuation of U.S. application Ser. No. 16/383,889, now U.S. Pat. No. 10,954,268, filed Apr. 15, 2019, which is a continuation of U.S. application Ser. No. 16/117,767, filed Aug. 30, 2018, which is a continuation of U.S. application Ser. No. 15/789,118, now U.S. Pat. No. 10,167,312, filed Oct. 20, 2017, which is a continuation of U.S. application Ser. No. 14/369,116, now U.S. Pat. No. 9,845,339, filed Jun. 26, 2014, which is a U.S. national stage application under 35 U.S.C. § 371 of International Application No. PCT/US2012/072026, filed Dec. 28, 2012, which claims the benefit of U.S. Provisional Application No. 61/581,391, filed Dec. 29, 2011. The entire teachings of the above applications are incorporated herein by reference.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Jul. 29, 2023, is entitled “R2054-700024_ST26.xml”, and is 19,356 bytes in size.

### BACKGROUND OF THE INVENTION

Melanocortin 4 receptor (MC4R) mutations can result in genetically derived cause of human obesity or metabolic syndrome. MC4R receptor is a heterotrimeric G-protein-coupled receptor, which transduces signals by activating adenylate cyclase. Expressed in hypothalamic nuclei and other neuronal and non-neuronal tissues, controlling feeding behavior and energy homeostasis, MC4R integrates an agonist (anorexigenic) signal provided by the α-melanocyte stimulating hormone (α-MSH), and an antagonist (orexigenic) signal provided by the agouti-related peptide (AGPR).

As shown in FIG. 1, MC4R is a part of the leptin-melanocortin pathway. Leptin is released from adipose tissue and binds to leptin receptors (LEPR) on AGPR- and pro-opiomelanocortin (POMC)-releasing neurons in the arcuate nucleus (ARC) of the hypothalamus. Leptin binding inhibits AGPR release and stimulates the release of POMC, which undergoes post-translational modification by the prohormone convertase PC1/3 to generate a range of peptides, including α-MSH. AGPR binding to MC4R suppresses MC4R activity, while α-MSH binding stimulates the MC4R. Suppressed receptor activity generates orexigenic signal, whereas stimulated receptor activity generates anorexigenic signal. Signals from MC4R modulate feeding behavior through secondary effector neurons.

Humans affected by a monogenic MC4R-caused disorders, e.g., obesity, are mostly heterozygous carriers of mutant human MC4R (hMC4R) gene with an autosomal dominant inheritance and penetrance and expressivity that varies with age and generational influences. The functional consequences of hMC4R mutations can be schematically divided into the following categories: nonfunctional receptor (e.g. due to missense or frameshift mutations), intracellular retention of the expressed receptor, altered basal activity of the receptor, and altered α-MSH stimulation of the receptor.

### SUMMARY OF THE INVENTION

The need exists for a method of treating disorders associated with MC4R mutations. It has now been discovered that certain individuals that carry an MC4R mutations can respond to pharmacological agents that activate MC4R-mediated signaling pathway. These individuals are heterozygous carriers of an MC4R mutation. Based on this discovery, it is now possible to treat MC4R-mediated disorders in a class of patients that was previously considered unresponsive to MC4R agonists.

Accordingly, an example embodiment of the present invention is a method of treating a disorder in a subject in need thereof. The method comprises administering to said subject an effective amount of an agonist of the melanocortin-4 receptor (MC4R). The subject is a heterozygous carrier of an MC4R mutation, and the disorder results from an attenuated response of MC4R to α-melanocortin stimulating hormone (α-MSH).

In a particular embodiment, the disorder is obesity (for example, obesity caused by an MC4R mutation, such as loss of function) and the subject is heterozygous with respect to the MC4R gene. In this embodiment, treatment of such a subject with a pharmacological agent that activates MC4R-mediated signaling pathway, such as described herein, may confer a number of unexpected advantages and benefits. For instance, most subjects heterozygous for MC4R may respond to treatment with sustained weight loss. A proportion of subjects may have MC4R functionality restored to wild type levels, resulting in body weight and body composition normalization. Additional benefits may include overcoming hyperinsulinemia, and improving glucose control and hyperphagia. A further benefit may be that weight loss is sustained throughout the treatment period as well as for prolonged periods of time on treatment cessation.

Additional unexpected benefits of treating an MC4R-mediated obesity in an MC4R-heterozygous subject by a a pharmacological agent that activates MC4R-mediated signaling pathway, when compared to an obese subject that is wild-type with respect to MC4R, may include one or more of: an unexpectedly long ability to sustain a drug holiday, without gaining weight; a more profound improvement in insulin and glucose management; a longer lasting and sustained reduction in meal size and food intake; a more profound effect on reducing sleep apnea and increasing quality of sleep; an unexpected and more profound improvement effect on parameters of male or female sexual dysfunction; a more profound reduction in the incidence of obesity-associated cancers; a more profoundly reduced incidence in obesity-associated inflammatory disease including rheumatoid arthritis and endothelial and micro-vascular dysfunction; a more profoundly reduced incidence of heart attack and stroke; more profound improvements in cardiovascular parameters including heart rate and blood pressure.

There are additional benefits to treatment of an MC4R-mediated obesity in an MC4R-heterozygous subject (MC4R+/−) by a pharmacological agent that activates MC4R-mediated signaling pathway, when compared to an obese subject that is wild-type with respect to MC4R. MC4R+/− obese individuals are more at risk than wild type obese individuals of the consequences of obesity because of the intractability of their obesity, and the duration of the MC4R-mediated obesity, that often has a high rate of childhood onset. For example, MC4+/−obese individuals are resistant to weight management by diet/exercise regimens. (Reinhhr et. al, “Lifestyle Intervention in Obese Children With Variations in the Melanocortin 4 Receptor Gene,” Obesity Journal, Vol. 17 No. 2, 2009). It is well-established, however, that higher childhood body-mass index (BMI) values elevate the risk of having a Coronary Heart Disease event in adulthood. (Baker et al., “Childhood Body-Mass Index and the Risk of Coronary Heart Disease in Adulthood,” N. Engl. J. Med 2007; 357:2329-2337 (2007).) Treatment of this higher risk patient group may provide a treatment option not previously available (e.g., a treatment that achieves long term weight management).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram of the leptin-melanocortin pathway.

FIGS. 2A and 2B show Tables 1 and 2 which list examples of the MC4R mutations that cause obesity in humans.

FIG. 3 is a bar plot showing the effect of administration of a compound of SEQ ID NO: 140 to mice as described in Example 1.

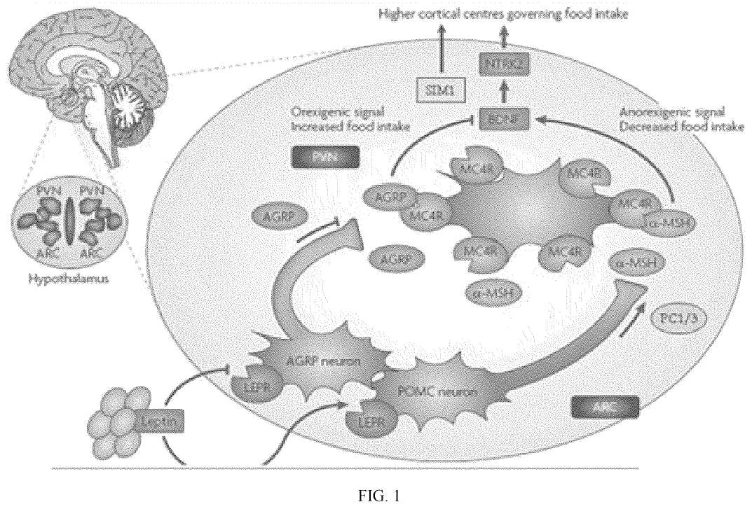

### DETAILED DESCRIPTION OF THE INVENTION

A description of example embodiments of the invention follows.

The present invention relates to a method of treating a disorder in a subject suffering from an attenuated response of MC4R to α-melanocortin stimulating hormone (α-MSH). The method comprises administering an effective amount of an agonist of the melanocortin-4 receptor (MC4R). In an example embodiment, the subject is a heterozygous carrier of an MC4R mutation resulting in the attenuated response of MC4R to α-melanocortin stimulating hormone (α-MSH). Because heterozygous carriers retain an ability to respond to the natural ligand of MC4R, treating MC4R-associated disorders in heterozygous carriers by administration of an MC4R agonist does not rely on the knowledge of the type of the MC4R mutation.

In one example embodiment, the disorder is obesity, for example, MC4R-associated obesity. In another example embodiment, the disorder is metabolic syndrome.

As used herein, the term “obese” refers to a subject having a body mass index (BMI) within the ranges defined as “obese” by the Center for Desease Control. See, URL http://www.cdc.gov/obesity/defining.html, last accessed on Oct. 28, 2011. For example, an adult who has a BMI of 30 or higher is considered obese,

As used herein, the term “metabolic syndrome” refers to a group of symptoms that occur together and increase the risk for coronary artery disease, stroke, and type 2 diabetes. According to the American Heart Association and the National Heart, Lung, and Blood Institute, metabolic syndrome also referred to as Syndrom X) is present if a subject has three or more of the following signs:
1) Blood pressure equal to or higher than 130/85 mmHg;
2) Fasting blood sugar (glucose) equal to or higher than 100 mg/dL;
3) Large waist circumference (length around the waist):
   Men—40 inches or more;
   Women—35 inches or more;
4) Low HDL cholesterol:
   Men—under 40 mg/dL;
   Women—under 50 mg/dL;
5) Triglycerides equal to or higher than 150 mg/dL.
   Metabolic syndrome can be diagnosed by testing subject's blood pressure, blood glucose level, HDL cholesterol level, LDL cholesterol level, total cholesterol level, and triglyceride level.

As used herein, the phrase “attenuated response” refers to reduction, but not complete abrogation, of a signaling activity of a receptor in response to its cognate naturally occurring or synthetic ligand.

As used herein, the term “agonist” refers to any chemical compound, either naturally occurring or synthetic, that, upon interacting with (e.g., binding to) its target, here, MC4R, raises the signaling activity of MC4R above its basal level. An agonist can be a superagonist (i.e. a compound that is capable of producing a greater maximal response than the endogenous agonist for the target receptor, and thus has an efficacy of more than 100%), a full agonist (i.e. a compound that elicits a maximal response following receptor occupation and activation) or a partial agonist (i.e. a compounds that can activate receptors but are unable to elicit the maximal response of the receptor system).

Examples of naturally occurring MC4R agonists include α-MSH, β-MSH, γ-MSH and adenocorticitropic hormone (ACTH) or a functional fragment thereof. Examples of synthetic MC4R agonists will be described in detail below.

As used herein, an “effective amount” is a therapeutically or prophylactically sufficient amount of the MC4R agonist to treat the target disorder. Examples of effective amounts typically range from about from 0.005 mg/kg of body weight to 500 mg/kg of body weight. In other examples, effective amounts range from about from 0.01 mg/kg of body weight to 50 mg/kg of body weight, or from 0.01 mg/kg of body weight to 20 mg/kg of body weight.

As used herein “treating” includes achieving, partially or substantially, one or more of the following results: partially or totally reducing the body weight (as measured, for example, by a body mass index, BMI); ameliorating or improving a clinical symptom or indicators associated with obesity, such as type-II diabetes, pre-diabetic condition, blood level of haemoglobin A1C (Hb1Ac) above 6%, hyperinsulimenia, hyperlipidemia, insulin insensitivity, glucose intolerance etc; delaying, inhibiting or preventing the progression of obesity and obesity related indication; or partially or totally delaying, inhibiting or preventing the onset or development of obesity or obesity related indication. Delaying, inhibiting or preventing the progression of the obesity includes for example, delaying, inhibiting or preventing the progression of a subject having normal weight to obesity.

The term “treating” further includes partially or totally reducing the risk for coronary artery disease, stroke, and type 2 diabetes associated with the metabolic syndrome as well as ameliorating or improving a clinical symptom or signs of metabolic syndrome associated with metabolic syndrome, such as any one or more of the five indicators listed above. For example, the term “treating” includes delaying, inhibiting or preventing the progression of parameters associated with the metabolic syndrome, including insulin resistance, glucose clearance and parameters of cardiovascular disease including heart rate and blood pressure.

“Prophylactic treatment” refers to treatment before onset of obesity to prevent, inhibit or reduce its occurrence.

As used herein, the term “subject” refers to a mammal, preferably a human, but can also mean an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

hMC4R is a well-characterized protein encoded by a genomic sequence having GenBank accession number CH471077.

Mutations in the MC4R receptor are an associated cause of severe childhood obesity. The carrier prevalence for MC4R mutations in a juvenile-onset obese population has been noted to be around 2.5% with a highest prevalence of 6% among severe obese children. Humans with MC4R mutations show a more or less similar phenotype as has been described for mice with mutations in the MC4 receptor gene. Those people show clear hyperphagia, hyperinsulinaemia, increased fat mass, accompanied by lean body mass, bone mineral density and linear growth rate, with no changes in cortisol levels, gonadotropin, thyroid and sex steroid levels. In contrast to MC4 receptor deletion, hyperphagia and hyperinsulinaemia tends to subside with age in human subjects. Similar to the MC4R knockout mice, the phenotype in heterozygote carriers is intermediate in comparison to homozygote carriers. The exhibited hyperphagia observed upon a test meal is less severe than that observed in people with a leptin deficiency. The severity of MC4 receptor dysfunction seen in assays in vitro can predict the amount of food ingested at a test meal by the subject harboring that particular mutation and correlates with the onset and severity of the obese phenotype. At east 90 different MC4 receptor mutations have been associated with obesity and additional mutations in the MC4 receptor are likely to be discovered, leading to a similar obesity phenotype.

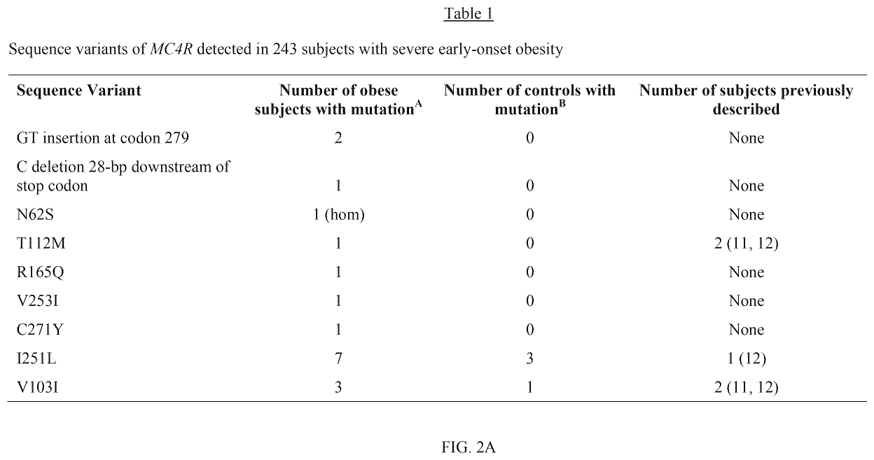

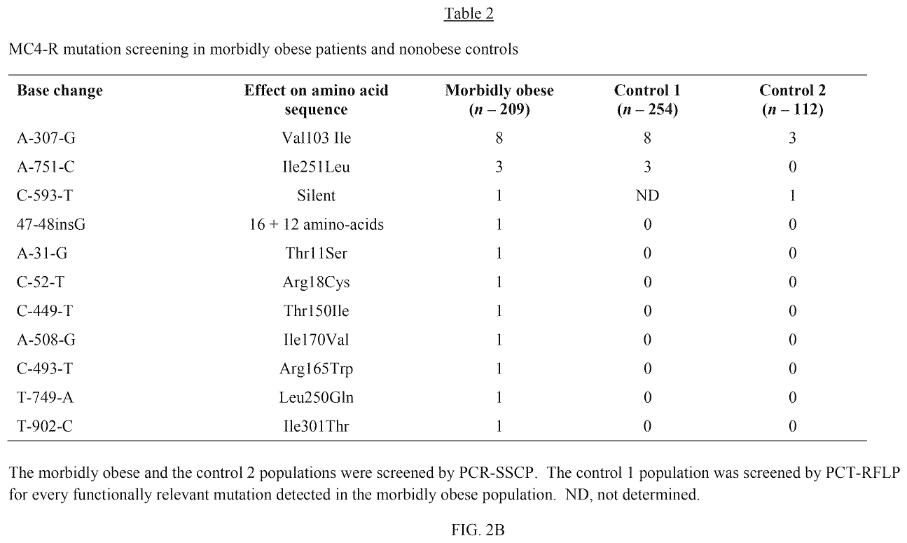

Examples of the MC4R mutations that cause obesity in humans are shown in FIGS. 2A and 2B as Table 1 and Table 2 (adopted from Farooqi et al., The Journal of Clinical Investigation, July 2000, vol. 106 (2), pp. 271-279 and Vaisse et al., The Journal of Clinical Investigation, July 2000, vol. 106(2), pp. 253-262, the relevant portions of which are incorporated herein by reference).

Additional mutations that potentially cause obesity in humans include, R18H, R18L, S36Y, P48S, V50M, F51L, E61K, I69T, D90N, S94R, G98R, I121T, A154D, Y157S, W174C, G181D, F202L, A219 V, 1226T, G231S, G238D, N240S, C271R, S295P, P299L, E308K, I317V, L325F, and 750DelGA, as described in Xiang et al., “Pharmacological characterization of 30 human melanocortin-4 receptor polymorphisms with the endogenous proopiomelanocortin-derived agonists, synthetic agonists, and the endogenous agouti-related protein antagonist.” Biochemistry, 2010 Jun. 8; 49(22):4583-600, the the relevant portions of which are incorporated herein by reference.

Further examples of mutations that potentially cause obesity in humans are those listed in Online Mendelian Inheritance in Man (OMIM), a database of human genes and genetic disorders, under the accession number 155541 (MC4R) (more precisely, accession nos. 155541.0001-155541.0023) at the URL http://omim.org/entry/155541. Representative examples include 4-BP DEL, NT631; 4-BP INS, NT732; TYR35TER; ASP37VAL; SER58CYS; ILE102SER; ASN274SER; 1-BP INS, 112A; 4-BP DEL, 211CTCT; ILE125LYS; ALA175THR; ILE316SER; TYR287TER; ASN97ASP; 15-BP DEL (delta88-92 codons); and SER127LEU. The relevant portions of the OMIM database are incorporated herein by reference.

In example embodiments, the MC4R mutation results in retention of the MC4R signaling activity.

Mutations in the genomic sequence encoding MC4R can be detected by the methods that are well known to a person of ordinary skill in the art. For example, the genomic sequence can be cloned using nucleotide primers, such as e.g., the primers described in Farooqi et al., The Journal of Clinical Investigation, July 2000, vol. 106 (2), pp. 271-279 and Vaisse et al., The Journal of Clinical Investigation, July 2000, vol. 106(2), pp. 253-262, and the cloned sequence analyzed using commercially available sequencers and software.

Activity of MC4R can be measured by the methods well known to a person of ordinary skill in the art. For example, cells can be transiently transfected with the cloned MC4R DNA, the transfected cells contacted by an agonist of MC4R (e.g. α-MSH), and the intracellular level of cAMP, the secondary messenger of MC4R, measured by an electrochemiluminescence assay described, e.g., in Roubert et al., Journal of Endocrinology (2010) 207, pp. 177-183. A reduction in MC4R signaling can be ascertained by comparing the intracellular level of cAMP produced in response to a given agonist by a wild type MC4R to that produced by a mutant MC4R.

In an example embodiment, an agonist employed by the methods of the present invention can be any known agonist of MC4R. In some example embodiment, the MC4R agonist is not an adrenocorticotropic hormone (ACTH) or a fragment thereof.

In an example embodiment, an MC4R agonist is any of the peptides disclosed in International Application published as WO/2005/000339. Specifically, examples include peptides of the following structural formula:

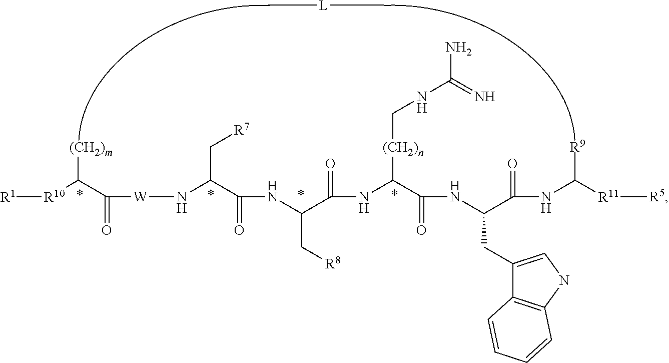

wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, pheylsulfonyl,
C₈-C₁₄ bicyclic arylsulfonyl, phenyl-(CH₂)_{q}C(O)—, C₈-C₁₄ bicyclic aryl-(CH₂)_{q}C(O)—,

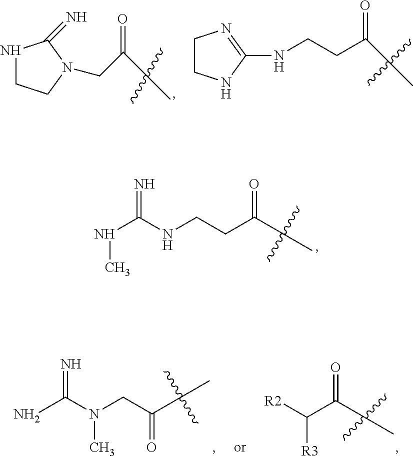

wherein
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr-NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile,

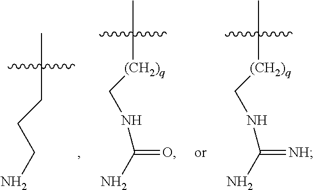

q is 0, 1, 2, or 3;
R⁶ is a phenyl or C₈-C₁₄ bicyclic aryl;
m is 1 or 2;
n is 1, 2, 3, or 4;
R⁹ is (CH₂)ₚ or (CH₃)₂C—;
p is 1 or 2;
R¹⁰ is NH— or is absent;
R⁷ is a 5- or 6-membered heteroaryl or a 5- or 6-membered heteroaryl ring optionally
   substituted with R⁴;
R⁴ is H, C₁-C₄ straight or branched alkyl, phenyl, benzyl, or (C₆H₅)—CH₂—O—CH₂—;
R⁸ is phenyl, a phenyl ring optionally substituted with X, or cyclohexyl;
X is H, Cl, F, Br, methyl, or methoxy;
R¹¹ is —C(O) or —CH₂;
R⁵ is —NH₂, —OH, glycinol, NH₂-Pro-Ser-, NH₂-Pro-Lys-, HO-Ser-, HO-Pro-Ser-, HO-Lys-, Ser alcohol, -Ser-Pro alcohol, -Lys-Pro alcohol, HOCH₂CH₂—O—CH₂CH₂NH—, NI-2-Phe-Arg-, NH₂-Glu-, NH₂CH₂RCH₂NH—, RHN—, RO— where R is a C₁-C₄ straight or branched alkyl; and
   L is —S—S— or —S—CH₂—S—.

Other examples of MC4R agonists include peptides of the following structural formula:

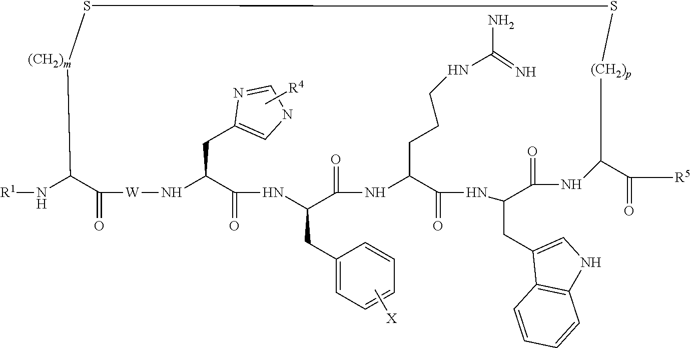

wherein:
W is a single bond, Glu, Gln, Asp, Asn, Ala, Gly, Thr, Ser, Pro, Met, Ile, Val, Arg, His, Tyr, Trp, or Phe;
R¹ is —H, —C(O)CH₃, —C(O)(CH₂)₁₋₄CH₃, —C(O)(CH₂)₁₋₄—NHC(NH)NH₂, Tyr-βArg, gluconoyl-Tyr-Arg, Ac-Dab, Ac-Dap, N-succinyl-Tyr-Arg, N-propionyl, N-valeryl, N-glutaryl-Tyr-Arg, N-butyryl,

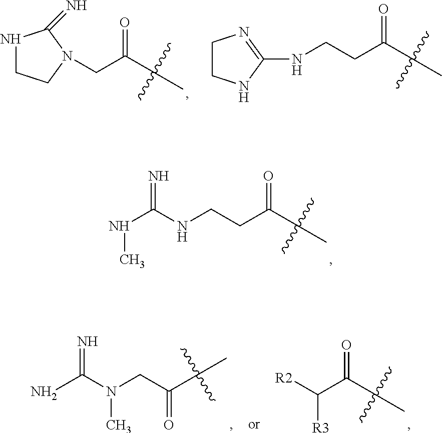

wherein
R² is —H, —NH₂, —NHC(O)CH₃, —NHC(O)(CH₂)₁₋₄CH₃, or —NH-TyrC(O)CH₃;
R³ is C₁-C₄ straight or branched alkyl, Ser, Ile,

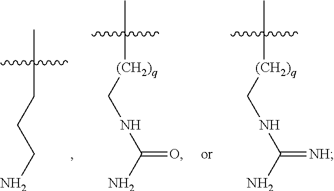

q is 0, 1, 2, or 3;
m is 1 or 2;
p is 1 or 2;
R⁴ is H or C₁-C₄ straight or branched alkyl;
X is H, Cl, F, Br, methyl, or methoxy; and
R⁵ is —NH₂, —OH, glycinol, -Ser-Pro-NH₂, -Lys-Pro-NH₂, -Ser-OH, -Ser-Pro-OH, -Lys-Pro-OH-Arg-Phe-NH₂, -Glu-NH₂, —NHR, or —OR, where R is a C₁-C₄ straight or branched alkyl.

In yet another example embodiment, the MC4R agonist can be represented by the following structural formula:

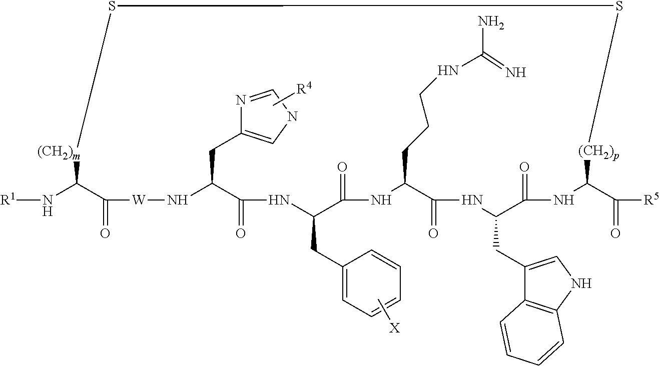

wherein
W is Glu, Gln, Asp, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH₂)_{q}C(O)—,

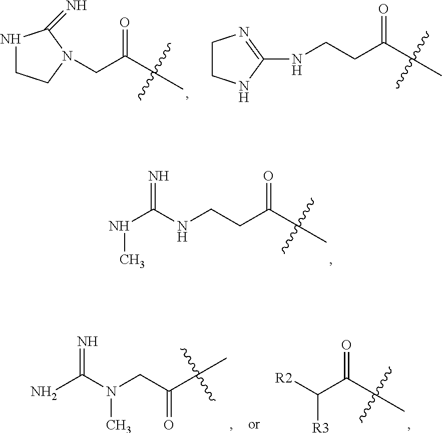

wherein
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr-NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile,

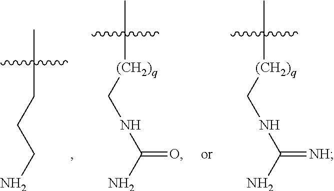

q is 0, 1, 2, or 3;
R⁶ is a phenyl or C₈-C₁₄ bicyclic aryl;
m is 1 or 2;
p is 1 or 2;
R⁴ is H, C₁-C₄ straight or branched alkyl, phenyl, benzyl, or (C₆H₅)—CH₂—O—CH₂—;
X is H, Cl, F, Br, methyl, or methoxy; and
R⁵ is —NH₂, —OH, glycinol, NH₂-Pro-Ser-, NH₂-Pro-Lys-, HO-Ser-, HO-Pro-Ser-, HO-Lys-, -Ser alcohol, -Ser-Pro alcohol, -Lys-Pro alcohol, HOCH₂CH₂—O—CH₂CH₂NH—, NH₂-Phe-Arg-, NH₂-Glu-, NH₂CH₂RCH₂NH—, or RO— where R is a C₁-C₄ straight or branched alkyl.

Additional examples of MC4R agonists useful to practice the present invention are found in WO2011104378; WO2011104379; WO201060901; WO200887189, WO200887188, WO200887187, WO200887186; US20110065652; WO2010144341; WO2010144344; WO201065799; WO201065800; WO201065801; WO201065802; WO201037081; WO2009152079; WO2009151383; US20100311648; US20100280079; WO201081666; WO201034500; WO200910299; WO2008116665; WO201052256; WO201052255; WO201126015; US20100120783; WO201096854; US20100190793; WO201025142; and WO201015972. Further examples of MC4R agonists useful to practice the present invention are found in U.S. Pat. Nos. 8,263,608; 8,247,530; 8,114,844; and 7,968,548. The entire teachings of these publications are incorporated herein by reference.

In one example embodiment, the agonist of MC4R is a tripeptide D-Phe-Arg-Trp (SEQ ID NO: 560) or a pharmaceutical salt thereof. In another example, the agonist is any peptide that includes SEQ ID NO: 560 or a pharmaceutical salt thereof. In yet another example, the MC4R agonist is an acetylated tripeptide Ac-D-Phe-Arg-Trp-NH₂ (SEQ ID NO: 561) or a pharmaceutical salt thereof.

In an example embodiment, the agonists of MC4R are those of Formula (I) or a pharmaceutically acceptable salt, hydrate, solvate or a prodrug thereof (see International Patent Application Publication Number WO 2007/008704, incorporated herein by reference in its entirety):

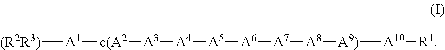

### In Formula (I):

A¹ is Acc, HN—(CH₂)ₘ—C(O), L- or D-amino acid, or deleted;
A² is Cys, D-Cys, hCys, D-hCys, Pen, D-Pen, Asp, or Glu;
A³ is Gly, Ala, β-Ala, Gaba, Aib, D-amino acid, or deleted;
A⁴ is H is, 2-Pal, 3-Pal, 4-Pal, Taz, 2-Thi, 3-Thi, or (X¹, X², X³, X⁴, X⁵)Phe;
A⁵ is D-Phe, D-1-Nal, D-2-Nal, D-Trp, D-Bal, D-(X¹, X², X³, X⁴, X⁵)Phe, L-Phe or D-(Et)Tyr;
A⁶ is Arg, hArg, Dab, Dap, Lys, Orn, or HN—CH((CH₂)ₙ—N(R⁴R⁵))—C(O);
A⁷ is Trp, 1-Nal, 2-Nal, Bal, Bip, D-Trp, D-2-Nal, D-Bal or D-Bip;
A⁸ is Gly, D-Ala, Acc, Ala, 13-Ala, Gaba, Apn, Ahx, Aha, HN—(CH₂)ₛ-C(O), or deleted;
A⁹ is Cys, D-Cys, hCys, D-hCys, Pen, D-Pen, Dab, Dap, Orn, or Lys;
A¹⁰ is Acc, HN—(CH₂)ₜ-C(O), L- or D-amino acid, or deleted;
R¹ is OH or NH₂;
each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X′, X², X³, X⁴, and X⁸ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN.

In exemplary embodiments of the agonists of Formula (I):
(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or —C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl;
(II) when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂—C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;
(III) either A³ or A⁸ or both must be present in said compound;
(IV) when A² is Cys, D-Cys, hCys, D-hCys, Pen, or D-Pen, then A⁹ is Cys, D-Cys, hCys, D-hCys, Pen, or D-Pen;
(V) when A² is Asp or Glu, then A⁹ is Dab, Dap, Orn, or Lys;
(VI) when A⁸ is Ala or Gly, then A¹ is not NIe; and
(VII) when A¹ is deleted, then R² and R³ cannot both be H.

In an example embodiment, the agonists employed by the methods described herein are the compounds of Formula I, wherein:
A¹ is A6c, Arg, D-Arg, Cha, D-Cha, hCha, Chg, D-Chg, Gaba, Ile, Leu, hLeu, Met, 3-hMet, 2-Nal, D-2-Nal, Nip, Nle, Oic, Phe, D-Phe, hPhe, hPro, Val, or deleted;
A² is Asp, Cys, D-Cys, hCys, D-hCys, Glu, Pen, or D-Pen;
A³ is D-Abu, Aib, Ala, β-Ala, D-Ala, D-Cha, Gaba, D-Glu, Gly, D-Ile, D-Leu, D-Tle, D-Val, or deleted;
A⁴ is H is or 3-Pal;
A⁵ is D-Bal, D-1-Nal, D-2-Nal, D-Phe, D-Trp, or D-(Et)Tyr;
A⁶ is Arg, or hArg;
A⁷ is Bal, Bip, 1-Nal, 2-Nal, Trp, D-Trp;
A⁸ is A6c, D-Ala, Aha, Ahx, Ala, β-Ala, Apn, Gaba, Gly or deleted;
A⁹ is Cys, D-Cys, hCys, D-hCys, Lys, Pen, or D-Pen;
A¹⁰ is Thr, or deleted,
wherein at least one of A³ or A⁸ is deleted, but not both, or pharmaceutically acceptable salts thereof.

In an example embodiments, agonists of Formula (I) useful in practicing the invention described herein are compounds of the following formula or a pharmaceutically acceptable salt thereof:

SEQ ID NO: 1
Ac-Nle-c(Asp-His-D-Phe-Arg-Trp-β-Ala-Lys)-NH₂;
SEQ ID NO: 2
Ac-Nle-c(Asp-His-D-Phe-Arg-Trp-A6c-Lys)-NH₂;
SEQ ID NO: 3
Ac-Nle-c(Cys-His-D-Phe-Arg-Trp-Ahx-Cys)-NH₂;
SEQ ID NO: 4
D-Phe-c(Cys-His-D-Phe-Arg-Trp-Ala-D-Cys)-Thr-NH₂;
SEQ ID NO: 5
D-Phe-c(Cys-His-D-Phe-Arg-Trp-β-Ala-D-Cys)-Thr-NH₂;
SEQ ID NO: 6
D-Phe-c(Cys-His-D-Phe-Arg-Trp-Gaba-D-Cys)-Thr-NH₂;
SEQ ID NO: 7
Ac-Nle-c(Cys-His-D-Phe-Arg-Trp-Apn-Cys)-NH₂;
SEQ ID NO: 8
Ac-Nle-c(Asp-His-D-Phe-Arg-Trp-Apn-Lys)-NH₂;
SEQ ID NO: 9
Ac-A6c-c(Asp-His-D-Phe-Arg-Trp-Gaba-Lys)-NH₂;
SEQ ID NO: 10
Ac-D-2-Nal-c(Asp-His-D-Phe-Arg-Trp-Gaba-Lys)-NH₂;
SEQ ID NO: 11
Ac-Cha-c(Asp-His-D-Phe-Arg-Trp-Gaba-Lys)-NH₂;
SEQ ID NO: 12
Ac-Nle-c(Asp-His-D-Phe-Arg-Trp-Gaba-Lys)-NH₂;
SEQ ID NO: 13
Ac-Nle-c(Cys-D-Ala-His-D-Phe-Arg-Trp-Cys)-NH₂;
SEQ ID NO: 14
Ac-Nle-c(Cys-β-Ala-His-D-Phe-Arg-Trp-Cys)-NH₂;
SEQ ID NO: 15
Ac-Nle-c(Cys-Gaba-His-D-Phe-Arg-Trp-Cys)-NH₂;
SEQ ID NO: 16
Ac-Nle-c(Cys-Aib-His-D-Phe-Arg-Trp-Cys)-NH₂;
SEQ ID NO: 17
Ac-Nle-c(Cys-Gly-His-D-Phe-Arg-Trp-Cys)-NH₂;
SEQ ID NO: 18
Ac-Nle-c(D-Cys-Ala-His-D-Phe-Arg-Trp-Cys)-NH₂;
SEQ ID NO: 19
Ac-Nle-c(D-Cys-D-Ala-His-D-Phe-Arg-Trp-Cys)-NH₂;
SEQ ID NO: 20
Ac-Nle-c(D-Cys-β-Ala-His-D-Phe-Arg-Trp-Cys)-NH₂;
SEQ ID NO: 21
Ac-Nle-c(D-Cys-Gaba-His-D-Phe-Arg-Trp-Cys)-NH₂;
SEQ ID NO: 22
Ac-Nle-c(D-Cys-Aib-His-D-Phe-Arg-Trp-Cys)-NH₂;
SEQ ID NO: 23
Ac-Nle-c(D-Cys-Gly-His-D-Phe-Arg-Trp-Cys)-NH₂;
SEQ ID NO: 24
Ac-Nle-c(Cys-D-Ala-His-D-Phe-Arg-Trp-D-Cys)-NH₂;
SEQ ID NO: 25
Ac-Nle-c(Cys-β-Ala-His-D-Phe-Arg-Trp-D-Cys)-NH₂;
SEQ ID NO: 26
Ac-Nle-c(Cys-Gaba-His-D-Phe-Arg-Trp-D-Cys)-NH₂;
SEQ ID NO: 27
Ac-Nle-c(Cys-Aib-His-D-Phe-Arg-Trp-D-Cys)-NH₂;
SEQ ID NO: 28
Ac-Nle-c(Cys-Gly-His-D-Phe-Arg-Trp-D-Cys)-NH₂;
SEQ ID NO: 29
Ac-Nle-c(D-Cys-Ala-His-D-Phe-Arg-Trp-D-Cys)-NH₂;
SEQ ID NO: 30
Ac-Nle-c(D-Cys-D-Ala-His-D-Phe-Arg-Trp-D-Cys)-NH₂;
SEQ ID NO: 31
Ac-Nle-c(D-Cys-β-Ala-His-D-Phe-Arg-Trp-D-Cys)-NH₂;
SEQ ID NO: 32
Ac-Nle-c(D-Cys-Gaba-His-D-Phe-Arg-Trp-D-Cys)-NH₂;
SEQ ID NO: 33
Ac-Nle-c(D-Cys-Aib-His-D-Phe-Arg-Trp-D-Cys)-NH₂;
SEQ ID NO: 34
Ac-Oic-c(Asp-His-D-Phe-Arg-Trp-Gaba-Lys)-NH₂;
SEQ ID NO: 35
Ac-Chg-c(Asp-His-D-Phe-Arg-Trp-Gaba-Lys)-NH₂;
SEQ ID NO: 36
Ac-hCha-c(Asp-His-D-Phe-Arg-Trp-Gaba-Lys)-NH₂;
SEQ ID NO: 37
Ac-D-Cha-c(Asp-His-D-Phe-Arg-Trp-Gaba-Lys)-NH₂;
SEQ ID NO: 38
Ac-D-hCha-c(Asp-His-D-Phe-Arg-Trp-Gaba-Lys)-NH₂;
SEQ ID NO: 39
Ac-Nip-c(Asp-His-D-Phe-Arg-Trp-Gaba-Lys)-NH₂;
SEQ ID NO: 40
Ac-hPro-c(Asp-His-D-Phe-Arg-Trp-Gaba-Lys)-NH₂;
SEQ ID NO: 41
Ac-hLeu-c(Asp-His-D-Phe-Arg-Trp-Gaba-Lys)-NH₂;
SEQ ID NO: 42
Ac-Phe-c(Asp-His-D-Phe-Arg-Trp-Gaba-Lys)-NH₂;
SEQ ID NO: 43
Ac-D-Phe-c(Asp-His-D-Phe-Arg-Trp-Gaba-Lys)-NH₂;
SEQ ID NO: 44
Ac-D-Chg-c(Asp-His-D-Phe-Arg-Trp-Gaba-Lys)-NH₂;
SEQ ID NO: 45
n-butanoyl-Cha-c(Asp-His-D-Phe-Arg-Trp-Gaba-Lys)-NH₂;
SEQ ID NO: 46
n-butyryl-Cha-c(Asp-His-D-Phe-Arg-Trp-Gaba-Lys)-NH₂;
SEQ ID NO: 47
Ac-hPhe-c(Asp-His-D-Phe-Arg-Trp-Gaba-Lys)-NH₂;
SEQ ID NO: 48
Ac-P-hMet-c(Asp-His-D-Phe-Arg-Trp-Gaba-Lys)-NH₂;
SEQ ID NO: 49
Ac-Gaba-c(Asp-His-D-Phe-Arg-Trp-Gaba-Lys)-NH₂;
SEQ ID NO: 50
Ac-Cha-c(Asp-His-D-Phe-Arg-D-Trp-Ala-Lys)-NH₂;
SEQ ID NO: 51
Ac-hCha-c(Asp-His-D-Phe-Arg-D-Trp-Ala-Lys)-NH₂;
SEQ ID NO: 52
Ac-Leu-c(Asp-His-D-Phe-Arg-D-Trp-Ala-Lys)-NH₂;
SEQ ID NO: 53
Ac-hLeu-c(Asp-His-D-Phe-Arg-D-Trp-Ala-Lys)-NH₂;
SEQ ID NO: 54
Ac-Phe-c(Asp-His-D-Phe-Arg-D-Trp-Ala-Lys)-NH₂;
SEQ ID NO: 55
Ac-Nle-c(Asp-His-D-Phe-Arg-D-Trp-D-Ala-Lys)-NH₂;
SEQ ID NO: 56
Ac-Nle-c(Asp-His-D-Phe-Arg-D-Trp-β-Ala-Lys)-NH₂;
SEQ ID NO: 57
Ac-Nle-c(Asp-His-D-Phe-Arg-D-Trp-Gaba-Lys)-NH₂;
SEQ ID NO: 58
Ac-Nle-c(Asp-His-D-Phe-Arg-D-Trp-Aha-Lys)-NH₂;
SEQ ID NO: 59
Ac-Nle-c(Asp-His-D-Phe-Arg-D-Trp-Apn-Lys)-NH₂;
SEQ ID NO: 60
Ac-Nle-c(Cys-His-D-Phe-Arg-D-Trp-Apn-Cys)-NH₂;
SEQ ID NO: 61
Ac-Nle-c(Cys-His-D-Phe-Arg-D-Trp-Gaba-Cys)-NH₂;
SEQ ID NO: 62
Ac-Nle-c(Cys-His-D-Phe-Arg-D-Trp-Ahx-Cys)-NH₂;
SEQ ID NO: 63
Ac-Nle-c(Cys-His-D-Phe-Arg-D-Trp-β-Ala-Cys)-NH₂;
SEQ ID NO: 64
Ac-Nle-c(Cys-His-D-Phe-Arg-D-Trp-D-Ala-Cys)-NH₂;
SEQ ID NO: 65
Ac-Nle-c(Cys-D-Ala-His-D-2-Nal-Arg-Trp-Cys)-NH₂;
SEQ ID NO: 66
Ac-Nle-c(Cys-D-Ala-His-D-2-Nal-Arg-2-Nal-Cys)-NH₂;
SEQ ID NO: 67
Ac-Nle-c(Cys-D-Ala-His-D-2-Nal-Arg-1-Nal-Cys)-NH₂;
SEQ ID NO: 68
n-butanoyl-Nle-c(Cys-D-Ala-His-D-Phe-Arg-2-Nal-Cys)-NH₂;
SEQ ID NO: 69
n-butanoyl-Nle-c(Cys-D-Ala-His-D-Phe-Arg-Trp-Cys)-NH₂;
SEQ ID NO: 70
Ac-Nle-c(Cys-D-Ala-His-D-Phe-Arg-2-Nal-Cys)-NH₂;
SEQ ID NO: 71
Ac-Nle-c(Cys-D-Ala-His-D-Phe-Arg-1-Nal-Cys)-NH₂;
SEQ ID NO: 72
Ac-Nle-c(Cys-D-Ala-His-D-Phe-Arg-Bal-Cys)-NH₂;
SEQ ID NO: 73
Ac-Nle-c(Cys-D-Glu-His-D-Phe-Arg-Trp-Cys)-NH₂;
SEQ ID NO: 74
Ac-Nle-c(Asp-His-D-Phe-Arg-Trp-D-Ala-Lys)-NH₂;
SEQ ID NO: 75
Ac-Nle-c(Cys-D-Ala-His-D-2-Nal-Arg-Bal-Cys)-NH₂;
SEQ ID NO: 76
Ac-Nle-c(Pen-D-Ala-His-D-Phe-Arg-Trp-Cys)-NH₂;
SEQ ID NO: 77
Ac-Nle-c(Cys-D-Ala-His-D-Phe-Arg-Trp-Pen)-NH₂;
SEQ ID NO: 78
Ac-Nle-c(Pen-D-Ala-His-D-Phe-Arg-Trp-Pen)-NH₂;
SEQ ID NO: 79
D-Phe-c(Cys-His-D-Phe-hArg-Trp-β-Ala-D-Cys)-Thr-NH₂;
SEQ ID NO: 80
D-Phe-c(Cys-His-D-(Et)Tyr-Arg-Trp-β-Ala-D-Cys)-Thr-NH₂;
SEQ ID NO: 81
D-Phe-c(Cys-His-D-Phe-Arg-Bip-β-Ala-D-Cys)-Thr-NH₂;
SEQ ID NO: 82
D-Phe-c(Cys-His-D-(Et)Tyr-hArg-Trp-β-Ala-D-Cys)-Thr-NH₂;
SEQ ID NO: 83
D-Phe-c(Cys-His-D-Phe-hArg-Bip-β-Ala-D-Cys)-Thr-NH₂;
SEQ ID NO: 84
D-Phe-c(Cys-His-D-(Et)Tyr-hArg-Bip-β-Ala-D-Cys)-Thr-NH₂;
SEQ ID NO: 85
Nle-c(Cys-His-D-Phe-Arg-Trp-Apn-Cys)-NH₂;
SEQ ID NO: 86
Ac-Nle-c(Asp-D-Ala-His-D-Phe-Arg-Trp-Lys)-NH₂;
SEQ ID NO: 87
Ac-Nle-c(Asp-D-Ala-His-D-Phe-Arg-Bal-Lys)-NH₂;
SEQ ID NO: 88
Ac-Nle-c(Cys-D-Ala-His-D-Phe-Arg-Trp-Pen)-OH;
SEQ ID NO: 89
Ac-Nle-c(Cys-D-Abu-His-D-Phe-Arg-Trp-Cys)-NH₂;
SEQ ID NO: 90
Ac-Nle-c(Cys-D-Val-His-D-Phe-Arg-Trp-Cys)-NH₂;
SEQ ID NO: 91
Ac-Nle-c(Cys-D-Ile-His-D-Phe-Arg-Trp-Cys)-NH₂;
SEQ ID NO: 92
Ac-Nle-c(Cys-D-Leu-His-D-Phe-Arg-Trp-Cys)-NH₂;
SEQ ID NO: 93
Ac-Nle-c(Cys-D-T1e-His-D-Phe-Arg-Trp-Cys)-NH₂;
SEQ ID NO: 94
Ac-Nle-c(Cys-D-Cha-His-D-Phe-Arg-Trp-Cys)-NH₂;
SEQ ID NO: 95
Ac-Nle-c(Pen-His-D-Phe-Arg-Trp-Gaba-Cys)-NH₂;
SEQ ID NO: 96
Ac-Nle-c(Cys-His-D-Phe-Arg-Trp-Gaba-Pen)-NH₂;
SEQ ID NO: 97
Ac-Nle-c(Pen-His-D-Phe-Arg-Trp-Gaba-Pen)-NH₂;
SEQ ID NO: 98
Ac-Leu-c(Cys-His-D-Phe-Arg-Trp-Gaba-Cys)-NH₂;
SEQ ID NO: 99
Ac-Cha-c(Cys-His-D-Phe-Arg-Trp-Gaba-Cys)-NH₂;
SEQ ID NO: 100
Ac-Ile-c(Cys-His-D-Phe-Arg-Trp-Gaba-Cys)-NH₂;
SEQ ID NO: 101
Ac-Phe-c(Cys-His-D-Phe-Arg-Trp-Gaba-Cys)-NH₂;
SEQ ID NO: 102
Ac-Val-c(Cys-His-D-Phe-Arg-Trp-Gaba-Cys)-NH₂;
SEQ ID NO: 103
Ac-2-Nal-c(Cys-His-D-Phe-Arg-Trp-Gaba-Cys)-NH₂;
SEQ ID NO: 104
Nle-c(Cys-His-D-Phe-Arg-Trp-Gaba-Cys)-NH₂;
SEQ ID NO: 105
Phe-c(Cys-His-D-Phe-Arg-Trp-Gaba-Cys)-NH₂;
SEQ ID NO: 106
Ac-Nle-c(Cys-β-Pal-D-Phe-Arg-Trp-Gaba-Cys)-NH₂;
SEQ ID NO: 107
Ac-Nle-c(Cys-D-Ala-His-D-Phe-Arg-Trp-Cys)-OH;
SEQ ID NO: 108
Ac-Nle-c(Cys-His-Phe-Arg-D-Trp-Gaba-Cys)-NH₂;
SEQ ID NO: 109
Ac-Nle-c(Asp-His-D-2-Nal-Arg-Trp-Ala-Lys)-NH₂;
SEQ ID NO: 110
Ac-Nle-c(Asp-His-D-2-Nal-Arg-Trp-β-Ala-Lys)-NH₂;
SEQ ID NO: 111
Ac-Nle-c(Cys-His-D-2-Nal-Arg-Trp-Gaba-Cys)-NH₂;
SEQ ID NO: 112
Ac-Nle-c(Cys-His-D-2-Nal-Arg-Trp-Ahx-Cys)-NH₂;
SEQ ID NO: 113
Ac-hPhe-c(Asp-His-D-2-Nal-Arg-Trp-Gaba-Lys)-NH₂;
SEQ ID NO: 114
Ac-Cha-c(Asp-His-D-2-Nal-Arg-Trp-Gaba-Lys)-NH₂;
SEQ ID NO: 115
Ac-Nle-c(Asp-His-D-Phe-Arg-Trp-β-Ala-Lys)-OH;
SEQ ID NO: 116
Ac-Nle-c(Cys-His-D-Phe-Arg-Trp-Ahx-Cys)-OH;
SEQ ID NO: 117
D-Phe-c(Cys-His-D-Phe-Arg-Trp-Ala-D-Cys)-Thr-OH;
SEQ ID NO: 118
D-Phe-c(Cys-His-D-Phe-Arg-Trp-β-Ala-D-Cys)-Thr-OH;
SEQ ID NO: 119
D-Phe-c(Cys-His-D-Phe-Arg-Trp-Gaba-D-Cys)-Thr-OH;
SEQ ID NO: 120
Ac-Nle-c(Cys-His-D-Phe-Arg-Trp-Apn-Cys)-OH;
SEQ ID NO: 121
Ac-Nle-c(Asp-His-D-Phe-Arg-Trp-Apn-Lys)-OH;
SEQ ID NO: 122
Ac-Cha-c(Asp-His-D-Phe-Arg-Trp-Gaba-Lys)-OH;
SEQ ID NO: 123
Ac-Nle-c(Asp-His-D-Phe-Arg-Trp-Gaba-Lys)-OH;
SEQ ID NO: 124
Ac-Chg-c(Asp-His-D-Phe-Arg-Trp-Gaba-Lys)-OH;
SEQ ID NO: 125
Ac-D-Cha-c(Asp-His-D-Phe-Arg-Trp-Gaba-Lys)-OH;
SEQ ID NO: 126
Ac-hCha-c(Asp-His-D-Phe-Arg-Trp-Gaba-Lys)-OH;
SEQ ID NO: 127
Ac-D-Chg-c(Asp-His-D-Phe-Arg-Trp-Gaba-Lys)-OH;
SEQ ID NO: 128
Ac-hPhe-c(Asp-His-D-Phe-Arg-Trp-Gaba-Lys)-OH;
SEQ ID NO: 129
Ac-Nle-c(Cys-His-D-Phe-Arg-D-Trp-Gaba-Cys)-OH;
SEQ ID NO: 130
Ac-Nle-c(Cys-His-D-Phe-Arg-D-Trp-Ahx-Cys)-OH;
SEQ ID NO: 131
Ac-Nle-c(Cys-His-D-Phe-Arg-D-Trp-β-Ala-Cys)-OH;
SEQ ID NO: 132
Ac-Nle-c(Cys-His-D-Phe-Arg-D-Trp-D-Ala-Cys)-OH;
SEQ ID NO: 133
Ac-Nle-c(Cys-D-Ala-His-D-2-Nal-Arg-Trp-Cys)-OH;
SEQ ID NO: 134
Ac-Nle-c(Cys-D-Ala-His-D-2-Nal-Arg-2-Nal-Cys)-OH;
SEQ ID NO: 135
Ac-Nle-c(Cys-D-Ala-His-D-2-Nal-Arg-1-Nal-Cys)-OH;
SEQ ID NO: 136
Ac-Nle-c(Cys-D-Ala-His-D-2-Nal-Arg-Bal-Cys)-OH;
SEQ ID NO: 137
Ac-Nle-c(Pen-D-Ala-His-D-Phe-Arg-Trp-Cys)-OH;
SEQ ID NO: 138
Ac-Nle-c(Cys-His-D-Phe-Arg-Trp-Gaba-Pen)-OH;
SEQ ID NO: 139
Ac-Arg-c(Cys-D-Ala-His-D-2-Nal-Arg-Trp-Cys)-NH₂;
SEQ ID NO: 140
Ac-Arg-c(Cys-D-Ala-His-D-Phe-Arg-Trp-Cys)-NH₂;
SEQ ID NO: 141
Ac-D-Arg-c(Cys-D-Ala-His-D-Phe-Arg-Trp-Cys)-NH₂;
SEQ ID NO: 142
Ac-D-Arg-c(Cys-D-Ala-His-D-Phe-Arg-Trp-Pen)-NH₂;
SEQ ID NO: 143
Ac-D-Arg-c(Cys-His-D-Phe-Arg-Trp-Gaba-Pen)-NH₂;
SEQ ID NO: 144
Ac-Arg-c(Cys-His-D-Phe-Arg-Trp-Gaba-Pen)-NH₂;
SEQ ID NO: 145
Ac-Arg-c(Cys-D-Ala-His-D-Phe-Arg-Trp-Pen)-NH₂;
SEQ ID NO: 146
Ac-D-Arg-c(Asp-His-D-Phe-Arg-Trp-Ala-Lys)-NH₂;
or
SEQ ID NO: 147
Ac-Arg-c(Asp-His-D-Phe-Arg-Trp-Ala-Lys)-NH₂;

or pharmaceutically acceptable salts thereof.

In an example embodiment, an agonist of MC4R receptor useful for practicing methods described herein is any of the compounds described by Formula (II) or a pharmaceutically acceptable salt, hydrate, solvate or a prodrug thereof (see International Patent Application Publication Number WO 2007/008704 incorporated herein by reference in its entirety):

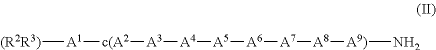

In formula (II):
A¹ is Ne or deleted;
A² is Cys or Asp;
A³ is Glu or D-Ala;
A⁴ is H is;
A⁵ is D-Phe;
A⁶ is Arg;
A⁷ is Trp, 2-Nal or Bal;
A⁸ is Gly, Ala, D-Ala, (3-Ala, Gaba or Apn;
A⁹ is Cys or Lys;
each of R² and R³ is independently selected from the group consisting of H or (C₁-C₆)acyl.

In exemplary embodiments of Formula (II):
(I) when R² is (C₁-C₆)acyl, then R³ is H; and
(II) when A² is Cys, then A⁹ is Cys.

In alternative example embodiments of the present invention, the compounds useful for practicing the methods disclosed herein are:

SEQ ID NO:148
Ac-Nle-c(Cys-D-Ala-His-D-Phe-Arg-Trp-Gly-Cys)-NH₂;
SEQ ID NO: 149
Ac-Nle-c(Cys-D-Ala-His-D-Phe-Arg-Trp-D-Ala-Cys)NH₂;
SEQ ID NO: 150
Ac-Nle-c(Cys-D-Ala-His-D-Phe-Arg-Trp-β-Ala-Cys)-NH₂;
SEQ ID NO: 151
Ac-Nle-c(Cys-D-Ala-His-D-Phe-Arg-Trp-Gaba-Cys)-NH₂;
SEQ ID NO: 152
Ac-Nle-c(Cys-D-Ala-His-D-Phe-Arg-Trp-Apn-Cys)-NH₂;
SEQ ID NO: 153
Ac-c(Cys-Glu-His-D-Phe-Arg-Trp-Ala-Cys)-NH₂;
SEQ ID NO: 154
Ac-c(Cys-Glu-His-D-Phe-Arg-2-Nal-Ala-Cys)-NH₂;
SEQ ID NO: 155
Ac-c(Cys-D-Ala-His-D-Phe-Arg-Trp-Ala-Cys)-NH₂;
SEQ ID NO: 156
Ac-c(Cys-D-Ala-His-D-Phe-Arg-2-Nal-Ala-Cys)-NH₂;
SEQ ID NO: 157
Ac-Nle-c(Cys-D-Ala-His-D-Phe-Arg-Trp-Ala-Cys)-NH₂;
or
SEQ ID NO: 158
Ac-Nle-c(Asp-D-Ala-His-D-Phe-Arg-Bal-Ala-Lys)-NH₂;

or a pharmaceutically acceptable salt thereof.

In an exemplary embodiment, the agonists of MC4R useful for practicing the methods described herein is any of the compounds of Formula (III), or a pharmaceutically acceptable salt, hydrate, solvate or a prodrug thereof (see International Application Publication Number WO 2007/008684, incorporated herein by reference in its entirety):

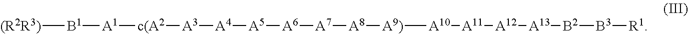

In Formula (III):

B¹ is a peptide moiety which contains 5, 6, 7, 8, 9, 10, 11, 12, 13, 14, or 15 amino acids, wherein at least 5 amino acids are independently selected from the group consisting of L-Arg, D-Arg, L-hArg and D-hArg, or B¹ is optionally deleted;
A¹ is Acc, HN—(CH₂)ₘ-C(O), L- or D-amino acid or deleted;
A² is Cys, D-Cys, hCys, D-hCys, Pen, D-Pen, Asp or Glu;
A³ is Gly, Glu, Ala, β-Ala, Gaba, Aib, D-amino acid or deleted;
A⁴ is H is, 2-Pal, 3-Pal, 4-Pal, Taz, 2-Thi, 3-Thi or (X′, X², X³, X⁴, X⁵)Phe;
A⁵ is D-Phe, D-1-Nal, D-2-Nal, D-Trp, D-Bal, D-(X¹, X², X³, X⁴, X⁵)Phe, D-(Et)Tyr, D-Dip, D-Bip or D-Bpa;
A⁶ is Arg, hArg, Dab, Dap, Lys, Orn or HN—CH((CH₂)ₙ—N(R⁴R⁵))—C(O);
A⁷ is Trp, 1-Nal, 2-Nal, Bal, Bip, Dip, Bpa, D-Trp, D-1-Nal, D-2-Nal, D-Bal, D-Bip, D-Dip or D-Bpa;
A⁸ is Gly, D-Ala, Acc, Ala, β-Ala, Gaba, Apn, Ahx, Aha, HN—(CH₂)ₛ—C(O) or deleted;
A⁹ is Cys, D-Cys, hCys, D-hCys, Pen, D-Pen, Dab, Dap, Orn or Lys;
A¹⁰ is Acc, HN—(CH₂)ᵣ—C(O), Pro, hPro, 3-Hyp, 4-Hyp, Thr, an L- or D-amino acid or deleted;
A¹¹ is Pro, hPro, 3-Hyp, 4-Hyp or deleted;
A¹² is Lys, Dab, Dap, Arg, hArg or deleted;
A¹³ is Asp, Glu or deleted;
B² is a peptide moiety containing 1, 2, 3, 4, or 5 amino acids or deleted,
B³ is a peptide moiety which contains 5, 6, 7, 8, 9, 10, 11, 12, 13, 14 or 15 amino acids wherein at least 5 amino acids are independently selected from the group consisting of L-Arg, D-Arg, L-hArg and D-hArg, or is deleted;
R¹ is OH or NH₂;
R² and R³ each is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl and substituted aryl(C₁-C₃₀)acyl;
R⁴ and R⁵ each is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl or C(NH)—NH₂;
n is, independently for each occurrence, 1, 2, 3, 4 or 5;
m is, independently for each occurrence, 1, 2, 3, 4, 5, 6 or 7;
s is, independently for each occurrence, 1, 2, 3, 4, 5, 6 or 7;
t is, independently for each occurrence, 1, 2, 3, 4, 5, 6 or 7;
X¹, X², X³, X⁴ and X⁵ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂ or CN.

In an example embodiments of Formula (III):
(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl or C(NH)—NH₂, then R⁵ is H, (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl or substituted aryl(C₁-C₄₀)alkyl;
(II) when R² is (C₁-C₃₀)acyl, aryl(C₁-C₃₀)acyl, substituted (C₁-C₃₀)acyl or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl or substituted aryl(C₁-C₃₀)alkyl;
(III) neither B¹ nor B² contains one or more of the following amino acid sequences: Arg-(Lys)₂-(Arg)₂-Gln-(Arg)₃, Tyr-Ala-Arg-Lys-Ala-(Arg)₂-Gln-Ala-(Arg)₂, Tyr-Ala-Arg-(Ala)₂-(Arg)₂-(Ala)₂-(Arg)₂, Tyr-Ala-(Arg)₉, Tyr-(Ala)₃-(Arg)₇, Tyr-Ala-Arg-Ala-Pro-(Arg)₂-Ala-(Arg)₃ or Tyr-Ala-Arg-Ala-Pro-(Arg)₂-Pro-(Arg)₂;
(IV) either B¹ or B² or both must be present in said compound;
(V) when A² is Cys, D-Cys, hCys, D-hCys, Pen or D-Pen, then A⁹ is Cys, D-Cys, hCys, D-hCys, Pen or D-Pen; and
(VI) when A² is Asp or Glu, then A⁹ is Dab, Dap, Orn or Lys.

In exemplary embodiments, in Formula (III);
B¹ is Arg-Lys-Gln-Lys-(Arg)₅, Arg-(Lys)₂-Arg-Gln-(Arg)₄, Arg-(Lys)₂-(Arg)₃-Gln-(Arg)₂, Arg-(Lys)₂-(Arg)₄-Gln-Arg, Arg-(Lys)₂-(Arg)₅-Gln, Arg-(Lys)₂-Gln-(Arg)₅, Arg-Gln-(Lys)₂-(Arg)₅, Arg-Gln-(Arg)₇, Arg-Gln-(Arg)₅, (Arg)₂-Gln-(Arg)₆, (Arg)₂-Gln-(Arg)₇, (Arg)₃-Gln-(Arg)₅, (Arg)₃-Gln-(Arg)₆, (Arg)₄-Gln-(Arg)₄, (Arg)₄-Gln-(Arg)₅, (Arg)₅, (Arg)₅-Gln-(Arg)₃, (Arg)₅-Gln-(Arg)₄, (Arg)₆, (Arg)₆-Gln-(Arg)₃, (Arg)₇, (Arg)₇-Gln-(Arg)₂, (Arg)₅, (Arg)₅-Gln-Arg, (Arg)₉, (Arg)₉-Gln, (D-Arg)₅, (D-Arg)₆, (D-Arg)₇, (D-Arg)₅, (D-Arg)₉, Gln-Arg-(Lys)₂-(Arg)₅, Gln-(Arg)₅, Gln-(Arg)₉, Tyr-Gly-Arg-(Lys)₂-(Arg)₂-Gln-(Arg)₃, Tyr-Gly-Arg-(Lys)₂-(Arg)₂-Gln-(Arg)₃-Doc; or deleted;
B² is β-Ala, β-Ala-Gly, β-Ala-Tyr, β-Ala-Tyr-Gly, (β-Ala)₂, (β-Ala)₂-Gly, (β-Ala)₂-Tyr, (β-Ala)₂-Tyr-Gly, Doc, Doc-Gly, Doc-Tyr, Doc-Tyr-Gly, (Doc)₂, (Doc)₂-Gly, (Doc)₂-Tyr, Doc)₂-Tyr-Gly, or deleted;
B³ is Arg-Lys-Gln-Lys-(Arg)₅, Arg-Lys-(Arg)₃-Gln-(Arg)₃, Arg-(Lys)₂-Arg-Gln-(Arg)₄, Arg-(Lys)₂-Gln-(Arg)₅, Arg-(Lys)₂-(Arg)₂-Gln-(Arg)₃, Arg-(Lys)₂-(Arg)₃-Gln-(Arg)₂, Arg-(Lys)₂-(Arg)₄-Gln-Arg, Arg-(Lys)₂-(Arg)₅-Gln, Arg-Gln-(Lys)₂-(Arg)₅, Arg-Gln-(Arg)₇, Arg-Gln-(Arg)₅, (Arg)₂-Lys-(Arg)₂-Gln-(Arg)₃, (Arg)₂-Gln-(Arg)₆, (Arg)₂-Gln-(Arg)₇, (Arg)₃-Gln-(Arg)₅, (Arg)₃-Gln-(Arg)₆, (Arg)₄-Gln-(Arg)₄, (Arg)₄-Gln-(Arg)₅, (Arg)₅, (Arg)₅-Gln-(Arg)₃, (Arg)₅-Gln-(Arg)₄, (Arg)₆, (Arg)₆-Gln-(Arg)₃, (Arg)₇, (Arg)₇-Gln-(Arg)₂, (Arg)₅, (Arg)₅-Gln-Arg, (Arg)₉, (Arg)₉-Gln, (D-Arg)₅, (D-Arg)₆, (D-Arg)₇, (D-Arg)₅, (D-Arg)₉, Gln-Arg-(Lys)₂-(Arg)₅, Gln-(Arg)₅, Gln-(Arg)₉, or deleted;
A¹ is A6c, Cha, hCha, Chg, D-Chg, hChg, Gaba, hLeu, Met, R-hMet, D-2-Nal, Nip, Nle, Oic, Phe, D-Phe, hPhe, hPro, or deleted;
A² is Cys;
A³ is D-Abu, Aib, Ala, β-Ala, D-Ala, D-Cha, Gaba, Glu, Gly, D-Ile, D-Leu, D-Met, D-Nle, D-Phe, D-Tle, D-Trp, D-Tyr, D-Val, or deleted;
A⁴ is H;
A⁵ is D-Bal, D-1-Nal, D-2-Nal, D-Phe, D-(X¹, X², X³, X⁴, X⁵)Phe, D-Trp, or D-(Et)Tyr;
A⁶ is Arg or hArg;
A⁷ is Bal, Bip, 1-Nal, 2-Nal, Trp, or D-Trp;
A⁸ is A5c, A6c, Aha, Ahx, Ala, β-Ala, Apn, Gaba, Gly, or deleted;
A⁹ is Cys, D-Cys, hCys, D-hCys, Lys, Pen, or D-Pen;
A¹⁰ is Pro, Thr or deleted;
A¹¹ is Pro or deleted;
A¹² is arg, Lys, or deleted;
A¹³ is Asp or deleted;
each of R² and R³ is, independently, H or acyl;
or pharmaceutically acceptable salts thereof.

In exemplary embodiments, the MC4R agonists useful for practicing the methods of the present invention are at least one of the following compounds:

(SEQ ID NO: 159)
Tyr-Gly-Arg-(Lys)₂-(Arg)₂-Gln-(Arg)₃-Nle-c(Asp-His-D-2-Nal-Arg-Trp-
Lys)-NH₂;
(SEQ ID NO: 160)
Tyr-Gly-Arg-(Lys)₂-(Arg)₂-Gln-(Arg)₃-Doc-Nle-c(Asp-His-D-2-Nal-Arg-
Trp-Lys)-NH₂;
(SEQ ID NO: 161)
Nle-c(Asp-His-D-2-Nal-Arg-Trp-Lys)-β-Ala-Tyr-Gly-Arg-(Lys)₂-(Arg)₂-
Gln-(Arg)₃-NH₂;
(SEQ ID NO: 162)
Ac-Nle-c(Asp-His-D-2-Nal-Arg-Trp-Lys)-β-Ala-Tyr-Gly-Arg-(Lys)₂-
(Arg)₂-Gln-(Arg)₃-NH₂;
(SEQ ID NO: 163)
Nle-c(Asp-His-D-2-Nal-Arg-Trp-Lys)-(Doc)₂-Tyr-Gly-Arg-(Lys)₂-(Arg)₂-
Gln-(Arg)₃-NH₂;
(SEQ ID NO: 164)
Ac-Nle-c(Asp-His-D-2-Nal-Arg-Trp-Lys)-(Pro)₂-Lys-Asp-Tyr-Gly-Arg-
(Lys)₂-(Arg)₂-Gln-(Arg)₃-NH₂;
(SEQ ID NO: 165)
Ac-c(Cys-Glu-His-D-2-Nal-Arg-Trp-Gly-Cys)-(Pro)₂-Lys-Asp-Tyr-Gly-
Arg-(Lys)₂-(Arg)₂-Gln-(Arg)₃-NH₂;
(SEQ ID NO: 166)
Ac-Nle-c(Asp-His-D-2-Nal-Arg-Trp-Lys)-(β-Ala)₂-Tyr-Gly-Arg-(Lys)₂-
(Arg)₂-Gln-(Arg)₃-NH₂;
(SEQ ID NO: 167)
Ac-Nle-c(Asp-His-D-2-Nal-Arg-Trp-Lys)-(Pro)₂-Lys-Asp-Doc-Tyr-Gly-
Arg-(Lys)₂-(Arg)₂-Gln-(Arg)₃-NH₂;
(SEQ ID NO: 168)
Ac-c(Cys-Glu-His-D-2-Nal-Arg-Trp-Gly-Cys)-(Pro)₂-Lys-Asp-Doc-Tyr-
Gly-Arg-(Lys)₂-(Arg)₂-Gln-(Arg)₃-NH₂;
(SEQ ID NO: 169)
Ac-c(Cys-Glu-His-D-2-Nal-Arg-Trp-Ala-Cys)-(Pro)₂-Lys-Asp-β-Ala-Tyr-
Gly-Arg-(Lys)₂-(Arg)₂-Gln-(Arg)₃-NH₂;
(SEQ ID NO: 170)
Ac-c(Cys-Glu-His-D-2-Nal-Arg-Trp-Ala-Cys)-(Pro)₂-Lys-Asp-Doc-Tyr-
Gly-Arg-(Lys)₂-(Arg)₂-Gln-(Arg)₃-NH₂;
(SEQ ID NO: 171)
Ac-Nle-c(Asp-His-D-2-Nal-Arg-Trp-Lys)-(Doc)₂-Tyr-Gly-Arg-(Lys)₂-
(Arg)₂-Gln-(Arg)₃-NH₂;
(SEQ ID NO: 172)
Ac-c(Cys-Glu-His-D-2-Nal-Arg-2-Nal-Ala-Cys)-(Pro)₂-Lys-Asp-β-Ala-Tyr-
Gly-Arg-(Lys)₂-(Arg)₂-Gln-(Arg)₃-NH₂;
(SEQ ID NO: 173)
Ac-c(Cys-Glu-His-D-2-Nal-Arg-Bal-Ala-Cys)-(Pro)₂-Lys-Asp-β-Ala-(Arg)₅-
Gln-(Arg)₃-NH₂;
(SEQ ID NO: 174)
Ac-c(Cys-Glu-His-D-2-Nal-Arg-Bal-Ala-Cys)-(Pro)₂-Lys-Asp-β-Ala-Gly-
(Arg)₅-Gln-(Arg)₃-NH₂;
(SEQ ID NO: 175)
Ac-c(Cys-Glu-His-D-2-Nal-Arg-Bal-Ala-Cys)-(Pro)₂-Lys-Asp-β-Ala-Tyr-
Gly-(Arg)₅-Gln-(Arg)₃-NH₂;
(SEQ ID NO: 176)
Ac-c(Cys-Glu-His-D-2-Nal-Arg-Trp-Ala-Cys)-(Pro)₂-Lys-Asp-β-Ala-Tyr-
Gly-Arg-(Lys)₂-Arg-Gln-(Arg)₄-NH₂;
(SEQ ID NO: 177)
Ac-c(Cys-Glu-His-D-2-Nal-Arg-Trp-Ala-Cys)-(Pro)₂-Lys-Asp-β-Ala-Tyr-
Gly-Arg-(Lys)₂-Gln-(Arg)₅-NH₂;
(SEQ ID NO: 178)
Ac-c(Cys-Glu-His-D-2-Nal-Arg-Trp-Ala-Cys)-(Pro)₂-Lys-Asp-β-Ala-Tyr-
Gly-Arg-Lys-Gln-Lys-(Arg)₅-NH₂;
(SEQ ID NO: 179)
Ac-c(Cys-Glu-His-D-2-Nal-Arg-Trp-Ala-Cys)-(Pro)₂-Lys-Asp-β-Ala-Tyr-
Gly-Arg-(Lys)₂-(Arg)₄-Gln-Arg-NH₂;
(SEQ ID NO: 180)
Ac-c(Cys-Glu-His-D-2-Nal-Arg-Bal-Ala-Cys)-(Pro)₂-Lys-Asp-β-Ala-Tyr-
Aib-Arg-(Lys)₂-(Arg)₂-Gln-(Arg)₃-NH₂;
(SEQ ID NO: 181)
Ac-c(Cys-Glu-His-D-2-Nal-Arg-1-Nal-Ala-Cys)-(Pro)₂-Arg-Asp-β-Ala-
(Arg)₅-Gln-(Arg)₃-NH₂;
(SEQ ID NO: 182)
Ac-c(Cys-Glu-His-D-2-Nal-Arg-1-Nal-Ala-Cys)-(Pro)₂-Lys-Asp-β-Ala-
(Arg)₅-Gln-(Arg)₃-NH₂;
(SEQ ID NO: 183)
Ac-c(Cys-Glu-His-D-2-Nal-Arg-1-Nal-Ala-Cys)-(Pro)₂-Lys-Asp-β-Ala-
(Arg)₆-Gln-(Arg)₃-NH₂;
(SEQ ID NO: 184)
Ac-c(Cys-Glu-His-D-2-Nal-Arg-2-Nal-Ala-Cys)-(Pro)₂-Arg-Asp-β-Ala-
(Arg)₅-Gln-(Arg)₃-NH₂;
(SEQ ID NO: 185)
Ac-c(Cys-Glu-His-D-2-Nal-Arg-2-Nal-Ala-Cys)-(Pro)₂-Lys-Asp-β-Ala-
(Arg)₅-Gln-(Arg)₃-NH₂;
(SEQ ID NO: 186)
Ac-c(Cys-Glu-His-D-2-Nal-Arg-2-Nal-Ala-Cys)-(Pro)₂-Lys-Asp-β-Ala-
(Arg)₆-Gln-(Arg)₃-NH₂;
(SEQ ID NO: 187)
Ac-c(Cys-Glu-His-D-2-Nal-Arg-Bal-Ala-Cys)-(Pro)₂-Arg-Asp-β-Ala-
(Arg)₆-Gln-(Arg)₃-NH₂;
(SEQ ID NO: 188)
Ac-c(Cys-Glu-His-D-2-Nal-Arg-Bal-Ala-Cys)-(Pro)₂-Arg-Asp-β-Ala-
(Arg)₅-Gln-(Arg)₃-NH₂;
(SEQ ID NO: 189)
Ac-c(Cys-Glu-His-D-2-Nal-Arg-Bal-Ala-Cys)-(Pro)₂-Lys-Asp-β-Ala-(Arg)₆-
Gln-(Arg)₃-NH₂;
(SEQ ID NO: 190)
Ac-c(Cys-Glu-His-D-2-Nal-Arg-Trp-Ala-Cys)-(Pro)₂-Lys-Asp-β-Ala-Tyr-
Gly-Arg-(Lys)₂-(Arg)₃-Gln-(Arg)₂-NH₂;
(SEQ ID NO: 191)
Ac-c(Cys-Glu-His-D-2-Nal-Arg-Trp-Ala-Cys)-(Pro)₂-Lys-Asp-β-Ala-Tyr-
Gly-Arg-Gln-(Lys)₂-(Arg)₅-NH₂;
(SEQ ID NO: 192)
Ac-c(Cys-Glu-His-D-2-Nal-Arg-Trp-Ala-Cys)-(Pro)₂-Lys-Asp-β-Ala-Tyr-
Gly-Arg-(Lys)₂-(Arg)₅-Gln-NH₂;
(SEQ ID NO: 193)
Ac-c(Cys-Glu-His-D-2-Nal-Arg-1-Nal-Ala-Cys)-(Pro)₂-Lys-Asp-β-Ala-Tyr-
Gly-Arg-(Lys)₂-(Arg)₂-Gln-(Arg)₃-NH₂;
(SEQ ID NO: 194)
Ac-c(Cys-Glu-His-D-2-Nal-Arg-Bal-Ala-Cys)-(Pro)₂-Lys-Asp-β-Ala-Tyr-
Gly-Arg-(Lys)₂-(Arg)₂-Gln-(Arg)₃-NH₂;
(SEQ ID NO: 195)
Ac-c(Cys-Glu-His-D-2-Nal-Arg-1-Nal-Ala-Cys)-(Pro)₂-Lys-Asp-β-Ala-
(Arg)₂-Lys-(Arg)₂-Gln-(Arg)₃-NH₂;
(SEQ ID NO: 196)
Ac-c(Cys-Glu-His-D-2-Nal-Arg-1-Nal-Ala-Cys)-(Pro)₂-Lys-Asp-β-Ala-Arg-
Lys-(Arg)₃-Gln-(Arg)₃-NH₂;
(SEQ ID NO: 197)
Ac-c(Cys-Glu-His-D-2-Nal-Arg-2-Nal-Ala-Cys)-(Pro)₂-Lys-Asp-β-Ala-
(Arg)₂-Lys-(Arg)₂-Gln-(Arg)₃-NH₂;
(SEQ ID NO: 198)
Ac-c(Cys-Glu-His-D-2-Nal-Arg-2-Nal-Ala-Cys)-(Pro)₂-Lys-Asp-β-Ala-Tyr-
Gly-(Arg)₂-Lys-(Arg)₂-Gln-(Arg)₃-NH₂;
(SEQ ID NO: 199)
Ac-c(Cys-Glu-His-D-2-Nal-Arg-2-Nal-Ala-Cys)-(Pro)₂-Lys-Asp-β-Ala-Gly-
(Arg)₂-Lys-(Arg)₂-Gln-(Arg)₃-NH₂;
(SEQ ID NO: 200)
Ac-c(Cys-Glu-His-D-2-Nal-Arg-2-Nal-Ala-Cys)-(Pro)₂-Lys-Asp-β-Ala-Gly-
Arg-Lys-(Arg)₃-Gln-(Arg)₃-NH₂;
(SEQ ID NO: 201)
Ac-c(Cys-Glu-His-D-2-Nal-Arg-1-Nal-Ala-Cys)-(Pro)₂-Lys-Asp-β-Ala-Tyr-
Gly-(Arg)₂-Lys-(Arg)₂-Gln-(Arg)₃-NH₂;
(SEQ ID NO: 202)
Ac-c(Cys-Glu-His-D-2-Nal-Arg-1-Nal-Ala-Cys)-(Pro)₂-Lys-Asp-β-Ala-Tyr-
Gly-Arg-Lys-(Arg)₃-Gln-(Arg)₃-NH₂;
(SEQ ID NO: 203)
Ac-c(Cys-Glu-His-D-2-Nal-Arg-1-Nal-Ala-Cys)-(Pro)₂-Lys-Asp-β-Ala-Gly-
(Arg)₂-Lys-(Arg)₂-Gln-(Arg)₃-NH₂;
(SEQ ID NO: 204)
Ac-c(Cys-Glu-His-D-2-Nal-Arg-1-Nal-Ala-Cys)-(Pro)₂-Lys-Asp-β-Ala-Gly-
Arg-Lys-(Arg)₃-Gln-(Arg)₃-NH₂;
(SEQ ID NO: 205)
Ac-c(Cys-Glu-His-D-2-Nal-Arg-2-Nal-Ala-Cys)-(Pro)₂-Lys-Asp-β-Ala-
(Arg)₂-Lys-(Arg)₂-Gln-(Arg)₃-NH₂;
(SEQ ID NO: 206)
Ac-c(Cys-Glu-His-D-2-Nal-Arg-2-Nal-Ala-Cys)-(Pro)₂-Lys-Asp-β-Ala-Arg-
Lys-(Arg)₃-Gln-(Arg)₃-NH₂;
(SEQ ID NO: 207)
Ac-c(Cys-Glu-His-D-2-Nal-Arg-2-Nal-Ala-Cys)-(Pro)₂-Lys-Asp-β-Ala-Tyr-
Gly-Arg-Lys-(Arg)₃-Gln-(Arg)₃-NH₂;
(SEQ ID NO: 208)
Ac-c(Cys-Glu-His-D-2-Nal-Arg-Bal-Ala-Cys)-(Pro)₂-Lys-Asp-β-Ala-(Arg)₂-
Lys-(Arg)₂-Gln-(Arg)₃-NH₂;
(SEQ ID NO: 209)
Ac-c(Cys-Glu-His-D-2-Nal-Arg-Bal-Ala-Cys)-(Pro)₂-Lys-Asp-β-Ala-Arg-
Lys-(Arg)₃-Gln-(Arg)₃-NH₂;
(SEQ ID NO: 210)
Ac-c(Cys-Glu-His-D-2-Nal-Arg-Bal-Ala-Cys)-(Pro)₂-Lys-Asp-β-Ala-Tyr-
Gly-(Arg)₂-Lys-(Arg)₂-Gln-(Arg)₃-NH₂;
(SEQ ID NO: 211)
Ac-c(Cys-Glu-His-D-2-Nal-Arg-Bal-Ala-Cys)-(Pro)₂-Lys-Asp-β-Ala-Tyr-
Gly-Arg-Lys-(Arg)₃-Gln-(Arg)₃-NH₂;
(SEQ ID NO: 212)
Ac-c(Cys-Glu-His-D-2-Nal-Arg-Bal-Ala-Cys)-(Pro)₂-Lys-Asp-β-Ala-Gly-
(Arg)₂-Lys-(Arg)₂-Gln-(Arg)₃-NH₂;
(SEQ ID NO: 213)
Ac-c(Cys-Glu-His-D-2-Nal-Arg-Bal-Ala-Cys)-(Pro)₂-Lys-Asp-β-Ala-Gly-
Arg-Lys-(Arg)₃-Gln-(Arg)₃-NH₂;
(SEQ ID NO: 214)
Ac-c(Cys-Glu-His-D-2-Nal-Arg-Trp-Ala-Cys)-(Pro)₂-Lys-Asp-β-Ala-
(Arg)₅-Gln-(Arg)₃-NH₂;
(SEQ ID NO: 215)
Ac-c(Cys-Glu-His-D-2-Nal-Arg-Trp-Ala-Cys)-(Pro)₂-Arg-Asp-β-Ala-
(Arg)₅-Gln-(Arg)₃-NH₂;
(SEQ ID NO: 216)
Ac-c(Cys-Glu-His-D-2-Nal-Arg-Trp-Ala-Cys)-(Pro)₂-Lys-Asp-β-Ala-Tyr-
Gly-(Arg)₅-Gln-(Arg)₃-NH₂;
(SEQ ID NO: 217)
Ac-c(Cys-Glu-His-D-2-Nal-Arg-Trp-Ala-Cys)-(Pro)₂-Arg-Asp-β-Ala-Tyr-
Gly-(Arg)₅-Gln-(Arg)₃-NH₂;
(SEQ ID NO: 218)
Ac-c(Cys-Glu-His-D-2-Nal-Arg-Trp-Ala-Cys)-(Pro)₂-Lys-Asp-β-Ala-
(Arg)₅-Gln-(Arg)₄-NH₂;
(SEQ ID NO: 219)
Ac-c(Cys-Glu-His-D-2-Nal-Arg-Trp-Ala-Cys)-(Pro)₂-Arg-Asp-β-Ala-
(Arg)₅-Gln-(Arg)₄-NH₂;
(SEQ ID NO: 220)
Ac-c(Cys-Glu-His-D-2-Nal-Arg-Trp-Ala-Cys)-(Pro)₂-Lys-Asp-β-Ala-Tyr-
Gly-(Arg)₅-Gln-(Arg)₄-NH₂;
(SEQ ID NO: 221)
Ac-c(Cys-Glu-His-D-2-Nal-Arg-Trp-Ala-Cys)-(Pro)₂-Arg-Asp-β-Ala-Tyr-
Gly-(Arg)₅-Gln-(Arg)₄-NH₂;
(SEQ ID NO: 222)
Ac-c(Cys-Glu-His-D-2-Nal-Arg-1-Nal-Ala-Cys)-(Pro)₂-Lys-Asp-β-Ala-
(Arg)₅-Gln-(Arg)₄-NH₂;
(SEQ ID NO: 223)
Ac-c(Cys-Glu-His-D-2-Nal-Arg-1-Nal-Ala-Cys)-(Pro)₂-Arg-Asp-β-Ala-
(Arg)₅-Gln-(Arg)₄-NH₂;
(SEQ ID NO: 224)
Ac-c(Cys-Glu-His-D-2-Nal-Arg-1-Nal-Ala-Cys)-(Pro)₂-Arg-Asp-β-Ala-
(Arg)₆-Gln-(Arg)₃-NH₂;
(SEQ ID NO: 225)
Ac-c(Cys-Glu-His-D-2-Nal-Arg-1-Nal-Ala-Cys)-(Pro)₂-Lys-Asp-β-Ala-Tyr-
Gly-(Arg)₅-Gln-(Arg)₃-NH₂;
(SEQ ID NO: 226)
Ac-c(Cys-Glu-His-D-2-Nal-Arg-1-Nal-Ala-Cys)-(Pro)₂-Arg-Asp-β-Ala-Tyr-
Gly-(Arg)₅-Gln-(Arg)₃-NH₂;
(SEQ ID NO: 227)
Ac-c(Cys-Glu-His-D-2-Nal-Arg-1-Nal-Ala-Cys)-(Pro)₂-Lys-Asp-β-Ala-Tyr-
Gly-(Arg)₅-Gln-(Arg)₄-NH₂;
(SEQ ID NO: 228)
Ac-c(Cys-Glu-His-D-2-Nal-Arg-1-Nal-Ala-Cys)-(Pro)₂-Arg-Asp-β-Ala-Tyr-
Gly-(Arg)₅-Gln-(Arg)₄-NH₂;
(SEQ ID NO: 229)
Ac-c(Cys-Glu-His-D-2-Nal-Arg-1-Nal-Ala-Cys)-(Pro)₂-Lys-Asp-β-Ala-Tyr-
Gly-(Arg)₆-Gln-(Arg)₃-NH₂;
(SEQ ID NO: 230)
Ac-c(Cys-Glu-His-D-2-Nal-Arg-1-Nal-Ala-Cys)-(Pro)₂-Arg-Asp-β-Ala-Tyr-
Gly-(Arg)₆-Gln-(Arg)₃-NH₂;
(SEQ ID NO: 231)
Ac-c(Cys-Glu-His-D-2-Nal-Arg-2-Nal-Ala-Cys)-(Pro)₂-Arg-Asp-β-Ala-
(Arg)₆-Gln-(Arg)₃-NH₂;
(SEQ ID NO: 232)
Ac-c(Cys-Glu-His-D-2-Nal-Arg-2-Nal-Ala-Cys)-(Pro)₂-Lys-Asp-β-Ala-
(Arg)₅-Gln-(Arg)₄-NH₂;
(SEQ ID NO: 233)
Ac-c(Cys-Glu-His-D-2-Nal-Arg-2-Nal-Ala-Cys)-(Pro)₂-Arg-Asp-β-Ala-
(Arg)₅-Gln-(Arg)₄-NH₂;
(SEQ ID NO: 234)
Ac-c(Cys-Glu-His-D-2-Nal-Arg-2-Nal-Ala-Cys)-(Pro)₂-Lys-Asp-β-Ala-Tyr-
Gly-(Arg)₅-Gln-(Arg)₃-NH₂;
(SEQ ID NO: 235)
Ac-c(Cys-Glu-His-D-2-Nal-Arg-2-Nal-Ala-Cys)-(Pro)₂-Arg-Asp-β-Ala-Tyr-
Gly-(Arg)₅-Gln-(Arg)₃-NH₂;
(SEQ ID NO: 236)
Ac-c(Cys-Glu-His-D-2-Nal-Arg-2-Nal-Ala-Cys)-(Pro)₂-Lys-Asp-β-Ala-Tyr-
Gly-(Arg)₆-Gln-(Arg)₃-NH₂;
(SEQ ID NO: 237)
Ac-c(Cys-Glu-His-D-2-Nal-Arg-2-Nal-Ala-Cys)-(Pro)₂-Arg-Asp-β-Ala-Tyr-
Gly-(Arg)₆-Gln-(Arg)₃-NH₂;
(SEQ ID NO: 238)
Ac-c(Cys-Glu-His-D-2-Nal-Arg-2-Nal-Ala-Cys)-(Pro)₂-Lys-Asp-β-Ala-Tyr-
Gly-(Arg)₅-Gln-(Arg)₄-NH₂;
(SEQ ID NO: 239)
Ac-c(Cys-Glu-His-D-2-Nal-Arg-2-Nal-Ala-Cys)-(Pro)₂-Arg-Asp-β-Ala-Tyr-
Gly-(Arg)₅-Gln-(Arg)₄-NH₂;
(SEQ ID NO: 240)
Ac-c(Cys-Glu-His-D-2-Nal-Arg-Bal-Ala-Cys)-(Pro)₂-Lys-Asp-β-Ala-(Arg)₅-
Gln-(Arg)₄-NH₂;
(SEQ ID NO: 241)
Ac-c(Cys-Glu-His-D-2-Nal-Arg-Bal-Ala-Cys)-(Pro)₂-Arg-Asp-β-Ala-
(Arg)₅-Gln-(Arg)₄-NH₂;
(SEQ ID NO: 242)
Ac-c(Cys-Glu-His-D-2-Nal-Arg-Bal-Ala-Cys)-(Pro)₂-Arg-Asp-β-Ala-Tyr-
Gly-(Arg)₅-Gln-(Arg)₃-NH₂;
(SEQ ID NO: 243)
Ac-c(Cys-Glu-His-D-2-Nal-Arg-Bal-Ala-Cys)-(Pro)₂-Lys-Asp-β-Ala-Tyr-
Gly-(Arg)₅-Gln-(Arg)₄-NH₂;
(SEQ ID NO: 244)
Ac-c(Cys-Glu-His-D-2-Nal-Arg-Bal-Ala-Cys)-(Pro)₂-Arg-Asp-β-Ala-Tyr-
Gly-(Arg)₅-Gln-(Arg)₄-NH₂;
(SEQ ID NO: 245)
Ac-c(Cys-Glu-His-D-2-Nal-Arg-Bal-Ala-Cys)-(Pro)₂-Lys-Asp-β-Ala-Tyr-
Gly-(Arg)₆-Gln-(Arg)₃-NH₂;
(SEQ ID NO: 246)
Ac-c(Cys-Glu-His-D-2-Nal-Arg-Bal-Ala-Cys)-(Pro)₂-Arg-Asp-β-Ala-Tyr-
Gly-(Arg)₆-Gln-(Arg)₃-NH₂;
(SEQ ID NO: 247)
Ac-Nle-c(Cys-D-Ala-His-D-Phe-Arg-Trp-Cys)-(Doc)₂-Tyr-Gly-Arg-(Lys)₂-
(Arg)₂-Gln-(Arg)₃-NH₂;
(SEQ ID NO: 248)
Ac-Nle-c(Cys-D-Ala-His-D-Phe-Arg-Trp-Cys)-β-Ala-Tyr-Gly-Arg-(Lys)₂-
Arg-Gln-(Arg)₄-NH₂;
(SEQ ID NO: 249)
Ac-Nle-c(Cys-D-Ala-His-D-Phe-Arg-Trp-Cys)-Doc-Tyr-Gly-Arg-(Lys)₂-
(Arg)₂-Gln-(Arg)₃-NH₂;
(SEQ ID NO: 250)
Ac-Nle-c(Cys-D-Ala-His-D-Phe-Arg-Trp-Cys)-β-Ala-(Arg)₅-Gln-(Arg)₃-
NH₂;
(SEQ ID NO: 251)
Ac-Nle-c(Cys-D-Ala-His-D-Phe-Arg-Trp-Cys)-β-Ala-Gly-(Arg)₅-Gln-
(Arg)₃-NH₂;
(SEQ ID NO: 252)
Ac-Nle-c(Cys-D-Ala-His-D-Phe-Arg-Trp-Cys)-β-Ala-Tyr-Gly-(Arg)₅-Gln-
(Arg)₃-NH₂;
(SEQ ID NO: 253)
Ac-Nle-c(Cys-D-Ala-His-D-Phe-Arg-Trp-Cys)-β-Ala-Gly-(Arg)₅-Gln-
(Arg)₄-NH₂;
(SEQ ID NO: 254)
Ac-Nle-c(Cys-D-Ala-His-D-Phe-Arg-Trp-Cys)-β-Ala-Tyr-Gly-(Arg)₂-Lys-
(Arg)₂-Gln-(Arg)₃-NH₂;
(SEQ ID NO: 255)
Ac-Nle-c(Cys-D-Ala-His-D-Phe-Arg-Trp-Cys)-β-Ala-Tyr-Gly-Arg-Lys-
(Arg)₃-Gln-(Arg)₃-NH₂;
(SEQ ID NO: 256)
Ac-Nle-c(Cys-D-Ala-His-D-Phe-Arg-Trp-Cys)-β-Ala-Gly-(Arg)₂-Lys-
(Arg)₂-Gln-(Arg)₃-NH₂;
(SEQ ID NO: 257)
Ac-Nle-c(Cys-D-Ala-His-D-Phe-Arg-Trp-Cys)-β-Ala-Gly-Arg-Lys-(Arg)₃-
Gln-(Arg)₃-NH₂;
(SEQ ID NO: 258)
Ac-Nle-c(Cys-D-Ala-His-D-Phe-Arg-Trp-Cys)-β-Ala-(Arg)₂-Lys-(Arg)₂-
Gln-(Arg)₃-NH₂;
(SEQ ID NO: 259)
Ac-Nle-c(Cys-D-Ala-His-D-Phe-Arg-Trp-Cys)-β-Ala-Arg-Lys-(Arg)₃-Gln-
(Arg)₃-NH₂;
(SEQ ID NO: 260)
Ac-Nle-c(Cys-D-Ala-His-D-Phe-Arg-Trp-Cys)-(β-Ala)₂-(Arg)₅-Gln-(Arg)₃-
NH₂;
(SEQ ID NO: 261)
Ac-Nle-c(Cys-D-Ala-His-D-Phe-Arg-Trp-Cys)-(β-Ala)₂-Gly-(Arg)₅-Gln-
(Arg)₃-NH₂;
(SEQ ID NO: 262)
Ac-Nle-c(Cys-D-Ala-His-D-Phe-Arg-Trp-Cys)-(β-Ala)₂-Tyr-Gly-(Arg)₅-
Gln-(Arg)₃-NH₂;
(SEQ ID NO: 263)
Ac-Nle-c(Cys-D-Ala-His-D-Phe-Arg-Trp-Cys)-Doc-(Arg)₅-Gln-(Arg)₃-
NH₂;
(SEQ ID NO: 264)
Ac-Nle-c(Cys-D-Ala-His-D-Phe-Arg-Trp-Cys)-Doc-Gly-(Arg)₅-Gln-(Arg)₃-
NH₂;
(SEQ ID NO: 265)
Ac-Nle-c(Cys-D-Ala-His-D-Phe-Arg-Trp-Cys)-Doc-Tyr-Gly-(Arg)₅-Gln-
(Arg)₃-NH₂;
(SEQ ID NO: 266)
Ac-Nle-c(Cys-D-Ala-His-D-Phe-Arg-Trp-Cys)-(Doc)₂-(Arg)₅-Gln-(Arg)₃-
NH₂;
(SEQ ID NO: 267)
Ac-Nle-c(Cys-D-Ala-His-D-Phe-Arg-Trp-Cys)-(Doc)₂-Gly-(Arg)₅-Gln-
(Arg)₃-NH₂;
(SEQ ID NO: 268)
Ac-Nle-c(Cys-D-Ala-His-D-Phe-Arg-Trp-Cys)-(Doc)₂-Tyr-Gly-(Arg)₅-
Gln-(Arg)₃-NH₂;
(SEQ ID NO: 269)
Ac-Nle-c(Cys-D-Ala-His-D-Phe-Arg-Trp-Cys)-β-Ala-(Arg)₅-Gln-(Arg)₄-
NH₂;
(SEQ ID NO: 270)
Ac-Nle-c(Cys-D-Ala-His-D-Phe-Arg-Trp-Cys)-β-Ala-Tyr-Gly-(Arg)₅-Gln-
(Arg)₄-NH₂;
(SEQ ID NO: 271)
Ac-Nle-c(Cys-D-Ala-His-D-Phe-Arg-Trp-Cys)-(β-Ala)₂-(Arg)₅-Gln-(Arg)₄-
NH₂;
(SEQ ID NO: 272)
Ac-Nle-c(Cys-D-Ala-His-D-Phe-Arg-Trp-Cys)-(β-Ala)₂-Gly-(Arg)₅-Gln-
(Arg)₄-NH₂;
(SEQ ID NO: 273)
Ac-Nle-c(Cys-D-Ala-His-D-Phe-Arg-Trp-Cys)-(β-Ala)₂-Tyr-Gly-(Arg)₅-
Gln-(Arg)₄-NH₂;
(SEQ ID NO: 274)
Ac-Nle-c(Cys-D-Ala-His-D-Phe-Arg-Trp-Cys)-Doc-(Arg)₅-Gln-(Arg)₄-
NH₂;
(SEQ ID NO: 275)
Ac-Nle-c(Cys-D-Ala-His-D-Phe-Arg-Trp-Cys)-Doc-Gly-(Arg)₅-Gln-(Arg)₄-
NH₂;
(SEQ ID NO: 276)
Ac-Nle-c(Cys-D-Ala-His-D-Phe-Arg-Trp-Cys)-Doc-Tyr-Gly-(Arg)₅-Gln-
(Arg)₄-NH₂;
(SEQ ID NO: 277)
Ac-Nle-c(Cys-D-Ala-His-D-Phe-Arg-Trp-Cys)-(Doc)₂-(Arg)₅-Gln-(Arg)₄-
NH₂;
(SEQ ID NO: 278)
Ac-Nle-c(Cys-D-Ala-His-D-Phe-Arg-Trp-Cys)-(Doc)₂-Gly-(Arg)₅-Gln-
(Arg)₄-NH₂;
(SEQ ID NO: 279)
Ac-Nle-c(Cys-D-Ala-His-D-Phe-Arg-Trp-Cys)-(Doc)₂-Tyr-Gly-(Arg)₅-
Gln-(Arg)₄-NH₂;
(SEQ ID NO: 280)
Ac-Nle-c(Cys-His-D-Phe-Arg-Trp-Gaba-Cys)-β-Ala-Tyr-Gly-(Arg)₅-Gln-
(Arg)₃-NH₂;
(SEQ ID NO: 281)
Ac-Nle-c(Cys-His-D-Phe-Arg-Trp-Gaba-Cys)-β-Ala-(Arg)₅-Gln-(Arg)₃-
NH₂;
(SEQ ID NO: 282)
Ac-Nle-c(Asp-His-D-Phe-Arg-Trp-Ala-Lys)-β-Ala-Tyr-Gly-(Arg)₅-Gln-
(Arg)₃-NH₂;
(SEQ ID NO: 283)
Ac-Nle-c(Asp-His-D-Phe-Arg-Trp-Ala-Lys)-β-Ala-(Arg)₅-Gln-(Arg)₃-NH₂;
(SEQ ID NO: 284)
Ac-Nle-c(Asp-His-D-Phe-Arg-Trp-Lys)-β-Ala-Tyr-Gly-(Arg)₅-Gln-(Arg)₃-
NH₂;
(SEQ ID NO: 285)
Ac-Nle-c(Asp-His-D-Phe-Arg-Trp-Lys)-β-Ala-Gly-(Arg)₅-Gln-(Arg)₃-NH₂;
(SEQ ID NO: 286)
Ac-Nle-c(Asp-His-D-Phe-Arg-Trp-Lys)-β-Ala-(Arg)₅-Gln-(Arg)₃-NH₂;
(SEQ ID NO: 287)
Ac-Nle-c(Asp-His-D-Phe-Arg-Trp-Lys)-(β-Ala)₂-Tyr-Gly-(Arg)₅-Gln-
(Arg)₃-NH₂;
(SEQ ID NO: 288)
Ac-Nle-c(Asp-His-D-Phe-Arg-Trp-Lys)-(β-Ala)₂-Gly-(Arg)₅-Gln-(Arg)₃-
NH₂;
(SEQ ID NO: 289)
Ac-Nle-c(Asp-His-D-Phe-Arg-Trp-Lys)-(β-Ala)₂-(Arg)₅-Gln-(Arg)₃-NH₂;
(SEQ ID NO: 290)
Ac-Nle-c(Asp-His-D-Phe-Arg-Trp-Lys)-Doc-Tyr-Gly-(Arg)₅-Gln-(Arg)₃-
NH₂;
(SEQ ID NO: 291)
Ac-Nle-c(Asp-His-D-Phe-Arg-Trp-Lys)-Doc-Gly-(Arg)₅-Gln-(Arg)₃-NH₂;
(SEQ ID NO: 292)
Ac-Nle-c(Asp-His-D-Phe-Arg-Trp-Lys)-Doc-(Arg)₅-Gln-(Arg)₃-NH₂;
(SEQ ID NO: 293)
Ac-Nle-c(Asp-His-D-Phe-Arg-Trp-Lys)-(Doc)₂-Tyr-Gly-(Arg)₅-Gln-(Arg)₃-
NH₂;
(SEQ ID NO: 294)
Ac-Nle-c(Asp-His-D-Phe-Arg-Trp-Lys)-(Doc)₂-Gly-(Arg)₅-Gln-(Arg)₃-
NH₂;
(SEQ ID NO: 295)
Ac-Nle-c(Asp-His-D-Phe-Arg-Trp-Lys)-(Doc)₂-(Arg)₅-Gln-(Arg)3-NH₂;
(SEQ ID NO: 296)
Ac-Nle-c(Asp-His-D-Phe-Arg-Trp-Lys)-β-Ala-Tyr-Gly-(Arg)₅-Gln-(Arg)₄-
NH₂;
(SEQ ID NO: 297)
Ac-Nle-c(Asp-His-D-Phe-Arg-Trp-Lys)-β-Ala-Gly-(Arg)₅-Gln-(Arg)₄-NH₂;
(SEQ ID NO: 298)
Ac-Nle-c(Asp-His-D-Phe-Arg-Trp-Lys)-β-Ala-(Arg)₅-Gln-(Arg)₄-NH₂;
(SEQ ID NO: 299)
Ac-Nle-c(Asp-His-D-Phe-Arg-Trp-Lys)-(β-Ala)₂-Tyr-Gly-(Arg)₅-Gln-
(Arg)₄-NH₂;
(SEQ ID NO: 300)
Ac-Nle-c(Asp-His-D-Phe-Arg-Trp-Lys)-(β-Ala)₂-Gly-(Arg)₅-Gln-(Arg)₄-
NH₂;
(SEQ ID NO: 301)
Ac-Nle-c(Asp-His-D-Phe-Arg-Trp-Lys)-(β-Ala)₂-(Arg)₅-Gln-(Arg)₄-NH₂;
(SEQ ID NO: 302)
Ac-Nle-c(Asp-His-D-Phe-Arg-Trp-Lys)-Doc-Tyr-Gly-(Arg)₅-Gln-(Arg)₄-
NH₂;
(SEQ ID NO: 303)
Ac-Nle-c(Asp-His-D-Phe-Arg-Trp-Lys)-Doc-Gly-(Arg)₅-Gln-(Arg)₄-NH₂;
(SEQ ID NO: 304)
Ac-Nle-c(Asp-His-D-Phe-Arg-Trp-Lys)-Doc-(Arg)₅-Gln-(Arg)₄-NH₂;
(SEQ ID NO: 305)
Ac-Nle-c(Asp-His-D-Phe-Arg-Trp-Lys)-(Doc)₂-Tyr-Gly-(Arg)₅-Gln-(Arg)₄-
NH₂;
(SEQ ID NO: 306)
Ac-Nle-c(Asp-His-D-Phe-Arg-Trp-Lys)-(Doc)₂-Gly-(Arg)₅-Gln-(Arg)₄-
NH₂;
(SEQ ID NO: 307)
Ac-Nle-c(Asp-His-D-Phe-Arg-Trp-Lys)-(Doc)₂-(Arg)₅-Gln-(Arg)₄-NH₂;
(SEQ ID NO: 308)
Ac-Nle-c(Asp-His-D-Phe-Arg-Trp-3-Ala-Lys)-β-Ala-Tyr-Gly-(Arg)₅-Gln-
(Arg)₃-NH₂;
(SEQ ID NO: 309)
Ac-Nle-c(Asp-His-D-Phe-Arg-Trp-3-Ala-Lys)-β-Ala-(Arg)₅-Gln-(Arg)₃-
NH₂;
(SEQ ID NO: 310)
Ac-Nle-c(Cys-His-D-Phe-Arg-Trp-Ahx-Cys)-β-Ala-Tyr-Gly-(Arg)₅-Gln-
(Arg)₃-NH₂;
(SEQ ID NO: 311)
Ac-Nle-c(Cys-His-D-Phe-Arg-Trp-Ahx-Cys)-β-Ala-(Arg)₅-Gln-(Arg)₃-
NH₂;
(SEQ ID NO: 312)
D-Phe-c(Cys-His-D-Phe-Arg-Trp-P-Ala-D-Cys)-Thr-β-Ala-Tyr-Gly-(Arg)₅-
Gln-(Arg)₃-NH₂;
(SEQ ID NO: 313)
D-Phe-c(Cys-His-D-Phe-Arg-Trp-P-Ala-D-Cys)-Thr-β-Ala-(Arg)₅-Gln-
(Arg)₃-NH₂;
(SEQ ID NO: 314)
Ac-Nle-c(Cys-His-D-Phe-Arg-Trp-Apn-Cys)-β-Ala-Tyr-Gly-(Arg)₅-Gln-
(Arg)₃-NH₂;
(SEQ ID NO: 315)
Ac-Nle-c(Cys-His-D-Phe-Arg-Trp-Apn-Cys)-β-Ala-(Arg)₅-Gln-(Arg)₃-
NH₂;
(SEQ ID NO: 316)
Ac-Cha-c(Asp-His-D-Phe-Arg-Trp-Gaba-Lys)-β-Ala-Tyr-Gly-(Arg)₅-Gln-
(Arg)₃-NH₂;
(SEQ ID NO: 317)
Ac-Cha-c(Asp-His-D-Phe-Arg-Trp-Gaba-Lys)-β-Ala-(Arg)₅-Gln-(Arg)₃-
NH₂;
(SEQ ID NO: 318)
Ac-Nle-c(Asp-His-D-Phe-Arg-Trp-Gaba-Lys)-β-Ala-Tyr-Gly-(Arg)₅-Gln-
(Arg)₃-NH₂;
(SEQ ID NO: 319)
Ac-Nle-c(Asp-His-D-Phe-Arg-Trp-Gaba-Lys)-β-Ala-(Arg)₅-Gln-(Arg)₃-
NH₂;
(SEQ ID NO: 320)
Ac-Chg-c(Asp-His-D-Phe-Arg-Trp-Gaba-Lys)-β-Ala-Tyr-Gly-(Arg)₅-Gln-
(Arg)₃-NH₂;
(SEQ ID NO: 321)
Ac-Chg-c(Asp-His-D-Phe-Arg-Trp-Gaba-Lys)-β-Ala-(Arg)₅-Gln-(Arg)₃-
NH₂;
(SEQ ID NO: 322)
Ac-hCha-c(Asp-His-D-Phe-Arg-Trp-Gaba-Lys)-β-Ala-Tyr-Gly-(Arg)₅-Gln-
(Arg)₃-NH₂;
(SEQ ID NO: 323)
Ac-hCha-c(Asp-His-D-Phe-Arg-Trp-Gaba-Lys)-β-Ala-(Arg)₅-Gln-(Arg)₃-
NH₂;
(SEQ ID NO: 324)
Ac-hCha-c(Asp-His-D-Phe-Arg-Trp-Gaba-Lys)-(β-Ala)₂-Tyr-Gly-(Arg)₅-
Gln-(Arg)₃-NH₂;
(SEQ ID NO: 325)
Ac-hCha-c(Asp-His-D-Phe-Arg-Trp-Gaba-Lys)-(β-Ala)₂-(Arg)₅-Gln-(Arg)₃-
NH₂;
(SEQ ID NO: 326)
Ac-hCha-c(Asp-His-D-Phe-Arg-Trp-Gaba-Lys)-Doc-Tyr-Gly-(Arg)₅-Gln-
(Arg)₃-NH₂;
(SEQ ID NO: 327)
Ac-hCha-c(Asp-His-D-Phe-Arg-Trp-Gaba-Lys)-Doc-(Arg)₅-Gln-(Arg)₃-
NH₂;
(SEQ ID NO: 328)
Ac-hCha-c(Asp-His-D-Phe-Arg-Trp-Gaba-Lys)-(Doc)₂-Tyr-Gly-(Arg)₅-
Gln-(Arg)₃-NH₂;
(SEQ ID NO: 329)
Ac-hCha-c(Asp-His-D-Phe-Arg-Trp-Gaba-Lys)-(Doc)₂-(Arg)₅-Gln-(Arg)₃-
NH₂;
(SEQ ID NO: 330)
Ac-hCha-c(Asp-His-D-Phe-Arg-Trp-Gaba-Lys)-β-Ala-Tyr-Gly-(Arg)₅-Gln-
(Arg)₄-NH₂;
(SEQ ID NO: 331)
Ac-hCha-c(Asp-His-D-Phe-Arg-Trp-Gaba-Lys)-β-Ala-(Arg)₅-Gln-(Arg)₄-
NH₂;
(SEQ ID NO: 332)
Ac-hCha-c(Asp-His-D-Phe-Arg-Trp-Gaba-Lys)-(β-Ala)₂-Tyr-Gly-(Arg)₅-
Gln-(Arg)₄-NH₂;
(SEQ ID NO: 333)
Ac-hCha-c(Asp-His-D-Phe-Arg-Trp-Gaba-Lys)-(β-Ala)₂-(Arg)₅-Gln-(Arg)₄-
NH₂;
(SEQ ID NO: 334)
Ac-hCha-c(Asp-His-D-Phe-Arg-Trp-Gaba-Lys)-Doc-Tyr-Gly-(Arg)₅-Gln-
(Arg)₄-NH₂;
(SEQ ID NO: 335)
Ac-hCha-c(Asp-His-D-Phe-Arg-Trp-Gaba-Lys)-Doc-(Arg)₅-Gln-(Arg)₄-
NH₂;
(SEQ ID NO: 336)
Ac-hCha-c(Asp-His-D-Phe-Arg-Trp-Gaba-Lys)-(Doc)₂-Tyr-Gly-(Arg)₅-
Gln-(Arg)₄-NH₂;
(SEQ ID NO: 337)
Ac-hCha-c(Asp-His-D-Phe-Arg-Trp-Gaba-Lys)-(Doc)₂-(Arg)₅-Gln-(Arg)₄-
NH₂;
(SEQ ID NO: 338)
Ac-D-Chg-c(Asp-His-D-Phe-Arg-Trp-Gaba-Lys)-β-Ala-Tyr-Gly-(Arg)₅-
Gln-(Arg)₃-NH₂;
(SEQ ID NO: 339)
Ac-D-Chg-c(Asp-His-D-Phe-Arg-Trp-Gaba-Lys)-β-Ala-(Arg)₅-Gln-(Arg)₃-
NH₂;
(SEQ ID NO: 340)
Ac-hPhe-c(Asp-His-D-Phe-Arg-Trp-Gaba-Lys)-β-Ala-Tyr-Gly-(Arg)₅-Gln-
(Arg)₃-NH₂;
(SEQ ID NO: 341)
Ac-hPhe-c(Asp-His-D-Phe-Arg-Trp-Gaba-Lys)-β-Ala-(Arg)₅-Gln-(Arg)₃-
NH₂;
(SEQ ID NO: 342)
Ac-Nle-c(Cys-His-D-Phe-Arg-D-Trp-Apn-Cys)-β-Ala-Tyr-Gly-(Arg)₅-Gln-
(Arg)₃-NH₂;
(SEQ ID NO: 343)
Ac-Nle-c(Cys-His-D-Phe-Arg-D-Trp-Apn-Cys)-β-Ala-(Arg)₅-Gln-(Arg)₃-
NH₂;
(SEQ ID NO: 344)
Ac-Nle-c(Cys-His-D-Phe-Arg-D-Trp-Ahx-Cys)-β-Ala-Tyr-Gly-(Arg)₅-Gln-
(Arg)₃-NH₂;
(SEQ ID NO: 345)
Ac-Nle-c(Cys-His-D-Phe-Arg-D-Trp-Ahx-Cys)-β-Ala-(Arg)₅-Gln-(Arg)₃-
NH₂;
(SEQ ID NO: 346)
Ac-Nle-c(Cys-His-D-Phe-Arg-D-Trp-3-Ala-Cys)-β-Ala-Tyr-Gly-(Arg)₅-
Gln-(Arg)₃-NH₂;
(SEQ ID NO: 347)
Ac-Nle-c(Cys-His-D-Phe-Arg-D-Trp-P-Ala-Cys)-β-Ala-(Arg)₅-Gln-(Arg)₃-
NH₂;
(SEQ ID NO: 348)
Ac-Nle-c(Cys-D-Ala-His-D-Phe-Arg-Trp-Pen)-β-Ala-Tyr-Gly-(Arg)₅-Gln-
(Arg)₃-NH₂;
(SEQ ID NO: 349)
Ac-Nle-c(Cys-D-Ala-His-D-Phe-Arg-Trp-Pen)-β-Ala-Gly-(Arg)₅-Gln-
(Arg)₃-NH₂;
(SEQ ID NO: 350)
Ac-Nle-c(Cys-D-Ala-His-D-Phe-Arg-Trp-Pen)-β-Ala-(Arg)₅-Gln-(Arg)₃-
NH₂;
(SEQ ID NO: 351)
Ac-Nle-c(Cys-D-Ala-His-D-Phe-Arg-Trp-Pen)-(β-Ala)₂-Tyr-Gly-(Arg)₅-
Gln-(Arg)₃-NH₂;
(SEQ ID NO: 352)
Ac-Nle-c(Cys-D-Ala-His-D-Phe-Arg-Trp-Pen)-(β-Ala)₂-Gly-(Arg)₅-Gln-
(Arg)₃-NH₂;
(SEQ ID NO: 353)
Ac-Nle-c(Cys-D-Ala-His-D-Phe-Arg-Trp-Pen)-(β-Ala)₂-(Arg)₅-Gln-(Arg)₃-
NH₂;
(SEQ ID NO: 354)
Ac-Nle-c(Cys-D-Ala-His-D-Phe-Arg-Trp-Pen)-Doc-Tyr-Gly-(Arg)₅-Gln-
(Arg)₃-NH₂;
(SEQ ID NO: 355)
Ac-Nle-c(Cys-D-Ala-His-D-Phe-Arg-Trp-Pen)-Doc-Gly-(Arg)₅-Gln-(Arg)₃-
NH₂;
(SEQ ID NO: 356)
Ac-Nle-c(Cys-D-Ala-His-D-Phe-Arg-Trp-Pen)-Doc-(Arg)₅-Gln-(Arg)₃-
NH₂;
(SEQ ID NO: 357)
Ac-Nle-c(Cys-D-Ala-His-D-Phe-Arg-Trp-Pen)-(Doc)₂-Tyr-Gly-(Arg)₅-Gln-
(Arg)₃-NH₂;
(SEQ ID NO: 358)
Ac-Nle-c(Cys-D-Ala-His-D-Phe-Arg-Trp-Pen)-(Doc)₂-Gly-(Arg)₅-Gln-
(Arg)₃-NH₂;
(SEQ ID NO: 359)
Ac-Nle-c(Cys-D-Ala-His-D-Phe-Arg-Trp-Pen)-(Doc)₂-(Arg)₅-Gln-(Arg)₃-
NH₂;
(SEQ ID NO: 360)
D-Phe-c(Cys-His-D-(Et)Tyr-Arg-Trp-β-Ala-D-Cys)-β-Ala-Tyr-Gly-(Arg)₅-
Gln-(Arg)₃-NH₂;
(SEQ ID NO: 361)
D-Phe-c(Cys-His-D-(Et)Tyr-Arg-Trp-β-Ala-D-Cys)-β-Ala-(Arg)₅-Gln-
(Arg)₃-NH₂;
(SEQ ID NO: 362)
D-Phe-c(Cys-His-D-(Et)Tyr-Arg-Trp-β-Ala-D-Cys)-β-Ala-Gly-(Arg)₅-Gln-
(Arg)₃-NH₂;
(SEQ ID NO: 363)
D-Phe-c(Cys-His-D-(Et)Tyr-Arg-Trp-β-Ala-D-Cys)-β-Ala-(Arg)₅-Gln-
(Arg)₄-NH₂;
(SEQ ID NO: 364)
D-Phe-c(Cys-His-D-((Et)Tyr-Arg-Trp-β-Ala-D-Cys)-(β-Ala)₂-Tyr-Gly-
(Arg)₅-Gln-(Arg)₃-NH₂;
(SEQ ID NO: 365)
D-Phe-c(Cys-His-D-(Et)Tyr-Arg-Trp-β-Ala-D-Cys)-(β-Ala)₂-(Arg)₅-Gln-
(Arg)₃-NH₂;
(SEQ ID NO: 366)
D-Phe-c(Cys-His-D-(Et)Tyr-Arg-Trp-β-Ala-D-Cys)-(β-Ala)₂-Gly-(Arg)₅-
Gln-(Arg)₃-NH₂;
(SEQ ID NO: 367)
D-Phe-c(Cys-His-D-(Et)Tyr-Arg-Trp-β-Ala-D-Cys)-(β-Ala)₂-(Arg)₅-Gln-
(Arg)₄-NH₂;
(SEQ ID NO: 368)
D-Phe-c(Cys-His-D-(Et)Tyr-Arg-Trp-β-Ala-D-Cys)-Doc-Tyr-Gly-(Arg)₅-
Gln-(Arg)₃-NH₂;
(SEQ ID NO: 369)
D-Phe-c(Cys-His-D-(Et)Tyr-Arg-Trp-β-Ala-D-Cys)-Doc-(Arg)₅-Gln-(Arg)₃-
NH₂;
(SEQ ID NO: 370)
D-Phe-c(Cys-His-D-(Et)Tyr-Arg-Trp-β-Ala-D-Cys)-Doc-Gly-(Arg)₅-Gln-
(Arg)₃-NH₂;
(SEQ ID NO: 371)
D-Phe-c(Cys-His-D-(Et)Tyr-Arg-Trp-β-Ala-D-Cys)-Doc-(Arg)₅-Gln-(Arg)₄-
NH₂;
(SEQ ID NO: 372)
D-Phe-c(Cys-His-D-(Et)Tyr-Arg-Trp-β-Ala-D-Cys)-(Doc)₂-Tyr-Gly-(Arg)₅-
Gln-(Arg)₃-NH₂;
(SEQ ID NO: 373)
D-Phe-c(Cys-His-D-(Et)Tyr-Arg-Trp-β-Ala-D-Cys)-(Doc)₂-(Arg)₅-Gln-
(Arg)₃-NH₂;
(SEQ ID NO: 374)
D-Phe-c(Cys-His-D-(Et)Tyr-Arg-Trp-β-Ala-D-Cys)-(Doc)₂-Gly-(Arg)₅-
Gln-(Arg)₃-NH₂;
(SEQ ID NO: 375)
D-Phe-c(Cys-His-D-(Et)Tyr-Arg-Trp-β-Ala-D-Cys)-(Doc)₂-(Arg)₅-Gln-
(Arg)₄-NH₂;
(SEQ ID NO: 376)
D-Phe-c(Cys-His-D-(Et)Tyr-hArg-Trp-β-Ala-D-Cys)-Thr-β-Ala-Tyr-Gly-
(Arg)₅-Gln-(Arg)₃-NH₂;
(SEQ ID NO: 377)
D-Phe-c(Cys-His-D-(Et)Tyr-hArg-Trp-β-Ala-D-Cys)-Thr-β-Ala-(Arg)₅-
Gln-(Arg)₃-NH₂;
(SEQ ID NO: 378)
D-Phe-c(Cys-His-D-(Et)Tyr-hArg-Trp-β-Ala-D-Cys)-Thr-(β-Ala)₂-Tyr-Gly-
(Arg)₅-Gln-(Arg)₃-NH₂;
(SEQ ID NO: 379)
D-Phe-c(Cys-His-D-(Et)Tyr-hArg-Trp-β-Ala-D-Cys)-Thr-(β-Ala)₂-(Arg)₅-
Gln-(Arg)₃-NH₂;
(SEQ ID NO: 380)
D-Phe-c(Cys-His-D-(Et)Tyr-hArg-Trp-β-Ala-D-Cys)-Thr-Doc-Tyr-Gly-
(Arg)₅-Gln-(Arg)₃-NH₂;
(SEQ ID NO: 381)
D-Phe-c(Cys-His-D-(Et)Tyr-hArg-Trp-β-Ala-D-Cys)-Thr-Doc-(Arg)₅-Gln-
(Arg)₃-NH₂;
(SEQ ID NO: 382)
D-Phe-c(Cys-His-D-(Et)Tyr-hArg-Trp-β-Ala-D-Cys)-Thr-(Doc)₂-Tyr-Gly-
(Arg)₅-Gln-(Arg)₃-NH₂;
(SEQ ID NO: 383)
D-Phe-c(Cys-His-D-(Et)Tyr-hArg-Trp-β-Ala-D-Cys)-Thr-β-Ala-Tyr-Gly-
(Arg)₅-Gln-(Arg)₄-NH₂;
(SEQ ID NO: 384)
D-Phe-c(Cys-His-D-(Et)Tyr-hArg-Trp-β-Ala-D-Cys)-Thr-β-Ala-(Arg)₅-
Gln-(Arg)₄-NH₂;
(SEQ ID NO: 385)
D-Phe-c(Cys-His-D-(Et)Tyr-hArg-Trp-β-Ala-D-Cys)-Thr-(β-Ala)₂-Tyr-Gly-
(Arg)₅-Gln-(Arg)₄-NH₂;
(SEQ ID NO: 386)
D-Phe-c(Cys-His-D-(Et)Tyr-hArg-Trp-β-Ala-D-Cys)-Thr-(β-Ala)₂-(Arg)₅-
Gln-(Arg)₄-NH₂;
(SEQ ID NO: 387)
D-Phe-c(Cys-His-D-(Et)Tyr-hArg-Trp-β-Ala-D-Cys)-Thr-Doc-Tyr-Gly-
(Arg)₅-Gln-(Arg)₄-NH₂;
(SEQ ID NO: 388)
D-Phe-c(Cys-His-D-(Et)Tyr-hArg-Trp-β-Ala-D-Cys)-Thr-Doc-(Arg)₅-Gln-
(Arg)₄-NH₂;
(SEQ ID NO: 389)
D-Phe-c(Cys-His-D-(Et)Tyr-hArg-Trp-β-Ala-D-Cys)-Thr-(Doc)₂-Tyr-Gly-
(Arg)₅-Gln-(Arg)₄-NH₂;
(SEQ ID NO: 390)
D-Phe-c(Cys-His-D-(Et)Tyr-hArg-Trp-β-Ala-D-Cys)-Thr-(Doc)₂-(Arg)₅-
Gln-(Arg)₄-NH₂;
(SEQ ID NO: 391)
D-Phe-c(Cys-His-D-(Et)Tyr-hArg-Bip-β-Ala-D-Cys)-Thr-β-Ala-Tyr-Gly-
(Arg)₅-Gln-(Arg)₃-NH₂;
(SEQ ID NO: 392)
D-Phe-c(Cys-His-D-(Et)Tyr-hArg-Bip-β-Ala-D-Cys)-Thr-β-Ala-Tyr-Gly-
(Arg)₅-Gln-(Arg)₄-NH₂;
(SEQ ID NO: 393)
D-Phe-c(Cys-His-D-(Et)Tyr-hArg-Bip-β-Ala-D-Cys)-Thr-β-Ala-(Arg)₅-
Gln-(Arg)₃-NH₂;
(SEQ ID NO: 394)
D-Phe-c(Cys-His-D-(Et)Tyr-hArg-Bip-β-Ala-D-Cys)-Thr-(β-Ala)₂-Tyr-Gly-
(Arg)₅-Gln-(Arg)₃-NH₂;
(SEQ ID NO: 395)
D-Phe-c(Cys-His-D-(Et)Tyr-hArg-Bip-β-Ala-D-Cys)-Thr-(β-Ala)₂-(Arg)₅-
Gln-(Arg)₃-NH₂;
(SEQ ID NO: 396)
D-Phe-c(Cys-His-D-(Et)Tyr-hArg-Bip-β-Ala-D-Cys)-Thr-Doc-Tyr-Gly-
(Arg)₅-Gln-(Arg)₃-NH₂;
(SEQ ID NO: 397)
D-Phe-c(Cys-His-D-(Et)Tyr-hArg-Bip-β-Ala-D-Cys)-Thr-Doc-Tyr-Gly-
(Arg)₅-Gln-(Arg)₄-NH₂;
(SEQ ID NO: 398)
D-Phe-c(Cys-His-D-(Et)Tyr-hArg-Bip-β-Ala-D-Cys)-Thr-Doc-(Arg)₅-Gln-
(Arg)₃-NH₂;
(SEQ ID NO: 399)
D-Phe-c(Cys-His-D-(Et)Tyr-hArg-Bip-β-Ala-D-Cys)-Thr-(Doc)₂-Tyr-Gly-
(Arg)₅-Gln-(Arg)₃-NH₂;
(SEQ ID NO: 400)
D-Phe-c(Cys-His-D-(Et)Tyr-hArg-Bip-β-Ala-D-Cys)-Thr-(Doc)₂-(Arg)₅-
Gln-(Arg)₃-NH₂;
(SEQ ID NO: 401)
Ac-Nle-c(Cys-D-Ala-His-D-Phe-Arg-Trp-Gly-Cys)-β-Ala-Tyr-Gly-(Arg)₅-
Gln-(Arg)₃-NH₂;
(SEQ ID NO: 402)
Ac-Nle-c(Cys-D-Ala-His-D-Phe-Arg-Trp-Gly-Cys)-β-Ala-(Arg)₅-Gln-
(Arg)₃-NH₂;
(SEQ ID NO: 403)
Nle-c(Cys-His-D-Phe-Arg-Trp-Apn-Cys)-β-Ala-Tyr-Gly-(Arg)₅-Gln-(Arg)₃-
NH₂;
(SEQ ID NO: 404)
Nle-c(Cys-His-D-Phe-Arg-Trp-Apn-Cys)-β-Ala-(Arg)₅-Gln-(Arg)₃-NH₂;;
(SEQ ID NO: 405)
Nle-c(Cys-His-D-Phe-Arg-Trp-Apn-Cys)-(β-Ala)₂-Tyr-Gly-(Arg)₅-Gln-
(Arg)₃-NH₂;
(SEQ ID NO: 406)
Nle-c(Cys-His-D-Phe-Arg-Trp-Apn-Cys)-(β-Ala)₂-(Arg)₅-Gln-(Arg)₃-NH₂;
(SEQ ID NO: 407)
Nle-c(Cys-His-D-Phe-Arg-Trp-Apn-Cys)-β-Ala-Tyr-Gly-(Arg)₅-Gln-(Arg)₄-
NH₂;
(SEQ ID NO: 408)
Nle-c(Cys-His-D-Phe-Arg-Trp-Apn-Cys)-β-Ala-(Arg)₅-Gln-(Arg)₄-NH₂;
(SEQ ID NO: 409)
Nle-c(Cys-His-D-Phe-Arg-Trp-Apn-Cys)-(β-Ala)₂-Tyr-Gly-(Arg)₅-Gln-
(Arg)₄-NH₂;
(SEQ ID NO: 410)
Nle-c(Cys-His-D-Phe-Arg-Trp-Apn-Cys)-(β-Ala)₂-(Arg)₅-Gln-(Arg)₄-NH₂;
(SEQ ID NO: 411)
Nle-c(Cys-His-D-Phe-Arg-Trp-Apn-Cys)-Doc-Tyr-Gly-(Arg)₅-Gln-(Arg)₃-
NH₂;
(SEQ ID NO: 412)
Nle-c(Cys-His-D-Phe-Arg-Trp-Apn-Cys)-Doc-(Arg)₅-Gln-(Arg)₃-NH₂;
(SEQ ID NO: 413)
Nle-c(Cys-His-D-Phe-Arg-Trp-Apn-Cys)-(Doc)₂-Tyr-Gly-(Arg)₅-Gln-
(Arg)₃-NH₂;
(SEQ ID NO: 414)
Nle-c(Cys-His-D-Phe-Arg-Trp-Apn-Cys)-(Doc)₂-(Arg)₅-Gln-(Arg)₃-NH₂;
(SEQ ID NO: 415)
Nle-c(Cys-His-D-Phe-Arg-Trp-Apn-Cys)-Doc-Tyr-Gly-(Arg)₅-Gln-(Arg)₄-
NH₂;
(SEQ ID NO: 416)
Nle-c(Cys-His-D-Phe-Arg-Trp-Apn-Cys)-Doc-(Arg)₅-Gln-(Arg)₄-NH₂;
(SEQ ID NO: 417)
Nle-c(Cys-His-D-Phe-Arg-Trp-Apn-Cys)-(Doc)₂-Tyr-Gly-(Arg)₅-Gln-
(Arg)₄-NH₂;
(SEQ ID NO: 418)
Nle-c(Cys-His-D-Phe-Arg-Trp-Apn-Cys)-(Doc)₂-(Arg)5-Gln-(Arg)₄-NH₂;
(SEQ ID NO: 419)
Ac-Nle-c(Cys-D-Leu-His-D-Phe-Arg-Trp-Cys)-β-Ala-Tyr-Gly-(Arg)₅-Gln-
(Arg)₃-NH₂;
(SEQ ID NO: 420)
Ac-Nle-c(Cys-D-Leu-His-D-Phe-Arg-Trp-Cys)-β-Ala-(Arg)₅-Gln-(Arg)₃-
NH₂;
(SEQ ID NO: 421)
Ac-Nle-c(Cys-D-Leu-His-D-Phe-Arg-Trp-Cys)-(β-Ala)₂-Tyr-Gly-(Arg)₅-
Gln-(Arg)₃-NH₂;
(SEQ ID NO: 422)
Ac-Nle-c(Cys-D-Leu-His-D-Phe-Arg-Trp-Cys)-(β-Ala)₂-(Arg)₅-Gln-(Arg)₃-
NH₂;
(SEQ ID NO: 423)
Ac-Nle-c(Cys-D-Leu-His-D-Phe-Arg-Trp-Cys)-Doc-Tyr-Gly-(Arg)₅-Gln-
(Arg)₃-NH₂;
(SEQ ID NO: 424)
Ac-Nle-c(Cys-D-Leu-His-D-Phe-Arg-Trp-Cys)-Doc-(Arg)₅-Gln-(Arg)₃-
NH₂;
(SEQ ID NO: 425)
Ac-Nle-c(Cys-D-Leu-His-D-Phe-Arg-Trp-Cys)-(Doc)₂-Tyr-Gly-(Arg)₅-
Gln-(Arg)₃-NH₂;
(SEQ ID NO: 426)
Ac-Nle-c(Cys-D-Leu-His-D-Phe-Arg-Trp-Cys)-(Doc)₂-(Arg)₅-Gln-(Arg)₃-
NH₂;
(SEQ ID NO: 427)
Ac-Nle-c(Cys-D-Leu-His-D-Phe-Arg-Trp-Cys)-β-Ala-Tyr-Gly-(Arg)₅-Gln-
(Arg)₄-NH₂;
(SEQ ID NO: 428)
Ac-Nle-c(Cys-D-Leu-His-D-Phe-Arg-Trp-Cys)-β-Ala-(Arg)₅-Gln-(Arg)₄-
NH₂;
(SEQ ID NO: 429)
Ac-Nle-c(Cys-D-Leu-His-D-Phe-Arg-Trp-Cys)-(β-Ala)₂-Tyr-Gly-(Arg)₅-
Gln-(Arg)₄-NH₂;
(SEQ ID NO: 430)
Ac-Nle-c(Cys-D-Leu-His-D-Phe-Arg-Trp-Cys)-(β-Ala)₂-(Arg)₅-Gln-(Arg)₄-
NH₂;
(SEQ ID NO: 431)
Ac-Nle-c(Cys-D-Leu-His-D-Phe-Arg-Trp-Cys)-Doc-Tyr-Gly-(Arg)₅-Gln-
(Arg)₄-NH₂;
(SEQ ID NO: 432)
Ac-Nle-c(Cys-D-Leu-His-D-Phe-Arg-Trp-Cys)-Doc-(Arg)₅-Gln-(Arg)₄-
NH₂;
(SEQ ID NO: 433)
Ac-Nle-c(Cys-D-Leu-His-D-Phe-Arg-Trp-Cys)-(Doc)₂-Tyr-Gly-(Arg)₅-
Gln-(Arg)₄-NH₂;
(SEQ ID NO: 434)
Ac-Nle-c(Cys-D-Leu-His-D-Phe-Arg-Trp-Cys)-(Doc)₂-(Arg)₅-Gln-(Arg)₄-
NH₂;
(SEQ ID NO: 435)
Ac-Nle-c(Cys-D-Cha-His-D-Phe-Arg-Trp-Cys)-β-Ala-Tyr-Gly-(Arg)₅-Gln-
(Arg)₃-NH₂;
(SEQ ID NO: 436)
Ac-Nle-c(Cys-D-Cha-His-D-Phe-Arg-Trp-Cys)-β-Ala-(Arg)₅-Gln-(Arg)₃-
NH₂;
(SEQ ID NO: 437)
Ac-Nle-c(Cys-D-Cha-His-D-Phe-Arg-Trp-Cys)-(β-Ala)₂-Tyr-Gly-(Arg)₅-
Gln-(Arg)₃-NH₂;
(SEQ ID NO: 438)
Ac-Nle-c(Cys-D-Cha-His-D-Phe-Arg-Trp-Cys)-(β-Ala)₂-(Arg)₅-Gln-(Arg)₃-
NH₂;
(SEQ ID NO: 439)
Ac-Nle-c(Cys-D-Cha-His-D-Phe-Arg-Trp-Cys)-Doc-Tyr-Gly-(Arg)₅-Gln-
(Arg)₃-NH₂;
(SEQ ID NO: 440)
Ac-Nle-c(Cys-D-Cha-His-D-Phe-Arg-Trp-Cys)-Doc-(Arg)₅-Gln-(Arg)₃-
NH₂;
(SEQ ID NO: 441)
Ac-Nle-c(Cys-D-Cha-His-D-Phe-Arg-Trp-Cys)-(Doc)₂-Tyr-Gly-(Arg)₅-
Gln-(Arg)₃-NH₂;
(SEQ ID NO: 442)
Ac-Nle-c(Cys-D-Cha-His-D-Phe-Arg-Trp-Cys)-(Doc)₂-(Arg)₅-Gln-(Arg)₃-
NH₂;
(SEQ ID NO: 443)
Ac-Nle-c(Cys-D-Cha-His-D-Phe-Arg-Trp-Cys)-β-Ala-Tyr-Gly-(Arg)₅-Gln-
(Arg)₄-NH₂;
(SEQ ID NO: 444)
Ac-Nle-c(Cys-D-Cha-His-D-Phe-Arg-Trp-Cys)-β-Ala-(Arg)₅-Gln-(Arg)₄-
NH₂;
(SEQ ID NO: 445)
Ac-Nle-c(Cys-D-Cha-His-D-Phe-Arg-Trp-Cys)-(β-Ala)₂-Tyr-Gly-(Arg)₅-
Gln-(Arg)4-NH₂;
(SEQ ID NO: 446)
Ac-Nle-c(Cys-D-Cha-His-D-Phe-Arg-Trp-Cys)-(β-Ala)₂-(Arg)₅-Gln-(Arg)₄-
NH₂;
(SEQ ID NO: 447)
Ac-Nle-c(Cys-D-Cha-His-D-Phe-Arg-Trp-Cys)-Doc-Tyr-Gly-(Arg)₅-Gln-
(Arg)₄-NH₂;
(SEQ ID NO: 448)
Ac-Nle-c(Cys-D-Cha-His-D-Phe-Arg-Trp-Cys)-Doc-(Arg)₅-Gln-(Arg)₄-
NH₂;
(SEQ ID NO: 449)
Ac-Nle-c(Cys-D-Cha-His-D-Phe-Arg-Trp-Cys)-(Doc)₂-Tyr-Gly-(Arg)₅-Gln-
(Arg)₄-NH₂;
(SEQ ID NO: 450)
Ac-Nle-c(Cys-D-Cha-His-D-Phe-Arg-Trp-Cys)-(Doc)₂-(Arg)₅-Gln-(Arg)₄-
NH₂;
(SEQ ID NO: 451)
Nle-c(Cys-His-D-Phe-Arg-Trp-Gaba-Cys)-β-Ala-Tyr-Gly-(Arg)₅-Gln-
(Arg)₃-NH₂;
(SEQ ID NO: 452)
Nle-c(Cys-His-D-Phe-Arg-Trp-Gaba-Cys)-β-Ala-(Arg)₅-Gln-(Arg)₃-NH₂;
(SEQ ID NO: 453)
Nle-c(Cys-His-D-Phe-Arg-Trp-Gaba-Cys)-(β-Ala)₂-Tyr-Gly-(Arg)₅-Gln-
(Arg)₃-NH₂;
(SEQ ID NO: 454)
Nle-c(Cys-His-D-Phe-Arg-Trp-Gaba-Cys)-(β-Ala)₂-(Arg)₅-Gln-(Arg)₃-NH₂;
(SEQ ID NO: 455)
Nle-c(Cys-His-D-Phe-Arg-Trp-Gaba-Cys)-β-Ala-Tyr-Gly-(Arg)₅-Gln-
(Arg)₄-NH₂;
(SEQ ID NO: 456)
Nle-c(Cys-His-D-Phe-Arg-Trp-Gaba-Cys)-β-Ala-(Arg)₅-Gln-(Arg)₄-NH₂;
(SEQ ID NO: 457)
Nle-c(Cys-His-D-Phe-Arg-Trp-Gaba-Cys)-(β-Ala)₂-Tyr-Gly-(Arg)₅-Gln-
(Arg)₄-NH₂;
(SEQ ID NO: 458)
Nle-c(Cys-His-D-Phe-Arg-Trp-Gaba-Cys)-(β-Ala)₂-(Arg)₅-Gln-(Arg)₄-NH₂;
(SEQ ID NO: 459)
Nle-c(Cys-His-D-Phe-Arg-Trp-Gaba-Cys)-Doc-Tyr-Gly-(Arg)₅-Gln-(Arg)₃-
NH₂;
(SEQ ID NO: 460)
Nle-c(Cys-His-D-Phe-Arg-Trp-Gaba-Cys)-Doc-(Arg)₅-Gln-(Arg)₃-NH₂;
(SEQ ID NO: 461)
Nle-c(Cys-His-D-Phe-Arg-Trp-Gaba-Cys)-(Doc)₂-Tyr-Gly-(Arg)₅-Gln-
(Arg)₃-NH₂;
(SEQ ID NO: 462)
Nle-c(Cys-His-D-Phe-Arg-Trp-Gaba-Cys)-(Doc)₂-(Arg)₅-Gln-(Arg)₃-NH₂;
(SEQ ID NO: 463)
Nle-c(Cys-His-D-Phe-Arg-Trp-Gaba-Cys)-Doc-Tyr-Gly-(Arg)₅-Gln-(Arg)₄-
NH₂;
(SEQ ID NO: 464)
Nle-c(Cys-His-D-Phe-Arg-Trp-Gaba-Cys)-Doc-(Arg)₅-Gln-(Arg)₄-NH₂;
(SEQ ID NO: 465)
Nle-c(Cys-His-D-Phe-Arg-Trp-Gaba-Cys)-(Doc)₂-Tyr-Gly-(Arg)₅-Gln-
(Arg)₄-NH₂;
or
(SEQ ID NO: 466)
Nle-c(Cys-His-D-Phe-Arg-Trp-Gaba-Cys)-(Doc)₂-(Arg)₅-Gln-(Arg)₄-NH₂,

or pharmaceutically acceptable salts thereof.

In an example embodiment, the compounds useful for practicing the methods described herein are the compounds of Formula (IV):

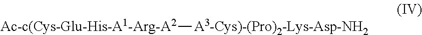

or pharmaceutically acceptable salts thereof. In Formula (IV):
A¹ is the D-isomer of X-Phe or 2-Nal where X is halogen;
A² is Bal, 1-Nal, 2-Nal, or Trp; and
A³ is Aib, Ala, β-Ala or Gly,

In an example embodiments, the at least one of the following compounds is used:

(SEQ ID NO: 467)
Ac-c(Cys-Glu-His-D-4-Br-Phe-Arg-Trp-Gly-Cys)-
(Pro)₂-Lys-Asp-NH₂;
(SEQ ID NO: 468)
Ac-c(Cys-Glu-His-D-2-Nal-Arg-Trp-Ala-Cys)-
(Pro)₂-Lys-Asp-NH₂;
(SEQ ID NO: 469)
Ac-c(Cys-Glu-His-D-2-Nal-Arg-2-Nal-Ala-Cys)-
(Pro)₂-Lys-Asp-NH₂;
(SEQ ID NO: 470)
Ac-c(Cys-Glu-His-D-2-Nal-Arg-1-Nal-Ala-Cys)-
(Pro)₂-Lys-Asp-NH₂;
(SEQ ID NO: 471)
Ac-c(Cys-Glu-His-D-2-Nal-Arg-Bal-Ala-Cys)-
(Pro)₂-Lys-Asp-NH₂;
(SEQ ID NO: 472)
Ac-c(Cys-Glu-His-D-2-Nal-Arg-2-Nal-β-Ala-Cys)-
(Pro)₂-Lys-Asp-NH₂;
or
(SEQ ID NO: 473)
Ac-c(Cys-Glu-His-D-2-Nal-Arg-2-Nal-Aib-Cys)-
(Pro)₂-Lys-Asp-NH₂;

or pharmaceutically acceptable salts thereof.

In example embodiments, an MC4R agonist useful for practicing the methods described herein is at least one compound modified with a hydantoin moiety according to Formula (V), (VI) or (VII), or a pharmaceutically acceptable salt, hydrate, solvate or a prodrug thereof.

Formula (V) is described below: (see International Patent Application Number PCT/US08/06675 incorporated herein by reference in its entirety).

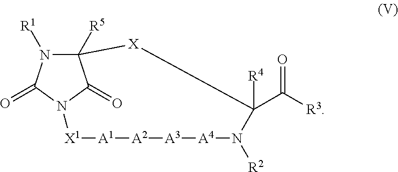

In Formula (V):
X is selected from the group consisting of —CH₂—S—S—CH₂—, —C(CH₃)₂—S—S—CH₂—, —CH₂—S—S—C(CH₃)₂—, —C(CH₃)₂—S—S—C(CH₃)₂—, —(CH₂)₂—S—S—CH₂—, —CH₂—S—S—(CH₂)₂—, (CH₂)₂—S—S—(CH₂)₂—, —C(CH₃)₂—S—S—(CH₂)₂—, —(CH₂)₂—S—S—C(CH₃)₂—, —(CH₂)ᵣ—C(O)—NR⁸—(CH₂)ᵣ— and —(CH₂)ᵣ—NR⁸—C(O)—(CH₂)ₜ—;
R² each is, independently, H, (C₁-C₁₀)alkyl or substituted (C₁-C₁₀)alkyl;
R³ is —OH or —NH₂;
R⁴ and R⁵ each is, independently, H, (C₁-C₁₀)alkyl or substituted (C₁-C₁₀)alkyl;
X¹ is

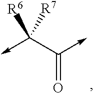

A¹ is H is, 2-Pal, 3-Pal, 4-Pal, (X¹, X², X³, X⁴, X⁵)Phe, Taz, 2-Thi, 3-Thi or is deleted;
A² is D-Bal, D-1-Nal, D-2-Nal, D-Phe or D-(X¹, X², X³, X⁴, X⁵)Phe;
A³ is Arg, hArg, Dab, Dap, Lys or Orn;
A⁴ is Bal, 1-Nal, 2-Nal, (X¹, X², X³, X⁴, X⁵)Phe or Trp;
R⁶ and R⁷ each is, independently for each occurrence thereof, H, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl provided that R⁶ and R⁷ may be joined together to form a ring;
R⁸ is H, (C₁-C₁₀)alkyl or substituted (C₁-C₁₀)alkyl;
r is, independently for each occurrence thereof, 1, 2, 3, 4 or 5; and
t is, independently for each occurrence thereof, 1 or 2.

Compounds according the foregoing formula can include compounds wherein X¹ is selected from the group consisting of;

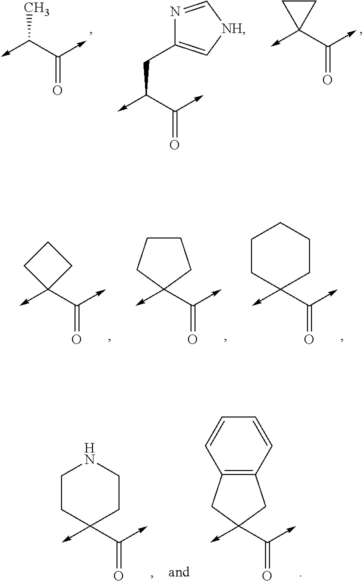

Representative embodiments of the foregoing class of compounds are as follows:

(SEQ ID NO: 474
c[Hydantoin(C(O)-(Cys-D-Ala))-
His-D-Phe-Arg-Trp-Cys]-NH₂;
(SEQ ID NO: 475)
c[Hydantoin(C(O)-(hCys-D-Ala))-
His-D-Phe-Arg-Trp-Cys]-NH₂;
(SEQ ID NO: 476)
c[Hydantoin(C(O)-(Cys-D-Ala))-
His-D-2-Nal-Arg-Trp-Cys]-NH₂;
(SEQ ID NO: 477)
c[Hydantoin(C(O)-(hCys-D-Ala))-
His-D-2-Nal-Arg-Trp-Cys]-NH₂;
(SEQ ID NO: 478)
c[Hydantoin(C(O)-(Asp-D-Ala))-
His-D-Phe-Arg-Trp-Lys]-NH₂;
(SEQ ID NO: 479)
c[Hydantoin(C(O)-(Asp-D-Ala))-
His-D-Phe-Arg-Trp-Orn]-NH₂;
(SEQ ID NO: 480)
c[Hydantoin(C(O)-(Asp-D-Ala))-
His-D-Phe-Arg-Trp-Dab]-NH₂;
(SEQ ID NO: 481)
c[Hydantoin(C(O)-(Asp-D-Ala))-
His-D-Phe-Arg-Trp-Dap]-NH₂;
(SEQ ID NO: 482)
c[Hydantoin(C(O)-(Asp-His))-
D-2-Nal-Arg-Trp-Lys]-NH₂;
(SEQ ID NO: 483)
c[Hydantoin(C(O)-(Asp-His))-
D-Phe-Arg-Trp-Lys]-NH₂;
(SEQ ID NO: 484)
c[Hydantoin(C(O)-(Asp-A3c))-
D-Phe-Arg-Trp-Lys]-NH₂;
(SEQ ID NO: 485)
c[Hydantoin(C(O)-(Asp-A5c))-
D-Phe-Arg-Trp-Lys]-NH₂;
(SEQ ID NO: 486)
c[Hydantoin(C(O)-(Asp-A6c))-
D-Phe-Arg-Trp-Lys]-NH₂;
(SEQ ID NO: 487)
c[Hydantoin(C(O)-(Asp-A3c))-
D-2-Nal-Arg-Trp-Lys]-NH₂;
(SEQ ID NO: 488)
c[Hydantoin(C(O)-(Asp-A5c))-
D-2-Nal-Arg-Trp-Lys]-NH₂;
(SEQ ID NO: 489)
c[Hydantoin(C(O)-(Asp-A6c))-
D-2-Nal-Arg-Trp-Lys]-NH₂;
(SEQ ID NO: 490)
c[Hydantoin(C(O)-(Asp-Aic))-
D-Phe-Arg-Trp-Lys]-NH₂;
(SEQ ID NO: 491)
c[Hydantoin(C(O)-(Asp-Apc))-
D-Phe-Arg-Trp-Lys]-NH₂;
(SEQ ID NO: 492)
c[Hydantoin(C(O)-(Asp-Aic))-
D-2-Nal-Arg-Trp-Lys]-NH₂;
(SEQ ID NO: 493)
c[Hydantoin(C(O)-(Asp-Apc))-
D-2-Nal-Arg-Trp-Lys]-NH₂;
(SEQ ID NO: 494)
c[Hydantoin(C(O)-(Glu-D-Ala))-
His-D-Phe-Arg-Trp-Orn]-NH₂;
(SEQ ID NO: 495)
c[Hydantoin(C(O)-(Glu-D-Ala))-
His-D-Phe-Arg-Trp-DaN-NH₂;
(SEQ ID NO: 496)
c[Hydantoin(C(O)-(Glu-D-Ala))-
His-D-Phe-Arg-Trp-Dap]-NH₂;
(SEQ ID NO: 497)
c[Hydantoin(C(O)-(Glu-D-Ala))-
His-D-Phe-Arg-Trp-Lys]-NH₂;
(SEQ ID NO: 498)
c[Hydantoin(C(O)-(Glu-His))-
D-Phe-Arg-Trp-Dap]-NH₂;
or
(SEQ ID NO: 499)
c[Hydantoin(C(O)-(Glu-His))-
D-Phe-Arg-Trp-Lys]-NH₂.

In an example embodiment, an MC4R agonist useful for practicing the methods described herein is at least one compound of Formula (VI), a pharmaceutically-acceptable salt, hydrate, solvate and/or prodrugs thereof (see International Patent Application Number PCT/US08/06675 which is incorporated herein by reference in its entirety):

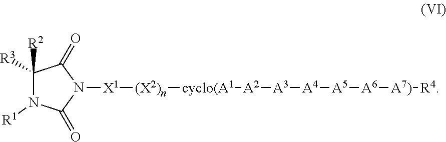

### In Formula (VI):

X¹ is;

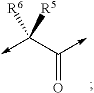

X² is

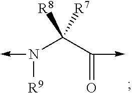

A¹ is Asp, Cys, D-Cys, Dab, Dap, Glu, Lys, Orn, Pen or D-Pen;
A² is an L- or D-amino acid;
A³ is H is, 2-Pal, 3-Pal, 4-Pal, (X¹, X², X³, X⁴, X⁵)Phe, Taz, 2-Thi or 3-Thi;
A⁴ is D-Bal, D-1-Nal, D-2-Nal, D-Phe or D-(X¹, X², X³, X⁴, X⁵)Phe;
A⁵ is Arg, hArg, Dab, Dap, Lys or Orn;
A⁶ is Bal, 1-Nal, 2-Nal, (X¹, X², X³, X⁴, X⁵)Phe or Trp;
A⁷ is Asp, Cys, D-Cys, Dab, Dap, Glu, Lys, Orn, Pen or D-Pen;
R¹ is H, (C₁-C₁₀)alkyl or substituted (C₁-C₁₀)alkyl;
R² and R³ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together form a cyclic moiety;
R⁴ is CO₂H or C(O)NH₂;
R⁵ and R⁶ each is, independently, H, (C₁-00)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R⁵ and R⁶ may be fused together form a cyclic moiety;
R⁷ and R⁸ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl; or R⁷ and R⁸ may be fused together form a cyclic moiety;
R⁹ is H, (C₁-C₁₀)alkyl or substituted (C₁-C₁₀)alkyl; and
n is, independently for each occurrence thereof, 1, 2, 3, 4, 5, 6 or 7;
or a pharmaceutically acceptable salt thereof.

Exemplary embodiments of the compounds of Formula (VI) are those compounds wherein:
A¹ is Cys;
A² is D-Ala, Asn, Asp, Gln, Glu or D-Phe;
A³ is H is;
A⁴ is D-2-Nal or D-Phe;
A⁵ is Arg;
A⁶ is Trp; and
A⁷ is Cys or Pen;
each of R¹, R², R³, and R⁹ is, independently, H;
R⁴ is C(O)NH₂;
each of R⁵ and R⁶ is, independently, H, (C₁-C₁₀)heteroalkyl, substituted (C₁-C₁₀)alkyl or substituted (C₁-C₁₀)heteroalkyl or R⁵ and R⁶ may be fused together form a cyclic moiety; and each of R⁷ and R⁸ is, independently, H, (C₁-C₁₀)alkyl, (C₁-C₁₀)heteroalkyl, substituted (C₁-C₁₀)alkyl or substituted (C₁-C₁₀)heteroalkyl; or pharmaceutically acceptable salts thereof.

Example compounds of the immediately foregoing Formula (VI) include:

(SEQ ID NO: 500)
Hydantoin(C(O)-(Arg-Gly))-c(Cys-Glu-His-D-Phe-Arg-Trp-Cys)-NH₂;
(SEQ ID NO: 501)
Hydantoin(C(O)-(Nle-Gly))-c(Cys-Glu-His-D-Phe-Arg-Trp-Cys)-NH₂;
(SEQ ID NO: 502)
Hydantoin(C(O)-(Gly-Gly))-c(Cys-Glu-His-D-Phe-Arg-Trp-Cys)-NH₂;
(SEQ ID NO: 503)
Hydantoin(C(O)-(Nle-Gly))-c(Cys-D-Ala-His-D-Phe-Arg-Trp-Cys)-NH₂;
(SEQ ID NO: 504)
Hydantoin(C(O)-(Gly-Gly))-c(Cys-D-Ala-His-D-Phe-Arg-Trp-Cys)-NH₂;
(SEQ ID NO: 505)
Hydantoin(C(O)-(Nle-Gly))-c(Cys-D-Ala-His-D-Phe-Arg-Trp-Pen)-NH₂;
(SEQ ID NO: 506)
Hydantoin(C(O)-(Gly-Gly))-c(Cys-D-Ala-His-D-Phe-Arg-Trp-Pen)-NH₂;
(SEQ ID NO: 507)
Hydantoin(C(O)-(Ala-Gly))-c(Cys-D-Ala-His-D-Phe-Arg-Trp-Cys)-NH₂;
(SEQ ID NO: 508)
Hydantoin(C(O)-(D-Ala-Gly))-c(Cys-D-Ala-His-D-Phe-Arg-Trp-Cys)-NH₂;
(SEQ ID NO: 509)
Hydantoin(C(O)-(Aib-Gly))-c(Cys-D-Ala-His-D-Phe-Arg-Trp-Cys)-NH₂;
(SEQ ID NO: 510)
Hydantoin(C(O)-(Val-Gly))-c(Cys-D-Ala-His-D-Phe-Arg-Trp-Cys)-NH₂;
(SEQ ID NO: 511)
Hydantoin(C(O)-(Ile-Gly))-c(Cys-D-Ala-His-D-Phe-Arg-Trp-Cys)-NH₂;
(SEQ ID NO: 512)
Hydantoin(C(O)-(Leu-Gly))-c(Cys-D-Ala-His-D-Phe-Arg-Trp-Cys)-NH₂;
(SEQ ID NO: 513)
Hydantoin(C(O)-(Gly-Gly))-c(Cys-Glu-His-D-2-Nal-Arg-Trp-Cys)-NH₂;
(SEQ ID NO: 514)
Hydantoin(C(O)-(Nle-Gly))-c(Cys-Glu-His-D-2-Nal-Arg-Trp-Cys)-NH₂;
(SEQ ID NO: 515)
Hydantoin(C(O)-(D-Arg-Gly))-c(Cys-Glu-His-D-Phe-Arg-Trp-Cys)-NH₂;
(SEQ ID NO: 516)
Hydantoin(C(O)-(D-Arg-Gly))-c(Cys-D-Ala-His-D-Phe-Arg-Trp-Cys)-NH₂;
(SEQ ID NO: 517)
Hydantoin(C(O)-(Arg-Gly))-c(Cys-D-Ala-His-D-Phe-Arg-Trp-Cys)-NH₂;
(SEQ ID NO: 518)
Hydantoin(C(O)-(D-Arg-Gly))-c(Cys-D-Ala-His-D-2-Nal-Arg-Trp-Cys)-NH₂;
(SEQ ID NO: 519)
Hydantoin(C(O)-(Arg-Gly))-c(Cys-D-Ala-His-D-2-Nal-Arg-Trp-Cys)-NH₂;
(SEQ ID NO: 520)
Hydantoin(C(O)-(Ala-Nle))-c(Cys-Glu-His-D-Phe-Arg-Trp-Cys)-NH₂;
(SEQ ID NO: 521)
Hydantoin(C(O)-(Val-Nle))-c(Cys-Glu-His-D-Phe-Arg-Trp-Cys)-NH₂;
(SEQ ID NO: 522)
Hydantoin(C(O)-(Gly-Nle))-c(Cys-Glu-His-D-Phe-Arg-Trp-Cys)-NH₂;
(SEQ ID NO: 523)
Hydantoin(C(O)-(A6c-Nle))-c(Cys-D-Ala-His-D-Phe-Arg-Trp-Cys)-NH₂;
(SEQ ID NO: 524)
Hydantoin(C(O)-(Gly-Nle))-c(Cys-D-Ala-His-D-Phe-Arg-Trp-Cys)-NH₂;
(SEQ ID NO: 525)
Hydantoin(C(O)-(A1a-Nle))-c(Cys-D-Ala-His-D-Phe-Arg-Trp-Cys)-NH₂;
(SEQ ID NO: 526)
Hydantoin(C(O)-(D-Ala-Nle))-c(Cys-D-Ala-His-D-Phe-Arg-Trp-Cys)-NH₂;
(SEQ ID NO: 527)
Hydantoin(C(O)-(Val-Nle))-c(Cys-D-Ala-His-D-Phe-Arg-Trp-Cys)-NH₂;
(SEQ ID NO: 528)
Hydantoin(C(O)-(Leu-Nle))-c(Cys-D-Ala-His-D-Phe-Arg-Trp-Cys)-NH₂;
(SEQ ID NO: 529)
Hydantoin(C(O)-(Cha-Nle))-c(Cys-D-Ala-His-D-Phe-Arg-Trp-Cys)-NH₂;
(SEQ ID NO: 530)
Hydantoin(C(O)-(Aib-Nle))-c(Cys-D-Ala-His-D-Phe-Arg-Trp-Cys)-NH₂;
(SEQ ID NO: 531)
Hydantoin(C(O)-(Gly-Arg))-c(Cys-Glu-His-D-Phe-Arg-Trp-Cys)-NH₂;
(SEQ ID NO: 532)
Hydantoin(C(O)-(Gly-Arg))-c(Cys-Glu-His-D-2-Nal-Arg-Trp-Cys)-NH₂;
(SEQ ID NO: 533)
Hydantoin(C(O)-(Gly-Arg))-c(Cys-D-Ala-His-D-Phe-Arg-Trp-Cys)-NH₂;
(SEQ ID NO: 534)
Hydantoin(C(O)-(Gly-Arg))-c(Cys-D-Ala-His-D-2-Nal-Arg-Trp-Cys)-NH₂;
(SEQ ID NO: 535)
Hydantoin(C(O)-(Gly-D-Arg))-c(Cys-Glu-His-D-Phe-Arg-Trp-Cys)-NH₂;
(SEQ ID NO: 536)
Hydantoin(C(O)-(Gly-D-Arg))-c(Cys-D-Ala-His-D-Phe-Arg-Trp-Cys)-NH₂;
(SEQ ID NO: 537)
Hydantoin(C(O)-(Gly-D-Arg))-c(Cys-D-Ala-His-D-2-Nal-Arg-Trp-Cys)-NH₂;
or
(SEQ ID NO: 538)
Hydantoin(C(O)-(Nle-Ala))-c(Cys-Glu-His-D-Phe-Arg-Trp-Cys)-NH₂;

or a pharmaceutically acceptable salt thereof.

In an example embodiment, the MC4R agonists useful for practicing the methods described herein are compounds having a structure according to Formula (VII) as depicted below (see International Patent Application Number PCT/US08/06675 which is incorporated herein by reference in its entirety):

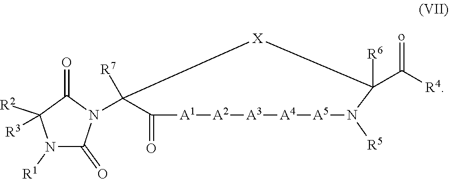

wherein:
X is selected from the group consisting of CH₂—S—S—CH₂—, —C(CH₃)₂—S—S—CH₂—, —CH₂—S—S—C(CH₃)₂—, —C(CH₃)₂—S—S—C(CH₃)₂—, —(CH₂)₂S—S—CH₂—, —CH₂—S—S—(CH₂)₂, (CH₂)₂—S—S—(CH₂)₂—, C(CH₃)₂—S—S—(CH₂)₂—, —(CH₂)₂—S—S—C(CH₃)₂—, —(CH₂)ₜ—C(O)—NR⁸—(CH₂)ᵣ— and —(CH₂)ᵣ—NR⁸—C(O)—(CH₂)ᵣ—;
each of R¹ and R⁵ is, independently, H, (C₁-C₁₀)alkyl or substituted (C₁-C₁₀)alkyl;
each of R² and R³ is, independently, H, (C₁-C₁₀)alkyl, (C₁₋₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together to form a ring;
R⁴ is OH or NH₂;
each of R⁶ and R⁷ is, independently, H, (C₁-C₁₀)alkyl or substituted (C₁-C₁₀)alkyl;
A¹ is an L- or D-amino acid or deleted;
A² is H is, 2-Pal, 3-Pal, 4-Pal, (X¹, X², X³, X⁴, X⁵)Phe, Taz, 2-Thi or 3-Thi;
A³ is D-Bal, D-1-Nal, D-2-Nal, D-Phe or D-(X¹, X², X³, X⁴, X⁵)Phe;
A⁴ is Arg, hArg, Dab, Dap, Lys or Orn;
A⁵ is Bal, 1-Nal, 2-Nal, (X¹, X², X³, X⁴, X⁵)Phe or Trp;
r is, independently for each occurrence thereof, 1, 2, 3, 4 or 5; and
t is, independently for each occurrence thereof, 1 or 2;
or pharmaceutically acceptable salts thereof.

In an example embodiment of the compounds of Formula (VII), A¹ is Ala, D-Ala, Asn, Asp, Gln, Glu or Gly.

Example compounds according to Formula (VII) include the following compounds:

(SEQ ID NO: 539)
c[Hydantoin(C(O)-(Nle-Cys))-D-Ala-
His-D-Phe-Arg-Trp-Cys]-NH₂;
(SEQ ID NO: 540)
c[Hydantoin(C(O)-(Ala-Cys))-D-Ala-
His-D-Phe-Arg-Trp-Cys]-NH₂;
(SEQ ID NO: 541)
c[Hydantoin(C(O)-(D-Ala-Cys))-D-
Ala-His-D-Phe-Arg-Trp-Cys]-NH₂;
(SEQ ID NO: 542)
c[Hydantoin(C(O)-(Aib-Cys))-D-Ala-
His-D-Phe-Arg-Trp-Cys]-NH₂;
(SEQ ID NO: 543)
c[Hydantoin(C(O)-(Val-Cys))-D-Ala-
His-D-Phe-Arg-Trp-Cys]-NH₂;
(SEQ ID NO: 544)
c[Hydantoin(C(O)-(Abu-Cys))-D-Ala-
His-D-Phe-Arg-Trp-Cys]-NH₂;
(SEQ ID NO: 545)
c[Hydantoin(C(O)-(Leu-Cys))-D-Ala-
His-D-Phe-Arg-Trp-Cys]-NH₂;
(SEQ ID NO: 546)
c[Hydantoin(C(O)-(Ile-Cys))-D-Ala-
His-D-Phe-Arg-Trp-Cys]-NH₂;
(SEQ ID NO: 547)
c[Hydantoin(C(O)-(Cha-Cys))-D-Ala-
His-D-Phe-Arg-Trp-Cys]-NH₂;
(SEQ ID NO: 548)
c[Hydantoin(C(O)-(A6c-Cys))-D-Ala-
His-D-Phe-Arg-Trp-Cys]-NH₂;
(SEQ ID NO: 549)
c[Hydantoin(C(O)-(Phe-Cys))-D-Ala-
His-D-Phe-Arg-Trp-Cys]-NH₂;
(SEQ ID NO: 550)
c[Hydantoin(C(O)-(Gly-Cys))-D-Ala-
His-D-Phe-Arg-Trp-Cys]-NH₂;
or
(SEQ ID NO: 551)
c[Hydantoin(C(O)-(Gly-Cys))-Glu-
His-D-Phe-Arg-Trp-Cys]-NH₂;

or pharmaceutically acceptable salts thereof.

In an example embodiment, the MC4R agonist useful for practicing the methods described herein is at least one compound according to Formula (VIII) (see International Patent Application Number PCT/US08/07411, incorporated herein by reference in its entirety):

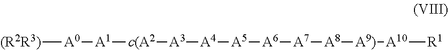

In Formula (VIII):
A⁰ is an aromatic amino acid
A¹ is Acc, HN—(CH₂)ₘ—C(O), an L- or D-amino acid;
A² is Asp, Cys, D-Cys, hCys, D-hCys, Glu, Pen, or D-Pen;
A³ is Aib, Ala, β-Ala, Gaba, Gly or a D-amino acid;
A⁴ is H is, 2-Pal, 3-Pal, 4-Pal, (X¹, X², X³, X⁴, X⁵)Phe, Taz, 2-Thi, or 3-Thi;
A⁵ is D-Bal, D-1-Nal, D-2-Nal, D-Phe, L-Phe, D-(X¹, X², X³, X⁴, X⁵)Phe, L-Phe, D-Trp or D-(Et)Tyr;
A⁶ is Arg, hArg, Dab, Dap, Lys, Orn, or HN—CH((CH₂)ₙ—N(R⁴R⁵))—C(O);
A⁷ is Bal, D-Bal, Bip, D-Bip, 1-Nal, D-1-Nal, 2-Nal, D-2-Nal, or D-Trp;
A⁸ is Acc, Aha, Ahx, Ala, D-Ala, β-Ala, Apn, Gaba, Gly, HN—(CH₂)ₛ—C(O), or deleted;
A⁹ is Cys, D-Cys, hCys, D-hCys, Dab, Dap, Lys, Orn, Pen, or D-Pen;
A¹⁰ is Acc, HN—(CH₂)ₜ-C(O), L- or D-amino acid, or deleted;
R¹ is OH, or NH₂;
each of R² and R³ is, independently for each occurrence selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)allyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X¹, X², X³, X⁴, and X⁵ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN.

In example embodiments of Formula (VIII),
(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl;
(II) when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;
(III) when A² is Cys, D-Cys, hCys, D-hCys, Pen, or D-Pen, then A⁹ is Cys, D-Cys, hCys, D-hCys, Pen, or D-Pen;
(IV) when A² is Asp or Glu, then A⁹ is Dab, Dap, Orn, or Lys;
(V) when A⁸ is Ala or Gly, then A¹ is not Nle; or pharmaceutically acceptable salts thereof.

In example embodiments of compounds of Formula (VIII):
A⁰ is 1-Nal, 2-Nal, H is, Pff, Phe, Trp, or Tyr;
A¹ is Arg;
A² is Cys;
A³ is D-Ala;
A⁴ is H is;
A⁵ is D-Phe
A⁶ is Arg;
A⁷ is Trp
A⁸ is deleted;
A⁹ is Cys; and
A¹⁰ is deleted;
or pharmaceutically acceptable salts thereof.

Particular compounds of the immediately foregoing group of compounds are of the formula:

(SEQ ID NO: 552)
Ac-Tyr-Arg-c(Cys-D-Ala-His-D-Phe-Arg-Trp-Cys)-NH₂;
(SEQ ID NO: 553)
Ac-2-Nal-Arg-c(Cys-D-Ala-His-D-Phe-Arg-Trp-Cys)-NH₂;
(SEQ ID NO: 554)
Ac-1-Nal-Arg-c(Cys-D-Ala-His-DPhe-Arg-Trp-Cys)-NH₂;
(SEQ ID NO: 555)
Ac-Phe-Arg-c(Cys-D-Ala-His-D-Phe-Arg-Trp-Cys)-NH₂;
(SEQ ID NO: 556)
Ac-Trp-Arg-c(Cys-D-Ala-His-D-Phe-Arg-Trp-Cys)-NH₂;
(SEQ ID NO: 557)
Ac-Pff-Arg-c(Cys-D-Ala-His-D-Phe-Arg-Trp-Cys)-NH₂;
(SEQ ID NO: 558)
H-His-Arg-c(Cys-D-Ala-His-D-Phe-Arg-Trp-Cys)-NH₂;
or
(SEQ ID NO: 559)
Ac-His-Arg-c(Cys-D-Ala-His-D-Phe-Arg-Trp-Cys)-NH₂;

or a pharmaceutically acceptable salt thereof.

In one example embodiment, the MC4R agonist is Ac-Arg-c(Cys-D-Ala-His-D-Phe-Arg-Trp-Cys)-NH₂ (SEQ ID NO: 140) or a pharmaceutically acceptable salt thereof. In another example embodiment, the MC4R agonist is Hydantoin(C(O)-(Arg-Gly))-c(Cys-Glu-His-D-Phe-Arg-Trp-Cys)-NH₂(SEQ ID NO: 500) or a pharmaceutically acceptable salt thereof.

Administration of a compound or pharmaceutically acceptable salt thereof or a composition comprising a compound or pharmaceutical salt of a compound of the invention useful to practice the methods described herein, can be continuous, hourly, four times daily, three time daily, twice daily, once daily, once every other day, twice weekly, once weekly, once every two weeks, once a month, or once every two months, or longer or some other intermittent dosing regimen.

Examples of administration of a compound or composition comprising a compound or pharmaceutical salt of a compound of the invention include peripheral administration. Examples of peripheral administration include oral, subcutaneous, intraperitoneal, intramuscular, intravenous, rectal, transdermal or intranasal forms of administration.

As used herein, peripheral administration includes all forms of administration of a compound or a composition comprising a compound of the instant invention which excludes intracranial administration. Examples of peripheral administration include, but are not limited to, oral, parenteral (e.g., intramuscular, intraperitoneal, intravenous or subcutaneous injection, extended release, slow release implant, depot and the like), nasal, vaginal, rectal, sublingual or topical routes of administration, including transdermal patch applications and the like.

The nomenclature used to define the peptides is that typically used in the art wherein the amino group at the N-terminus appears to the left and the carboxyl group at the C-terminus appears to the right. Where the amino acid has D and L isomeric forms, it is the L form of the amino acid that is represented unless otherwise explicitly indicated.

The compounds of the invention useful for practicing the methods described herein may possess one or more chiral centers and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the invention.

The compounds described herein may exist in one or more tautomeric forms. All tautomers and mixtures thereof are included in the scope of the present invention. For example, a claim to 2-hydroxypyridinyl would also cover its tautomeric form, α-pyridonyl.

Unless defined otherwise, all technical and scientific terms used herein have the same meaning as commonly understood by one of ordinary skill in the art to which this invention belongs. Also, all publications, patent applications, patents and other references mentioned herein are incorporated by reference in their entirety.

| Symbol | Meaning |
|---|---|
| Abu | α-aminobutyric acid |
| Ac | acyl group |
| Acc | 1-amino-1-cyclo(C₃-C₉)alkyl carboxylic acid |
| A3c | 1-amino-1cyclopropanecarboxylic acid |
| A4c | 1-amino-1-cyclobutanecarboxylic acid |
| A5c | 1-amino-1-cyclopentanecarboxylic acid |
| A6c | 1-amino-1-cyclohexanecarboxylic acid |
| Aha | 7-aminoheptanoic acid |
| Ahx | 6-aminohexanoic acid |
| Aib | α-aminoisobutyric acid |
| Aic | 2-aminoindan-2-carboxylic acid |
| Ala or A | alanine |
| β-Ala | β-alanine |
| Apc | denotes the structure: |
| | 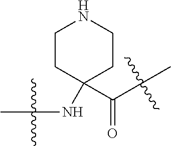 [SMILES: *NC1(C(*)=O)CCNCC1] |
| Apn | 5-aminopentanoic acid (HN—(CH2)₄—C(O) |
| Arg or R | arginine |
| hArg | homoarginine |
| Asn or N | asparagine |
| Asp or D | aspartic acid |
| Bal | 3-benzothienylalanine |
| Bip | 4,4′-biphenylalanine, represented by the structure |
| | 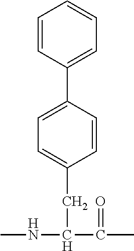 [SMILES: CNC(Cc1ccc(-c2ccccc2)cc1)C(C)=O] |
| Bpa | 4-benzoylphenylalanine |
| 4-Br-Phe | 4-bromo-phenylalanine |
| Cha | β-cyclohexylalanine |
| hCha | homo-cyclohexylalanine |
| Chg | cyclohexylglycine |
| Cys or C | cysteine |
| hCys | homocysteine |
| Dab | 2,4-diaminobutyric acid |
| Dap | 2,3-diaminopropionic acid |
| Dip | β,β-diphenylalanine |
| Doc | 8-amino-3,6-dioxaoctanoic acid with the structure of: |
| | 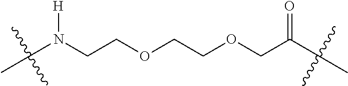 [SMILES: *NCCOCCOCC(*)=O] |
| 2-Fua | β-(2-furyl)-alanine |
| Gaba | 4-aminobutyric acid |
| Gln or Q | glutamine |
| Glu or E | glutamic acid |
| Gly or G | glycine |
| His or H | histidine |
| 3-Hyp | trans-3-hydroxy-L-proline, i.e., (2S,3S)-3- |
| | hydroxy-pyrrolidine-2-carboxylic acid |
| 4-Hyp | 4-hydroxyproline, i.e., (2S,4R)-4- |
| | hydorxypyrrolidine-2-carboxylic acid |
| Ile or 1 | isoleucine |
| Leu or L | leucine |
| hLeu | homoleucine |
| Lys or K | lysine |
| Met or M | methionine |
| β-hMet | β-homomethionine |
| 1-Nal | β-(1-naphthyl)alanine |
| 2-Nal | β-(2-naphthyl)alanine |
| Nip | nipecotic acid |
| Nle | norleucine |
| Ole | octahydroindole-2-carboxylic acid |
| Orn | ornithine |
| 2-Pal | β-(2-pyridiyl)alanine |
| 3-Pal | β-(3-pyridiyl)alanine |
| 4-Pal | β-(4-pyridiyl)alanine |
| Pen | penicillamine |
| Pff | (S)-pentafluorophenylalanine |
| Phe or F | phenylalanine |
| hPhe | homophenylalanine |
| Pro or P | proline |
| hProP | homoproline |
| Ser or S | Serine |
| Tle | tert-Leucine |
| Taz | β-(4-thiazolyl)alanine |
| 2-Thi | β-(2-thienyl)alanine |
| 3-Thi | β-(3-thienyl)alanine |
| Thr or T | threonine |
| Trp or W | tryptopham |
| Tyr or Y | tyrosine |
| D-(Et) Tyr | has a structure of |
| | 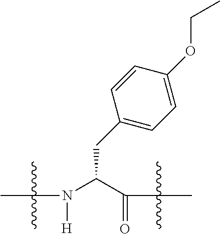 [SMILES: *N[C@H](Cc1ccc(OCC)cc1)C(*)=O] |
| Val or V | Valine |

Certain other abbriviations used herein are defined as follows:Boc: tert-butyloxycabonylBzl: benzylDCM: dichloromethaneDIC: N,N-diisopropylcarbodiimideDIEA: diisopropylethyl amineDmab: 4-{N-,(1-(4,4-dimethyl-2,6-dioxocyclohexylidene)-3-methylbutyl)-amino}benzyl
DMAP: 4-(dimethylamino)pyridine
DMF: dimethylflormamide
DNP: 2,4-dinitrophenyl
Fm: fluorenylmethyl
Fmoc: fluoroenylmethyloxycarbonyl
For: formyl
HBTU: 2-(1H-benzotriazole-1-yl)-1,1,3,3-tetramethyluronium hexafluorophosphate
cHex: cyclohexyl
HOAT: O-(7-azabenzotriazol-1-yl)-1,1,3,3-tetramethyluronium hexafluorophosphate
HOBt: 1-hydroxy-benzotriazole
MBNA 4-methylbenzhydrylamine
Mmt: 4-methoxytrityl
NMP: N-methylpyrrolidone
O-tBu: oxy-tert-butyl
Pbf: 2,2,4,6,7-pentamethyldihydrobenzofuran-5-sulfonyl
PyBroP bromo-tris-pyrrolidino-phosphonium hexafluorophosphage
tBu: tert-butyl
TIS: triisopropyIsilane
TOS: tosyl
Trt trityl
TFA: trifluoro acetic acide
TFFH: tetramethylfluoroforamidiaium hexafluorophosphate
Z: benzyloxycarbonyl

Unless otherwise indicated, with the exception of the N-terminal amino acid, all abbreviations (e.g. Ala) of amino acids in this disclosure stand for the structure of —NH—C(R)(R′)—CO—, wherein R and R′ each is, independently, hydrogen or the side chain of an amino acid (e.g., R═CH₃ and R′═H for Ala), or R and R′ may be joined to form a ring system.

For the N-terminal amino acid, the abbreviation stands for the structure of:

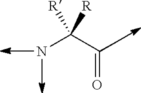

The designation “NH₂” in e.g., Ac-Nle-c(Cys-D-Ala-His-D-Phe-Arg-Trp-Cys)-NH₂ (SEQ ID NO:13), indicates that the C-terminus of the peptide is amidated. Ac-Nle-c(Cys-D-Ala-His-D-Phe-Arg-Trp-Cys) (SEQ ID NO:107), or alternatively Ac-Nle-c(Cys-D-Ala-His-D-Phe-Arg-Trp-Cys)-OH (SEQ ID NO:107), indicates that the C-terminus is the free acid.
“-c(Cys-Cys)-” or “-cyclo(Cys-Cys)-” denotes the structure:

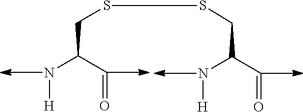

“-c(Cys-Pen)-” or “-cyclo(Cys-Pen)-” denotes the structure:

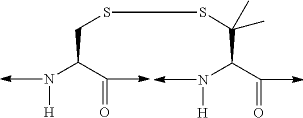

“-c(Asp-Lys)-” or “-cyclo(Asp-Lys)-” denotes the structure:

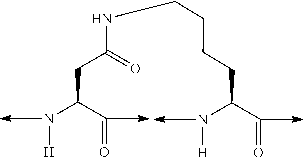 [SMILES: N[C@H](C=O)CCCCNC(=O)C[C@H](N)C=O]

Applicants have devised the following shorthand used in naming the specific embodiments and/or species:

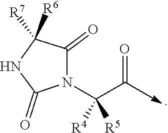

“HydantoinC(O)-(A^{a}-A^{b})” denotes the structure:
wherein amino acid “A^{a}” has the structure:

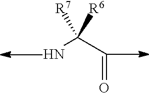

and amino acid “A^{b}” the structure:

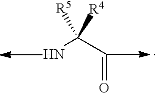

For example, a compound represented as “c[Hydantoin(C(O)-(Cys-A^{b}))-A¹-A²-A³-A⁴-Cys]-” would have the following the structure:

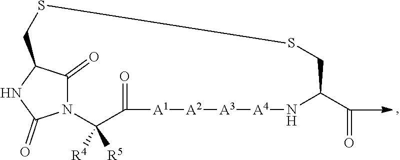

whereas a compound represented as “c[Hydantoin(C(O)-(A^{b}-Cys))-A¹-A²-A³-A⁴-Cys]-” would have the structure:

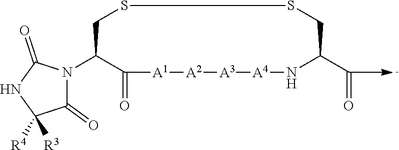

For further guidance, “c[Hydantoin(C(O)-(Asp-A^{b}))-A¹-A²-A³-A⁴-Lys]-” represents the following compound:

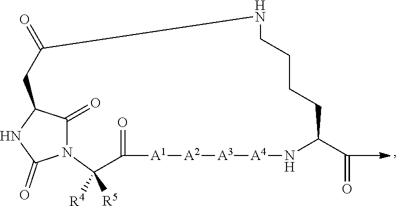

whereas “c[Hydantoin(C(O)-(Dap-A^{b}))-A¹-A²-A³-A⁴-Asp]-” has the following formula:

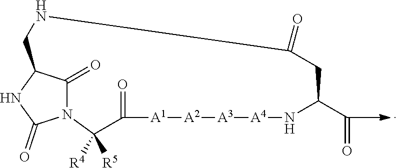

“Acyl” refers to R″-C(O)—, where R″ is H, alkyl, substituted alkyl, heteroalkyl, substituted heteroalkyl, alkenyl, substituted alkenyl, aryl, alkylaryl, or substituted alklyaryl, and is indicated in the general formula of a particular embodiment as “Ac”.

“Alkyl” refers to a hydrocarbon group containing one or more carbon atoms, where multiple carbon atoms if present are joined by single bonds. The alkyl hydrocarbon group may be straight-chain or contain one or more branches or cyclic groups.

“Hydroxyalkyl” refers to an alkyl group wherein one or more hydrogen atoms of the hydrocarbon group are substituted with one or more hydroxy radicals, such as hydroxymethyl, hydroxyethyl, hydroxypropyl, hydroxybutyl, hydroxypentyl, hydroxyhexyl and the like.

“Substituted alkyl” refers to an alkyl wherein one or more hydrogen atoms of the hydrocarbon group are replaced with one or more substituents selected from the group consisting of halogen, (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present. The presence of —(CH₂)₀₋₂₀—COOH results in the production of an alkyl acid. Non-limiting examples of alkyl acids containing, or consisting of, —(CH₂)₀₋₂₀—COOH include 2-norbornane acetic acid, tert-butyric acid, 3-cyclopentyl propionic acid, and the like.

The term “halo” encompasses fluoro, chloro, bromo and iodo.

“Heteroalkyl” refers to an alkyl wherein one of more of the carbon atoms in the hydrocarbon group is replaced with one or more of the following groups: amino, amido, —O—, —S— or carbonyl. In different embodiments 1 or 2 heteroatoms are present.

“Substituted heteroalkyl” refers to a heteroalkyl wherein one or more hydrogen atoms of the hydrocarbon group are replaced with one or more substituents selected from the group consisting of halogen, (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.

“Alkenyl” refers to a hydrocarbon group made up of two or more carbons where one or more carbon-carbon double bonds are present. The alkenyl hydrocarbon group may be straight-chain or contain one or more branches or cyclic groups.

“Substituted alkenyl” refers to an alkenyl wherein one or more hydrogens are replaced with one or more substituents selected from the group consisting of halogen (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.

“Aryl” refers to an optionally substituted aromatic group with at least one ring having a conjugated pi-electron system, containing up to three conjugated or fused ring systems. Aryl includes carbocyclic aryl, heterocyclic aryl and biaryl groups. Preferably, the aryl is a 5- or 6-membered ring. Preferred atoms for a heterocyclic aryl are one or more sulfur, oxygen, and/or nitrogen. Non-limiting examples of aryl include phenyl, 1-naphthyl, 2-naphthyl, indole, quinoline, 2-imidazole, 9-anthracene, and the like. Aryl substituents are selected from the group consisting of —C₁₋₂₀ alkyl, —C₁₋₂₀ alkoxy, halogen (i.e., fluorine, chlorine, bromine, and iodine), —OH, —CN, —SH, —NH₂, —NO₂, —C₁₋₂₀ alkyl substituted with halogens, —CF₃, —OCF₃, and —(CH₂)₀₋₂₀—COH. In different embodiments the aryl contains 0, 1, 2, 3, or 4 substituents.

“Alkylaryl” refers to an “alkyl” joined to an “aryl”.

The term “(C₁₋₁₂)hydrocarbon moiety” encompasses alkyl, alkenyl and alkynyl and in the case of alkenyl and alkynyl there is C₂-C₁₂.

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

The pharmaceutically acceptable salts of the compounds of the invention which contain a basic center are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. Compounds of the invention can also provide pharmaceutically acceptable metal salts, in particular non-toxic alkali and alkaline earth metal salts, with bases. Examples include the sodium, potassium, aluminum, calcium, magnesium, zinc and diethanolamine salts (Berge, S. M. et al., J. Pharm. Sci., 66:1-19 (1977); Gould, P. L., Int'l J. Pharmaceutics, 33:201-17 (1986); and Bighley, L. D. et al., Encyclo. Pharma. Tech., Marcel Dekker Inc, New York, 13:453-97 (1996).

The pharmaceutically acceptable solvates of the compounds of the invention include the hydrates thereof. Also included within the scope of the invention and various salts of the invention are polymorphs thereof. Hereinafter, compounds their pharmaceutically acceptable salts, their solvates or polymorphs, defined in any aspect of the invention (except intermediate compounds in chemical processes) are referred to as “compounds of the invention”.

Designation “(amino acid)ₙ” means that an amino acid is repeated n times. For example, designation “(Pro)₂” or “(Arg)₃” mean that proline or arginine residues are repeated, respectively, two or three times.

MC4R agonists and pharmaceutically acceptable salts thereof described herein can also be used to treat individuals, including human subjects sdefective melanocortin receptor signaling, due to mutations/defects upstream of the MC4R. MC4R agonists and pharmaceutically acceptable salts thereof described herein can also be used to treat individuals, including human subjects that carry mutations in the genes coding for pro-opiomelanocortin (POMC) and leptin such that these mutations result in POMC haplo-insufficiency or haplo-deficiency and/or leptin haplo-insufficiency or haplo-deficiency.

In one example embodiment, an MC4R agonist is a compound represented by structural formula (X):

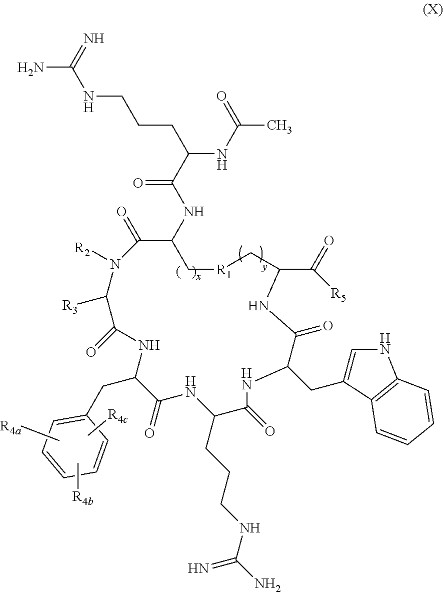

or a pharmaceutically acceptable salt thereof. In structural formula (X), the chemical substituents are defined as follows.
R₁ is —NH—C(O)— or —C(O)—NH—;
R₂ is —H, —CH₂—, or, R₂, together with R₃, forms a pyrrolidine ring optionally substituted with —OH;
R₃ is —(CH₂)₂— if R₂ is —CH₂—, and otherwise R₃ is selected from

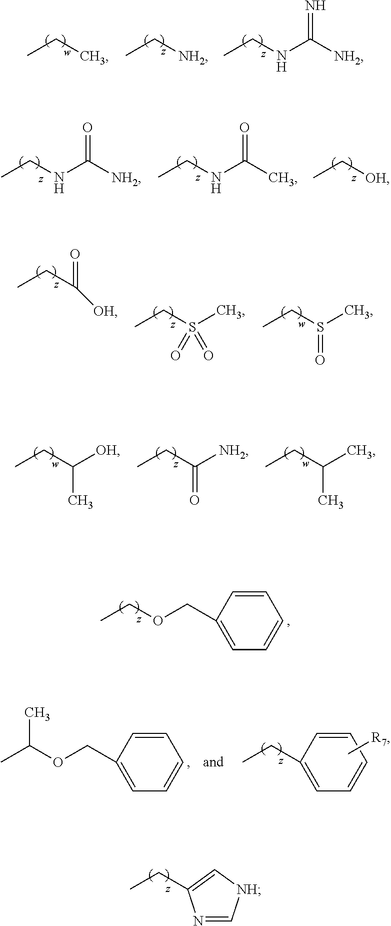

R₄ₐ, R_{4b}, and R_{4c} are each independently selected from hydrogen, halo, (C₁-C₁₀)alkyl-halo, (C₁-C₁₀)alkyl-dihalo, (C₁-C₁₀)alkyl-trihalo, (C₁-C₁₀)alkyl, (C₁-C₁₀)alkoxy, (C₁-C₁₀)alkylthio, aryl, aryloxy, nitro, nitrile, sulfonamide, amino, hydroxyl, carboxy, and alkoxy-carbonyl. In one example embodiment, R₄ₐ, R_{4b}, and R_{4c} is not hydrogen.
R₅ is —OH or —N(R₆ₐ)(R_{6b});
R₆ₐ and R_{6b} are each independently H or C₁ to C₄ linear, branched or cyclic alkyl chain;
R₇ is —H or —C(O)—NH₂;
w is in each instance independently 0 to 5;
x is 1 to 5;
y is 1 to 5;
z is in each instance independently 1 to 5.

An example of a compound of structural formula (X) is a cyclic peptide defined by structural formula (XI):

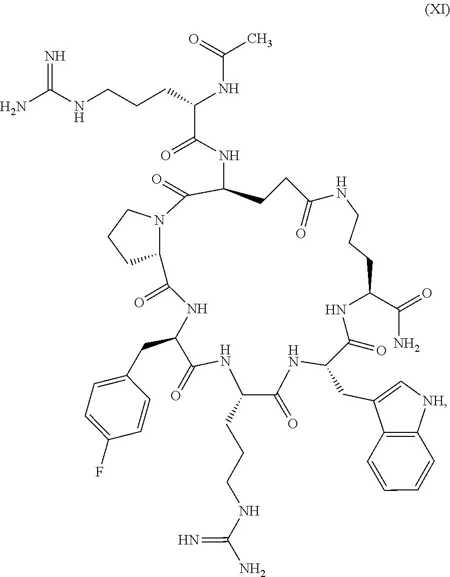 [SMILES: CC(=O)N[C@@H](CCCNC(=N)N)C(=O)N[C@H]1CCC(=O)NCCC[C@@H](C(N)=O)NC(=O)[C@H](Cc2c[nH]c3ccccc23)NC(=O)[C@H](CCCNC(=N)N)NC(=O)[C@@H](Cc2ccc(F)cc2)NC(=O)[C@@H]2CCCN2C1=O]

or a pharmaceutically acceptable salt thereof.

### EXEMPLIFICATION

### Example 1: Model for Evaluating Whether Obese MC4R+/−Heterozygotic Mice are Responsive to Treatment with Compound of SEQ ID NO: 140

The effect of MC4R agonist administration on a subject can be evaluated according to the following procedure.

The effects of a MC4 agonist in heterozygous MC4+/− mice and in weight-matched diet-induced-obesity (DIO) mice is evaluated. Heterozygous MC4+/− mice express a mild hyperphagic and obese phenotype when compared to the homozygous MC4−/− mice while retaining a putative response to MC4 stimulation. Weight-matched DIO mice are expressing the MC4 receptor (wild-type). In the course of the study, the effect of MC4 agonism on food intake and body weight in mice that are phenotypically obese but differ genetically in terms of the expression of the MC4 receptor is being characterized.

Pre-Study activities: C57BL/6 mice (N=50, males, 4 weeks of age) are pre-fed a high fat (HF) diet, commercially available from Research Diets Inc, New Brunswick, NJ, for 10 weeks prior to enrollment onto study. The HF diet (D12492) is fed to the animals ad libitum.

Species (number, sex, age/weight): C57BL/6 mice (N=40, males, 14 weeks of age at initiation of dose administration). Study criteria for animal enrollment based on body weight. B6-129/S-MC4+/− heterozygous mice (Jackson Labs or Taconic; N=40, males, body weight matching the DIO mice, 12-14 weeks of age).

Formulations: all test materials are formulated once weekly.

Treatment: All animals are surgically implanted with a subcutaneous osmotic minipump (infusion duration of 14 days).

The design of this study is symmarized in Table A:

TABLE A
Study Design:

| | | | | Dose Level | | Obser- |
|---|---|---|---|---|---|---|
| Group | | Animals | Treat- | and | Treatment | vation |
| No. | Mice | per Group | ment | Volume | Regimen | Period |
| 1 | MC4^{+/-} | 10 | Vehicle | 0 | Chronic | 14 |
| 2 | DIO | 10 | | | Constant | days |
| 3 | MC4^{+/-} | 10 | Peptide | Low | Infusion by | |
| 4 | DIO | 10 | drug | | Osmotic | |
| 5 | MC4^{+/-} | 10 | | Mid | Minipump | |
| 6 | DIO | 10 | | | (Option 2) | |
| 7 | MC4^{+/-} | 10 | | High | | |
| 8 | DIO | 10 | | | | |

Cage side and clinical observations are performed daily, clinical observations are noted per exception. Food intake by mice is permitted daily. Body Weights: All animals have body weights measured once weekly during the pre-feed and twice weekly during administration, initiating prior to the initial dose administration. Doses are based on most recently collected body weight.

Fasting Whole Blood Glucose Levels and Plasma Sample Collection: Following an overnight fast, all animals have a fasting whole blood glucose level (via glucometer) and blood sample collected (˜100 L) on Days −1 and 14.

Euthanasia and Tissue Collection: All animals are scheduled for euthanasia on Day 15 in the AM. All animals have a maximum terminal blood collection made. Blood samples are processed for plasma for insulin measurement. All animals have the retroperitoneal adipose tissue and liver excised and weighed.

Insulin measurement: Insulin levels are determined in terminal plasma samples using a mouse insulin ELISA assay by the testing facility.

Reporting: Data submission including clinical observations, food intakes, body weights, insulin levels, fasting blood glucose and plasma collections, mortality record (if applicable), the study protocol and associated amendments, and all protocol deviations.

### Example 2: Models for Clinical Evaluation of the Efficacy of Treatment of MC4R-Mediated Obesity Using Compounds Disclosed Herein

### 1. Randomized, Double-Blind, Placebo-Controlled, Multiple Ascending Dose Study to Evaluate the Safety, Tolerability, Pharmacokinetics and Pharmacodynamics of Compounds of the Present Invention Administered to Healthy Obese Non-Diabetic Volunteers

### Objectives

### Primary:

Investigate the safety and tolerability of multiple dose levels of the compounds of the present invention when given by continuous subcutaneous (SC) infusion for 14 or 28 days.

### Secondary:

Evaluate the pharmacokinetics (PK) of multiple dose levels of the compounds of the present invention when given by SC continuous infusion for 14 or 28 days. Exploratory pharmacodynamic (PD) objectives of this study are to evaluate the effect of multiple dose levels of the compounds of the present invention when given by SC continuous infusion for 14 or 28 days on:
Caloric intake, weight and waist circumference.
Insulin sensitivity as measured by a Meal Tolerance Test (MTT).
Hunger and satiety as measured by a Hunger/Satiety Questionnaire.
Resting energy expenditure (REE) as measured by indirect calorimetry (to be performed at select centers with this capability).

### Study Design

The study is designed to evaluate safety and tolerability of the compounds of the present invention administered up to 1 mg/kg/day for 14 or 28 days as a SC continuous infusion. The highest dose proposed to use in the study is no higher than 1 mg/kg. This is a randomized, double-blind, placebo-controlled, multiple ascending dose study during which 5 sequential cohorts of subjects will receive the compounds of the present invention or placebo by SC continuous infusion for 14 (Cohorts dosed for 14 days) or 28 days (Cohorts dosed for 28 days). Nine subjects will be enrolled in each cohort and subjects will be randomly allocated to receive the compounds of the present invention or placebo in a 6:3 ratio.

All subjects will remain confined to the Phase 1 clinical unit during treatment and under observation for at least 24 hours after the end of the study drug infusion.

A Clinical Safety Committee (CSC) will review blinded interim safety data from each dose level. Dose escalation will be recommended only if the previous dose level was deemed to be safe and well tolerated. Where appropriate, for safety reasons, additional interim dose levels (lower than the next scheduled dose) may be administered. Additionally, a sub-set of the general obese population may be enrolled. These subjects will meet all inclusion and exclusion criteria outlined in below, as well as one additional criterion: subjects must be heterozygous with a loss-of-function mutation in one of their two copies of the MC4 receptor gene. These subjects will have been pre-identified as having an MC4 receptor mutation. The rationale for this cohort is the lesser MC4 tone that is seen in heterozygous subjects, may give an altered sensitivity for these subjects to MC4 agonists such as the compounds of the present invention. If this cohort is enrolled, it is anticipated to be at a select site, nearer the end of the study.

### Number of Subjects Planned

A sufficient number of healthy obese adult male and female subjects will be screened so that approximately 45 eligible subjects qualify for the study and are randomized. It is expected that approximately 45 subjects will be enrolled in approximately 5 dose groups to evaluate multiple days of dosing (14 or 28 days) of the compounds of the present invention administered by SC continuous infusion. Up to an additional 63 subjects may be enrolled to further characterize the compounds of the present invention with a maximum of approximately 108 subjects planned for treatment in the study. The additional subjects will be recruited in the event a subject needs to be replaced, a cohort is to be expanded or an intermediate dose is recommended by the CSC. It is intended that most cohorts will consist of 9 subjects (in a ratio of 2 active:1 placebo). However some cohorts may be increased in order to enhance the sample size and further define any prior findings.

### Diagnosis and Main Criteria for Inclusion

Subjects must meet all of the following inclusion criteria to be eligible for the study.

Inclusion criteria
Able to provide voluntary, written informed consent with comprehension of all aspects of the protocol, prior to any study procedures.
Healthy obese male and female volunteers aged 18 to 55 years, inclusive.
In good general health, without significant medical history, physical examination findings, or clinical laboratory abnormalities.
Body Mass Index of 30-40 kg/m², inclusive.
Stable body weight during the previous 6 months, based on Investigator judgment.
Blood pressure <140/90 mmHg at Screening and D-1. Measurement may be repeated once within 24 hours, based on Investigator judgment.
Females must not be pregnant and must have a negative serum pregnancy test result at the Screening Visit and Day −1.
Females of childbearing potential must agree to be abstinent or else use any two of the following medically acceptable forms of contraception from the Screening Period through the Final Study Visit: hormonal, condom with spermicidal jelly, diaphragm or cervical cap with spermicidal jelly, or IUD. Hormonal contraception must have started at least 3 months prior to screening. A female whose male partner has had a vasectomy must agree to use one additional form of medically acceptable contraception. Subjects must agree to practice the above birth control methods for 30 days from the final visit as a safety precaution.
Females of non-childbearing potential, defined as surgically sterile (status post hysterectomy, bilateral oophorectomy, or bilateral tubal ligation) or post-menopausal for at least 12 months (and confirmed with a screening FSH level in the post-menopausal range), do not require contraception during the study.
Males with female partners of childbearing potential must agree to use two medically acceptable forms of contraception as described above, with one of the two forms being condom with spermicide, from the Screening Period through the Final Study Visit. Males with female partners of childbearing potential who themselves are surgically sterile (status post vasectomy) must agree to use condoms with spermicide over the same period of time. Male subjects must agree to practice the above birth control methods for 30 days from the final visit as a safety precaution.

### Additional Inclusion Criteria for Heterozygous MC4 Receptor Mutation Cohort:

Mutation of MC4R gene resulting in partial loss or complete loss of function of one of the MC4 receptor genes.

### Exclusion Criteria

Fasting blood glucose >126 mg/dL at screening.
Resting heart rate <45 bpm or >90 bpm at screening.
Abnormal thyroid stimulating hormone (TSH) or thyroxine (T₄) levels on screening.
Elevated ALT or serum creatinine on screening or any clinically significant abnormalities on screening laboratory tests as determined by the Investigator.
History of diabetes or of treated or medically diagnosed hypertension.
Presence of a skin lesion suspicious for malignancy.
History of malignancy except for treated cervical carcinoma in situ in the past 5 years.
Active or history of any clinically significant medical condition including renal, hepatic, pulmonary, gastrointestinal, cardiovascular, genitourinary, endocrine, immunologic, metabolic, neurologic, psychiatric or hematological disease, based on Investigator judgment.
Acute illness or history of illness, which in the opinion of the Investigator, could pose a threat or harm to the subject or obscure interpretation of laboratory test results or interpretation of study data.
Positive hepatitis B surface antigen, positive hepatitis C antibody or positive HIV test at screening or a history of positive testing (e.g. liver biopsy, serology) suggesting acute or chronic hepatitis.
Abnormal 12-lead electrocardiogram (ECG) at screening or pre-dose (Day −1 or Day 1), except minor deviations deemed to be of no clinical significance by the Investigator.
Received any experimental drugs or devices within 30 days or 5 half lives, whichever is longer, prior to dosing.
Ongoing participation in a prior clinical study at the time of screening.
Blood donation within 60 days prior to screening or intent to donate within 60 days after Final Study Visit.
Hospitalization for major surgery including but not limited to abdominal, thoracic, or cardiovascular surgery within the past 3 months prior to screening, or for a clinically significant non-surgical illness, based on Investigator judgment, within the past 3 months.
Planned elective surgery within 30 days of the Final Study Visit.
Poor venous access or inability to tolerate venipuncture.
History of drug hypersensitivity or anaphylaxis.
History of hypersensitivity to proteins (e.g., allergy shots).
Use of prescription medications on a regular basis. The last use of any prescription medication must have been greater than 5 half-lives for the specific medication or at least 14 days prior to admission (Day −1), whichever is longer. Hormonal contraception is allowed for female subjects.
Use of a non-prescription drug and herbal substances during the study (through the Final Study Visit). The last dose of any non-prescription drug must have been taken greater than 5 half-lives for that drug before receiving study drug.
Inability to attend all study visits or to comply with protocol requirements including fasting and restrictions on alcohol, caffeine, nicotine and concomitant medication intake.
A significant history of drug/solvent abuse within 5 years of screening or a positive test for drugs of abuse test at screening or on Day −1.
Positive alcohol (breath test) or nicotine screen at Screening Visit or Day −1.
History of alcohol abuse (defined as average intake of three or more units of alcohol per day) within 5 years of the Screening Visit.
History of tobacco or tobacco product use unless abstinent for at least one year prior to the Screening Visit.
Previously randomized and dosed in this study.
Any other reason, which in the opinion of the Investigator would confound proper evaluation of the study.

### Test Products, Doses, and Mode of Administration

The compounds of the present invention and the placebo are formulated for administration by SC continuous infusion using an infusion pump.

The 5 dose levels planned, in ascending order, are:
0.01 mg/kg/24 hrs
0.1 mg/kg/24 hrs
0.25 mg/kg/24 hrs
0.5 mg/kg/24 hrs
1.0 mg/kg/24 hrs

The compounds of the present invention or placebo will be given by SC continuous infusion for 14 or 28 days. The dose levels evaluated may be modified based upon data from the single ascending dose study, or the prior MAD cohort.

### Duration of Treatment

Overall study duration will be approximately 7 months. Individual subject participation in the study (screening, dosing, post-dosing assessments, follow-up) will be approximately 72 and 86 days for Cohorts dosed for 14 days and dosed for 28 days respectively.

The study will consist of a Screening Period, a Treatment Period and a Follow-up Period. The Screening Period will occur within 30 days prior to enrollment. The Treatment Period will consist of administration of a SC continuous infusion initiated on Day 1 and completed on Day 15 or Day 29 for Cohorts dosed for 14 days and Cohorts dosed for 28 days, respectively. Subjects will remain confined in the clinical research center (CRC) for approximately 24 hours following completion of the infusion and will be discharged from the CRC on Day 16 or 30 after all study procedures have been completed. Follow-up study visits are scheduled 1 and 4 weeks after the end of the study drug infusion.

### Study Procedures

The procedures for each study period are briefly outlined below and are depicted in detail in the Schedule of Assessments (SOA).

### Screening Period (Days −30 to −1)

After informed consent is obtained and eligibility assessed, screening assessments will be performed including: medical history; pregnancy test (all females); drug, nicotine and alcohol screen; safety laboratory tests (including clinical chemistry, hematology and urinalysis), HbA1c and fructosamine, full physical examination (including weight, waist circumference and height), comprehensive skin examination performed by a Dermatologist, vital signs (including supine systolic and diastolic blood pressure, pulse rate, respiratory rate and body temperature); 12-lead electrocardiogram (ECG); HBsAg, HCV-Ab, HIV screening; samples of antibodies against the compounds of the present invention; Fitzpatrick scale; dietary recall review, indirect calorimetry (within 3 days of Day 1); previous and concomitant medication use.

### Treatment Period

Subjects will be admitted to the research unit on Day −1. After continued eligibility is confirmed, the following assessments will be performed: abbreviated physical exams including weight and waist circumference; vital signs; 12 lead ECG; safety laboratory tests (including clinical chemistry, hematology and urinalysis); lipid profile; level of antibodies against the compounds of the present invention; serum sample for storage; quantitative skin color measurement; photographic skin evaluation; Hunger/Satiety questionnaire; initiation of cardiac telemetry and ambulatory blood pressure (ABPM) monitoring; sample collection for 24 hour urine catecholamine and cortisol level determination; estimated caloric intake; Meal Tolerance Test (MTT), randomization; monitoring for AEs and concomitant medications.

Upon initiation of study treatment on Day 1, the following assessments will be performed on Days 1-16 (Cohorts dosed for 14 days) or Days 1-29 (Cohorts dosed for 28 days) according to the SOA: abbreviated physical exam including weight and waist circumference; vital signs; cardiac telemetry, ABPM, 12-lead ECG, safety laboratory tests; lipid profile; sample collection for 24 hour urine catecholamine and cortisol level determination; sample collection for plasma free metanephrine levels, PK blood and urine sampling, melanocortin receptor genotyping; infusion site evaluation; quantitative skin color measurement; photographic skin evaluation; estimated caloric intake; MTT; HbA1c and fructosamine; Hunger/Satiety questionnaire; indirect calorimetry, monitoring for AEs and concomitant medications. Prior to discharge from the research unit, a serum pregnancy test will be performed on all females, and a comprehensive skin evaluation will be performed by a Dermatologist.

### Follow-Up Period

One and 4 weeks after completion of the study treatment infusion, subjects will return to the research unit for the following assessments: complete physical exam including weight and waist circumference; comprehensive skin exam performed by a Dermatologist, quantitative skin color measurement; photographic skin evaluation; infusion site evaluation; vital signs; safety laboratory tests; lipid profile; HbA1c and fructosamine; levels of antibodies against the compounds of the present invention; Hunger/Satiety questionnaire, monitoring for AEs and concomitant medications.

### Study Endpoints

### Safety

Safety will be evaluated by assessment of adverse events, ECGs, cardiac telemetry, ambulatory blood pressure monitoring, clinical laboratory evaluations (hematology, clinical chemistry including fasting blood glucose levels and urinalysis), lipid profile; levels of antibodies against the compounds of the present invention, urinary catecholamine levels, urinary free cortisol levels, plasma free metanephrine levels, vital signs (including blood pressure, respiratory rate, heart rate, and body temperature), physical examinations including infusion site evaluations and concomitant medication review.

### Pharmacokinetic

Serial blood sampling and urine collections for measurement of plasma and urinary levels of the compounds of the present invention will be conducted. All samples will be assayed for the compounds of the present invention from which the following PK parameters will be computed for each subject: AUC_{0-τ}, Cₐᵥₑ, Cₘₐₓ, Tₘₐₓ, λz, T_{1/2}, CL/F, Vz/F, accumulation ratios, total urinary excretion and renal clearance.

### Pharmacodynamic

Caloric intake, weight and waist circumference, insulin sensitivity (as measured by MTT), hunger and satiety (using a Hunger/Satiety Questionnaire) and REE (using indirect calorimetry) will be assessed as exploratory PD endpoints. HbAc1 and fructosamine levels will also be assessed.

### Sample Size Determination

The sample size for this Phase 1 first multiple-dose study in humans was not based on formal statistical determinations. The sample size for this study was chosen in consideration of limiting exposure to this new compound while providing information to evaluate the safety and effect of the compounds of the present invention in a Phase 1 first multiple-dose study.

### Statistical Methods

Continuous variables will be summarized by dose (all placebo pooled) with descriptive statistics (number of observations, mean, SD, median, maximum, and minimum). Categorical variables will be tabulated by frequency of subjects by dose (all placebo pooled) and for the active treatment doses combined. The PD endpoints may be analyzed via analysis of variance if appropriate. All subject information and safety measurements will be based on the Safety Population.

### CSC Data Review and Stopping Rules

The study design is such that successively higher doses will be administered to different groups of subjects after the safety and tolerability of the preceding dose has been established. Dose escalation recommendations are to be made by the CSC based upon a review of clinical safety data through Day 16 (Cohorts dosed for 14 days) or Day 30 (Cohorts dosed for 28 days).

### Rules for Suspension of Dosing for a Subject:

An increase in SBP, sustained for a minimum of 30 minutes, either to >35 mmHg above mean baseline pre-dose SBP, or to >165 mmHg;
An increase in DBP, sustained for a minimum of 30 minutes, either to >20 mmHg above mean baseline pre-dose DBP, or to >100 mmHg;
Any increase in BP that is judged to be symptomatic, per the Investigator, regardless of duration;
An increase in HR, sustained for a minimum of 30 minutes (or less in the judgment of the Investigator), to >35 bpm above mean baseline pre-dose HR;
A prolonged spontaneous erection lasting more than 60 minutes, or a spontaneous painful erection of any duration based on Investigator judgment;
Any other treatment-emergent AE that in the judgment of the Investigator poses a significant safety risk for that subject in the context of continued infusion of study drug.

### Rules for Suspension or Termination of Dose Escalation:

An SAE that is deemed by the Investigator to be possibly or probably related to study drug occurs in any subject treated with the compounds of the present invention;
A CTCAE Grade 3 (severe treatment emergent AE) or higher that is possibly or probably related occurs in a subject treated with the compounds of the present invention;
A possibly or probably related treatment emergent AE not listed by the CTCAE occurs in a subject treated with the compounds of the present invention that is graded as severe or life threatening.

The CSC may also recommend suspension of the compounds of the present invention dose escalation based upon other conditions as deemed medically appropriate.

### Rules for Suspension of Further Dosing:

The study may be immediately suspended and no additional doses administered if one or more subjects at any dose level develop any of the following adverse events deemed to be possibly or probably attributable to study drug:
Anaphylaxis (i.e., angioedema, hypotension, bronchospasm, hypoxia or respiratory distress) in a subject treated with the compounds of the present invention;
Any clinically significant treatment-related AE that poses an undue risk to subjects in the opinion of the CSC.
   2. Randomized, Double-Blind, Placebo-Controlled Study to Evaluate the Safety and Efficacy of the Compound of the Present Invention Inpatients with Obesity Due to an MC4R Mutation

### Objectives

The primary objective is to assess the effect of the compounds of the present invention vs. placebo on mean percent body weight loss when administered for 90 days by continuous SC infusion.

### Secondary Objectives are to Assess:

The mean body weight (BW) loss in the active treatment group compared to the placebo group from baseline to Day 90.
The proportion of patients who lose ≥5% of their baseline body weight in the active treatment group compared to the placebo group from baseline to Day 90.
The pharmacokinetics (PK) of the compounds of the present invention when given by continuous SC infusion for 90 days.
The safety and tolerability of the compounds of the present invention when given by continuous SC infusion for 90 days.
The effect of the compounds of the present invention versus placebo on ambulatory blood pressure monitoring parameters (ABPM) when given by continuous SC infusion for 90 days (sub-study).
The mean percent weight loss, mean weight loss, and proportion of patients who lose ≥5% of their baseline body weight in the active treatment group compared to the placebo group from baseline to Day 90 in patients who are severely obese (e.g., BMI≥40 Kg/m²; sub-study).

Exploratory pharmacodynamic objectives of this study are to evaluate the effect of the compounds of the present invention when given by SC continuous infusion for 90 days in all patients, and those in the severely obese sub-study, on:
The proportion of patients who achieve a >10% decrease in body weight in the active treatment group compared to placebo from baseline to Day 90.
The change in glucose and insulin during a Meal Tolerance Test (MTT) from Baseline to Day 90.
Change in fasting glucose, insulin, insulin sensitivity, triglycerides, cholesterol, HDL, LDL, hs-CRP and HbA1c from Baseline to Day 90.
Change in waist circumference from Baseline to Day 90.
Change in body composition (assessed by Dual Energy X-Ray Absorptiometry (DXA)) from Baseline to Day 90 (sub-study).
Change in hunger and satiety from Baseline to Day 90.
Change in Impact of Weight on Quality of Life—Lite questionnaire (IWQOL-Lite) total score from Baseline to Day 90.
Change in depression/suicidality score (assessed by PHQ-9 and C—SSRS) from Baseline to Day 90.
Change in skin pigmentation (assessed by mexameter) from Baseline to Day 90.

### Study Design

This is a randomized, double-blind, placebo-controlled study designed to evaluate the efficacy and safety of the compounds of the present invention when administered for 90 days to obese patients, inclusive of a sub-set of patients who are severely obese (BMI≥40 Kg/m²).

Patients who are obese (BMI between 35-50 Kg/m²), but otherwise healthy, will be enrolled. This study will be conducted on an outpatient basis. All patients will be required to self-administer study drug via an approved insulin infusion pump (OmniPod®) during the ˜7 day placebo practice period. Patients with continued eligibility who have demonstrated the ability to successfully manage self-administration of placebo during the practice period will be randomized to the double blind 90 day Treatment Period.

### Number of Patients Planned

Approximately 70 patients will be enrolled into the study. There will be three sub-studies within the protocol. The first will include those patients who are severely obese (BMI≥40 Kg/m², who will be stratified separately). Approximately 20 severely obese patients will be enrolled into this sub-study; these subjects will be recruited at all sites. The two remaining sub-studies will be enrolled at select sites. The first will be an ABPM sub-study where approximately 30 patients will be enrolled, and the final sub-study will include DXA scans on approximately 20 patients.

### Diagnosis and Main Criteria for Inclusion

Patients must meet all of the following inclusion criteria to be eligible for the study:
1. MC4R heterozygous patients: mutation of MC4R gene.
2. Be between the age of 18 and 65.
3. Able to provide voluntary, written informed consent with comprehension of all aspects of the protocol, prior to any study procedures.
4. In good general health, without significant medical history, physical examination findings, or clinical laboratory abnormalities.
5. Body Mass Index: 35-50 Kg/m², inclusive. It is planned that approximately of these patients will have a BMI≥40 Kg/m².
6. Stable body weight (+/−5 Kg) during previous 6 months.
7. Blood pressure (<140/90 mmHg); may include stable dose (≥30 days of use) of up to two anti-hypertensive medications to achieve control that are intended to remain on a stable dose during the protocol.
8. Willingness and demonstrates ability to self administer study medication subcutaneously via a continuous infusion pump during the placebo practice period.
9. Willing to maintain a healthy diet and exercise regime throughout study as recommended by counseling at study start.
10. Females of childbearing potential must agree to be abstinent or else use any two of the following medically acceptable forms of contraception from the Screening Period through the completion of study treatment: hormonal, condom with spermicidal jelly, diaphragm or cervical cap with spermicidal jelly, or IUD. Hormonal contraception must have started at least 3 months prior to screening. A female whose male partner has had a vasectomy must agree to use one additional form of medically acceptable contraception. Patients must agree to practice the above birth control methods for 30 days after completion of study treatment as a safety precaution.
11. Females of non-childbearing potential, defined as surgically sterile (status post hysterectomy, bilateral oophorectomy, or bilateral tubal ligation) or post-menopausal for at least 12 months (and confirmed with a screening FSH level in the post-menopausal range), do not require contraception during the study.
12. Males with female partners of childbearing potential must agree to use two medically acceptable forms of contraception as described above, with one of the two forms being condom with spermicide, from the Screening Period through 90 days after completion of study treatment. Males with female partners of childbearing potential who themselves are surgically sterile (status post vasectomy) must agree to use condoms with spermicide over the same period of time.

If any of the following exclusion criteria are met, the patient is not eligible for the study:
1. Fasting blood glucose greater than 140 mg/dL.
2. HbA1c≥6.5%.
3. TSH level outside the normal range.
4. Creatinine >1.5 times the upper limit of normal.
5. Liver function tests >2 times the upper limit of normal.
6. Active or history of any significant medical condition including renal, hepatic, pulmonary, gastrointestinal, cardiovascular, genitourinary, endocrine, immunologic, metabolic, neurologic or hematological disease.
7. Patients with a history of the following:
   a. Uncontrolled hypertension;
   b. Diabetes requiring medical treatment, presently or in the past;
   c. Major depressive disorder within the last 2 years;
   d. Any lifetime history of a suicide attempt;
   e. Any suicidal behavior in the last month;
   f. Other severe psychiatric disorders (e.g. schizophrenia, bipolar disorder, severe eating disorders including bulimia).
8. A PHQ-9 score of ≥15.
9. Any suicidal ideation of type 4 or 5 on the C—SSRS.
10. Prior bariatric surgery.
11. History or close family history (parents or siblings) of melanoma.
12. Significant dermatologic findings as part of the Screening comprehensive skin evaluation performed by the dermatologist. Any concerning lesions identified during the screening period will be biopsied and results known to be benign prior to randomization. If the pre-treatment biopsy results are of concern, the patient will be excluded from the study.
13. Treated with anorectic agents or drugs with anorexia as a frequent side event.
14. Taking 3 or more anti-hypertensive medications.
15. Acute illness or history of illness, which in the opinion of the Investigator, could pose a threat or harm to the patient or obscure interpretation of laboratory test results or interpretation of study data.
16. History of any malignancy, past or present, including skin cancer, multiple severely dysplastic nevi, or nevoid basal cell carcinoma.
17. History of HIV infection.
18. History of significant drug hypersensitivity or anaphylaxis.
19. History of hypersensitivity to proteins (e.g., allergy shots).
20. Any clinically significant abnormalities on screening laboratories as determined by the Investigator.
21. Abnormal 12-lead electrocardiogram (ECG) at screening or pre-dose (Day 1), except minor deviations deemed to be of no clinical significance by the Investigator. QTc must be <450 ms.
22. Received any experimental drugs or devices or have participated in a clinical study within 30 days prior to dosing.
23. Blood donation within 60 days prior to screening or intent to donate up to 60 days after Final Study Visit.
24. Hospitalization for surgery within the 3 months prior to screening except for minor outpatient procedures, or any planned hospitalizations during the study period.
25. Poor venous access or inability to tolerate venipuncture.
26. Inability to attend all study visits or comply with protocol requirements including fasting and restrictions on concomitant medication intake.
27. Participation in weight loss programs during the study period, including nutritional supplements/replacements other than as recommended by nutritional counseling provided at study start.
28. Use of prescription medications on a regular basis with the following exceptions:
   a. Contraceptives (must be on for ≥3 months);
   b. Hormone replacement therapy (must be on stable dose for ≥3 months);
   c. Antihypertensives (<3 medications on a stable dose for ≥30 days);
   d. Statins (dose must be ≤half the maximum dose; must be on a stable dose ≥3 months);
   e. Fibrates (must be on stable dose for ≥3 months);
   f. Niacin (must be on stable dose for ≥3 months);
   g. Thyroxin (stable dose for >30 days);
   h. The last use of any other prescription medication must have been greater than 5 half-lives for the specific medication or at least 14 days prior to randomization, whichever is longer.
29. Women who are pregnant or are breast feeding.
30. Previously randomized and dosed in this study or previously exposed to the compounds of the present invention.
31. History of alcohol or drug abuse within 5 years of Screening Visit.
32. Any other reason, which in the opinion of the Investigator would confound proper evaluation of the study.

### Test Products, Doses, and Mode of Administration

The compounds of the present invention will be supplied as sterile solutions for infusion. The product will be manufactured at a concentration of 2.0 mg/mL at pH 5 with a fill volume of 11 mL/vial. Placebo will be vehicle. Drug products and placebo consist of sodium phosphate and citric acid, including 0.5% phenol as a preservative. Both the compounds of the present invention and placebo multiuse vials may be punctured multiple times under sterile conditions. The compounds of the present invention and placebo will be administered as a continuous subcutaneous infusion using the FDA approved insulin infusion pump, Insulet's OmniPod® (infusion pump which is wireless/tubeless and does not require a traditional infusion set, inclusive of an auto-injector whereby the patient never sees the needle or cannula). A total daily dose of 1 mg/24 hours of the compounds of the present invention, or equivalent volume of placebo, will be self-administered via continuous SC infusion during the treatment period.

### Duration of Treatment

The overall study duration will be approximately 9 months, as currently planned. Individual patient participation in the study (Screening Period, Treatment Period and Follow-up Period) will be approximately 7 months. Screening, inclusive of the placebo practice period, will occur within 30 days prior to randomization. Patients who successfully complete the open label placebo practice period will be randomized to double blind treatment for 90 days. The Final Visit will occur approximately 90 days after the last dose of study drug is administered (Day 180).

### Study Procedures

The study will consist of a Screening Period inclusive of 2 visits. Patients who demonstrate compliance with the continuous infusion will be randomized to a double-blind treatment regimen (at Visit 3) and will begin 90 days of double-blind, self-administered SC continuous infusion, outpatient treatment. Additional clinic visits are scheduled on approximately Day 7 (Visit 4), Day 14 (Visit 5), Day 28 (Visit 6), Day 56 (Visit 7) and at the end of treatment (Day 90, Visit 8). Patients will also be contacted by telephone weekly during the first month of treatment, followed by bi-weekly contact during the remaining Treatment Period to encourage compliance and to assess adverse events. Follow up Visits will be scheduled monthly for 3 months after completion of the 90-day Treatment Period. The Final Visit will occur ˜90 days after the last dose of study drug is administered (Day 180, Visit 11).

### Screening Period (Days −30 to −1)

The Screening Period consists of 2 visits; the first where patients will be assessed for study qualification. Eligible patients will then proceed onto the second screening visit which will consist of an open label placebo practice period to ensure study patients can self-administer placebo drug via an FDA approved SC insulin infusion pump for approximately 1 week.

### Visit 1

During Visit 1, following signed, written informed consent, confirmation of eligibility will be performed. Medical history, physical examination (including vital signs, height and weight and waist circumference measurements), a comprehensive skin exam will be conducted by the Dermatologist, quantitative skin measurement, Fitzpatrick scale and Edmond Obesity Staging System (EOSS) assessments, concomitant medication review, clinical laboratory tests including HbA1c, serum pregnancy test or follicle-stimulating hormone test, and a 12-lead ECG will be performed at this visit. The PHQ-9 and C—SSRS will be administered. Hunger and satiety questionnaire will also be administered.

### Visit 2

During visit 2, patients confirmed to be eligible at Visit 1 and who continue to meet the inclusion and exclusion criteria upon review of medical history since the prior visit as well as AE and concomitant medication review, will have their weight and waist circumference measured and vital signs measured. Study staff will train patients and instruct them on proper technique of how to use the OmniPod® at this visit. Patients will be required to demonstrate understanding by successfully filling the OmniPod® with placebo, successfully placing the pod on an appropriate body area, and starting the infusion while at site. The study patients will change the OmniPod® approximately 2-3 times during the ˜7 day period between Visits 2 and 3.

For those patients participating in the ABPM sub-study, an additional clinic visit will be necessary.

### Treatment Period (Days 1-90)

Patients will return to the clinic approximately 7 days after starting the placebo practice period. Study patients who successfully complete the open label placebo practice period will return for Visit 3 (Day 1), and be randomized to 90 days of double-blind study treatment. Additional clinic visits are scheduled on approximately Day 7 (Visit 4), Day 14 (Visit 5), Day 28 (Visit 6), Day 56 (Visit 7) and at the end of treatment (Day 90, Visit 8). During these visits, a variety of efficacy, safety and exploratory assessments will be performed, according to the SOA.

Efficacy will be evaluated by measuring body weight. Safety will be evaluated by assessment of adverse events, vital signs (including blood pressure, respiratory rate, heart rate, and body temperature), ECGs, ABPM (sub-study), clinical laboratory evaluations (hematology, clinical chemistry including fasting blood glucose and insulin levels and urinalysis), lipid profile; levels of antibodies against the compounds of the present invention, quantitative skin assessments (mexameter) and photographic skin evaluation, protocol defined pigmented skin lesion biopsies, physical examinations including infusion site evaluations and concomitant medication review. Additionally, changes in depression/suicidality as assessed by the C—SSRS and PHQ-9 will be monitored. Plasma concentrations of the compounds of the present invention will be summarized and may be compared to PD parameters.

Exploratory measurements will be assessed by insulin sensitivity (as measured by MTT and HOMA-IR), effects on Hs—CRP and HbA1c, hunger and satiety (using a Hunger/Satiety Questionnaire), body composition (using DXA at select sites), changes in waist circumference, and changes in IWQOL-Lite, PHQ-9 and C—SSRS will be assessed as exploratory endpoints.

For patients who do not complete the full 90 day treatment period, attempts will be made to have the patient return for continued follow-up visits in order to monitor patient safety, as well as any effects on pharmacodynamic assessments.

### Follow-up Period (Days 91-180)

Upon completion of the 90 day Treatment Period, Patients will enter a 90 day post-treatment Follow-up Period consisting of 3 monthly visits, where a variety of safety and efficacy assessments according to the SOA. The Final Study Visit will occur on approximately Day 180.

In the event an AE is ongoing at the time of the Final Visit, additional visits should be scheduled, at a frequency deemed appropriate by the Investigator, in order to follow the event to resolution. If a patient experiences a Serious Adverse Event for which follow-up laboratories and review are required, the Investigator will schedule additional post-treatment visits as necessary.

### Study Endpoints

The primary endpoint will be evaluated by assessment of mean percent body weight loss. Secondary endpoints will be evaluated by assessments of weight, as well as safety and tolerability, including the ABPM sub-study. Plasma concentrations of the compounds of the present invention will be summarized and may be compared to various endpoints. In addition, weight loss parameters will be summarized in the severely obese patient sub-study.

### Safety

Safety will be evaluated by assessment of adverse events, vital signs (including blood pressure, respiratory rate, heart rate, and body temperature), ECGs, clinical laboratory evaluations (hematology, clinical chemistry including fasting blood glucose and insulin levels and urinalysis), lipid profile; levels of antibodies against the compounds of the present invention, quantitative skin assessments (mexameter) and photographic skin evaluation, protocol defined pigmented skin lesion biopsies, physical examinations including infusion site evaluations and concomitant medication review. Additionally, changes in depression/suicidality as assessed by the C—SSRS and PHQ-9 will be monitored.

### Pharmacokinetic

Plasma concentrations of the compounds of the present invention will summarized and may be compared to PD endpoints.

### Exploratory

Exploratory measurements will be assessed by insulin sensitivity (as measured by MTT and HOMA-IR), effects on Hs—CRP and HbA1c, hunger and satiety (using a Hunger/Satiety Questionnaire), body composition (using DXA at select sites), changes in waist circumference, and changes in IWQOL-Lite and C—SSRS will be assessed as exploratory endpoints.

### Sample Size Determination

Sample size per arm was calculated to target a 5 percentage point difference in mean weight change between a treatment arm and the placebo arm. From data reported by Gadde (2011), an SD of 5.7% was computed for weight change after 16 weeks of treatment. Assuming the SD in this study will be 5% to 6%, the sample size of N=30 completing subjects (accounting for 5 dropouts per dose group) has 97% power to yield a statistically significant (alpha=0.025, 1-sided) difference between an active dose group and placebo if the true underlying difference in means is 5 percentage points, and the SD is 5%. If the SD is 6%, there is 89% power.

### Statistical Methods

Continuous variables will be summarized by dose group with descriptive statistics (e.g., number of observations, mean, SD, median, maximum, and minimum). Categorical variables will be tabulated by frequency of patients per dose group. All patient information and safety measurements will be based on the Safety Population, which will include all patients who receive a dose of study drug and have a post baseline observation.

Analyses will be based on observed data only; no data will be imputed.

Continuous efficacy endpoints will be assessed via a longitudinal mixed analysis of variance model which will include fixed effects terms for treatment, timepoint, treatment-by-timepoint interaction, and baseline covariate, and random effect for subjects. The assumption of normality will be assessed via the Shapiro-Wilk statistic. If substantial departure from normality is observed, a transformation such as log (post/pre) or rank may be used to analyze the data.

The comparison of the compounds of the present invention with placebo will be carried out via 1-sided statistical test at alpha=0.025.

### Guidelines for Additional Safety Monitoring and Suspension of Dosing of a Patient

Patients will be monitored carefully during the treatment period during on site clinic visits as well as periodic telephone calls made to the patients by the study staff. In the event a patient is withdrawn from treatment due to an AE, the patient should be encouraged to complete the remaining study visits in order to monitor the event to resolution and obtain additional protocol defined safety assessments. Additionally, guidance will be provided for any worsening of depression or suicidality during the study. At all times, this guidance is subject to the clinical judgment of the Investigator and study consultants (if applicable).

The Investigator shall notify the Medical Monitor in the event any study participant fulfills any of the criteria defined in the appendices noted above, or undergoes additional monitoring for any of the events defined herein.

### Example 3: Treatment of Obese MC4R+/− Heterozygotic Mice with Compound of SEQ ID NO: 140

Diet induced obesity (DIO) littermate C57Bl/6J mice that were either wild type with respect to MC4R gene (+/+), or heterozygous for the MC4R gene (+/−), or homozygous MC4R knockout mice that do not express the MC4R gene at all (−/−) were exposed to the compound of SEQ ID NO: 140: Ac-Arg-c(Cys-D-Ala-His-D-Phe-Arg-Trp-Cys)-NH₂ by Alzet pump infusion of at a concentration of 1200 nmol/kg/day for 8 days. Body weight was measured.

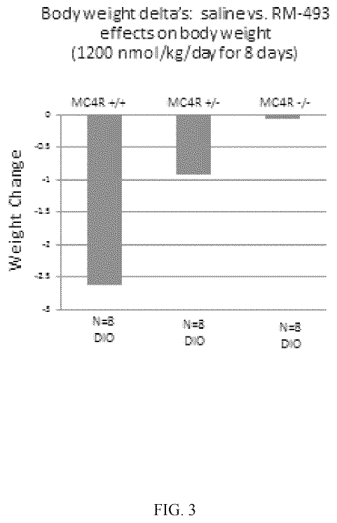

The data is presented in FIG. 3. The data shows that mice that were heterozygous for the MC4R gene (+/−) lost significant body weight (about 1 gram) over the treatment period while rodents that did not express the MC4R gene, did not show significant weight loss over this time period.

The weight change due to the exposure to the compound of SEQ ID NO: 140 in mice that are either wild type for the MC4R gene, or express only a single MC4R allele, or mice without any MC4R protein expression were compared. The data suggests that human patients with one functional MC4R allele, where their obesity is caused by the loss of function of the MC4R allele, will respond to the SEQ ID NO: 140, resulting in weight loss.

While this invention has been particularly shown and described with references to example embodiments thereof, it will be understood by those skilled in the art that various changes in form and details may be made therein without departing from the scope of the invention encompassed by the appended claims.

1. A method of treating a disorder in a subject in need thereof, comprising:
administering to said subject an effective amount of an agonist of the melanocortin-4 receptor (MC4R),
wherein the subject is a heterozygous carrier of an MC4R mutation, and the disorder results from an attenuated response of MC4R to α-melanocortin stimulating hormone (α-MSH).

2. The method of claim 1, wherein the disorder is obesity.

3. The method of claim 1, wherein the disorder is a metabolic syndrome.

4. The method of any one claims 1 to 3, wherein the MC4R agonist is not an adrenocorticotropic hormone (ACTH).

5. The method of any one of claims 1-3, wherein said MC4R agonist is Ac-Arg-c(Cys-D-Ala-His-D-Phe-Arg-Trp-Cys)-NH₂ (SEQ ID NO: 140) or a pharmaceutically acceptable salt thereof.

6. The method of any one of claims 1-3, wherein said MC4R agonist is Hydantoin(C(O)-(Arg-Gly))-c(Cys-Glu-His-D-Phe-Arg-Trp-Cys)-NH₂ (SEQ ID NO: 500) or a pharmaceutically acceptable salt thereof.

7. The method of any one of claims 1-3, wherein said MC4R agonist is a tripeptide D-Phe-Arg-Trp (SEQ ID NO: 560) or a pharmaceutical salt thereof.

8. The method of any one of claims 1-3, wherein said MC4R agonist is a peptide that includes D-Phe-Arg-Trp (SEQ ID NO: 560) or a pharmaceutical salt thereof.

9. The method of any one of claims 1-3, wherein said MC4R agonist is a peptide that includes an acetylated tripeptide Ac-D-Phe-Arg-Trp-NH₂ (SEQ ID NO: 561) or a pharmaceutical salt thereof.

10. The method of any one of claims 1-3, wherein said MC4R agonist is a peptide selected form:
(SEQ ID NO: 1)
Ac-Nle-c(Asp-His-D-Phe-Arg-Trp-β-Ala-Lys)-NH₂;
(SEQ ID NO: 2)
Ac-Nle-c(Asp-His-D-Phe-Arg-Trp-A6c-Lys)-NH₂;
(SEQ ID NO: 3)
Ac-Nle-c(Cys-His-D-Phe-Arg-Trp-Ahx-Cys)-NH₂;
(SEQ ID NO: 4)
D-Phe-c(Cys-His-D-Phe-Arg-Trp-Ala-D-Cys)-Thr-NH₂;
(SEQ ID NO: 5)
D-Phe-c(Cys-His-D-Phe-Arg-Trp-β-Ala-D-Cys)-Thr-NH₂;
(SEQ ID NO: 6)
D-Phe-c(Cys-His-D-Phe-Arg-Trp-Gaba-D-Cys)-Thr-NH₂;
(SEQ ID NO: 7)
Ac-Nle-c(Cys-His-D-Phe-Arg-Trp-Apn-Cys)-NH₂;
(SEQ ID NO: 8)
Ac-Nle-c(Asp-His-D-Phe-Arg-Trp-Apn-Lys)-NH₂;
(SEQ ID NO: 9)
Ac-A6c-c(Asp-His-D-Phe-Arg-Trp-Gaba-Lys)-NH₂;
(SEQ ID NO: 10)
Ac-D-2-Nal-c(Asp-His-D-Phe-Arg-Trp-Gaba-Lys)-NH₂;
(SEQ ID NO: 11)
Ac-Cha-c(Asp-His-D-Phe-Arg-Trp-Gaba-Lys)-NH₂;
(SEQ ID NO: 12)
Ac-Nle-c(Asp-His-D-Phe-Arg-Trp-Gaba-Lys)-NH₂;
(SEQ ID NO: 13)
Ac-Nle-c(Cys-D-Ala-His-D-Phe-Arg-Trp-Cys)-NH₂;
(SEQ ID NO: 14)
Ac-Nle-c(Cys-β-Ala-His-D-Phe-Arg-Trp-Cys)-NH₂;
(SEQ ID NO: 15)
Ac-Nle-c(Cys-Gaba-His-D-Phe-Arg-Trp-Cys)-NH₂;
(SEQ ID NO: 16)
Ac-Nle-c(Cys-Aib-His-D-Phe-Arg-Trp-Cys)-NH₂;
(SEQ ID NO: 17)
Ac-Nle-c(Cys-Gly-His-D-Phe-Arg-Trp-Cys)-NH₂;
(SEQ ID NO: 18)
Ac-Nle-c(D-Cys-Ala-His-D-Phe-Arg-Trp-Cys)-NH₂;
(SEQ ID NO: 19)
Ac-Nle-c(D-Cys-D-Ala-His-D-Phe-Arg-Trp-Cys)-NH₂;
(SEQ ID NO: 20)
Ac-Nle-c(D-Cys-β-Ala-His-D-Phe-Arg-Trp-Cys)-NH₂;
(SEQ ID NO: 21)
Ac-Nle-c(D-Cys-Gaba-His-D-Phe-Arg-Trp-Cys)-NH₂;
(SEQ ID NO: 22)
Ac-Nle-c(D-Cys-Aib-His-D-Phe-Arg-Trp-Cys)-NH₂;
(SEQ ID NO: 23)
Ac-Nle-c(D-Cys-Gly-His-D-Phe-Arg-Trp-Cys)-NH₂;
(SEQ ID NO: 24)
Ac-Nle-c(Cys-D-Ala-His-D-Phe-Arg-Trp-D-Cys)-NH₂;
(SEQ ID NO: 25)
Ac-Nle-c(Cys-β-Ala-His-D-Phe-Arg-Trp-D-Cys)-NH₂;
(SEQ ID NO: 26)
Ac-Nle-c(Cys-Gaba-His-D-Phe-Arg-Trp-D-Cys)-NH₂;
(SEQ ID NO: 27)
Ac-Nle-c(Cys-Aib-His-D-Phe-Arg-Trp-D-Cys)-NH₂;
(SEQ ID NO: 28)
Ac-Nle-c(Cys-Gly-His-D-Phe-Arg-Trp-D-Cys)-NH₂;
(SEQ ID NO: 29)
Ac-Nle-c(D-Cys-Ala-His-D-Phe-Arg-Trp-D-Cys)-NH₂;
(SEQ ID NO: 30)
Ac-Nle-c(D-Cys-D-Ala-His-D-Phe-Arg-Trp-D-Cys)-NH₂;
(SEQ ID NO: 31)
Ac-Nle-c(D-Cys-β-Ala-His-D-Phe-Arg-Trp-D-Cys)-NH₂;
(SEQ ID NO: 32)
Ac-Nle-c(D-Cys-Gaba-His-D-Phe-Arg-Trp-D-Cys)-NH₂;
(SEQ ID NO: 33)
Ac-Nle-c(D-Cys-Aib-His-D-Phe-Arg-Trp-D-Cys)-NH₂;
(SEQ ID NO: 34)
Ac-Oic-c(Asp-His-D-Phe-Arg-Trp-Gaba-Lys)-NH₂;
(SEQ ID NO: 35)
Ac-Chg-c(Asp-His-D-Phe-Arg-Trp-Gaba-Lys)-NH₂;
(SEQ ID NO: 36)
Ac-hCha-c(Asp-His-D-Phe-Arg-Trp-Gaba-Lys)-NH₂;
(SEQ ID NO: 37)
Ac-D-Cha-c(Asp-His-D-Phe-Arg-Trp-Gaba-Lys)-NH₂;
(SEQ ID NO: 38)
Ac-D-hCha-c(Asp-His-D-Phe-Arg-Trp-Gaba-Lys)-NH₂;
(SEQ ID NO: 39)
Ac-Nip-c(Asp-His-D-Phe-Arg-Trp-Gaba-Lys)-NH₂;
(SEQ ID NO: 40)
Ac-hPro-c(Asp-His-D-Phe-Arg-Trp-Gaba-Lys)-NH₂;
(SEQ ID NO: 41)
Ac-hLeu-c(Asp-His-D-Phe-Arg-Trp-Gaba-Lys)-NH₂;
(SEQ ID NO: 42)
Ac-Phe-c(Asp-His-D-Phe-Arg-Trp-Gaba-Lys)-NH₂;
(SEQ ID NO: 43)
Ac-D-Phe-c(Asp-His-D-Phe-Arg-Trp-Gaba-Lys)-NH₂;
(SEQ ID NO: 44)
Ac-D-Chg-c(Asp-His-D-Phe-Arg-Trp-Gaba-Lys)-NH₂;
(SEQ ID NO: 45)
n-butanoyl-Cha-c(Asp-His-D-Phe-Arg-Trp-Gaba-Lys)-NH₂;
(SEQ ID NO: 46)
n-butyryl-Cha-c(Asp-His-D-Phe-Arg-Trp-Gaba-Lys)-NH₂;
(SEQ ID NO: 47)
Ac-hPhe-c(Asp-His-D-Phe-Arg-Trp-Gaba-Lys)-NH₂;
(SEQ ID NO: 48)
Ac-β-hMet-c(Asp-His-D-Phe-Arg-Trp-Gaba-Lys)-NH₂;
(SEQ ID NO: 49)
Ac-Gaba-c(Asp-His-D-Phe-Arg-Trp-Gaba-Lys)-NH₂;
(SEQ ID NO: 50)
Ac-Cha-c(Asp-His-D-Phe-Arg-D-Trp-Ala-Lys)-NH₂;
(SEQ ID NO: 51)
Ac-hCha-c(Asp-His-D-Phe-Arg-D-Trp-Ala-Lys)-NH₂;
(SEQ ID NO: 52)
Ac-Leu-c(Asp-His-D-Phe-Arg-D-Trp-Ala-Lys)-NH₂;
(SEQ ID NO: 53)
Ac-hLeu-c(Asp-His-D-Phe-Arg-D-Trp-Ala-Lys)-NH₂;
(SEQ ID NO: 54)
Ac-Phe-c(Asp-His-D-Phe-Arg-D-Trp-Ala-Lys)-NH₂;
(SEQ ID NO: 55)
Ac-Nle-c(Asp-His-D-Phe-Arg-D-Trp-D-Ala-Lys)-NH₂;
(SEQ ID NO: 56)
Ac-Nle-c(Asp-His-D-Phe-Arg-D-Trp-β-Ala-Lys)-NH₂;
(SEQ ID NO: 57)
Ac-Nle-c(Asp-His-D-Phe-Arg-D-Trp-Gaba-Lys)-NH₂;
(SEQ ID NO: 58)
Ac-Nle-c(Asp-His-D-Phe-Arg-D-Trp-Aha-Lys)-NH₂;
(SEQ ID NO: 59)
Ac-Nle-c(Asp-His-D-Phe-Arg-D-Trp-Apn-Lys)-NH₂;
(SEQ ID NO: 60)
Ac-Nle-c(Cys-His-D-Phe-Arg-D-Trp-Apn-Cys)-NH₂;
(SEQ ID NO: 61)
Ac-Nle-c(Cys-His-D-Phe-Arg-D-Trp-Gaba-Cys)-NH₂;
(SEQ ID NO: 62)
Ac-Nle-c(Cys-His-D-Phe-Arg-D-Trp-Ahx-Cys)-NH₂;
(SEQ ID NO: 63)
Ac-Nle-c(Cys-His-D-Phe-Arg-D-Trp-β-Ala-Cys)-NH₂;
(SEQ ID NO: 64)
Ac-Nle-c(Cys-His-D-Phe-Arg-D-Trp-D-Ala-Cys)-NH₂;
(SEQ ID NO: 65)
Ac-Nle-c(Cys-D-Ala-His-D-2-Nal-Arg-Trp-Cys)-NH₂;
(SEQ ID NO: 66)
Ac-Nle-c(Cys-D-Ala-His-D-2-Nal-Arg-2-Nal-Cys)-NH₂;
(SEQ ID NO: 67)
Ac-Nle-c(Cys-D-Ala-His-D-2-Nal-Arg-1-Nal-Cys)-NH₂;
(SEQ ID NO: 68)
n-butanoyl-Nle-c(Cys-D-Ala-His-D-Phe-Arg-2-Nal-Cys)-NH₂;
(SEQ ID NO: 69)
n-butanoyl-Nle-c(Cys-D-Ala-His-D-Phe-Arg-Trp-Cys)-NH₂;
(SEQ ID NO: 70)
Ac-Nle-c(Cys-D-Ala-His-D-Phe-Arg-2-Nal-Cys)-NH₂;
(SEQ ID NO: 71)
Ac-Nle-c(Cys-D-Ala-His-D-Phe-Arg-1-Nal-Cys)-NH₂;
(SEQ ID NO: 72)
Ac-Nle-c(Cys-D-Ala-His-D-Phe-Arg-Bal-Cys)-NH₂;
(SEQ ID NO: 73)
Ac-Nle-c(Cys-D-Glu-His-D-Phe-Arg-Trp-Cys)-NH₂;
(SEQ ID NO: 74)
Ac-Nle-c(Asp-His-D-Phe-Arg-Trp-D-Ala-Lys)-NH₂;
(SEQ ID NO: 75)
Ac-Nle-c(Cys-D-Ala-His-D-2-Nal-Arg-Bal-Cys)-NH₂;
(SEQ ID NO: 76)
Ac-Nle-c(Pen-D-Ala-His-D-Phe-Arg-Trp-Cys)-NH₂;
(SEQ ID NO: 77)
Ac-Nle-c(Cys-D-Ala-His-D-Phe-Arg-Trp-Pen)-NH₂;
(SEQ ID NO: 78)
Ac-Nle-c(Pen-D-Ala-His-D-Phe-Arg-Trp-Pen)-NH₂;
(SEQ ID NO: 79)
D-Phe-c(Cys-His-D-Phe-hArg-Trp-β-Ala-D-Cys)-Thr-NH₂;
(SEQ ID NO: 80)
D-Phe-c(Cys-His-D-(Et)Tyr-Arg-Trp-β-Ala-D-Cys)-Thr-NH₂;
(SEQ ID NO: 81)
D-Phe-c(Cys-His-D-Phe-Arg-Bip-β-Ala-D-Cys)-Thr-NH₂;
(SEQ ID NO: 82)
D-Phe-c(Cys-His-D-(Et)Tyr-hArg-Trp-β-Ala-D-Cys)-Thr-NH₂;
(SEQ ID NO: 83)
D-Phe-c(Cys-His-D-Phe-hArg-Bip-β-Ala-D-Cys)-Thr-NH₂;
(SEQ ID NO: 84)
D-Phe-c(Cys-His-D-(Et)Tyr-hArg-Bip-β-Ala-D-Cys)-Thr-NH₂;
(SEQ ID NO: 85)
Nle-c(Cys-His-D-Phe-Arg-Trp-Apn-Cys)-NH₂;
(SEQ ID NO: 86)
Ac-Nle-c(Asp-D-Ala-His-D-Phe-Arg-Trp-Lys)-NH₂;
(SEQ ID NO: 87)
Ac-Nle-c(Asp-D-Ala-His-D-Phe-Arg-Bal-Lys)-NH₂;
(SEQ ID NO: 88)
Ac-Nle-c(Cys-D-Ala-His-D-Phe-Arg-Trp-Pen)-OH;
(SEQ ID NO: 89)
Ac-Nle-c(Cys-D-Abu-His-D-Phe-Arg-Trp-Cys)-NH₂;
(SEQ ID NO: 90)
Ac-Nle-c(Cys-D-Val-His-D-Phe-Arg-Trp-Cys)-NH₂;
(SEQ ID NO: 91)
Ac-Nle-c(Cys-D-Ile-His-D-Phe-Arg-Trp-Cys)-NH₂;
(SEQ ID NO: 92)
Ac-Nle-c(Cys-D-Leu-His-D-Phe-Arg-Trp-Cys)-NH₂;
(SEQ ID NO: 93)
Ac-Nle-c(Cys-D-T1e-His-D-Phe-Arg-Trp-Cys)-NH₂;
(SEQ ID NO: 94)
Ac-Nle-c(Cys-D-Cha-His-D-Phe-Arg-Trp-Cys)-NH₂;
(SEQ ID NO: 95)
Ac-Nle-c(Pen-His-D-Phe-Arg-Trp-Gaba-Cys)-NH₂;
(SEQ ID NO: 96)
Ac-Nle-c(Cys-His-D-Phe-Arg-Trp-Gaba-Pen)-NH₂;
(SEQ ID NO: 97)
Ac-Nle-c(Pen-His-D-Phe-Arg-Trp-Gaba-Pen)-NH₂;
(SEQ ID NO: 98)
Ac-Leu-c(Cys-His-D-Phe-Arg-Trp-Gaba-Cys)-NH₂;
(SEQ ID NO: 99)
Ac-Cha-c(Cys-His-D-Phe-Arg-Trp-Gaba-Cys)-NH₂;
(SEQ ID NO: 100)
Ac-Ile-c(Cys-His-D-Phe-Arg-Trp-Gaba-Cys)-NH₂;
(SEQ ID NO: 101)
Ac-Phe-c(Cys-His-D-Phe-Arg-Trp-Gaba-Cys)-NH₂;
(SEQ ID NO: 102)
Ac-Val-c(Cys-His-D-Phe-Arg-Trp-Gaba-Cys)-NH₂;
(SEQ ID NO: 103)
Ac-2-Nal-c(Cys-His-D-Phe-Arg-Trp-Gaba-Cys)-NH₂;
(SEQ ID NO: 104)
Nle-c(Cys-His-D-Phe-Arg-Trp-Gaba-Cys)-NH₂;
(SEQ ID NO: 105)
Phe-c(Cys-His-D-Phe-Arg-Trp-Gaba-Cys)-NH₂;
(SEQ ID NO: 106)
Ac-Nle-c(Cys-β-Pal-D-Phe-Arg-Trp-Gaba-Cys)-NH₂;
(SEQ ID NO: 107)
Ac-Nle-c(Cys-D-Ala-His-D-Phe-Arg-Trp-Cys)-OH;
(SEQ ID NO: 108)
Ac-Nle-c(Cys-His-Phe-Arg-D-Trp-Gaba-Cys)-NH₂;
(SEQ ID NO: 109)
Ac-Nle-c(Asp-His-D-2-Nal-Arg-Trp-Ala-Lys)-NH₂;
(SEQ ID NO: 110)
Ac-Nle-c(Asp-His-D-2-Nal-Arg-Trp-β-Ala-Lys)-NH₂;
(SEQ ID NO: 111)
Ac-Nle-c(Cys-His-D-2-Nal-Arg-Trp-Gaba-Cys)-NH₂;
(SEQ ID NO: 112)
Ac-Nle-c(Cys-His-D-2-Nal-Arg-Trp-Ahx-Cys)-NH₂;
(SEQ ID NO: 113)
Ac-hPhe-c(Asp-His-D-2-Nal-Arg-Trp-Gaba-Lys)-NH₂;
(SEQ ID NO: 114)
Ac-Cha-c(Asp-His-D-2-Nal-Arg-Trp-Gaba-Lys)-NH₂;
(SEQ ID NO: 115)
Ac-Nle-c(Asp-His-D-Phe-Arg-Trp-β-Ala-Lys)-OH;
(SEQ ID NO: 116)
Ac-Nle-c(Cys-His-D-Phe-Arg-Trp-Ahx-Cys)-OH;
(SEQ ID NO: 117)
D-Phe-c(Cys-His-D-Phe-Arg-Trp-Ala-D-Cys)-Thr-OH;
(SEQ ID NO: 118)
D-Phe-c(Cys-His-D-Phe-Arg-Trp-β-Ala-D-Cys)-Thr-OH;
(SEQ ID NO: 119)
D-Phe-c(Cys-His-D-Phe-Arg-Trp-Gaba-D-Cys)-Thr-OH;
(SEQ ID NO: 120)
Ac-Nle-c(Cys-His-D-Phe-Arg-Trp-Apn-Cys)-OH;
(SEQ ID NO: 121)
Ac-Nle-c(Asp-His-D-Phe-Arg-Trp-Apn-Lys)-OH;
(SEQ ID NO: 122)
Ac-Cha-c(Asp-His-D-Phe-Arg-Trp-Gaba-Lys)-OH;
(SEQ ID NO: 123)
Ac-Nle-c(Asp-His-D-Phe-Arg-Trp-Gaba-Lys)-OH;
(SEQ ID NO: 124)
Ac-Chg-c(Asp-His-D-Phe-Arg-Trp-Gaba-Lys)-OH;
(SEQ ID NO: 125)
Ac-D-Cha-c(Asp-His-D-Phe-Arg-Trp-Gaba-Lys)-OH;
(SEQ ID NO: 126)
Ac-hCha-c(Asp-His-D-Phe-Arg-Trp-Gaba-Lys)-OH;
(SEQ ID NO: 127)
Ac-D-Chg-c(Asp-His-D-Phe-Arg-Trp-Gaba-Lys)-OH;
(SEQ ID NO: 128)
Ac-hPhe-c(Asp-His-D-Phe-Arg-Trp-Gaba-Lys)-OH;
(SEQ ID NO: 129)
Ac-Nle-c(Cys-His-D-Phe-Arg-D-Trp-Gaba-Cys)-OH;
(SEQ ID NO: 130)
Ac-Nle-c(Cys-His-D-Phe-Arg-D-Trp-Ahx-Cys)-OH;
(SEQ ID NO: 131)
Ac-Nle-c(Cys-His-D-Phe-Arg-D-Trp-β-Ala-Cys)-OH;
(SEQ ID NO: 132)
Ac-Nle-c(Cys-His-D-Phe-Arg-D-Trp-D-Ala-Cys)-OH;
(SEQ ID NO: 133)
Ac-Nle-c(Cys-D-Ala-His-D-2-Nal-Arg-Trp-Cys)-OH;
(SEQ ID NO: 134)
Ac-Nle-c(Cys-D-Ala-His-D-2-Nal-Arg-2-Nal-Cys)-OH;
(SEQ ID NO: 135)
Ac-Nle-c(Cys-D-Ala-His-D-2-Nal-Arg-1-Nal-Cys)-OH;
(SEQ ID NO: 136)
Ac-Nle-c(Cys-D-Ala-His-D-2-Nal-Arg-Bal-Cys)-OH;
(SEQ ID NO: 137)
Ac-Nle-c(Pen-D-Ala-His-D-Phe-Arg-Trp-Cys)-OH;
(SEQ ID NO: 138)
Ac-Nle-c(Cys-His-D-Phe-Arg-Trp-Gaba-Pen)-OH;
(SEQ ID NO: 139)
Ac-Arg-c(Cys-D-Ala-His-D-2-Nal-Arg-Trp-Cys)-NH₂;
(SEQ ID NO: 140)
Ac-Arg-c(Cys-D-Ala-His-D-Phe-Arg-Trp-Cys)-NH₂;
(SEQ ID NO: 141)
Ac-D-Arg-c(Cys-D-Ala-His-D-Phe-Arg-Trp-Cys)-NH₂;
(SEQ ID NO: 142)
Ac-D-Arg-c(Cys-D-Ala-His-D-Phe-Arg-Trp-Pen)-NH₂;
(SEQ ID NO: 143)
Ac-D-Arg-c(Cys-His-D-Phe-Arg-Trp-Gaba-Pen)-NH₂;
(SEQ ID NO: 144)
Ac-Arg-c(Cys-His-D-Phe-Arg-Trp-Gaba-Pen)-NH₂;
(SEQ ID NO: 145)
Ac-Arg-c(Cys-D-Ala-His-D-Phe-Arg-Trp-Pen)-NH₂;
(SEQ ID NO: 146)
Ac-D-Arg-c(Asp-His-D-Phe-Arg-Trp-Ala-Lys)-NH₂;
(SEQ ID NO: 147)
Ac-Arg-c(Asp-His-D-Phe-Arg-Trp-Ala-Lys)-NH₂;
(SEQ ID NO: 148)
Ac-Nle-c(Cys-D-Ala-His-D-Phe-Arg-Trp-Gly-Cys)-NH₂;
(SEQ ID NO: 149)
Ac-Nle-c(Cys-D-Ala-His-D-Phe-Arg-Trp-D-Ala-Cys)-NH₂;
(SEQ ID NO: 150)
Ac-Nle-c(Cys-D-Ala-His-D-Phe-Arg-Trp-β-Ala-Cys)-NH₂;
(SEQ ID NO: 151)
Ac-Nle-c(Cys-D-Ala-His-D-Phe-Arg-Trp-Gaba-Cys)-NH₂;
(SEQ ID NO: 152)
Ac-Nle-c(Cys-D-Ala-His-D-Phe-Arg-Trp-Apn-Cys)-NH₂;
(SEQ ID NO: 153)
Ac-c(Cys-Glu-His-D-Phe-Arg-Trp-Ala-Cys)-NH₂;
(SEQ ID NO: 154)
Ac-c(Cys-Glu-His-D-Phe-Arg-2-Nal-Ala-Cys)-NH₂;
(SEQ ID NO: 155)
Ac-c(Cys-D-Ala-His-D-Phe-Arg-Trp-Ala-Cys)-NH₂;
(SEQ ID NO: 156)
Ac-c(Cys-D-Ala-His-D-Phe-Arg-2-Nal-Ala-Cys)-NH₂;
(SEQ ID NO: 157)
Ac-Nle-c(Cys-D-Ala-His-D-Phe-Arg-Trp-Ala-Cys)-NH₂;
(SEQ ID NO: 158)
Ac-Nle-c(Asp-D-Ala-His-D-Phe-Arg-Bal-Ala-Lys)-NH₂;
(SEQ ID NO: 159)
Tyr-Gly-Arg-(Lys)₂-(Arg)₂-Gln-(Arg)₃-Nle-c(Asp-His-D-2-Nal-Arg-Trp-
Lys)-NH₂;
(SEQ ID NO: 160)
Tyr-Gly-Arg-(Lys)₂-(Arg)₂-Gln-(Arg)₃-Doc-Nle-c(Asp-His-D-2-Nal-Arg-
Trp-Lys)-NH₂;
(SEQ ID NO: 161)
Nle-c(Asp-His-D-2-Nal-Arg-Trp-Lys)-β-Ala-Tyr-Gly-Arg-(Lys)₂-(Arg)₂-
Gln-(Arg)₃-NH₂;
(SEQ ID NO: 162)
Ac-Nle-c(Asp-His-D-2-Nal-Arg-Trp-Lys)-β-Ala-Tyr-Gly-Arg-(Lys)₂-
(Arg)₂-Gln-(Arg)₃-NH₂;
(SEQ ID NO: 163)
Nle-c(Asp-His-D-2-Nal-Arg-Trp-Lys)-(Doc)₂-Tyr-Gly-Arg-(Lys)₂-(Arg)₂-
Gln-(Arg)₃-NH₂;
(SEQ ID NO: 164)
Ac-Nle-c(Asp-His-D-2-Nal-Arg-Trp-Lys)-(Pro)₂-Lys-Asp-Tyr-Gly-Arg-
(Lys)₂-(Arg)₂-Gln-(Arg)₃-NH₂;
(SEQ ID NO: 165)
Ac-c(Cys-Glu-His-D-2-Nal-Arg-Trp-Gly-Cys)-(Pro)₂-Lys-Asp-Tyr-Gly-
Arg-(Lys)₂-(Arg)₂-Gln-(Arg)₃-NH₂;
(SEQ ID NO: 166)
Ac-Nle-c(Asp-His-D-2-Nal-Arg-Trp-Lys)-(β-Ala)2-Tyr-Gly-Arg-(Lys)₂-
(Arg)₂-Gln-(Arg)₃-NH₂;
(SEQ ID NO: 167)
Ac-Nle-c(Asp-His-D-2-Nal-Arg-Trp-Lys)-(Pro)₂-Lys-Asp-Doc-Tyr-Gly-
Arg-(Lys)₂-(Arg)₂-Gln-(Arg)₃-NH₂;
(SEQ ID NO: 168)
Ac-c(Cys-Glu-His-D-2-Nal-Arg-Trp-Gly-Cys)-(Pro)₂-Lys-Asp-Doc-Tyr-
Gly-Arg-(Lys)₂-(Arg)₂-Gln-(Arg)₃-NH₂;
(SEQ ID NO: 169)
Ac-c(Cys-Glu-His-D-2-Nal-Arg-Trp-Ala-Cys)-(Pro)₂-Lys-Asp-β-Ala-Tyr-
Gly-Arg-(Lys)₂-(Arg)₂-Gln-(Arg)₃-NH₂;
(SEQ ID NO: 170)
Ac-c(Cys-Glu-His-D-2-Nal-Arg-Trp-Ala-Cys)-(Pro)₂₋Lys-Asp-Doc-Tyr-
Gly-Arg-(Lys)₂-(Arg)₂-Gln-(Arg)₃-NH₂;
(SEQ ID NO: 171)
Ac-Nle-c(Asp-His-D-2-Nal-Arg-Trp-Lys)-(Doc)₂-Tyr-Gly-Arg-(Lys)₂-
(Arg)₂-Gln-(Arg)₃-NH₂;
(SEQ ID NO: 172)
Ac-c(Cys-Glu-His-D-2-Nal-Arg-2-Nal-Ala-Cys)-(Pro)₂-Lys-Asp-β-Ala-Tyr-
Gly-Arg-(Lys)₂-(Arg)₂-Gln-(Arg)₃-NH₂;
(SEQ ID NO: 173)
Ac-c(Cys-Glu-His-D-2-Nal-Arg-Bal-Ala-Cys)-(Pro)₂-Lys-Asp-β-Ala-(Arg)₅-
Gln-(Arg)₃-NH₂;
(SEQ ID NO: 174)
Ac-c(Cys-Glu-His-D-2-Nal-Arg-Bal-Ala-Cys)-(Pro)₂-Lys-Asp-β-Ala-Gly-
(Arg)₅-Gln-(Arg)₃-NH₂;
(SEQ ID NO: 175)
Ac-c(Cys-Glu-His-D-2-Nal-Arg-Bal-Ala-Cys)-(Pro)₂-Lys-Asp-β-Ala-Tyr-
Gly-(Arg)₅-Gln-(Arg)₃-NH₂;
(SEQ ID NO: 176)
Ac-c(Cys-Glu-His-D-2-Nal-Arg-Trp-Ala-Cys)-(Pro)₂-Lys-Asp-β-Ala-Tyr-
Gly-Arg-(Lys)₂-Arg-Gln-(Arg)₄-NH₂;
(SEQ ID NO: 177)
Ac-c(Cys-Glu-His-D-2-Nal-Arg-Trp-Ala-Cys)-(Pro)₂-Lys-Asp-β-Ala-Tyr-
Gly-Arg-(Lys)₂-Gln-(Arg)₅-NH₂;
(SEQ ID NO: 178)
Ac-c(Cys-Glu-His-D-2-Nal-Arg-Trp-Ala-Cys)-(Pro)₂-Lys-Asp-β-Ala-Tyr-
Gly-Arg-Lys-Gln-Lys-(Arg)₅-NH₂;
(SEQ ID NO: 179)
Ac-c(Cys-Glu-His-D-2-Nal-Arg-Trp-Ala-Cys)-(Pro)₂-Lys-Asp-β-Ala-Tyr-
Gly-Arg-(Lys)₂-(Arg)₄-Gln-Arg-NH₂;
(SEQ ID NO: 180)
Ac-c(Cys-Glu-His-D-2-Nal-Arg-Bal-Ala-Cys)-(Pro)₂-Lys-Asp-β-Ala-Tyr-
Aib-Arg-(Lys)₂-(Arg)₂-Gln-(Arg)₃-NH₂;
(SEQ ID NO: 181)
Ac-c(Cys-Glu-His-D-2-Nal-Arg-1-Nal-Ala-Cys)-(Pro)₂-Arg-Asp-β-Ala-
(Arg)₅-Gln-(Arg)₃-NH₂;
(SEQ ID NO: 182)
Ac-c(Cys-Glu-His-D-2-Nal-Arg-1-Nal-Ala-Cys)-(Pro)₂-Lys-Asp-β-Ala-
(Arg)₅-Gln-(Arg)₃-NH₂;
(SEQ ID NO: 183)
Ac-c(Cys-Glu-His-D-2-Nal-Arg-1-Nal-Ala-Cys)-(Pro)₂-Lys-Asp-β-Ala-
(Arg)₆-Gln-(Arg)₃-NH₂;
(SEQ ID NO: 184)
Ac-c(Cys-Glu-His-D-2-Nal-Arg-2-Nal-Ala-Cys)-(Pro)₂-Arg-Asp-β-Ala-
(Arg)₅-Gln-(Arg)₃-NH₂;
(SEQ ID NO: 185)
Ac-c(Cys-Glu-His-D-2-Nal-Arg-2-Nal-Ala-Cys)-(Pro)₂-Lys-Asp-β-Ala-
(Arg)₅-Gln-(Arg)₃-NH₂;
(SEQ ID NO: 186)
Ac-c(Cys-Glu-His-D-2-Nal-Arg-2-Nal-Ala-Cys)-(Pro)₂-Lys-Asp-β-Ala-
(Arg)₆-Gln-(Arg)₃-NH₂;
(SEQ ID NO: 187)
Ac-c(Cys-Glu-His-D-2-Nal-Arg-Bal-Ala-Cys)-(Pro)₂-Arg-Asp-β-Ala-
(Arg)₆-Gln-(Arg)₃-NH₂;
(SEQ ID NO: 188)
Ac-c(Cys-Glu-His-D-2-Nal-Arg-Bal-Ala-Cys)-(Pro)₂-Arg-Asp-β-Ala-
(Arg)₅-Gln-(Arg)₃-NH₂;
(SEQ ID NO: 189)
Ac-c(Cys-Glu-His-D-2-Nal-Arg-Bal-Ala-Cys)-(Pro)₂-Lys-Asp-β-Ala-(Arg)₆-
Gln-(Arg)₃-NH₂;
(SEQ ID NO: 190)
Ac-c(Cys-Glu-His-D-2-Nal-Arg-Trp-Ala-Cys)-(Pro)₂-Lys-Asp-β-Ala-Tyr-
Gly-Arg-(Lys)₂-(Arg)₃-Gln-(Arg)₂-NH₂;
(SEQ ID NO: 191)
Ac-c(Cys-Glu-His-D-2-Nal-Arg-Trp-Ala-Cys)-(Pro)₂-Lys-Asp-β-Ala-Tyr-
Gly-Arg-Gln-(Lys)₂-(Arg)₅-NH₂;
(SEQ ID NO: 192)
Ac-c(Cys-Glu-His-D-2-Nal-Arg-Trp-Ala-Cys)-(Pro)₂-Lys-Asp-β-Ala-Tyr-
Gly-Arg-(Lys)2-(Arg)₅-Gln-NH₂;
(SEQ ID NO: 193)
Ac-c(Cys-Glu-His-D-2-Nal-Arg-1-Nal-Ala-Cys)-(Pro)₂-Lys-Asp-β-Ala-Tyr-
Gly-Arg-(Lys)₂-(Arg)₂-Gln-(Arg)3-NH₂;;
(SEQ ID NO: 194)
Ac-c(Cys-Glu-His-D-2-Nal-Arg-Bal-Ala-Cys)-(Pro)₂-Lys-Asp-β-Ala-Tyr-
Gly-Arg-(Lys)₂-(Arg)₂-Gln-(Arg)₃-NH₂;
(SEQ ID NO: 195)
Ac-c(Cys-Glu-His-D-2-Nal-Arg-1-Nal-Ala-Cys)-(Pro)₂-Lys-Asp-β-Ala-
(Arg)₂-Lys-(Arg)₂-Gln-(Arg)3-NH₂;
(SEQ ID NO: 196)
Ac-c(Cys-Glu-His-D-2-Nal-Arg-1-Nal-Ala-Cys)-(Pro)₂-Lys-Asp-β-Ala-Arg-
Lys-(Arg)₃-Gln-(Arg)₃-NH₂;
(SEQ ID NO: 197)
Ac-c(Cys-Glu-His-D-2-Nal-Arg-2-Nal-Ala-Cys)-(Pro)₂-Lys-Asp-β-Ala-
(Arg)₂-Lys-(Arg)₂-Gln-(Arg)₃-NH₂;
(SEQ ID NO: 198)
Ac-c(Cys-Glu-His-D-2-Nal-Arg-2-Nal-Ala-Cys)-(Pro)₂-Lys-Asp-β-Ala-Tyr-
Gly-(Arg)₂-Lys-(Arg)₂-Gln-(Arg)₃-NH₂;
(SEQ ID NO: 199)
Ac-c(Cys-Glu-His-D-2-Nal-Arg-2-Nal-Ala-Cys)-(Pro)₂-Lys-Asp-β-Ala-Gly-
(Arg)₂-Lys-(Arg)₂-Gln-(Arg)₃-NH₂;
(SEQ ID NO: 200)
Ac-c(Cys-Glu-His-D-2-Nal-Arg-2-Nal-Ala-Cys)-(Pro)₂-Lys-Asp-β-Ala-Gly-
Arg-Lys-(Arg)₃-Gln-(Arg)₃-NH₂;
(SEQ ID NO: 201)
Ac-c(Cy s-Glu-His-D-2-Nal-Arg-1-Nal-Ala-Cys)-(Pro)₂-Lys-Asp-β-Ala-Tyr-
Gly-(Arg)₂-Lys-(Arg)₂-Gln-(Arg)₃-NH₂;
(SEQ ID NO: 202)
Ac-c(Cys-Glu-His-D-2-Nal-Arg-1-Nal-Ala-Cys)-(Pro)₂-Lys-Asp-β-Ala-Tyr-
Gly-Arg-Lys-(Arg)₃-Gln-(Arg)₃-NH₂;
(SEQ ID NO: 203)
Ac-c(Cys-Glu-His-D-2-Nal-Arg-1-Nal-Ala-Cys)-(Pro)₂-Lys-Asp-β-Ala-Gly-
(Arg)₂-Lys-(Arg)₂-Gln-(Arg)₃-NH₂;
(SEQ ID NO: 204)
Ac-c(Cys-Glu-His-D-2-Nal-Arg-1-Nal-Ala-Cys)-(Pro)₂-Lys-Asp-β-Ala-Gly-
Arg-Lys-(Arg)₃-Gln-(Arg)₃-NH₂;
(SEQ ID NO: 205)
Ac-c(Cys-Glu-His-D-2-Nal-Arg-2-Nal-Ala-Cys)-(Pro)₂-Lys-Asp-β-Ala-
(Arg)₂-Lys-(Arg)₂-Gln-(Arg)₃-NH₂;
(SEQ ID NO: 206)
Ac-c(Cys-Glu-His-D-2-Nal-Arg-2-Nal-Ala-Cys)-(Pro)₂-Lys-Asp-β-Ala-Arg-
Lys-(Arg)₃-Gln-(Arg)₃-NH₂;
(SEQ ID NO: 207)
Ac-c(Cys-Glu-His-D-2-Nal-Arg-2-Nal-Ala-Cys)-(Pro)₂-Lys-Asp-β-Ala-Tyr-
Gly-Arg-Lys-(Arg)₃-Gln-(Arg)₃-NH₂;
(SEQ ID NO: 208)
Ac-c(Cys-Glu-His-D-2-Nal-Arg-Bal-Ala-Cys)-(Pro)₂-Lys-Asp-β-Ala-(Arg)₂-
Lys-(Arg)₂-Gln-(Arg)₃-NH₂;
(SEQ ID NO: 209)
Ac-c(Cys-Glu-His-D-2-Nal-Arg-Bal-Ala-Cys)-(Pro)₂-Lys-Asp-β-Ala-Arg-
Lys-(Arg)₃-Gln-(Arg)₃-NH₂;
(SEQ ID NO: 210)
Ac-c(Cys-Glu-His-D-2-Nal-Arg-Bal-Ala-Cys)-(Pro)₂-Lys-Asp-β-Ala-Tyr-
Gly-(Arg)₂-Lys-(Arg)₂-Gln-(Arg)₃-NH₂;
(SEQ ID NO: 211)
Ac-c(Cys-Glu-His-D-2-Nal-Arg-Bal-Ala-Cys)-(Pro)₂-Lys-Asp-β-Ala-Tyr-
Gly-Arg-Lys-(Arg)₃-Gln-(Arg)₃-NH₂;
(SEQ ID NO: 212)
Ac-c(Cys-Glu-His-D-2-Nal-Arg-Bal-Ala-Cys)-(Pro)₂-Lys-Asp-β-Ala-Gly-
(Arg)₂-Lys-(Arg)₂-Gln-(Arg)₃-NH₂;
(SEQ ID NO: 213)
Ac-c(Cys-Glu-His-D-2-Nal-Arg-Bal-Ala-Cys)-(Pro)₂-Lys-Asp-β-Ala-Gly-
Arg-Lys-(Arg)₃-Gln-(Arg)₃-NH₂;
(SEQ ID NO: 214)
Ac-c(Cys-Glu-His-D-2-Nal-Arg-Trp-Ala-Cys)-(Pro)₂-Lys-Asp-β-Ala-
(Arg)₅-Gln-(Arg)₃-NH₂;
(SEQ ID NO: 215)
Ac-c(Cys-Glu-His-D-2-Nal-Arg-Trp-Ala-Cys)-(Pro)₂-Arg-Asp-β-Ala-
(Arg)₅-Gln-(Arg)₃-NH₂;
(SEQ ID NO: 216)
Ac-c(Cys-Glu-His-D-2-Nal-Arg-Trp-Ala-Cys)-(Pro)₂-Lys-Asp-β-Ala-Tyr-
Gly-(Arg)₅-Gln-(Arg)₃-NH₂;
(SEQ ID NO: 217)
Ac-c(Cys-Glu-His-D-2-Nal-Arg-Trp-Ala-Cys)-(Pro)₂-Arg-Asp-β-Ala-Tyr-
Gly-(Arg)₅-Gln-(Arg)₃-NH₂;
(SEQ ID NO: 218)
Ac-c(Cys-Glu-His-D-2-Nal-Arg-Trp-Ala-Cys)-(Pro)₂-Lys-Asp-β-Ala-
(Arg)₅-Gln-(Arg)₄-NH₂;
(SEQ ID NO: 219)
Ac-c(Cys-Glu-His-D-2-Nal-Arg-Trp-Ala-Cys)-(Pro)₂-Arg-Asp-β-Ala-
(Arg)₅-Gln-(Arg)₄-NH₂;
(SEQ ID NO: 220)
Ac-c(Cys-Glu-His-D-2-Nal-Arg-Trp-Ala-Cys)-(Pro)₂-Lys-Asp-β-Ala-Tyr-
Gly-(Arg)₅-Gln-(Arg)₄-NH₂;
(SEQ ID NO: 221)
Ac-c(Cys-Glu-His-D-2-Nal-Arg-Trp-Ala-Cys)-(Pro)₂-Arg-Asp-β-Ala-Tyr-
Gly-(Arg)₅-Gln-(Arg)₄-NH₂;
(SEQ ID NO: 222)
Ac-c(Cys-Glu-His-D-2-Nal-Arg-1-Nal-Ala-Cys)-(Pro)₂-Lys-Asp-β-Ala-
(Arg)₅-Gln-(Arg)₄-NH₂;
(SEQ ID NO: 223)
Ac-c(Cys-Glu-His-D-2-Nal-Arg-1-Nal-Ala-Cys)-(Pro)₂-Arg-Asp-β-Ala-
(Arg)₅-Gln-(Arg)₄-NH₂;
(SEQ ID NO: 224)
Ac-c(Cys-Glu-His-D-2-Nal-Arg-1-Nal-Ala-Cys)-(Pro)₂-Arg-Asp-β-Ala-
(Arg)₆-Gln-(Arg)₃-NH₂;
(SEQ ID NO: 225)
Ac-c(Cys-Glu-His-D-2-Nal-Arg-1-Nal-Ala-Cys)-(Pro)₂-Lys-Asp-β-Ala-Tyr-
Gly-(Arg)₅-Gln-(Arg)₃-NH₂;
(SEQ ID NO: 226)
Ac-c(Cys-Glu-His-D-2-Nal-Arg-1-Nal-Ala-Cys)-(Pro)₂-Arg-Asp-β-Ala-Tyr-
Gly-(Arg)₅-Gln-(Arg)₃-NH₂;
(SEQ ID NO: 227)
Ac-c(Cys-Glu-His-D-2-Nal-Arg-1-Nal-Ala-Cys)-(Pro)₂-Lys-Asp-β-Ala-Tyr-
Gly-(Arg)₅-Gln-(Arg)₄-NH₂;
(SEQ ID NO: 228)
Ac-c(Cys-Glu-His-D-2-Nal-Arg-1-Nal-Ala-Cys)-(Pro)₂-Arg-Asp-β-Ala-Tyr-
Gly-(Arg)₅-Gln-(Arg)₄-NH₂;
(SEQ ID NO: 229)
Ac-c(Cys-Glu-His-D-2-Nal-Arg-1-Nal-Ala-Cys)-(Pro)₂-Lys-Asp-β-Ala-Tyr-
Gly-(Arg)₆-Gln-(Arg)₃-NH₂;
(SEQ ID NO: 230)
Ac-c(Cys-Glu-His-D-2-Nal-Arg-1-Nal-Ala-Cys)-(Pro)₂-Arg-Asp-β-Ala-Tyr-
Gly-(Arg)6-Gln-(Arg)₃-NH₂;
(SEQ ID NO: 231)
Ac-c(Cys-Glu-His-D-2-Nal-Arg-2-Nal-Ala-Cys)-(Pro)₂-Arg-Asp-β-Ala-
(Arg)₆-Gln-(Arg)₃-NH₂;
(SEQ ID NO: 232)
Ac-c(Cys-Glu-His-D-2-Nal-Arg-2-Nal-Ala-Cys)-(Pro)₂-Lys-Asp-β-Ala-
(Arg)₅-Gln-(Arg)₄-NH₂;
(SEQ ID NO: 233)
Ac-c(Cys-Glu-His-D-2-Nal-Arg-2-Nal-Ala-Cys)-(Pro)₂-Arg-Asp-β-Ala-
(Arg)₅-Gln-(Arg)₄-NH₂;
(SEQ ID NO: 234)
Ac-c(Cys-Glu-His-D-2-Nal-Arg-2-Nal-Ala-Cys)-(Pro)₂-Lys-Asp-β-Ala-Tyr-
Gly-(Arg)₅-Gln-(Arg)₃-NH₂;
(SEQ ID NO: 235)
Ac-c(Cys-Glu-His-D-2-Nal-Arg-2-Nal-Ala-Cys)-(Pro)₂-Arg-Asp-β-Ala-Tyr-
Gly-(Arg)₅-Gln-(Arg)₃-NH₂;
(SEQ ID NO: 236)
Ac-c(Cys-Glu-His-D-2-Nal-Arg-2-Nal-Ala-Cys)-(Pro)₂-Lys-Asp-β-Ala-Tyr-
Gly-(Arg)₆-Gln-(Arg)₃-NH₂;
(SEQ ID NO: 237)
Ac-c(Cys-Glu-His-D-2-Nal-Arg-2-Nal-Ala-Cys)-(Pro)₂-Arg-Asp-β-Ala-Tyr-
Gly-(Arg)₆-Gln-(Arg)₃-NH₂;
(SEQ ID NO: 238)
Ac-c(Cys-Glu-His-D-2-Nal-Arg-2-Nal-Ala-Cys)-(Pro)₂-Lys-Asp-β-Ala-Tyr-
Gly-(Arg)₅-Gln-(Arg)₄-NH₂;
(SEQ ID NO: 239)
Ac-c(Cys-Glu-His-D-2-Nal-Arg-2-Nal-Ala-Cys)-(Pro)₂-Arg-Asp-β-Ala-Tyr-
Gly-(Arg)₅-Gln-(Arg)₄-NH₂;
(SEQ ID NO: 240)
Ac-c(Cys-Glu-His-D-2-Nal-Arg-Bal-Ala-Cys)-(Pro)₂-Lys-Asp-β-Ala-(Arg)₅-
Gln-(Arg)₄-NH₂;
(SEQ ID NO: 241)
Ac-c(Cys-Glu-His-D-2-Nal-Arg-Bal-Ala-Cys)-(Pro)₂-Arg-Asp-β-Ala-
(Arg)₅-Gln-(Arg)₄-NH₂;
(SEQ ID NO: 242)
Ac-c(Cys-Glu-His-D-2-Nal-Arg-Bal-Ala-Cys)-(Pro)₂-Arg-Asp-β-Ala-Tyr-
Gly-(Arg)₅-Gln-(Arg)₃-NH₂;
(SEQ ID NO: 243)
Ac-c(Cys-Glu-His-D-2-Nal-Arg-Bal-Ala-Cys)-(Pro)₂-Lys-Asp-β-Ala-Tyr-
Gly-(Arg)₅-Gln-(Arg)₄-NH₂;
(SEQ ID NO: 244)
Ac-c(Cys-Glu-His-D-2-Nal-Arg-Bal-Ala-Cys)-(Pro)₂-Arg-Asp-β-Ala-Tyr-
Gly-(Arg)₅-Gln-(Arg)₄-NH₂;
(SEQ ID NO: 245)
Ac-c(Cys-Glu-His-D-2-Nal-Arg-Bal-Ala-Cys)-(Pro)₂-Lys-Asp-β-Ala-Tyr-
Gly-(Arg)₆-Gln-(Arg)₃-NH₂;
(SEQ ID NO: 246)
Ac-c(Cys-Glu-His-D-2-Nal-Arg-Bal-Ala-Cys)-(Pro)₂-Arg-Asp-β-Ala-Tyr-
Gly-(Arg)₆-Gln-(Arg)₃-NH₂;
(SEQ ID NO: 247)
Ac-Nle-c(Cys-D-Ala-His-D-Phe-Arg-Trp-Cys)-(Doc)₂-Tyr-Gly-Arg-(Lys)₂-
(Arg)₂-Gln-(Arg)₃-NH₂;
(SEQ ID NO: 248)
Ac-Nle-c(Cys-D-Ala-His-D-Phe-Arg-Trp-Cys)-β-Ala-Tyr-Gly-Arg-(Lys)₂-
Arg-Gln-(Arg)₄-NH₂;
(SEQ ID NO: 249)
Ac-Nle-c(Cys-D-Ala-His-D-Phe-Arg-Trp-Cys)-Doc-Tyr-Gly-Arg-(Lys)₂-
(Arg)₂-Gln-(Arg)₃-NH₂;
(SEQ ID NO: 250)
Ac-Nle-c(Cys-D-Ala-His-D-Phe-Arg-Trp-Cys)-β-Ala-(Arg)₅-Gln-(Arg)₃-
NH₂;
(SEQ ID NO: 251)
Ac-Nle-c(Cys-D-Ala-His-D-Phe-Arg-Trp-Cys)-β-Ala-Gly-(Arg)₅-Gln-
(Arg)₃-NH₂;
(SEQ ID NO: 252)
Ac-Nle-c(Cys-D-Ala-His-D-Phe-Arg-Trp-Cys)-β-Ala-Tyr-Gly-(Arg)₅-Gln-
(Arg)₃-NH₂;
(SEQ ID NO: 253)
Ac-Nle-c(Cys-D-Ala-His-D-Phe-Arg-Trp-Cys)-β-Ala-Gly-(Arg)₅-Gln-
(Arg)₄-NH₂;
(SEQ ID NO: 254)
Ac-Nle-c(Cys-D-Ala-His-D-Phe-Arg-Trp-Cys)-β-Ala-Tyr-Gly-(Arg)₂-Lys-
(Arg)₂-Gln-(Arg)₃-NH₂;
(SEQ ID NO: 255)
Ac-Nle-c(Cys-D-Ala-His-D-Phe-Arg-Trp-Cys)-β-Ala-Tyr-Gly-Arg-Lys-
(Arg)₃-Gln-(Arg)₃-NH₂;
(SEQ ID NO: 256)
Ac-Nle-c(Cys-D-Ala-His-D-Phe-Arg-Trp-Cys)-β-Ala-Gly-(Arg)₂-Lys-
(Arg)₂-Gln-(Arg)₃-NH₂;
(SEQ ID NO: 257)
Ac-Nle-c(Cys-D-Ala-His-D-Phe-Arg-Trp-Cys)-β-Ala-Gly-Arg-Lys-(Arg)₃-
Gln-(Arg)₃-NH₂;
(SEQ ID NO: 258)
Ac-Nle-c(Cys-D-Ala-His-D-Phe-Arg-Trp-Cys)-β-Ala-(Arg)₂-Lys-(Arg)₂-
Gln-(Arg)₃-NH₂;
(SEQ ID NO: 259)
Ac-Nle-c(Cys-D-Ala-His-D-Phe-Arg-Trp-Cys)-β-Ala-Arg-Lys-(Arg)₃-Gln-
(Arg)₃-NH₂;
(SEQ ID NO: 260)
Ac-Nle-c(Cys-D-Ala-His-D-Phe-Arg-Trp-Cys)-(β-Ala)₂-(Arg)₅-Gln-(Arg)₃-
NH₂;
(SEQ ID NO: 261)
Ac-Nle-c(Cys-D-Ala-His-D-Phe-Arg-Trp-Cys)-(β-Ala)₂-Gly-(Arg)₅-Gln-
(Arg)₃-NH₂;
(SEQ ID NO: 262)
Ac-Nle-c(Cys-D-Ala-His-D-Phe-Arg-Trp-Cys)-(β-Ala)₂-Tyr-Gly-(Arg)₅-
Gln-(Arg)₃-NH₂;
(SEQ ID NO: 263)
Ac-Nle-c(Cys-D-Ala-His-D-Phe-Arg-Trp-Cys)-Doc-(Arg)₅-Gln-(Arg)₃-NH₂;
(SEQ ID NO: 264)
Ac-Nle-c(Cys-D-Ala-His-D-Phe-Arg-Trp-Cys)-Doc-Gly-(Arg)₅-Gln-(Arg)₃-
NH₂;
(SEQ ID NO: 265)
Ac-Nle-c(Cys-D-Ala-His-D-Phe-Arg-Trp-Cys)-Doc-Tyr-Gly-(Arg)₅-Gln-
(Arg)₃-NH₂;
(SEQ ID NO: 266)
Ac-Nle-c(Cys-D-Ala-His-D-Phe-Arg-Trp-Cys)-(Doc)₂-(Arg)₅-Gln-(Arg)₃-
NH₂;
(SEQ ID NO: 267)
Ac-Nle-c(Cys-D-Ala-His-D-Phe-Arg-Trp-Cys)-(Doc)₂-Gly-(Arg)-Gln-
(Arg)₃-NH₂;
(SEQ ID NO: 268)
Ac-Nle-c(Cys-D-Ala-His-D-Phe-Arg-Trp-Cys)-(Doc)₂-Tyr-Gly-(Arg)₅-
Gln-(Arg)₃-NH₂;
(SEQ ID NO: 269)
Ac-Nle-c(Cys-D-Ala-His-D-Phe-Arg-Trp-Cys)-β-Ala-(Arg)₅-Gln-(Arg)₄-
NH₂;
(SEQ ID NO: 270)
Ac-Nle-c(Cys-D-Ala-His-D-Phe-Arg-Trp-Cys)-β-Ala-Tyr-Gly-(Arg)₅-Gln-
(Arg)₄-NH₂;
(SEQ ID NO: 271)
Ac-Nle-c(Cys-D-Ala-His-D-Phe-Arg-Trp-Cys)-(β-Ala)₂-(Arg)₅-Gln-(Arg)₄-
NH₂;
(SEQ ID NO: 272)
Ac-Nle-c(Cys-D-Ala-His-D-Phe-Arg-Trp-Cys)-(β-Ala)₂-Gly-(Arg)₅-Gln-
(Arg)₄-NH₂;
(SEQ ID NO: 273)
Ac-Nle-c(Cys-D-Ala-His-D-Phe-Arg-Trp-Cys)-(β-Ala)₂-Tyr-Gly-(Arg)₅-
Gln-(Arg)₄-NH₂;
(SEQ ID NO: 274)
Ac-Nle-c(Cys-D-Ala-His-D-Phe-Arg-Trp-Cys)-Doc-(Arg)₅-Gln-(Arg)₄-
NH₂;
(SEQ ID NO: 275)
Ac-Nle-c(Cys-D-Ala-His-D-Phe-Arg-Trp-Cys)-Doc-Gly-(Arg)₅-Gln-(Arg)₄-
NH₂;
(SEQ ID NO: 276)
Ac-Nle-c(Cys-D-Ala-His-D-Phe-Arg-Trp-Cys)-Doc-Tyr-Gly-(Arg)₅-Gln-
(Arg)₄-NH₂;
(SEQ ID NO: 277)
Ac-Nle-c(Cys-D-Ala-His-D-Phe-Arg-Trp-Cys)-(Doc)₂-(Arg)₅-Gln-(Arg)₄-
NH₂;
(SEQ ID NO: 278)
Ac-Nle-c(Cys-D-Ala-His-D-Phe-Arg-Trp-Cys)-(Doc)₂-Gly-(Arg)₅-Gln-
(Arg)₄-NH₂;
(SEQ ID NO: 279)
Ac-Nle-c(Cys-D-Ala-His-D-Phe-Arg-Trp-Cys)-(Doc)₂-Tyr-Gly-(Arg)₅-
Gln-(Arg)₄-NH₂;
(SEQ ID NO: 280)
Ac-Nle-c(Cys-His-D-Phe-Arg-Trp-Gaba-Cys)-β-Ala-Tyr-Gly-(Arg)₅-Gln-
(Arg)₃-NH₂;
(SEQ ID NO: 281)
Ac-Nle-c(Cys-His-D-Phe-Arg-Trp-Gaba-Cys)-β-Ala-(Arg)₅-Gln-(Arg)₃-NH₂;
(SEQ ID NO: 282)
Ac-Nle-c(Asp-His-D-Phe-Arg-Trp-Ala-Lys)-β-Ala-Tyr-Gly-(Arg)₅-Gln-
(Arg)₃-NH₂;
(SEQ ID NO: 283)
Ac-Nle-c(Asp-His-D-Phe-Arg-Trp-Ala-Lys)-β-Ala-(Arg)₅-Gln-(Arg)₃-NH₂;
(SEQ ID NO: 284)
Ac-Nle-c(Asp-His-D-Phe-Arg-Trp-Lys)-β-Ala-Tyr-Gly-(Arg)₅-Gln-(Arg)₃-
NH₂;
(SEQ ID NO: 285)
Ac-Nle-c(Asp-His-D-Phe-Arg-Trp-Lys)-β-Ala-Gly-(Arg)₅-Gln-(Arg)₃-NH₂;
(SEQ ID NO: 286)
Ac-Nle-c(Asp-His-D-Phe-Arg-Trp-Lys)-β-Ala-(Arg)₅-Gln-(Arg)₃-NH₂;
(SEQ ID NO: 287)
Ac-Nle-c(Asp-His-D-Phe-Arg-Trp-Lys)-(β-Ala)₂-Tyr-Gly-(Arg)₅-Gln-
(Arg)₃-NH₂;
(SEQ ID NO: 288)
Ac-Nle-c(Asp-His-D-Phe-Arg-Trp-Lys)-(β-Ala)₂-Gly-(Arg)₅-Gln-(Arg)₃-
NH₂;
(SEQ ID NO: 289)
Ac-Nle-c(Asp-His-D-Phe-Arg-Trp-Lys)-(β-Ala)₂-(Arg)₅-Gln-(Arg)₃-NH₂;
(SEQ ID NO: 290)
Ac-Nle-c(Asp-His-D-Phe-Arg-Trp-Lys)-Doc-Tyr-Gly-(Arg)₅-Gln-(Arg)₃-
NH₂;
(SEQ ID NO: 291)
Ac-Nle-c(Asp-His-D-Phe-Arg-Trp-Lys)-Doc-Gly-(Arg)₅-Gln-(Arg)₃-NH₂;
(SEQ ID NO: 292)
Ac-Nle-c(Asp-His-D-Phe-Arg-Trp-Lys)-Doc-(Arg)₅-Gln-(Arg)₃-NH₂;
(SEQ ID NO: 293)
Ac-Nle-c(Asp-His-D-Phe-Arg-Trp-Lys)-(Doc)₂-Tyr-Gly-(Arg)₅-Gln-(Arg)₃-
NH₂;
(SEQ ID NO: 294)
Ac-Nle-c(Asp-His-D-Phe-Arg-Trp-Lys)-(Doc)₂-Gly-(Arg)₅-Gln-(Arg)₃-NH₂;
(SEQ ID NO: 295)
Ac-Nle-c(Asp-His-D-Phe-Arg-Trp-Lys)-(Doc)₂-(Arg)₅-Gln-(Arg)₃-NH₂;
(SEQ ID NO: 296)
Ac-Nle-c(Asp-His-D-Phe-Arg-Trp-Lys)-β-Ala-Tyr-Gly-(Arg)₅-Gln-(Arg)₄-
NH₂;
(SEQ ID NO: 297)
Ac-Nle-c(Asp-His-D-Phe-Arg-Trp-Lys)-β-Ala-Gly-(Arg)₅-Gln-(Arg)₄-NH₂;
(SEQ ID NO: 298)
Ac-Nle-c(Asp-His-D-Phe-Arg-Trp-Lys)-β-Ala-(Arg)₅-Gln-(Arg)₄-NH₂;
(SEQ ID NO: 299)
Ac-Nle-c(Asp-His-D-Phe-Arg-Trp-Lys)-(β-Ala)₂-Tyr-Gly-(Arg)₅-Gln-
(Arg)₄-NH₂;
(SEQ ID NO: 300)
Ac-Nle-c(Asp-His-D-Phe-Arg-Trp-Lys)-(β-Ala)₂-Gly-(Arg)₅-Gln-(Arg)₄-
NH₂;
(SEQ ID NO: 301)
Ac-Nle-c(Asp-His-D-Phe-Arg-Trp-Lys)-(β-Ala)₂-(Arg)₅-Gln-(Arg)₄-NH₂;
(SEQ ID NO: 302)
Ac-Nle-c(Asp-His-D-Phe-Arg-Trp-Lys)-Doc-Tyr-Gly-(Arg)₅-Gln-(Arg)₄-
NH₂;
(SEQ ID NO: 303)
Ac-Nle-c(Asp-His-D-Phe-Arg-Trp-Lys)-Doc-Gly-(Arg)₅-Gln-(Arg)₄-NH₂;
(SEQ ID NO: 304)
Ac-Nle-c(Asp-His-D-Phe-Arg-Trp-Lys)-Doc-(Arg)₅-Gln-(Arg)₄-NH₂;
(SEQ ID NO: 305)
Ac-Nle-c(Asp-His-D-Phe-Arg-Trp-Lys)-(Doc)₂-Tyr-Gly-(Arg)₅-Gln-
(Arg)₄-NH₂;
(SEQ ID NO: 306)
Ac-Nle-c(Asp-His-D-Phe-Arg-Trp-Lys)-(Doc)₂-Gly-(Arg)₅-Gln-(Arg)₄-
NH₂;
(SEQ ID NO: 307)
Ac-Nle-c(Asp-His-D-Phe-Arg-Trp-Lys)-(Doc)₂-(Arg)₅-Gln-(Arg)₄-NH₂;
(SEQ ID NO: 308)
Ac-Nle-c(Asp-His-D-Phe-Arg-Trp-β-Ala-Lys)-β-Ala-Tyr-Gly-(Arg)₅-Gln-
(Arg)₃-NH₂;
(SEQ ID NO: 309)
Ac-Nle-c(Asp-His-D-Phe-Arg-Trp-β-Ala-Lys)-β-Ala-(Arg)₅-Gln-(Arg)₃-
NH₂;
(SEQ ID NO: 310)
Ac-Nle-c(Cys-His-D-Phe-Arg-Trp-Ahx-Cys)-β-Ala-Tyr-Gly-(Arg)₅-Gln-
(Arg)₃-NH₂;
(SEQ ID NO: 311)
Ac-Nle-c(Cys-His-D-Phe-Arg-Trp-Ahx-Cys)-β-Ala-(Arg)₅-Gln-(Arg)₃-NH₂;
(SEQ ID NO: 312)
D-Phe-c(Cys-His-D-Phe-Arg-Trp-β-Ala-D-Cys)-Thr-β-Ala-Tyr-Gly-(Arg)₅-
Gln-(Arg)₃-NH₂;
(SEQ ID NO: 313)
D-Phe-c(Cys-His-D-Phe-Arg-Trp-β-Ala-D-Cys)-Thr-β-Ala-(Arg)₅-Gln-
(Arg)₃-NH₂;
(SEQ ID NO: 314)
Ac-Nle-c(Cys-His-D-Phe-Arg-Trp-Apn-Cys)-β-Ala-Tyr-Gly-(Arg)₅-Gln-
(Arg)₃-NH₂;
(SEQ ID NO: 315)
Ac-Nle-c(Cys-His-D-Phe-Arg-Trp-Apn-Cys)-β-Ala-(Arg)₅-Gln-(Arg)₃-NH₂;
(SEQ ID NO: 316)
Ac-Cha-c(Asp-His-D-Phe-Arg-Trp-Gaba-Lys)-β-Ala-Tyr-Gly-(Arg)₅-Gln-
(Arg)₃-NH₂;
(SEQ ID NO: 317)
Ac-Cha-c(Asp-His-D-Phe-Arg-Trp-Gaba-Lys)-β-Ala-(Arg)₅-Gln-(Arg)₃-
NH₂;
(SEQ ID NO: 318)
Ac-Nle-c(Asp-His-D-Phe-Arg-Trp-Gaba-Lys)-β-Ala-Tyr-Gly-(Arg)₅-Gln-
(Arg)₃-NH₂;
(SEQ ID NO: 319)
Ac-Nle-c(Asp-His-D-Phe-Arg-Trp-Gaba-Lys)-β-Ala-(Arg)₅-Gln-(Arg)₃-
NH₂;
(SEQ ID NO: 320)
Ac-Chg-c(Asp-His-D-Phe-Arg-Trp-Gaba-Lys)-β-Ala-Tyr-Gly-(Arg)₅-Gln-
(Arg)₃-NH₂;
(SEQ ID NO: 321)
Ac-Chg-c(Asp-His-D-Phe-Arg-Trp-Gaba-Lys)-β-Ala-(Arg)₅-Gln-(Arg)₃-
NH₂;
(SEQ ID NO: 322)
Ac-hCha-c(Asp-His-D-Phe-Arg-Trp-Gaba-Lys)-β-Ala-Tyr-Gly-(Arg)₅-Gln-
(Arg)₃-NH₂;
(SEQ ID NO: 323)
Ac-hCha-c(Asp-His-D-Phe-Arg-Trp-Gaba-Lys)-β-Ala-(Arg)₅-Gln-(Arg)₃-
NH₂;
(SEQ ID NO: 324)
Ac-hCha-c(Asp-His-D-Phe-Arg-Trp-Gaba-Lys)-(β-Ala)₂-Tyr-Gly-(Arg)₅-
Gln-(Arg)₃-NH₂;
(SEQ ID NO: 325)
Ac-hCha-c(Asp-His-D-Phe-Arg-Trp-Gaba-Lys)-(β-Ala)₂-(Arg)₅-Gln-(Arg)₃-
NH₂;
(SEQ ID NO: 326)
Ac-hCha-c(Asp-His-D-Phe-Arg-Trp-Gaba-Lys)-Doc-Tyr-Gly-(Arg)5-Gln-
(Arg)3-NH₂;
(SEQ ID NO: 327)
Ac-hCha-c(Asp-His-D-Phe-Arg-Trp-Gaba-Lys)-Doc-(Arg)₅-Gln-(Arg)₃-
NH₂;
(SEQ ID NO: 328)
Ac-hCha-c(Asp-His-D-Phe-Arg-Trp-Gaba-Lys)-(Doc)₂-Tyr-Gly-(Arg)₅-
Gln-(Arg)₃-NH₂;
(SEQ ID NO: 329)
Ac-hCha-c(Asp-His-D-Phe-Arg-Trp-Gaba-Lys)-(Doc)₂-(Arg)₅-Gln-(Arg)₃-
NH₂;
(SEQ ID NO: 330)
Ac-hCha-c(Asp-His-D-Phe-Arg-Trp-Gaba-Lys)-β-Ala-Tyr-Gly-(Arg)₅-Gln-
(Arg)₄-NH₂;
(SEQ ID NO: 331)
Ac-hCha-c(Asp-His-D-Phe-Arg-Trp-Gaba-Lys)-β-Ala-(Arg)₅-Gln-(Arg)₄-
NH₂;
(SEQ ID NO: 332)
Ac-hCha-c(Asp-His-D-Phe-Arg-Trp-Gaba-Lys)-(β-Ala)₂-Tyr-Gly-(Arg)₅-
Gln-(Arg)₄-NH₂;
(SEQ ID NO: 333)
Ac-hCha-c(Asp-His-D-Phe-Arg-Trp-Gaba-Lys)-(β-Ala)₂-(Arg)₅-Gln-(Arg)₄-
NH₂;
(SEQ ID NO: 334)
Ac-hCha-c(Asp-His-D-Phe-Arg-Trp-Gaba-Lys)-Doc-Tyr-Gly-(Arg)₅-Gln-
(Arg)₄-NH₂;
(SEQ ID NO: 335)
Ac-hCha-c(Asp-His-D-Phe-Arg-Trp-Gaba-Lys)-Doc-(Arg)₅-Gln-(Arg)₄-
NH₂;
(SEQ ID NO: 336)
Ac-hCha-c(Asp-His-D-Phe-Arg-Trp-Gaba-Lys)-(Doc)₂-Tyr-Gly-(Arg)₅-
Gln-(Arg)₄-NH₂;
(SEQ ID NO: 337)
Ac-hCha-c(Asp-His-D-Phe-Arg-Trp-Gaba-Lys)-(Doc)₂-(Arg)5-Gln-(Arg)₄-
NH₂;
(SEQ ID NO: 338)
Ac-D-Chg-c(Asp-His-D-Phe-Arg-Trp-Gaba-Lys)-β-Ala-Tyr-Gly-(Arg)₅-
Gln-(Arg)₃-NH₂;
(SEQ ID NO: 339)
Ac-D-Chg-c(Asp-His-D-Phe-Arg-Trp-Gaba-Lys)-β-Ala-(Arg)₅-Gln-(Arg)₃-
NH₂;
(SEQ ID NO: 340)
Ac-hPhe-c(Asp-His-D-Phe-Arg-Trp-Gaba-Lys)-β-Ala-Tyr-Gly-(Arg)₅-Gln-
(Arg)₃-NH₂;
(SEQ ID NO: 341)
Ac-hPhe-c(Asp-His-D-Phe-Arg-Trp-Gaba-Lys)-β-Ala-(Arg)₅-Gln-(Arg)₃-
NH₂;
(SEQ ID NO: 342)
Ac-Nle-c(Cys-His-D-Phe-Arg-D-Trp-Apn-Cys)-β-Ala-Tyr-Gly-(Arg)₅-Gln-
(Arg)₃-NH₂;
(SEQ ID NO: 343)
Ac-Nle-c(Cys-His-D-Phe-Arg-D-Trp-Apn-Cys)-β-Ala-(Arg)₅-Gln-(Arg)₃-
NH₂;
(SEQ ID NO: 344)
Ac-Nle-c(Cys-His-D-Phe-Arg-D-Trp-Ahx-Cys)-β-Ala-Tyr-Gly-(Arg)₅-Gln-
(Arg)₃-NH₂;
(SEQ ID NO: 345)
Ac-Nle-c(Cys-His-D-Phe-Arg-D-Trp-Ahx-Cys)-β-Ala-(Arg)₅-Gln-(Arg)₃-
NH₂;
(SEQ ID NO: 346)
Ac-Nle-c(Cys-His-D-Phe-Arg-D-Trp-β-Ala-Cys)-β-Ala-Tyr-Gly-(Arg)₅-
Gln-(Arg)₃-NH₂;
(SEQ ID NO: 347)
Ac-Nle-c(Cys-His-D-Phe-Arg-D-Trp-β-Ala-Cys)-β-Ala-(Arg)₅-Gln-(Arg)₃-
NH₂;
(SEQ ID NO: 348)
Ac-Nle-c(Cys-D-Ala-His-D-Phe-Arg-Trp-Pen)-β-Ala-Tyr-Gly-(Arg)₅-Gln-
(Arg)₃-NH₂;
(SEQ ID NO: 349)
Ac-Nle-c(Cys-D-Ala-His-D-Phe-Arg-Trp-Pen)-β-Ala-Gly-(Arg)₅-Gln-
(Arg)₃-NH₂;
(SEQ ID NO: 350)
Ac-Nle-c(Cys-D-Ala-His-D-Phe-Arg-Trp-Pen)-β-Ala-(Arg)₅-Gln-(Arg)₃-
NH₂;
(SEQ ID NO: 351)
Ac-Nle-c(Cys-D-Ala-His-D-Phe-Arg-Trp-Pen)-(β-Ala)₂-Tyr-Gly-(Arg)₅-
Gln-(Arg)₃-NH₂;
(SEQ ID NO: 352)
Ac-Nle-c(Cys-D-Ala-His-D-Phe-Arg-Trp-Pen)-(β-Ala)₂-Gly-(Arg)₅-Gln-
(Arg)₃-NH₂;
(SEQ ID NO: 353)
Ac-Nle-c(Cys-D-Ala-His-D-Phe-Arg-Trp-Pen)-(β-Ala)2-(Arg)₅-Gln-(Arg)₃-
NH₂;
(SEQ ID NO: 354)
Ac-Nle-c(Cys-D-Ala-His-D-Phe-Arg-Trp-Pen)-Doc-Tyr-Gly-(Arg)₅-Gln-
(Arg)₃-NH₂;
(SEQ ID NO: 355)
Ac-Nle-c(Cys-D-Ala-His-D-Phe-Arg-Trp-Pen)-Doc-Gly-(Arg)₅-Gln-(Arg)₃-
NH₂;
(SEQ ID NO: 356)
Ac-Nle-c(Cys-D-Ala-His-D-Phe-Arg-Trp-Pen)-Doc-(Arg)₅-Gln-(Arg)₃-
NH₂;
(SEQ ID NO: 357)
Ac-Nle-c(Cys-D-Ala-His-D-Phe-Arg-Trp-Pen)-(Doc)₂-Tyr-Gly-(Arg)₅-
Gln-(Arg)₃-NH₂;
(SEQ ID NO: 358)
Ac-Nle-c(Cys-D-Ala-His-D-Phe-Arg-Trp-Pen)-(Doc)₂-Gly-(Arg)₅-Gln-
(Arg)₃-NH₂;
(SEQ ID NO: 359)
Ac-Nle-c(Cys-D-Ala-His-D-Phe-Arg-Trp-Pen)-(Doc)₂-(Arg)₅-Gln-(Arg)₃-
NH₂;
(SEQ ID NO: 360)
D-Phe-c(Cys-His-D-(Et)Tyr-Arg-Trp-β-Ala-D-Cys)-β-Ala-Tyr-Gly-(Arg)₅-
Gln-(Arg)₃-NH₂;
(SEQ ID NO: 361)
D-Phe-c(Cys-His-D-(Et)Tyr-Arg-Trp-β-Ala-D-Cys)-β-Ala-(Arg)₅-Gln-
(Arg)₃-NH₂;
(SEQ ID NO: 362)
D-Phe-c(Cys-His-D-(Et)Tyr-Arg-Trp-β-Ala-D-Cys)-β-Ala-Gly-(Arg)₅-Gln-
(Arg)₃-NH₂;
(SEQ ID NO: 363)
D-Phe-c(Cys-His-D-(Et)Tyr-Arg-Trp-β-Ala-D-Cys)-β-Ala-(Arg)₅-Gln-
(Arg)₄-NH₂;
(SEQ ID NO: 364)
D-Phe-c(Cys-His-D-(Et)Tyr-Arg-Trp-β-Ala-D-Cys)-(β-Ala)₂-Tyr-Gly-
(Arg)5-Gln-(Arg)₃-NH₂;
(SEQ ID NO: 365)
D-Phe-c(Cys-His-D-(Et)Tyr-Arg-Trp-β-Ala-D-Cys)-(β-Ala)₂-(Arg)₅-Gln-
(Arg)₃-NH₂;
(SEQ ID NO: 366)
D-Phe-c(Cys-His-D-(Et)Tyr-Arg-Trp-β-Ala-D-Cys)-(β-Ala)₂-Gly-(Arg)₅-
Gln-(Arg)₃-NH₂;
(SEQ ID NO: 367)
D-Phe-c(Cys-His-D-(Et)Tyr-Arg-Trp-β-Ala-D-Cys)-(β-Ala)₂-(Arg)₅-Gln-
(Arg)₄-NH₂;
(SEQ ID NO: 368)
D-Phe-c(Cys-His-D-(Et)Tyr-Arg-Trp-β-Ala-D-Cys)-Doc-Tyr-Gly-(Arg)₅-
Gln-(Arg)₃-NH₂;
(SEQ ID NO: 369)
D-Phe-c(Cys-His-D-(Et)Tyr-Arg-Trp-β-Ala-D-Cys)-Doc-(Arg)₅-Gln-(Arg)₃-
NH₂;
(SEQ ID NO: 370)
D-Phe-c(Cys-His-D-(Et)Tyr-Arg-Trp-β-Ala-D-Cys)-Doc-Gly-(Arg)₅-Gln-
(Arg)₃-NH₂;
(SEQ ID NO: 371)
D-Phe-c(Cys-His-D-(Et)Tyr-Arg-Trp-β-Ala-D-Cys)-Doc-(Arg)₅-Gln-(Arg)₄-
NH₂;
(SEQ ID NO: 372)
D-Phe-c(Cys-His-D-(Et)Tyr-Arg-Trp-β-Ala-D-Cys)-(Doc)₂-Tyr-Gly-(Arg)₅-
Gln-(Arg)₃-NH₂;
(SEQ ID NO: 373)
D-Phe-c(Cys-His-D-(Et)Tyr-Arg-Trp-β-Ala-D-Cys)-(Doc)₂-(Arg)₅-Gln-
(Arg)₃-NH₂;
(SEQ ID NO: 374)
D-Phe-c(Cys-His-D-(Et)Tyr-Arg-Trp-β-Ala-D-Cys)-(Doc)₂-Gly-(Arg)₅-
Gln-(Arg)₃-NH₂;
(SEQ ID NO: 375)
D-Phe-c(Cys-His-D-(Et)Tyr-Arg-Trp-β-Ala-D-Cys)-(Doc)₂-(Arg)₅-Gln-
(Arg)₄-NH₂;
(SEQ ID NO: 376)
D-Phe-c(Cys-His-D-(Et)Tyr-hArg-Trp-β-Ala-D-Cys)-Thr-β-Ala-Tyr-Gly-
(Arg)₅-Gln-(Arg)₃-NH₂;
(SEQ ID NO: 377)
D-Phe-c(Cys-His-D-(Et)Tyr-hArg-Trp-β-Ala-D-Cys)-Thr-β-Ala-(Arg)₅-
Gln-(Arg)₃-NH₂;
(SEQ ID NO: 378)
D-Phe-c(Cys-His-D-(Et)Tyr-hArg-Trp-β-Ala-D-Cys)-Thr-(β-Ala)₂-Tyr-Gly-
(Arg)₅-Gln-(Arg)₃-NH₂;
(SEQ ID NO: 379)
D-Phe-c(Cys-His-D-(Et)Tyr-hArg-Trp-β-Ala-D-Cys)-Thr-(β-Ala)₂-(Arg)₅-
Gln-(Arg)₃-NH₂;
(SEQ ID NO: 380)
D-Phe-c(Cys-His-D-(Et)Tyr-hArg-Trp-β-Ala-D-Cys)-Thr-Doc-Tyr-Gly-
(Arg)₅-Gln-(Arg)₃-NH₂;
(SEQ ID NO: 381)
D-Phe-c(Cys-His-D-(Et)Tyr-hArg-Trp-β-Ala-D-Cys)-Thr-Doc-(Arg)₅-Gln-
(Arg)₃-NH₂;
(SEQ ID NO: 382)
D-Phe-c(Cys-His-D-(Et)Tyr-hArg-Trp-β-Ala-D-Cys)-Thr-(Doc)₂-Tyr-Gly-
(Arg)₅-Gln-(Arg)₃-NH₂;
(SEQ ID NO: 383)
D-Phe-c(Cys-His-D-(Et)Tyr-hArg-Trp-β-Ala-D-Cys)-Thr-β-Ala-Tyr-Gly-
(Arg)₅-Gln-(Arg)₄-NH₂;
(SEQ ID NO: 384)
D-Phe-c(Cys-His-D-(Et)Tyr-hArg-Trp-β-Ala-D-Cys)-Thr-β-Ala-(Arg)₅-
Gln-(Arg)₄-NH₂;
(SEQ ID NO: 385)
D-Phe-c(Cys-His-D-(Et)Tyr-hArg-Trp-β-Ala-D-Cys)-Thr-(β-Ala)₂-Tyr-Gly-
(Arg)₅-Gln-(Arg)₄-NH₂;
(SEQ ID NO: 386)
D-Phe-c(Cys-His-D-(Et)Tyr-hArg-Trp-β-Ala-D-Cys)-Thr-(β-Ala)₂-(Arg)₅-
Gln-(Arg)₄-NH₂;
(SEQ ID NO: 387)
D-Phe-c(Cys-His-D-(Et)Tyr-hArg-Trp-β-Ala-D-Cys)-Thr-Doc-Tyr-Gly-
(Arg)₅-Gln-(Arg)₄-NH₂;
(SEQ ID NO: 388)
D-Phe-c(Cys-His-D-(Et)Tyr-hArg-Trp-β-Ala-D-Cys)-Thr-Doc-(Arg)₅-Gln-
(Arg)₄-NH₂;
(SEQ ID NO: 389)
D-Phe-c(Cys-His-D-(Et)Tyr-hArg-Trp-β-Ala-D-Cys)-Thr-(Doc)₂-Tyr-Gly-
(Arg)₅-Gln-(Arg)₄-NH₂;
(SEQ ID NO: 390)
D-Phe-c(Cys-His-D-(Et)Tyr-hArg-Trp-β-Ala-D-Cys)-Thr-(Doc)₂-(Arg)₅-
Gln-(Arg)₄-NH₂;
(SEQ ID NO: 391)
D-Phe-c(Cys-His-D-(Et)Tyr-hArg-Bip-β-Ala-D-Cys)-Thr-β-Ala-Tyr-Gly-
(Arg)₅-Gln-(Arg)₃-NH₂;
(SEQ ID NO: 392)
D-Phe-c(Cys-His-D-(Et)Tyr-hArg-Bip-β-Ala-D-Cys)-Thr-β-Ala-Tyr-Gly-
(Arg)₅-Gln-(Arg)₄-NH₂;
(SEQ ID NO: 393)
D-Phe-c(Cys-His-D-(Et)Tyr-hArg-Bip-β-Ala-D-Cys)-Thr-β-Ala-(Arg)₅-
Gln-(Arg)₃-NH₂;
(SEQ ID NO: 394)
D-Phe-c(Cys-His-D-(Et)Tyr-hArg-Bip-β-Ala-D-Cys)-Thr-(β-Ala)₂-Tyr-Gly-
(Arg)₅-Gln-(Arg)₃-NH₂;
(SEQ ID NO: 395)
D-Phe-c(Cys-His-D-(Et)Tyr-hArg-Bip-β-Ala-D-Cys)-Thr-(β-Ala)₂-(Arg)₅-
Gln-(Arg)₃-NH₂;
(SEQ ID NO: 396)
D-Phe-c(Cys-His-D-(Et)Tyr-hArg-Bip-β-Ala-D-Cys)-Thr-Doc-Tyr-Gly-
(Arg)₅-Gln-(Arg)₃-NH₂;
(SEQ ID NO: 397)
D-Phe-c(Cys-His-D-(Et)Tyr-hArg-Bip-β-Ala-D-Cys)-Thr-Doc-Tyr-Gly-
(Arg)₅-Gln-(Arg)₄-NH₂;
(SEQ ID NO: 398)
D-Phe-c(Cys-His-D-(Et)Tyr-hArg-Bip-β-Ala-D-Cys)-Thr-Doc-(Arg)₅-Gln-
(Arg)₃-NH₂;
(SEQ ID NO: 399)
D-Phe-c(Cys-His-D-(Et)Tyr-hArg-Bip-β-Ala-D-Cys)-Thr-(Doc)₂-Tyr-Gly-
(Arg)₅-Gln-(Arg)₃-NH₂;
(SEQ ID NO: 400)
D-Phe-c(Cys-His-D-(Et)Tyr-hArg-Bip-β-Ala-D-Cys)-Thr-(Doc)₂-(Arg)₅-
Gln-(Arg)₃-NH₂;
(SEQ ID NO: 401)
Ac-Nle-c(Cys-D-Ala-His-D-Phe-Arg-Trp-Gly-Cys)-(β-Ala-Tyr-Gly-(Arg)₅-
Gln-(Arg)₃-NH₂;
(SEQ ID NO: 402)
Ac-Nle-c(Cys-D-Ala-His-D-Phe-Arg-Trp-Gly-Cys)-(β-Ala-(Arg)₅-Gln-
(Arg)₃-NH₂;
(SEQ ID NO: 403)
Nle-c(Cys-His-D-Phe-Arg-Trp-Apn-Cys)-β-Ala-Tyr-Gly-(Arg)₅-Gln-(Arg)₃-
NH₂;
(SEQ ID NO: 404)
Nle-c(Cys-His-D-Phe-Arg-Trp-Apn-Cys)-β-Ala-(Arg)₅-Gln-(Arg)₃-NH₂;
(SEQ ID NO: 405)
Nle-c(Cys-His-D-Phe-Arg-Trp-Apn-Cys)-(β-Ala)₂-Tyr-Gly-(Arg)₅-Gln-
(Arg)₃-NH₂;
(SEQ ID NO: 406)
Nle-c(Cys-His-D-Phe-Arg-Trp-Apn-Cys)-(β-Ala)₂-(Arg)₅-Gln-(Arg)₃-NH₂;
(SEQ ID NO: 407)
Nle-c(Cys-His-D-Phe-Arg-Trp-Apn-Cys)-β-Ala-Tyr-Gly-(Arg)₅-Gln-(Arg)₄-
NH₂;
(SEQ ID NO: 408)
Nle-c(Cys-His-D-Phe-Arg-Trp-Apn-Cys)-β-Ala-(Arg)₅-Gln-(Arg)₄-NH₂;
(SEQ ID NO: 409)
Nle-c(Cys-His-D-Phe-Arg-Trp-Apn-Cys)-(β-Ala)₂-Tyr-Gly-(Arg)₅-Gln-
(Arg)₄-NH₂;
(SEQ ID NO: 410)
Nle-c(Cys-His-D-Phe-Arg-Trp-Apn-Cys)-(β-Ala)₂-(Arg)₅-Gln-(Arg)₄-NH₂;
(SEQ ID NO: 411)
Nle-c(Cys-His-D-Phe-Arg-Trp-Apn-Cys)-Doc-Tyr-Gly-(Arg)₅-Gln-(Arg)₃-
NH₂;
(SEQ ID NO: 412)
Nle-c(Cys-His-D-Phe-Arg-Trp-Apn-Cys)-Doc-(Arg)₅-Gln-(Arg)₃-NH₂;
(SEQ ID NO: 413)
Nle-c(Cys-His-D-Phe-Arg-Trp-Apn-Cys)-(Doc)₂-Tyr-Gly-(Arg)₅-Gln-
(Arg)₃-NH₂;
(SEQ ID NO: 414)
Nle-c(Cys-His-D-Phe-Arg-Trp-Apn-Cys)-(Doc)₂-(Arg)₅-Gln-(Arg)₃-NH₂;
(SEQ ID NO: 415)
Nle-c(Cys-His-D-Phe-Arg-Trp-Apn-Cys)-Doc-Tyr-Gly-(Arg)₅-Gln-(Arg)₄-
NH₂;
(SEQ ID NO: 416)
Nle-c(Cys-His-D-Phe-Arg-Trp-Apn-Cys)-Doc-(Arg)₅-Gln-(Arg)₄-NH₂;
(SEQ ID NO: 417)
Nle-c(Cys-His-D-Phe-Arg-Trp-Apn-Cys)-(Doc)₂-Tyr-Gly-(Arg)₅-Gln-(Arg)₄-
NH₂;
(SEQ ID NO: 418)
Nle-c(Cys-His-D-Phe-Arg-Trp-Apn-Cys)-(Doc)₂-(Arg)₅-Gln-(Arg)₄-NH₂;
(SEQ ID NO: 419)
Ac-Nle-c(Cys-D-Leu-His-D-Phe-Arg-Trp-Cys)-β-Ala-Tyr-Gly-(Arg)₅-Gln-
(Arg)₃-NH₂;
(SEQ ID NO: 420)
Ac-Nle-c(Cys-D-Leu-His-D-Phe-Arg-Trp-Cys)-β-Ala-(Arg)₅-Gln-(Arg)₃-
NH₂;
(SEQ ID NO: 421)
Ac-Nle-c(Cys-D-Leu-His-D-Phe-Arg-Trp-Cys)-(β-Ala)₂-Tyr-Gly-(Arg)₅-
Gln-(Arg)₃-NH₂;
(SEQ ID NO: 422)
Ac-Nle-c(Cys-D-Leu-His-D-Phe-Arg-Trp-Cys)-(β-Ala)₂-(Arg)₅-Gln-(Arg)₃-
NH₂;
(SEQ ID NO: 423)
Ac-Nle-c(Cys-D-Leu-His-D-Phe-Arg-Trp-Cys)-Doc-Tyr-Gly-(Arg)₅-Gln-
(Arg)₃-NH₂;
(SEQ ID NO: 424)
Ac-Nle-c(Cys-D-Leu-His-D-Phe-Arg-Trp-Cys)-Doc-(Arg)₅-Gln-(Arg)₃-
NH₂;
(SEQ ID NO: 425)
Ac-Nle-c(Cys-D-Leu-His-D-Phe-Arg-Trp-Cys)-(Doc)₂-Tyr-Gly-(Arg)₅-
Gln-(Arg)₃-NH₂;
(SEQ ID NO: 426)
Ac-Nle-c(Cys-D-Leu-His-D-Phe-Arg-Trp-Cys)-(Doc)₂-(Arg)₅-Gln-(Arg)₃-
NH₂;
(SEQ ID NO: 427)
Ac-Nle-c(Cys-D-Leu-His-D-Phe-Arg-Trp-Cys)-β-Ala-Tyr-Gly-(Arg)₅-Gln-
(Arg)₄-NH₂;
(SEQ ID NO: 428)
Ac-Nle-c(Cys-D-Leu-His-D-Phe-Arg-Trp-Cys)-β-Ala-(Arg)₅-Gln-(Arg)₄-
NH₂;
(SEQ ID NO: 429)
Ac-Nle-c(Cys-D-Leu-His-D-Phe-Arg-Trp-Cys)-(β-Ala)₂-Tyr-Gly-(Arg)₅-
Gln-(Arg)₄-NH₂;
(SEQ ID NO: 430)
Ac-Nle-c(Cys-D-Leu-His-D-Phe-Arg-Trp-Cys)-(β-Ala)₂-(Arg)₅-Gln-(Arg)₄-
NH₂;
(SEQ ID NO: 431)
Ac-Nle-c(Cys-D-Leu-His-D-Phe-Arg-Trp-Cys)-Doc-Tyr-Gly-(Arg)₅-Gln-
(Arg)₄-NH₂;
(SEQ ID NO: 432)
Ac-Nle-c(Cys-D-Leu-His-D-Phe-Arg-Trp-Cys)-Doc-(Arg)₅-Gln-(Arg)₄-
NH₂;
(SEQ ID NO: 433)
Ac-Nle-c(Cys-D-Leu-His-D-Phe-Arg-Trp-Cys)-(Doc)₂-Tyr-Gly-(Arg)₅-
Gln-(Arg)₄-NH₂;
(SEQ ID NO: 434)
Ac-Nle-c(Cys-D-Leu-His-D-Phe-Arg-Trp-Cys)-(Doc)₂-(Arg)₅-Gln-(Arg)₄-
NH₂;
(SEQ ID NO: 435)
Ac-Nle-c(Cys-D-Cha-His-D-Phe-Arg-Trp-Cys)-β-Ala-Tyr-Gly-(Arg)₅-Gln-
(Arg)₃-NH₂;
(SEQ ID NO: 436)
Ac-Nle-c(Cys-D-Cha-His-D-Phe-Arg-Trp-Cys)-β-Ala-(Arg)₅-Gln-(Arg)₃-
NH₂;
(SEQ ID NO: 437)
Ac-Nle-c(Cys-D-Cha-His-D-Phe-Arg-Trp-Cys)-(β-Ala)₂-Tyr-Gly-(Arg)₅-
Gln-(Arg)₃-NH₂;
(SEQ ID NO: 438)
Ac-Nle-c(Cys-D-Cha-His-D-Phe-Arg-Trp-Cys)-(β-Ala)₂-(Arg)₅-Gln-(Arg)₃-
NH₂;
(SEQ ID NO: 439)
Ac-Nle-c(Cys-D-Cha-His-D-Phe-Arg-Trp-Cys)-Doc-Tyr-Gly-(Arg)₅-Gln-
(Arg)₃-NH₂;
(SEQ ID NO: 440)
Ac-Nle-c(Cys-D-Cha-His-D-Phe-Arg-Trp-Cys)-Doc-(Arg)₅-Gln-(Arg)₃-
NH₂;
(SEQ ID NO: 441)
Ac-Nle-c(Cys-D-Cha-His-D-Phe-Arg-Trp-Cys)-(Doc)₂-Tyr-Gly-(Arg)₅-
Gln-(Arg)₃-NH₂;
(SEQ ID NO: 442)
Ac-Nle-c(Cys-D-Cha-His-D-Phe-Arg-Trp-Cys)-(Doc)₂-(Arg)₅-Gln-(Arg)₃-
NH₂;
(SEQ ID NO: 443)
Ac-Nle-c(Cys-D-Cha-His-D-Phe-Arg-Trp-Cys)-β-Ala-Tyr-Gly-(Arg)₅-Gln-
(Arg)₄-NH₂;
(SEQ ID NO: 444)
Ac-Nle-c(Cys-D-Cha-His-D-Phe-Arg-Trp-Cys)-β-Ala-(Arg)₅-Gln-(Arg)-
NH₂;
(SEQ ID NO: 445)
Ac-Nle-c(Cys-D-Cha-His-D-Phe-Arg-Trp-Cys)-(β-Ala)₂-Tyr-Gly-(Arg)₅-
Gln-(Arg)₄-NH₂;
(SEQ ID NO: 446)
Ac-Nle-c(Cys-D-Cha-His-D-Phe-Arg-Trp-Cys)-(β-Ala)₂-(Arg)₅-Gln-(Arg)₄-
NH₂;
(SEQ ID NO: 447)
Ac-Nle-c(Cys-D-Cha-His-D-Phe-Arg-Trp-Cys)-Doc-Tyr-Gly-(Arg)₅-Gln-
(Arg)₄-NH₂;
(SEQ ID NO: 448)
Ac-Nle-c(Cys-D-Cha-His-D-Phe-Arg-Trp-Cys)-Doc-(Arg)₅-Gln-(Arg)₄-
NH₂;
(SEQ ID NO: 449)
Ac-Nle-c(Cys-D-Cha-His-D-Phe-Arg-Trp-Cys)-(Doc)₂-Tyr-Gly-(Arg)₅-
Gln-(Arg)₄-NH₂;
(SEQ ID NO: 450)
Ac-Nle-c(Cys-D-Cha-His-D-Phe-Arg-Trp-Cys)-(Doc)₂-(Arg)₅-Gln-(Arg)₄-
NH₂;
(SEQ ID NO: 451)
Nle-c(Cys-His-D-Phe-Arg-Trp-Gaba-Cys)-β-Ala-Tyr-Gly-(Arg)₅-Gln-
(Arg)₃-NH₂;
(SEQ ID NO: 452)
Nle-c(Cys-His-D-Phe-Arg-Trp-Gaba-Cys)-β-Ala-(Arg)₅-Gln-(Arg)₃-NH₂;
(SEQ ID NO: 453)
Nle-c(Cys-His-D-Phe-Arg-Trp-Gaba-Cys)-(β-Ala)₂-Tyr-Gly-(Arg)₅-Gln-
(Arg)₃-NH₂;
(SEQ ID NO: 454)
Nle-c(Cys-His-D-Phe-Arg-Trp-Gaba-Cys)-(β-Ala)₂-(Arg)₅-Gln-(Arg)₃-
NH₂;
(SEQ ID NO: 455)
Nle-c(Cys-His-D-Phe-Arg-Trp-Gaba-Cys)-β-Ala-Tyr-Gly-(Arg)₅-Gln-
(Arg)₄-NH₂;
(SEQ ID NO: 456)
Nle-c(Cys-His-D-Phe-Arg-Trp-Gaba-Cys)-β-Ala-(Arg)₅-Gln-(Arg)₄-NH₂;
(SEQ ID NO: 457)
Nle-c(Cys-His-D-Phe-Arg-Trp-Gaba-Cys)-(β-Ala)₂-Tyr-Gly-(Arg)₅-Gln-
(Arg)₄-NH₂;
(SEQ ID NO: 458)
Nle-c(Cys-His-D-Phe-Arg-Trp-Gaba-Cys)-(β-Ala)₂-(Arg)₅-Gln-(Arg)₄-
NH₂;
(SEQ ID NO: 459)
Nle-c(Cys-His-D-Phe-Arg-Trp-Gaba-Cys)-Doc-Tyr-Gly-(Arg)₅-Gln-(Arg)₃-
NH₂;
(SEQ ID NO: 460)
Nle-c(Cys-His-D-Phe-Arg-Trp-Gaba-Cys)-Doc-(Arg)₅-Gln-(Arg)₃-NH₂;
(SEQ ID NO: 461)
Nle-c(Cys-His-D-Phe-Arg-Trp-Gaba-Cys)-(Doc)₂-Tyr-Gly-(Arg)₅-Gln-
(Arg)₃-NH₂;
(SEQ ID NO: 462)
Nle-c(Cys-His-D-Phe-Arg-Trp-Gaba-Cys)-(Doc)₂-(Arg)₅-Gln-(Arg)₃-NH₂;
(SEQ ID NO: 463)
Nle-c(Cys-His-D-Phe-Arg-Trp-Gaba-Cys)-Doc-Tyr-Gly-(Arg)₅-Gln-(Arg)₄-
NH₂;
(SEQ ID NO: 464)
Nle-c(Cys-His-D-Phe-Arg-Trp-Gaba-Cys)-Doc-(Arg)₅-Gln-(Arg)₄-NH₂;
(SEQ ID NO: 465)
Nle-c(Cys-His-D-Phe-Arg-Trp-Gaba-Cys)-(Doc)₂-Tyr-Gly-(Arg)₅-Gln-
(Arg)₄-NH₂;
(SEQ ID NO: 466)
Nle-c(Cys-His-D-Phe-Arg-Trp-Gaba-Cys)-(Doc)₂-(Arg)₅-Gln-(Arg)₄-NH₂;
(SEQ ID NO: 467)
Ac-c(Cys-Glu-His-D-4-Br-Phe-Arg-Trp-Gly-Cys)-(Pro)₂-Lys-Asp-NH₂;
(SEQ ID NO: 468)
Ac-c(Cys-Glu-His-D-2-Nal-Arg-Trp-Ala-Cys)-(Pro)₂-Lys-Asp-NH₂;
(SEQ ID NO: 469)
Ac-c(Cys-Glu-His-D-2-Nal-Arg-2-Nal-Ala-Cys)-(Pro)₂-Lys-Asp-NH₂;
(SEQ ID NO: 470)
Ac-c(Cys-Glu-His-D-2-Nal-Arg-1-Nal-Ala-Cys)-(Pro)₂-Lys-Asp-NH₂;
(SEQ ID NO: 471)
Ac-c(Cys-Glu-His-D-2-Nal-Arg-Bal-Ala-Cys)-(Pro)₂-Lys-Asp-NH₂;
(SEQ ID NO: 472)
Ac-c(Cys-Glu-His-D-2-Nal-Arg-2-Nal-β-Ala-Cys)-(Pro)₂-Lys-Asp-NH₂;
(SEQ ID NO: 473)
Ac-c(Cys-Glu-His-D-2-Nal-Arg-2-Nal-Aib-Cys)-(Pro)₂-Lys-Asp-NH₂;
(SEQ ID NO: 474)
c[Hydantoin(C(O)-(Cys-D-Ala))-His-D-Phe-Arg-Trp-Cys]-NH₂;
(SEQ ID NO: 475)
c[Hydantoin(C(O)-(hCys-D-Ala))-His-D-Phe-Arg-Trp-Cys]-NH₂;
(SEQ ID NO: 476)
c[Hydantoin(C(O)-(Cys-D-Ala))-His-D-2-Nal-Arg-Trp-Cys]-NH₂;
(SEQ ID NO: 477)
c[Hydantoin(C(O)-(hCys-D-Ala))-His-D-2-Nal-Arg-Trp-Cys]-NH₂;
(SEQ ID NO: 478)
c[Hydantoin(C(O)-(Asp-D-Ala))-His-D-Phe-Arg-Trp-Lys]-NH₂;
(SEQ ID NO: 479)
c[Hydantoin(C(O)-(Asp-D-Ala))-His-D-Phe-Arg-Trp-Orn]-NH₂;
(SEQ ID NO: 480)
c[Hydantoin(C(O)-(Asp-D-Ala))-His-D-Phe-Arg-Trp-DaN-NH₂;
(SEQ ID NO: 481)
c[Hydantoin(C(O)-(Asp-D-Ala))-His-D-Phe-Arg-Trp-Dap]-NH₂;
(SEQ ID NO: 482)
c[Hydantoin(C(O)-(Asp-His))-D-2-Nal-Arg-Trp-Lys]-NH₂;
(SEQ ID NO: 483)
c[Hydantoin(C(O)-(Asp-His))-D-Phe-Arg-Trp-Lys]-NH₂;
(SEQ ID NO: 484)
c[Hydantoin(C(O)-(Asp-A3c))-D-Phe-Arg-Trp-Lys]-NH₂;
(SEQ ID NO: 485)
c[Hydantoin(C(O)-(Asp-A5c))-D-Phe-Arg-Trp-Lys]-NH₂;
(SEQ ID NO: 486)
c[Hydantoin(C(O)-(Asp-A6c))-D-Phe-Arg-Trp-Lys]-NH₂;
(SEQ ID NO: 487)
c[Hydantoin(C(O)-(Asp-A3c))-D-2-Nal-Arg-Trp-Lys]-NH₂;
(SEQ ID NO: 488)
c[Hydantoin(C(O)-(Asp-A5c))-D-2-Nal-Arg-Trp-Lys]-NH₂;
(SEQ ID NO: 489)
c[Hydantoin(C(O)-(Asp-A6c))-D-2-Nal-Arg-Trp-Lys]-NH₂;
(SEQ ID NO: 490)
c[Hydantoin(C(O)-(Asp-Aic))-D-Phe-Arg-Trp-Lys]-NH₂;
(SEQ ID NO: 491)
c[Hydantoin(C(O)-(Asp-Apc))-D-Phe-Arg-Trp-Lys]-NH₂;
(SEQ ID NO: 492)
c[Hydantoin(C(O)-(Asp-Aic))-D-2-Nal-Arg-Trp-Lys]-NH₂;
(SEQ ID NO: 493)
c[Hydantoin(C(O)-(Asp-Apc))-D-2-Nal-Arg-Trp-Lys]-NH₂;
(SEQ ID NO: 494)
c[Hydantoin(C(O)-(Glu-D-Ala))-His-D-Phe-Arg-Trp-Orn]-NH₂;
(SEQ ID NO: 495)
c[Hydantoin(C(O)-(Glu-D-Ala))-His-D-Phe-Arg-Trp-DaN-NH₂;
(SEQ ID NO: 496)
c[Hydantoin(C(O)-(Glu-D-Ala))-His-D-Phe-Arg-Trp-Dap]-NH₂;
(SEQ ID NO: 497)
c[Hydantoin(C(O)-(Glu-D-Ala))-His-D-Phe-Arg-Trp-Lys]-NH₂;
(SEQ ID NO: 498)
c[Hydantoin(C(O)-(Glu-His))-D-Phe-Arg-Trp-Dap]-NH₂;
(SEQ ID NO: 499)
c[Hydantoin(C(O)-(Glu-His))-D-Phe-Arg-Trp-Lys]-NH₂;
(SEQ ID NO: 500)
Hydantoin(C(O)-(Arg-Gly))-c(Cys-Glu-His-D-Phe-Arg-Trp-Cys)-NH₂;
(SEQ ID NO: 501)
Hydantoin(C(O)-(Nle-Gly))-c(Cys-Glu-His-D-Phe-Arg-Trp-Cys)-NH₂;
(SEQ ID NO: 502)
Hydantoin(C(O)-(Gly-Gly))-c(Cys-Glu-His-D-Phe-Arg-Trp-Cys)-NH₂;
(SEQ ID NO: 503)
Hydantoin(C(O)-(Nle-Gly))-c(Cys-D-Ala-His-D-Phe-Arg-Trp-Cys)-NH₂;
(SEQ ID NO: 504)
Hydantoin(C(O)-(Gly-Gly))-c(Cys-D-Ala-His-D-Phe-Arg-Trp-Cys)-NH₂;
(SEQ ID NO: 505)
Hydantoin(C(O)-(Nle-Gly))-c(Cys-D-Ala-His-D-Phe-Arg-Trp-Pen)-NH₂;
(SEQ ID NO: 506)
Hydantoin(C(O)-(Gly-Gly))-c(Cys-D-Ala-His-D-Phe-Arg-Trp-Pen)-NH₂;
(SEQ ID NO: 507)
Hydantoin(C(O)-(Ala-Gly))-c(Cys-D-Ala-His-D-Phe-Arg-Trp-Cys)-NH₂;
(SEQ ID NO: 508)
Hydantoin(C(O)-(D-Ala-Gly))-c(Cys-D-Ala-His-D-Phe-Arg-Trp-Cys)-NH₂;
(SEQ ID NO: 509)
Hydantoin(C(O)-(Aib-Gly))-c(Cys-D-Ala-His-D-Phe-Arg-Trp-Cys)-NH₂;
(SEQ ID NO: 510)
Hydantoin(C(O)-(Val-Gly))-c(Cys-D-Ala-His-D-Phe-Arg-Trp-Cys)-NH₂;
(SEQ ID NO: 511)
Hydantoin(C(O)-(Ile-Gly))-c(Cys-D-Ala-His-D-Phe-Arg-Trp-Cys)-NH₂;
(SEQ ID NO: 512)
Hydantoin(C(O)-(Leu-Gly))-c(Cys-D-Ala-His-D-Phe-Arg-Trp-Cys)-NH₂;
(SEQ ID NO: 513)
Hydantoin(C(O)-(Gly-Gly))-c(Cys-Glu-His-D-2-Nal-Arg-Trp-Cys)-NH₂;
(SEQ ID NO: 514)
Hydantoin(C(O)-(Nle-Gly))-c(Cys-Glu-His-D-2-Nal-Arg-Trp-Cys)-NH₂;
(SEQ ID NO: 515)
Hydantoin(C(O)-(D-Arg-Gly))-c(Cys-Glu-His-D-Phe-Arg-Trp-Cys)-NH₂;
(SEQ ID NO: 516)
Hydantoin(C(O)-(D-Arg-Gly))-c(Cys-D-Ala-His-D-Phe-Arg-Trp-Cys)-NH₂;
(SEQ ID NO: 517)
Hydantoin(C(O)-(Arg-Gly))-c(Cys-D-Ala-His-D-Phe-Arg-Trp-Cys)-NH₂;
(SEQ ID NO: 518)
Hydantoin(C(O)-(D-Arg-Gly))-c(Cys-D-Ala-His-D-2-Nal-Arg-Trp-Cys)-NH₂;
(SEQ ID NO: 519)
Hydantoin(C(O)-(Arg-Gly))-c(Cys-D-Ala-His-D-2-Nal-Arg-Trp-Cys)-NH₂;
(SEQ ID NO: 520)
Hydantoin(C(O)-(Ala-Nle))-c(Cys-Glu-His-D-Phe-Arg-Trp-Cys)-NH₂;
(SEQ ID NO: 521)
Hydantoin(C(O)-(Val-Nle))-c(Cys-Glu-His-D-Phe-Arg-Trp-Cys)-NH₂;
(SEQ ID NO: 522)
Hydantoin(C(O)-(Gly-Nle))-c(Cys-Glu-His-D-Phe-Arg-Trp-Cys)-NH₂;
(SEQ ID NO: 523)
Hydantoin(C(O)-(A6c-Nle))-c(Cys-D-Ala-His-D-Phe-Arg-Trp-Cys)-NH₂;
(SEQ ID NO: 524)
Hydantoin(C(O)-(Gly-Nle))-c(Cys-D-Ala-His-D-Phe-Arg-Trp-Cys)-NH₂;
(SEQ ID NO: 525)
Hydantoin(C(O)-(Ala-Nle))-c(Cys-D-Ala-His-D-Phe-Arg-Trp-Cys)-NH₂;
(SEQ ID NO: 526)
Hydantoin(C(O)-(D-Ala-Nle))-c(Cys-D-Ala-His-D-Phe-Arg-Trp-Cys)-NH₂;
(SEQ ID NO: 527)
Hydantoin(C(O)-(Val-Nle))-c(Cys-D-Ala-His-D-Phe-Arg-Trp-Cys)-NH₂;
(SEQ ID NO: 528)
Hydantoin(C(O)-(Leu-Nle))-c(Cys-D-Ala-His-D-Phe-Arg-Trp-Cys)-NH₂;
(SEQ ID NO: 529)
Hydantoin(C(O)-(Cha-Nle))-c(Cys-D-Ala-His-D-Phe-Arg-Trp-Cys)-NH₂;
(SEQ ID NO: 530)
Hydantoin(C(O)-(Aib-Nle))-c(Cys-D-Ala-His-D-Phe-Arg-Trp-Cys)-NH₂;
(SEQ ID NO: 531)
Hydantoin(C(O)-(Gly-Arg))-c(Cys-Glu-His-D-Phe-Arg-Trp-Cys)-NH₂;
(SEQ ID NO: 532)
Hydantoin(C(O)-(Gly-Arg))-c(Cys-Glu-His-D-2-Nal-Arg-Trp-Cys)-NH₂;
(SEQ ID NO: 533)
Hydantoin(C(O)-(Gly-Arg))-c(Cys-D-Ala-His-D-Phe-Arg-Trp-Cys)-NH₂;
(SEQ ID NO: 534)
Hydantoin(C(O)-(Gly-Arg))-c(Cys-D-Ala-His-D-2-Nal-Arg-Trp-Cys)-NH₂;
(SEQ ID NO: 535)
Hydantoin(C(O)-(Gly-D-Arg))-c(Cys-Glu-His-D-Phe-Arg-Trp-Cys)-NH₂;
(SEQ ID NO: 536)
Hydantoin(C(O)-(Gly-D-Arg))-c(Cys-D-Ala-His-D-Phe-Arg-Trp-Cys)-NH₂;
(SEQ ID NO: 537)
Hydantoin(C(O)-(Gly-D-Arg))-c(Cys-D-Ala-His-D-2-Nal-Arg-Trp-Cys)-NH₂;
(SEQ ID NO: 538)
Hydantoin(C(O)-(Nle-Ala))-c(Cys-Glu-His-D-Phe-Arg-Trp-Cys)-NH₂;
(SEQ ID NO: 539)
c[Hydantoin(C(O)-(Nle-Cys))-D-Ala-His-D-Phe-Arg-Trp-Cys]-NH₂;
(SEQ ID NO: 540)
c[Hydantoin(C(O)-(Ala-Cys))-D-Ala-His-D-Phe-Arg-Trp-Cys]-NH₂;
(SEQ ID NO: 541)
c[Hydantoin(C(O)-(D-Ala-Cys))-D-Ala-His-D-Phe-Arg-Trp-Cys]-NH₂;
(SEQ ID NO: 542)
c[Hydantoin(C(O)-(Aib-Cys))-D-Ala-His-D-Phe-Arg-Trp-Cys]-NH₂;
(SEQ ID NO: 543)
c[Hydantoin(C(O)-(Val-Cys))-D-Ala-His-D-Phe-Arg-Trp-Cys]-NH₂;
(SEQ ID NO: 544)
c[Hydantoin(C(O)-(Abu-Cys))-D-Ala-His-D-Phe-Arg-Trp-Cys]-NH₂;
(SEQ ID NO: 545)
c[Hydantoin(C(O)-(Leu-Cys))-D-Ala-His-D-Phe-Arg-Trp-Cys]-NH₂;
(SEQ ID NO: 546)
c[Hydantoin(C(O)-(Ile-Cys))-D-Ala-His-D-Phe-Arg-Trp-Cys]-NH₂;
(SEQ ID NO: 547)
c[Hydantoin(C(O)-(Cha-Cys))-D-Ala-His-D-Phe-Arg-Trp-Cys]-NH₂;
(SEQ ID NO: 548)
c[Hydantoin(C(O)-(A6c-Cys))-D-Ala-His-D-Phe-Arg-Trp-Cys]-NH₂;
(SEQ ID NO: 549)
c[Hydantoin(C(O)-(Phe-Cys))-D-Ala-His-D-Phe-Arg-Trp-Cys]-NH₂;
(SEQ ID NO: 550)
c[Hydantoin(C(O)-(Gly-Cys))-D-Ala-His-D-Phe-Arg-Trp-Cys]-NH₂;
(SEQ ID NO: 551)
c[Hydantoin(C(O)-(Gly-Cys))-Glu-His-D-Phe-Arg-Trp-Cys]-NH₂;
(SEQ ID NO: 552)
Ac-Tyr-Arg-c(Cys-D-Ala-His-D-Phe-Arg-Trp-Cys)-NH₂;
(SEQ ID NO: 553)
Ac-2-Nal-Arg-c(Cys-D-Ala-His-D-Phe-Arg-Trp-Cys)-NH₂;
(SEQ ID NO: 554)
Ac-1-Nal-Arg-c(Cys-D-Ala-His-DPhe-Arg-Trp-Cys)-NH₂;
(SEQ ID NO: 555)
Ac-Phe-Arg-c(Cys-D-Ala-His-D-Phe-Arg-Trp-Cys)-NH₂;
(SEQ ID NO: 556)
Ac-Trp-Arg-c(Cys-D-Ala-His-D-Phe-Arg-Trp-Cys)-NH₂;
(SEQ ID NO: 557);
Ac-Pff-Arg-c(Cys-D-Ala-His-D-Phe-Arg-Trp-Cys)-NH₂;
(SEQ ID NO: 558)
H-His-Arg-c(Cys-D-Ala-His-D-Phe-Arg-Trp-Cys)-NH₂;
and
(SEQ ID NO: 559)
Ac-His-Arg-c(Cys-D-Ala-His-D-Phe-Arg-Trp-Cys)-NH₂;
or a pharmaceutically acceptable salt thereof.


SEQUENCE LISTING (559 sequences)

SEQ ID NO: 1 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine,
  MOD_RES 7: note = bAla
XDHFRWAK

SEQ ID NO: 2 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = A6c
XDHFRWXK

SEQ ID NO: 3 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 6-aminohexanoic acid
XCHFRWXC

SEQ ID NO: 4 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-Phenylalanine
  SITE 8: note = D-Cystine
FCHFRWACT

SEQ ID NO: 5 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-Phenylalanine
  MOD_RES 7: note = bAla
  SITE 8: note = D-Cystine
FCHFRWACT

SEQ ID NO: 6 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-Phenylalanine
  MOD_RES 7: note = 4abu
  SITE 8: note = D-Cystine
FCHFRWXCT

SEQ ID NO: 7 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 5-aminopentanoic acid
XCHFRWXC

SEQ ID NO: 8 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 5-aminopentanoic acid
XDHFRWXK

SEQ ID NO: 9 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = A6c
  REGION 2..8: note = Cyclic
  SITE 4: note = D-Phenylalanine
  MOD_RES 7: note = 4abu
XDHFRWXK

SEQ ID NO: 10 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = D-P-(2-naphthyl)alanine
  REGION 2..8: note = Cyclic
  SITE 4: note = D-Phenylalanine
  MOD_RES 7: note = 4abu
XDHFRWXK

SEQ ID NO: 11 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Beta-cyclohexylalanine
  REGION 2..8: note = Cyclic
  SITE 4: note = D-Phenylalanine
  MOD_RES 7: note = 4abu
XDHFRWXK

SEQ ID NO: 12 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
XDHFRWXK

SEQ ID NO: 13 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
XCAHFRWC

SEQ ID NO: 14 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = bAla
  SITE 5: note = D-Phenylalanine
XCAHFRWC

SEQ ID NO: 15 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = 4abu
  SITE 5: note = D-Phenylalanine
XCXHFRWC

SEQ ID NO: 16 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = a-aminoisobutyric acid
  SITE 5: note = D-Phenylalanine
XCXHFRWC

SEQ ID NO: 17 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 5: note = D-Phenylalanine
XCGHFRWC

SEQ ID NO: 18 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  SITE 5: note = D-Phenylalanine
XCAHFRWC

SEQ ID NO: 19 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  SITE 3: note = D-Alanine
  SITE 5: note = D-Phenylalanine
XCAHFRWC

SEQ ID NO: 20 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  MOD_RES 3: note = bAla
  SITE 5: note = D-Phenylalanine
XCAHFRWC

SEQ ID NO: 21 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  MOD_RES 3: note = 4abu
  SITE 5: note = D-Phenylalanine
XCXHFRWC

SEQ ID NO: 22 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  MOD_RES 3: note = a-aminoisobutyric acid
  SITE 5: note = D-Phenylalanine
XCXHFRWC

SEQ ID NO: 23 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  SITE 5: note = D-Phenylalanine
XCGHFRWC

SEQ ID NO: 24 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  SITE 8: note = D-Cystine
XCAHFRWC

SEQ ID NO: 25 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = bAla
  SITE 5: note = D-Phenylalanine
  SITE 8: note = D-Cystine
XCAHFRWC

SEQ ID NO: 26 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = 4abu
  SITE 5: note = D-Phenylalanine
  SITE 8: note = D-Cystine
XCXHFRWC

SEQ ID NO: 27 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = a-aminoisobutyric acid
  SITE 5: note = D-Phenylalanine
  SITE 8: note = D-Cystine
XCXHFRWC

SEQ ID NO: 28 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 5: note = D-Phenylalanine
  SITE 8: note = D-Cystine
XCGHFRWC

SEQ ID NO: 29 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  SITE 5: note = D-Phenylalanine
  SITE 8: note = D-Cystine
XCAHFRWC

SEQ ID NO: 30 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  SITE 3: note = D-Alanine
  SITE 5: note = D-Phenylalanine
  SITE 8: note = D-Cystine
XCAHFRWC

SEQ ID NO: 31 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  MOD_RES 3: note = bAla
  SITE 5: note = D-Phenylalanine
  SITE 8: note = D-Cystine
XCAHFRWC

SEQ ID NO: 32 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  MOD_RES 3: note = 4abu
  SITE 5: note = D-Phenylalanine
  SITE 8: note = D-Cystine
XCXHFRWC

SEQ ID NO: 33 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 2: note = D-Cystine
  MOD_RES 3: note = a-aminoisobutyric acid
  SITE 5: note = D-Phenylalanine
  SITE 8: note = D-Cystine
XCXHFRWC

SEQ ID NO: 34 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Octahydroindole-2-carboxylic acid
  REGION 2..8: note = Cyclic
  SITE 4: note = D-Phenylalanine
  MOD_RES 7: note = 4abu
XDHFRWXK

SEQ ID NO: 35 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Cyclohexylglycine
  REGION 2..8: note = Cyclic
  SITE 4: note = D-Phenylalanine
  MOD_RES 7: note = 4abu
XDHFRWXK

SEQ ID NO: 36 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
XDHFRWXK

SEQ ID NO: 37 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = D-Beta-cyclohexylalanine
  REGION 2..8: note = Cyclic
  SITE 4: note = D-Phenylalanine
  MOD_RES 7: note = 4abu
XDHFRWXK

SEQ ID NO: 38 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = D-homo-cyclohexylalanine
  REGION 2..8: note = Cyclic
  SITE 4: note = D-Phenylalanine
  MOD_RES 7: note = 4abu
XDHFRWXK

SEQ ID NO: 39 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Nipecotic acid
  REGION 2..8: note = Cyclic
  SITE 4: note = D-Phenylalanine
  MOD_RES 7: note = 4abu
XDHFRWXK

SEQ ID NO: 40 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Homoproline
  REGION 2..8: note = Cyclic
  SITE 4: note = D-Phenylalanine
  MOD_RES 7: note = 4abu
PDHFRWXK

SEQ ID NO: 41 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Homoleucine
  REGION 2..8: note = Cyclic
  SITE 4: note = D-Phenylalanine
  MOD_RES 7: note = 4abu
LDHFRWXK

SEQ ID NO: 42 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  MOD_RES 7: note = 4abu
FDHFRWXK

SEQ ID NO: 43 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = D-Phenylalanine
  REGION 2..8: note = Cyclic
  SITE 4: note = D-Phenylalanine
  MOD_RES 7: note = 4abu
FDHFRWXK

SEQ ID NO: 44 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = D-Chg
  REGION 2..8: note = Cyclic
  SITE 4: note = D-Phenylalanine
  MOD_RES 7: note = 4abu
XDHFRWXK

SEQ ID NO: 45 - protein, 8 aa, synthetic construct
  SITE >8: note = C-terminal is: NH2
  REGION 1..8: note = Description of Sequence: MC4R agonist
  SITE <1: note = N-term is: n-butanoyl
  MOD_RES 1: note = Beta-cyclohexylalanine
  REGION 2..8: note = Cyclic
  SITE 4: note = D-Phenylalanine
  MOD_RES 7: note = 4abu
XDHFRWXK

SEQ ID NO: 46 - protein, 8 aa, synthetic construct
  SITE >8: note = C-terminal is: NH2
  REGION 1..8: note = Description of Sequence: MC4R agonist
  SITE <1: note = N-term is: n-butyryl
  MOD_RES 1: note = Beta-cyclohexylalanine
  REGION 2..8: note = Cyclic
  SITE 4: note = D-Phenylalanine
  MOD_RES 7: note = 4abu
XDHFRWXK

SEQ ID NO: 47 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Homophenylalanine
  REGION 2..8: note = Cyclic
  SITE 4: note = D-Phenylalanine
  MOD_RES 7: note = 4abu
FDHFRWXK

SEQ ID NO: 48 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Beta-hMet
  REGION 2..8: note = Cyclic
  SITE 4: note = D-Phenylalanine
  MOD_RES 7: note = 4abu
MDHFRWXK

SEQ ID NO: 49 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = 4abu
  REGION 2..8: note = Cyclic
  SITE 4: note = D-Phenylalanine
  MOD_RES 7: note = 4abu
XDHFRWXK

SEQ ID NO: 50 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Beta-cyclohexylalanine
  REGION 2..8: note = Cyclic
  SITE 4: note = D-Phenylalanine
  SITE 6: note = D-Trp
XDHFRWAK

SEQ ID NO: 51 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  SITE 6: note = D-Trp
XDHFRWAK

SEQ ID NO: 52 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  SITE 6: note = D-Trp
LDHFRWAK

SEQ ID NO: 53 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Homoleucine
  REGION 2..8: note = Cyclic
  SITE 4: note = D-Phenylalanine
  SITE 6: note = D-Trp
LDHFRWAK

SEQ ID NO: 54 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  SITE 6: note = D-Trp
FDHFRWAK

SEQ ID NO: 55 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  SITE 7: note = D-Alanine
XDHFRWAK

SEQ ID NO: 56 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = bAla
XDHFRWAK

SEQ ID NO: 57 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 4abu
XDHFRWXK

SEQ ID NO: 58 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = Aha
XDHFRWXK

SEQ ID NO: 59 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 5-aminopentanoic acid
XDHFRWXK

SEQ ID NO: 60 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 5-aminopentanoic acid
XCHFRWXC

SEQ ID NO: 61 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 4abu
XCHFRWXC

SEQ ID NO: 62 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 6-aminohexanoic acid
XCHFRWXC

SEQ ID NO: 63 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = bAla
XCHFRWAC

SEQ ID NO: 64 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  SITE 7: note = D-Alanine
XCHFRWAC

SEQ ID NO: 65 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
XCAHXRWC

SEQ ID NO: 66 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
  MOD_RES 7: note = P-(2-naphthyl)alanine
XCAHXRXC

SEQ ID NO: 67 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
  MOD_RES 7: note = 1-Nal
XCAHXRXC

SEQ ID NO: 68 - protein, 8 aa, synthetic construct
  SITE >8: note = C-terminal is: NH2
  REGION 1..8: note = Description of Sequence: MC4R agonist
  SITE <1: note = N-term is: n-butanoyl
  SITE 1: note = Nle
  REGION 2..8: note = Cyclic
  SITE 3: note = D-Alanine
  SITE 5: note = D-Phenylalanine
  MOD_RES 7: note = P-(2-naphthyl)alanine
XCAHFRXC

SEQ ID NO: 69 - protein, 8 aa, synthetic construct
  SITE >8: note = C-terminal is: NH2
  REGION 1..8: note = Description of Sequence: MC4R agonist
  SITE <1: note = N-term is: n-butanoyl
  SITE 1: note = Nle
  REGION 2..8: note = Cyclic
  SITE 3: note = D-Alanine
  SITE 5: note = D-Phenylalanine
XCAHFRWC

SEQ ID NO: 70 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 7: note = P-(2-naphthyl)alanine
XCAHFRXC

SEQ ID NO: 71 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 7: note = 1-Nal
XCAHFRXC

SEQ ID NO: 72 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 7: note = 3-benzothienylaaanine
XCAHFRXC

SEQ ID NO: 73 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Glu
  SITE 5: note = D-Phenylalanine
XCEHFRWC

SEQ ID NO: 74 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 7: note = D-Alanine
XDHFRWAK

SEQ ID NO: 75 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
  MOD_RES 7: note = 3-benzothienylaaanine
XCAHXRXC

SEQ ID NO: 76 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 2: note = Penicillamine
  SITE 3: note = D-Alanine
  SITE 5: note = D-Phenylalanine
XXAHFRWC

SEQ ID NO: 77 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
XCAHFRWX

SEQ ID NO: 78 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 2: note = Penicillamine
  SITE 3: note = D-Alanine
  SITE 5: note = D-Phenylalanine
  MOD_RES 8: note = Penicillamine
XXAHFRWX

SEQ ID NO: 79 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-Phenylalanine
  SITE 5: note = Homoarginine
  MOD_RES 7: note = bAla
  SITE 8: note = D-Cystine
FCHFRWACT

SEQ ID NO: 80 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
FCHYRWACT

SEQ ID NO: 81 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-Phenylalanine
  MOD_RES 6: note = 4,4'-biphenylalanine
  MOD_RES 7: note = bAla
  SITE 8: note = D-Cystine
FCHFRXACT

SEQ ID NO: 82 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
FCHYRWACT

SEQ ID NO: 83 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-Phenylalanine
  SITE 5: note = Homoarginine
  MOD_RES 6: note = 4,4'-biphenylalanine
  MOD_RES 7: note = bAla
  SITE 8: note = D-Cystine
FCHFRXACT

SEQ ID NO: 84 - protein, 9 aa, synthetic construct
  SITE >9: note = C-terminal is: NH2
  REGION 1..9: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
FCHYRXACT

SEQ ID NO: 85 - protein, 8 aa, synthetic construct
  SITE >8: note = C-terminal is: NH2
  REGION 1..8: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
XCHFRWXC

SEQ ID NO: 86 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
XDAHFRWK

SEQ ID NO: 87 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 7: note = 3-benzothienylaaanine
XDAHFRXK

SEQ ID NO: 88 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
XCAHFRWX

SEQ ID NO: 89 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Abu
  SITE 5: note = D-Phenylalanine
XCXHFRWC

SEQ ID NO: 90 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Val
  SITE 5: note = D-Phenylalanine
XCVHFRWC

SEQ ID NO: 91 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Ile
  SITE 5: note = D-Phenylalanine
XCIHFRWC

SEQ ID NO: 92 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
XCLHFRWC

SEQ ID NO: 93 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Tle
  SITE 5: note = D-Phenylalanine
XCXHFRWC

SEQ ID NO: 94 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
XCXHFRWC

SEQ ID NO: 95 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 2: note = Penicillamine
  SITE 4: note = D-Phenylalanine
  MOD_RES 7: note = 4abu
XXHFRWXC

SEQ ID NO: 96 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
  MOD_RES 8: note = Penicillamine
XCHFRWXX

SEQ ID NO: 97 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 2: note = Penicillamine
  SITE 4: note = D-Phenylalanine
  MOD_RES 7: note = 4abu
  MOD_RES 8: note = Penicillamine
XXHFRWXX

SEQ ID NO: 98 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  MOD_RES 7: note = 4abu
LCHFRWXC

SEQ ID NO: 99 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Beta-cyclohexylalanine
  REGION 2..8: note = Cyclic
  SITE 4: note = D-Phenylalanine
  MOD_RES 7: note = 4abu
XCHFRWXC

SEQ ID NO: 100 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  MOD_RES 7: note = 4abu
ICHFRWXC

SEQ ID NO: 101 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  MOD_RES 7: note = 4abu
FCHFRWXC

SEQ ID NO: 102 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  MOD_RES 7: note = 4abu
VCHFRWXC

SEQ ID NO: 103 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = P-(2-naphthyl)alanine
  REGION 2..8: note = Cyclic
  SITE 4: note = D-Phenylalanine
  MOD_RES 7: note = 4abu
XCHFRWXC

SEQ ID NO: 104 - protein, 8 aa, synthetic construct
  SITE >8: note = C-terminal is: NH2
  REGION 1..8: note = Description of Sequence: MC4R agonist
  SITE 1: note = Nle
  REGION 2..8: note = Cyclic
  SITE 4: note = D-Phenylalanine
  MOD_RES 7: note = 4abu
XCHFRWXC

SEQ ID NO: 105 - protein, 8 aa, synthetic construct
  SITE >8: note = C-terminal is: NH2
  REGION 1..8: note = Description of Sequence: MC4R agonist
  REGION 2..8: note = Cyclic
  SITE 4: note = D-Phenylalanine
  MOD_RES 7: note = 4abu
FCHFRWXC

SEQ ID NO: 106 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 3: note = 3-Pal
  SITE 4: note = D-Phenylalanine
  MOD_RES 7: note = 4abu
XCXFRWXC

SEQ ID NO: 107 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
XCAHFRWC

SEQ ID NO: 108 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  SITE 6: note = D-Trp
  MOD_RES 7: note = 4abu
XCHFRWXC

SEQ ID NO: 109 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 4: note = D-P-(2-naphthyl)alanine
XDHXRWAK

SEQ ID NO: 110 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 4: note = D-P-(2-naphthyl)alanine
  MOD_RES 7: note = bAla
XDHXRWAK

SEQ ID NO: 111 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 4: note = D-P-(2-naphthyl)alanine
  MOD_RES 7: note = 4abu
XCHXRWXC

SEQ ID NO: 112 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Nle
  REGION 2..8: note = Cyclic
  MOD_RES 4: note = D-P-(2-naphthyl)alanine
  MOD_RES 7: note = 6-aminohexanoic acid
XCHXRWXC

SEQ ID NO: 113 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = Homophenylalanine
  REGION 2..8: note = Cyclic
  MOD_RES 4: note = D-P-(2-naphthyl)alanine
  MOD_RES 7: note = 4abu
FDHXRWXK

SEQ ID NO: 114 - protein, 8 aa, synthetic construct
  SITE >8: note = C-terminal is: NH2
  REGION 1..8: note = Description of Sequence: MC4R agonist
  SITE <1: note = N-terminal is: Acetylated
  MOD_RES 1: note = Beta-cyclohexylalanine
  REGION 2..8: note = Cyclic
  MOD_RES 4: note = D-P-(2-naphthyl)alanine
  MOD_RES 7: note = 4abu
XDHXRWXK

SEQ ID NO: 115 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = bAla
XDHFRWAK

SEQ ID NO: 116 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 6-aminohexanoic acid
XCHFRWXC

SEQ ID NO: 117 - protein, 9 aa, synthetic construct
  SITE >9: note = C-terminal is: OH
  REGION 1..9: note = Description of Sequence: MC4R agonist
  SITE 1: note = D-Phenylalanine
  REGION 2..8: note = Cyclic
  SITE 4: note = D-Phenylalanine
  SITE 8: note = D-Cystine
FCHFRWACT

SEQ ID NO: 118 - protein, 9 aa, synthetic construct
  SITE >9: note = C-terminal is: OH
  REGION 1..9: note = Description of Sequence: MC4R agonist
  SITE 1: note = D-Phenylalanine
  REGION 2..8: note = Cyclic
  SITE 4: note = D-Phenylalanine
  MOD_RES 7: note = bAla
  SITE 8: note = D-Cystine
FCHFRWACT

SEQ ID NO: 119 - protein, 9 aa, synthetic construct
  SITE >9: note = C-terminal is: OH
  REGION 1..9: note = Description of Sequence: MC4R agonist
  SITE 1: note = D-Phenylalanine
  REGION 2..8: note = Cyclic
  SITE 4: note = D-Phenylalanine
  MOD_RES 7: note = 4abu
  SITE 8: note = D-Cystine
FCHFRWXCT

SEQ ID NO: 120 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 5-aminopentanoic acid
XCHFRWXC

SEQ ID NO: 121 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 5-aminopentanoic acid
XDHFRWXK

SEQ ID NO: 122 - protein, 8 aa, synthetic construct
  SITE >8: note = C-terminal is: OH
  REGION 1..8: note = Description of Sequence: MC4R agonist
  SITE <1: note = N-terminal is: Acetylated
  MOD_RES 1: note = Beta-cyclohexylalanine
  REGION 2..8: note = Cyclic
  SITE 4: note = D-Phenylalanine
  MOD_RES 7: note = 4abu
XDHFRWXK

SEQ ID NO: 123 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
XDHFRWXK

SEQ ID NO: 124 - protein, 8 aa, synthetic construct
  SITE >8: note = C-terminal is: OH
  REGION 1..8: note = Description of Sequence: MC4R agonist
  SITE <1: note = N-terminal is: Acetylated
  MOD_RES 1: note = Cyclohexylglycine
  REGION 2..8: note = Cyclic
  SITE 4: note = D-Phenylalanine
  MOD_RES 7: note = 4abu
XDHFRWXK

SEQ ID NO: 125 - protein, 8 aa, synthetic construct
  SITE >8: note = C-terminal is: OH
  REGION 1..8: note = Description of Sequence: MC4R agonist
  SITE <1: note = N-terminal is: Acetylated
  MOD_RES 1: note = D-Beta-cyclohexylalanine
  REGION 2..8: note = Cyclic
  SITE 4: note = D-Phenylalanine
  MOD_RES 7: note = 4abu
XDHFRWXK

SEQ ID NO: 126 - protein, 8 aa, synthetic construct
  SITE >8: note = C-terminal is: OH
  REGION 1..8: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
XDHFRWXK

SEQ ID NO: 127 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = D-Chg
  REGION 2..8: note = Cyclic
  SITE 4: note = D-Phenylalanine
  MOD_RES 7: note = 4abu
XDHFRWXK

SEQ ID NO: 128 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Homophenylalanine
  REGION 2..8: note = Cyclic
  SITE 4: note = D-Phenylalanine
  MOD_RES 7: note = 4abu
FDHFRWXK

SEQ ID NO: 129 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 4abu
XCHFRWXC

SEQ ID NO: 130 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 6-aminohexanoic acid
XCHFRWXC

SEQ ID NO: 131 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = bAla
XCHFRWAC

SEQ ID NO: 132 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  SITE 7: note = D-Alanine
XCHFRWAC

SEQ ID NO: 133 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
XCAHXRWC

SEQ ID NO: 134 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
  MOD_RES 7: note = P-(2-naphthyl)alanine
XCAHXRXC

SEQ ID NO: 135 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
  MOD_RES 7: note = 1-Nal
XCAHXRXC

SEQ ID NO: 136 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-P-(2-naphthyl)alanine
  MOD_RES 7: note = 3-benzothienylaaanine
XCAHXRXC

SEQ ID NO: 137 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  MOD_RES 2: note = Penicillamine
  SITE 3: note = D-Alanine
  SITE 5: note = D-Phenylalanine
XXAHFRWC

SEQ ID NO: 138 - protein, 8 aa, synthetic construct
  SITE >8: note = C-terminal is: OH
  REGION 1..8: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
  MOD_RES 8: note = Penicillamine
XCHFRWXX

SEQ ID NO: 139 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 3: note = D-Alanine
  MOD_RES 5: note = D-P-(2-naphthyl)alanine
RCAHXRWC

SEQ ID NO: 140 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 3: note = D-Alanine
  SITE 5: note = D-Phenylalanine
RCAHFRWC

SEQ ID NO: 141 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = D-Arg
  REGION 2..8: note = Cyclic
  MOD_RES 3: note = D-Alanine
  SITE 5: note = D-Phenylalanine
RCAHFRWC

SEQ ID NO: 142 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = D-Arg
  REGION 2..8: note = Cyclic
  SITE 3: note = D-Alanine
  SITE 5: note = D-Phenylalanine
  MOD_RES 8: note = Penicillamine
RCAHFRWX

SEQ ID NO: 143 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = D-Arg
  REGION 2..8: note = Cyclic
  SITE 4: note = D-Phenylalanine
  MOD_RES 7: note = 4abu
  MOD_RES 8: note = Penicillamine
RCHFRWXX

SEQ ID NO: 144 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
  MOD_RES 7: note = 4abu
  MOD_RES 8: note = Penicillamine
RCHFRWXX

SEQ ID NO: 145 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 3: note = D-Alanine
  SITE 5: note = D-Phenylalanine
  MOD_RES 8: note = Penicillamine
RCAHFRWX

SEQ ID NO: 146 - protein, 8 aa, synthetic construct
  SITE >8: note = C-terminal is: NH2
  REGION 1..8: note = Description of Sequence: MC4R agonist
  SITE <1: note = N-terminal is: Acetylated
  SITE 1: note = D-Arg
  REGION 2..8: note = Cyclic
  SITE 4: note = D-Phenylalanine
RDHFRWAK

SEQ ID NO: 147 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 2..8: note = Cyclic
  SITE 4: note = D-Phenylalanine
RDHFRWAK

SEQ ID NO: 148 - protein, 9 aa, synthetic construct
  SITE >9: note = C-terminal is: NH2
  REGION 1..9: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
XCAHFRWGC

SEQ ID NO: 149 - protein, 9 aa, synthetic construct
  SITE >9: note = C-terminal is: NH2
  REGION 1..9: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
  MOD_RES 8: note = D-Alanine
XCAHFRWAC

SEQ ID NO: 150 - protein, 9 aa, synthetic construct
  SITE >9: note = C-terminal is: NH2
  REGION 1..9: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
  MOD_RES 8: note = bAla
XCAHFRWAC

SEQ ID NO: 151 - protein, 9 aa, synthetic construct
  SITE >9: note = C-terminal is: NH2
  REGION 1..9: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
  MOD_RES 8: note = 4abu
XCAHFRWXC

SEQ ID NO: 152 - protein, 9 aa, synthetic construct
  SITE >9: note = C-terminal is: NH2
  REGION 1..9: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
  MOD_RES 8: note = 5-aminopentanoic acid
XCAHFRWXC

SEQ ID NO: 153 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 1..8: note = Cyclic
  SITE 4: note = D-Phenylalanine
CEHFRWAC

SEQ ID NO: 154 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 1..8: note = Cyclic
  SITE 4: note = D-Phenylalanine
  MOD_RES 6: note = P-(2-naphthyl)alanine
CEHFRXAC

SEQ ID NO: 155 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 1..8: note = Cyclic
  SITE 2: note = D-Alanine
  SITE 4: note = D-Phenylalanine
CAHFRWAC

SEQ ID NO: 156 - protein, 8 aa, synthetic construct
  SITE >8: note = C-terminal is: NH2
  REGION 1..8: note = Description of Sequence: MC4R agonist
  SITE <1: note = N-terminal is: Acetylated
  REGION 1..8: note = Cyclic
  SITE 2: note = D-Alanine
  SITE 4: note = D-Phenylalanine
  MOD_RES 6: note = P-(2-naphthyl)alanine
CAHFRXAC

SEQ ID NO: 157 - protein, 9 aa, synthetic construct
  SITE >9: note = C-terminal is: NH2
  REGION 1..9: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
XCAHFRWAC

SEQ ID NO: 158 - protein, 9 aa, synthetic construct
  SITE >9: note = C-terminal is: NH2
  REGION 1..9: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
  MOD_RES 7: note = 3-benzothienylaaanine
XDAHFRXAK

SEQ ID NO: 159 - protein, 18 aa, synthetic construct
  SITE >18: note = C-terminal is: NH2
  REGION 1..18: note = Description of Sequence: MC4R agonist
  SITE 12: note = Nle
  REGION 13..18: note = Cyclic
  MOD_RES 15: note = D-2-Nal
YGRKKRRQRRRXDHXRWK

SEQ ID NO: 160 - protein, 19 aa, synthetic construct
  SITE >19: note = C-terminal is: NH2
  REGION 1..19: note = Description of Sequence: MC4R agonist
  MOD_RES 12: note = 8-amino-3,6-dioxaoctanoic acid
  SITE 13: note = Nle
  REGION 14..19: note = Cyclic
  MOD_RES 16: note = D-2-Nal
YGRKKRRQRRRXXDHXRWK

SEQ ID NO: 161 - protein, 19 aa, synthetic construct
  SITE >19: note = C-terminal is: NH2
  REGION 1..19: note = Description of Sequence: MC4R agonist
  SITE 1: note = Nle
  REGION 2..7: note = Cyclic
  MOD_RES 4: note = D-P-(2-naphthyl)alanine
  MOD_RES 8: note = bAla
XDHXRWKAYGRKKRRQRRR

SEQ ID NO: 162 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  MOD_RES 4: note = D-P-(2-naphthyl)alanine
  MOD_RES 8: note = bAla
XDHXRWKAYGRKKRRQRRR

SEQ ID NO: 163 - protein, 20 aa, synthetic construct
  SITE >20: note = C-terminal is: NH2
  REGION 1..20: note = Description of Sequence: MC4R agonist
  SITE 1: note = Nle
  REGION 2..7: note = Cyclic
  MOD_RES 4: note = D-P-(2-naphthyl)alanine
  MOD_RES 8..9: note = 8-amino-3,6-dioxaoctanoic acid
XDHXRWKXXYGRKKRRQRRR

SEQ ID NO: 164 - protein, 22 aa, synthetic construct
  SITE >22: note = C-terminal is: NH2
  REGION 1..22: note = Description of Sequence: MC4R agonist
  SITE <1: note = N-terminal is: Acetylated
  SITE 1: note = Nle
  REGION 2..7: note = Cyclic
  MOD_RES 4: note = D-P-(2-naphthyl)alanine
XDHXRWKPPKDYGRKKRRQRRR

SEQ ID NO: 165 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
CEHXRWGCPPKDYGRKKRRQRRR

SEQ ID NO: 166 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  MOD_RES 4: note = D-P-(2-naphthyl)alanine
  MOD_RES 8..9: note = bAla
XDHXRWKAAYGRKKRRQRRR

SEQ ID NO: 167 - protein, 23 aa, synthetic construct
  SITE >23: note = C-terminal is: NH2
  REGION 1..23: note = Description of Sequence: MC4R agonist
  SITE <1: note = N-terminal is: Acetylated
  SITE 1: note = Nle
  REGION 2..7: note = Cyclic
  MOD_RES 4: note = D-P-(2-naphthyl)alanine
  MOD_RES 12: note = 8-amino-3,6-dioxaoctanoic acid
XDHXRWKPPKDXYGRKKRRQRRR

SEQ ID NO: 168 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = 8-amino-3,6-dioxaoctanoic acid
CEHXRWGCPPKDXYGRKKRRQRRR

SEQ ID NO: 169 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDAYGRKKRRQRRR

SEQ ID NO: 170 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = 8-amino-3,6-dioxaoctanoic acid
CEHXRWACPPKDXYGRKKRRQRRR

SEQ ID NO: 171 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  MOD_RES 4: note = D-P-(2-naphthyl)alanine
  MOD_RES 8..9: note = 8-amino-3,6-dioxaoctanoic acid
XDHXRWKXXYGRKKRRQRRR

SEQ ID NO: 172 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDAYGRKKRRQRRR

SEQ ID NO: 173 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDARRRRRQRRR

SEQ ID NO: 174 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAGRRRRRQRRR

SEQ ID NO: 175 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAYGRRRRRQRRR

SEQ ID NO: 176 - protein, 24 aa, synthetic construct
  SITE >24: note = C-terminal is: NH2
  REGION 1..24: note = Description of Sequence: MC4R agonist
  REGION 1..8: note = Cyclic
  MOD_RES 4: note = D-P-(2-naphthyl)alanine
  MOD_RES 13: note = bAla
CEHXRWACPPKDAYGRKKRQRRRR

SEQ ID NO: 177 - protein, 24 aa, synthetic construct
  SITE >24: note = C-terminal is: NH2
  REGION 1..24: note = Description of Sequence: MC4R agonist
  REGION 1..8: note = Cyclic
  MOD_RES 4: note = D-P-(2-naphthyl)alanine
  MOD_RES 13: note = bAla
CEHXRWACPPKDAYGRKKQRRRRR

SEQ ID NO: 178 - protein, 24 aa, synthetic construct
  SITE >24: note = C-terminal is: NH2
  REGION 1..24: note = Description of Sequence: MC4R agonist
  REGION 1..8: note = Cyclic
  MOD_RES 4: note = D-P-(2-naphthyl)alanine
  MOD_RES 13: note = bAla
CEHXRWACPPKDAYGRKQKRRRRR

SEQ ID NO: 179 - protein, 24 aa, synthetic construct
  SITE >24: note = C-terminal is: NH2
  REGION 1..24: note = Description of Sequence: MC4R agonist
  REGION 1..8: note = Cyclic
  MOD_RES 4: note = D-P-(2-naphthyl)alanine
  MOD_RES 13: note = bAla
CEHXRWACPPKDAYGRKKRRRRQR

SEQ ID NO: 180 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
  MOD_RES 15: note = a-aminoisobutyric acid
CEHXRXACPPKDAYXRKKRRQRRR

SEQ ID NO: 181 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPRDARRRRRQRRR

SEQ ID NO: 182 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDARRRRRQRRR

SEQ ID NO: 183 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDARRRRRRQRRR

SEQ ID NO: 184 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPRDARRRRRQRRR

SEQ ID NO: 185 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDARRRRRQRRR

SEQ ID NO: 186 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDARRRRRRQRRR

SEQ ID NO: 187 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPRDARRRRRRQRRR

SEQ ID NO: 188 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPRDARRRRRQRRR

SEQ ID NO: 189 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDARRRRRRQRRR

SEQ ID NO: 190 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDAYGRKKRRRQRR

SEQ ID NO: 191 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDAYGRQKKRRRRR

SEQ ID NO: 192 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDAYGRKKRRRRRQ

SEQ ID NO: 193 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAYGRKKRRQRRR

SEQ ID NO: 194 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAYGRKKRRQRRR

SEQ ID NO: 195 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDARRKRRQRRR

SEQ ID NO: 196 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDARKRRRQRRR

SEQ ID NO: 197 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDARRKRRQRRR

SEQ ID NO: 198 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDAYGRRKRRQRRR

SEQ ID NO: 199 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDAGRRKRRQRRR

SEQ ID NO: 200 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDAGRKRRRQRRR

SEQ ID NO: 201 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAYGRRKRRQRRR

SEQ ID NO: 202 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAYGRKRRRQRRR

SEQ ID NO: 203 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAGRRKRRQRRR

SEQ ID NO: 204 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAGRKRRRQRRR

SEQ ID NO: 205 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDARRKRRQRRR

SEQ ID NO: 206 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDARKRRRQRRR

SEQ ID NO: 207 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDAYGRKRRRQRRR

SEQ ID NO: 208 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDARRKRRQRRR

SEQ ID NO: 209 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDARKRRRQRRR

SEQ ID NO: 210 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAYGRRKRRQRRR

SEQ ID NO: 211 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAYGRKRRRQRRR

SEQ ID NO: 212 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAGRRKRRQRRR

SEQ ID NO: 213 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAGRKRRRQRRR

SEQ ID NO: 214 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDARRRRRQRRR

SEQ ID NO: 215 - protein, 22 aa, synthetic construct
  SITE >22: note = C-terminal is: NH2
  REGION 1..22: note = Description of Sequence: MC4R agonist
  SITE <1: note = N-terminal is: Acetylated
  REGION 1..8: note = Cyclic
  MOD_RES 4: note = D-2-Nal
  MOD_RES 13: note = bAla
CEHXRWACPPRDARRRRRQRRR

SEQ ID NO: 216 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDAYGRRRRRQRRR

SEQ ID NO: 217 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPRDAYGRRRRRQRRR

SEQ ID NO: 218 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDARRRRRQRRRR

SEQ ID NO: 219 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPRDARRRRRQRRRR

SEQ ID NO: 220 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPKDAYGRRRRRQRRRR

SEQ ID NO: 221 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 13: note = bAla
CEHXRWACPPRDAYGRRRRRQRRRR

SEQ ID NO: 222 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDARRRRRQRRRR

SEQ ID NO: 223 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPRDARRRRRQRRRR

SEQ ID NO: 224 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPRDARRRRRRQRRR

SEQ ID NO: 225 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAYGRRRRRQRRR

SEQ ID NO: 226 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPRDAYGRRRRRQRRR

SEQ ID NO: 227 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAYGRRRRRQRRRR

SEQ ID NO: 228 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPRDAYGRRRRRQRRRR

SEQ ID NO: 229 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPKDAYGRRRRRRQRRR

SEQ ID NO: 230 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
  MOD_RES 13: note = bAla
CEHXRXACPPRDAYGRRRRRRQRRR

SEQ ID NO: 231 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPRDARRRRRRQRRR

SEQ ID NO: 232 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDARRRRRQRRRR

SEQ ID NO: 233 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPRDARRRRRQRRRR

SEQ ID NO: 234 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDAYGRRRRRQRRR

SEQ ID NO: 235 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPRDAYGRRRRRQRRR

SEQ ID NO: 236 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-2-Nal
  MOD_RES 6: note = P-(2-naphthyl)alanine
  MOD_RES 13: note = bAla
CEHXRXACPPKDAYGRRRRRRQRRR

SEQ ID NO: 237 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPRDAYGRRRRRRQRRR

SEQ ID NO: 238 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPKDAYGRRRRRQRRRR

SEQ ID NO: 239 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 13: note = bAla
CEHXRXACPPRDAYGRRRRRQRRRR

SEQ ID NO: 240 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDARRRRRQRRRR

SEQ ID NO: 241 - protein, 23 aa, synthetic construct
  SITE >23: note = C-terminal is: NH2
  REGION 1..23: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPRDARRRRRQRRRR

SEQ ID NO: 242 - protein, 24 aa, synthetic construct
  SITE >24: note = C-terminal is: NH2
  REGION 1..24: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPRDAYGRRRRRQRRR

SEQ ID NO: 243 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAYGRRRRRQRRRR

SEQ ID NO: 244 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPRDAYGRRRRRQRRRR

SEQ ID NO: 245 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPKDAYGRRRRRRQRRR

SEQ ID NO: 246 - protein, 25 aa, synthetic construct
  SITE >25: note = C-terminal is: NH2
  REGION 1..25: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
  MOD_RES 13: note = bAla
CEHXRXACPPRDAYGRRRRRRQRRR

SEQ ID NO: 247 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = 8-amino-3,6-dioxaoctanoic acid
XCAHFRWCXXYGRKKRRQRRR

SEQ ID NO: 248 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  MOD_RES 5: note = D-Phenylalanine
  MOD_RES 9: note = bAla
XCAHFRWCAYGRKKRQRRRR

SEQ ID NO: 249 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = 8-amino-3,6-dioxaoctanoic acid
XCAHFRWCXYGRKKRRQRRR

SEQ ID NO: 250 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCARRRRRQRRR

SEQ ID NO: 251 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAGRRRRRQRRR

SEQ ID NO: 252 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAYGRRRRRQRRR

SEQ ID NO: 253 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAGRRRRRQRRRR

SEQ ID NO: 254 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAYGRRKRRQRRR

SEQ ID NO: 255 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAYGRKRRRQRRR

SEQ ID NO: 256 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAGRRKRRQRRR

SEQ ID NO: 257 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAGRKRRRQRRR

SEQ ID NO: 258 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCARRKRRQRRR

SEQ ID NO: 259 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCARKRRRQRRR

SEQ ID NO: 260 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = bAla
XCAHFRWCAARRRRRQRRR

SEQ ID NO: 261 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = bAla
XCAHFRWCAAGRRRRRQRRR

SEQ ID NO: 262 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = bAla
XCAHFRWCAAYGRRRRRQRRR

SEQ ID NO: 263 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = 8-amino-3,6-dioxaoctanoic acid
XCAHFRWCXRRRRRQRRR

SEQ ID NO: 264 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = 8-amino-3,6-dioxaoctanoic acid
XCAHFRWCXGRRRRRQRRR

SEQ ID NO: 265 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = 8-amino-3,6-dioxaoctanoic acid
XCAHFRWCXYGRRRRRQRRR

SEQ ID NO: 266 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = 8-amino-3,6-dioxaoctanoic acid
XCAHFRWCXXRRRRRQRRR

SEQ ID NO: 267 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = 8-amino-3,6-dioxaoctanoic acid
XCAHFRWCXXGRRRRRQRRR

SEQ ID NO: 268 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = 8-amino-3,6-dioxaoctanoic acid
XCAHFRWCXXYGRRRRRQRRR

SEQ ID NO: 269 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCARRRRRQRRRR

SEQ ID NO: 270 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = bAla
XCAHFRWCAYGRRRRRQRRRR

SEQ ID NO: 271 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = bAla
XCAHFRWCAARRRRRQRRRR

SEQ ID NO: 272 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = bAla
XCAHFRWCAAGRRRRRQRRRR

SEQ ID NO: 273 - protein, 22 aa, synthetic construct
  SITE >22: note = C-terminal is: NH2
  REGION 1..22: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = bAla
XCAHFRWCAAYGRRRRRQRRRR

SEQ ID NO: 274 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = 8-amino-3,6-dioxaoctanoic acid
XCAHFRWCXRRRRRQRRRR

SEQ ID NO: 275 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = 8-amino-3,6-dioxaoctanoic acid
XCAHFRWCXGRRRRRQRRRR

SEQ ID NO: 276 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9: note = 8-amino-3,6-dioxaoctanoic acid
XCAHFRWCXYGRRRRRQRRRR

SEQ ID NO: 277 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = 8-amino-3,6-dioxaoctanoic acid
XCAHFRWCXXRRRRRQRRRR

SEQ ID NO: 278 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = 8-amino-3,6-dioxaoctanoic acid
XCAHFRWCXXGRRRRRQRRRR

SEQ ID NO: 279 - protein, 22 aa, synthetic construct
  SITE >22: note = C-terminal is: NH2
  REGION 1..22: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 9..10: note = 8-amino-3,6-dioxaoctanoic acid
XCAHFRWCXXYGRRRRRQRRRR

SEQ ID NO: 280 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
  MOD_RES 9: note = bAla
XCHFRWXCAYGRRRRRQRRR

SEQ ID NO: 281 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
  MOD_RES 9: note = bAla
XCHFRWXCARRRRRQRRR

SEQ ID NO: 282 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 9: note = bAla
XDHFRWAKAYGRRRRRQRRR

SEQ ID NO: 283 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 9: note = bAla
XDHFRWAKARRRRRQRRR

SEQ ID NO: 284 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = bAla
XDHFRWKAYGRRRRRQRRR

SEQ ID NO: 285 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = bAla
XDHFRWKAGRRRRRQRRR

SEQ ID NO: 286 - protein, 17 aa, synthetic construct
  SITE >17: note = C-terminal is: NH2
  REGION 1..17: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = bAla
XDHFRWKARRRRRQRRR

SEQ ID NO: 287 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = bAla
XDHFRWKAAYGRRRRRQRRR

SEQ ID NO: 288 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = bAla
XDHFRWKAAGRRRRRQRRR

SEQ ID NO: 289 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = bAla
XDHFRWKAARRRRRQRRR

SEQ ID NO: 290 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = 8-amino-3,6-dioxaoctanoic acid
XDHFRWKXYGRRRRRQRRR

SEQ ID NO: 291 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = 8-amino-3,6-dioxaoctanoic acid
XDHFRWKXGRRRRRQRRR

SEQ ID NO: 292 - protein, 17 aa, synthetic construct
  SITE >17: note = C-terminal is: NH2
  REGION 1..17: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = 8-amino-3,6-dioxaoctanoic acid
XDHFRWKXRRRRRQRRR

SEQ ID NO: 293 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = 8-amino-3,6-dioxaoctanoic acid
XDHFRWKXXYGRRRRRQRRR

SEQ ID NO: 294 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = 8-amino-3,6-dioxaoctanoic acid
XDHFRWKXXGRRRRRQRRR

SEQ ID NO: 295 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = 8-amino-3,6-dioxaoctanoic acid
XDHFRWKXXRRRRRQRRR

SEQ ID NO: 296 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = bAla
XDHFRWKAYGRRRRRQRRRR

SEQ ID NO: 297 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = bAla
XDHFRWKAGRRRRRQRRRR

SEQ ID NO: 298 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = bAla
XDHFRWKARRRRRQRRRR

SEQ ID NO: 299 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = bAla
XDHFRWKAAYGRRRRRQRRRR

SEQ ID NO: 300 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = bAla
XDHFRWKAAGRRRRRQRRRR

SEQ ID NO: 301 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = bAla
XDHFRWKAARRRRRQRRRR

SEQ ID NO: 302 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = 8-amino-3,6-dioxaoctanoic acid
XDHFRWKXYGRRRRRQRRRR

SEQ ID NO: 303 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = 8-amino-3,6-dioxaoctanoic acid
XDHFRWKXGRRRRRQRRRR

SEQ ID NO: 304 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8: note = 8-amino-3,6-dioxaoctanoic acid
XDHFRWKXRRRRRQRRRR

SEQ ID NO: 305 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = 8-amino-3,6-dioxaoctanoic acid
XDHFRWKXXYGRRRRRQRRRR

SEQ ID NO: 306 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = 8-amino-3,6-dioxaoctanoic acid
XDHFRWKXXGRRRRRQRRRR

SEQ ID NO: 307 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..7: note = Cyclic
  SITE 4: note = D-Phenylalanine
  MOD_RES 8..9: note = 8-amino-3,6-dioxaoctanoic acid
XDHFRWKXXRRRRRQRRRR

SEQ ID NO: 308 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = bAla
  MOD_RES 9: note = bAla
XDHFRWAKAYGRRRRRQRRR

SEQ ID NO: 309 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = bAla
  MOD_RES 9: note = bAla
XDHFRWAKARRRRRQRRR

SEQ ID NO: 310 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 6-aminohexanoic acid
  MOD_RES 9: note = bAla
XCHFRWXCAYGRRRRRQRRR

SEQ ID NO: 311 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 6-aminohexanoic acid
  MOD_RES 9: note = bAla
XCHFRWXCARRRRRQRRR

SEQ ID NO: 312 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-Phenylalanine
  MOD_RES 7: note = bAla
  SITE 8: note = D-Cystine
  MOD_RES 10: note = bAla
FCHFRWACTAYGRRRRRQRRR

SEQ ID NO: 313 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-Phenylalanine
  MOD_RES 7: note = bAla
  SITE 8: note = D-Cystine
  MOD_RES 10: note = bAla
FCHFRWACTARRRRRQRRR

SEQ ID NO: 314 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 5-aminopentanoic acid
  MOD_RES 9: note = bAla
XCHFRWXCAYGRRRRRQRRR

SEQ ID NO: 315 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 5-aminopentanoic acid
  MOD_RES 9: note = bAla
XCHFRWXCARRRRRQRRR

SEQ ID NO: 316 - protein, 20 aa, synthetic construct
  SITE >20: note = C-terminal is: NH2
  REGION 1..20: note = Description of Sequence: MC4R agonist
  SITE <1: note = N-terminal is: Acetylated
  MOD_RES 1: note = Beta-cyclohexylalanine
  REGION 2..8: note = Cyclic
  SITE 4: note = D-Phenylalanine
  MOD_RES 7: note = 4abu
  MOD_RES 9: note = bAla
XDHFRWXKAYGRRRRRQRRR

SEQ ID NO: 317 - protein, 18 aa, synthetic construct
  SITE >18: note = C-terminal is: NH2
  REGION 1..18: note = Description of Sequence: MC4R agonist
  SITE <1: note = N-terminal is: Acetylated
  MOD_RES 1: note = Beta-cyclohexylalanine
  REGION 2..8: note = Cyclic
  SITE 4: note = D-Phenylalanine
  MOD_RES 7: note = 4abu
  MOD_RES 9: note = bAla
XDHFRWXKARRRRRQRRR

SEQ ID NO: 318 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
  MOD_RES 9: note = bAla
XDHFRWXKAYGRRRRRQRRR

SEQ ID NO: 319 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  MOD_RES 7: note = 4abu
  MOD_RES 9: note = bAla
XDHFRWXKARRRRRQRRR

SEQ ID NO: 320 - protein, 20 aa, synthetic construct
  SITE >20: note = C-terminal is: NH2
  REGION 1..20: note = Description of Sequence: MC4R agonist
  SITE <1: note = N-terminal is: Acetylated
  MOD_RES 1: note = Cyclohexylglycine
  REGION 2..8: note = Cyclic
  SITE 4: note = D-Phenylalanine
  MOD_RES 7: note = 4abu
  MOD_RES 9: note = bAla
XDHFRWXKAYGRRRRRQRRR

SEQ ID NO: 321 - protein, 18 aa, synthetic construct
  SITE >18: note = C-terminal is: NH2
  REGION 1..18: note = Description of Sequence: MC4R agonist
  SITE <1: note = N-terminal is: Acetylated
  MOD_RES 1: note = Cyclohexylglycine
  REGION 2..8: note = Cyclic
  SITE 4: note = D-Phenylalanine
  MOD_RES 7: note = 4abu
  MOD_RES 9: note = bAla
XDHFRWXKARRRRRQRRR

SEQ ID NO: 322 - protein, 20 aa, synthetic construct
  SITE >20: note = C-terminal is: NH2
  REGION 1..20: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = bAla
XDHFRWXKAYGRRRRRQRRR

SEQ ID NO: 323 - protein, 18 aa, synthetic construct
  SITE >18: note = C-terminal is: NH2
  REGION 1..18: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = bAla
XDHFRWXKARRRRRQRRR

SEQ ID NO: 324 - protein, 21 aa, synthetic construct
  SITE >21: note = C-terminal is: NH2
  REGION 1..21: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = bAla
XDHFRWXKAAYGRRRRRQRRR

SEQ ID NO: 325 - protein, 19 aa, synthetic construct
  SITE >19: note = C-terminal is: NH2
  REGION 1..19: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = bAla
XDHFRWXKAARRRRRQRRR

SEQ ID NO: 326 - protein, 20 aa, synthetic construct
  SITE >20: note = C-terminal is: NH2
  REGION 1..20: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = 8-amino-3,6-dioxaoctanoic acid
XDHFRWXKXYGRRRRRQRRR

SEQ ID NO: 327 - protein, 18 aa, synthetic construct
  SITE >18: note = C-terminal is: NH2
  REGION 1..18: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = 8-amino-3,6-dioxaoctanoic acid
XDHFRWXKXRRRRRQRRR

SEQ ID NO: 328 - protein, 21 aa, synthetic construct
  SITE >21: note = C-terminal is: NH2
  REGION 1..21: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XDHFRWXKXXYGRRRRRQRRR

SEQ ID NO: 329 - protein, 19 aa, synthetic construct
  SITE >19: note = C-terminal is: NH2
  REGION 1..19: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XDHFRWXKXXRRRRRQRRR

SEQ ID NO: 330 - protein, 21 aa, synthetic construct
  SITE >21: note = C-terminal is: NH2
  REGION 1..21: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = bAla
XDHFRWXKAYGRRRRRQRRRR

SEQ ID NO: 331 - protein, 19 aa, synthetic construct
  SITE >19: note = C-terminal is: NH2
  REGION 1..19: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = bAla
XDHFRWXKARRRRRQRRRR

SEQ ID NO: 332 - protein, 22 aa, synthetic construct
  SITE >22: note = C-terminal is: NH2
  REGION 1..22: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = bAla
XDHFRWXKAAYGRRRRRQRRRR

SEQ ID NO: 333 - protein, 20 aa, synthetic construct
  SITE >20: note = C-terminal is: NH2
  REGION 1..20: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = bAla
XDHFRWXKAARRRRRQRRRR

SEQ ID NO: 334 - protein, 21 aa, synthetic construct
  SITE >21: note = C-terminal is: NH2
  REGION 1..21: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = 8-amino-3,6-dioxaoctanoic acid
XDHFRWXKXYGRRRRRQRRRR

SEQ ID NO: 335 - protein, 19 aa, synthetic construct
  SITE >19: note = C-terminal is: NH2
  REGION 1..19: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9: note = 8-amino-3,6-dioxaoctanoic acid
XDHFRWXKXRRRRRQRRRR

SEQ ID NO: 336 - protein, 22 aa, synthetic construct
  SITE >22: note = C-terminal is: NH2
  REGION 1..22: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XDHFRWXKXXYGRRRRRQRRRR

SEQ ID NO: 337 - protein, 20 aa, synthetic construct
  SITE >20: note = C-terminal is: NH2
  REGION 1..20: note = Description of Sequence: MC4R agonist
  SITE <1: note = N-terminal is: Acetylated
  MOD_RES 1: note = Homo-cyclohexylalanine
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XDHFRWXKXXRRRRRQRRRR

SEQ ID NO: 338 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = D-Chg
  REGION 2..8: note = Cyclic
  SITE 4: note = D-Phenylalanine
  MOD_RES 7: note = 4abu
  MOD_RES 9: note = bAla
XDHFRWXKAYGRRRRRQRRR

SEQ ID NO: 339 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = D-Chg
  REGION 2..8: note = Cyclic
  SITE 4: note = D-Phenylalanine
  MOD_RES 7: note = 4abu
  MOD_RES 9: note = bAla
XDHFRWXKARRRRRQRRR

SEQ ID NO: 340 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Homophenylalanine
  REGION 2..8: note = Cyclic
  SITE 4: note = D-Phenylalanine
  MOD_RES 7: note = 4abu
  MOD_RES 9: note = bAla
FDHFRWXKAYGRRRRRQRRR

SEQ ID NO: 341 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Homophenylalanine
  REGION 2..8: note = Cyclic
  SITE 4: note = D-Phenylalanine
  MOD_RES 7: note = 4abu
  MOD_RES 9: note = bAla
FDHFRWXKARRRRRQRRR

SEQ ID NO: 342 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 5-aminopentanoic acid
  MOD_RES 9: note = bAla
XCHFRWXCAYGRRRRRQRRR

SEQ ID NO: 343 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 5-aminopentanoic acid
  MOD_RES 9: note = bAla
XCHFRWXCARRRRRQRRR

SEQ ID NO: 344 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 6-aminohexanoic acid
  MOD_RES 9: note = bAla
XCHFRWXCAYGRRRRRQRRR

SEQ ID NO: 345 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = 6-aminohexanoic acid
  MOD_RES 9: note = bAla
XCHFRWXCARRRRRQRRR

SEQ ID NO: 346 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = bAla
  MOD_RES 9: note = bAla
XCHFRWACAYGRRRRRQRRR

SEQ ID NO: 347 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 4: note = D-Phenylalanine
  SITE 6: note = D-Trp
  MOD_RES 7: note = bAla
  MOD_RES 9: note = bAla
XCHFRWACARRRRRQRRR

SEQ ID NO: 348 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9: note = bAla
XCAHFRWXAYGRRRRRQRRR

SEQ ID NO: 349 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9: note = bAla
XCAHFRWXAGRRRRRQRRR

SEQ ID NO: 350 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9: note = bAla
XCAHFRWXARRRRRQRRR

SEQ ID NO: 351 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9..10: note = bAla
XCAHFRWXAAYGRRRRRQRRR

SEQ ID NO: 352 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9..10: note = bAla
XCAHFRWXAAGRRRRRQRRR

SEQ ID NO: 353 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9..10: note = bAla
XCAHFRWXAARRRRRQRRR

SEQ ID NO: 354 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9: note = 8-amino-3,6-dioxaoctanoic acid
XCAHFRWXXYGRRRRRQRRR

SEQ ID NO: 355 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9: note = 8-amino-3,6-dioxaoctanoic acid
XCAHFRWXXGRRRRRQRRR

SEQ ID NO: 356 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9: note = 8-amino-3,6-dioxaoctanoic acid
XCAHFRWXXRRRRRQRRR

SEQ ID NO: 357 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9..10: note = 8-amino-3,6-dioxaoctanoic acid
XCAHFRWXXXYGRRRRRQRRR

SEQ ID NO: 358 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9..10: note = 8-amino-3,6-dioxaoctanoic acid
XCAHFRWXXXGRRRRRQRRR

SEQ ID NO: 359 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Alanine
  SITE 5: note = D-Phenylalanine
  MOD_RES 8: note = Penicillamine
  MOD_RES 9..10: note = 8-amino-3,6-dioxaoctanoic acid
XCAHFRWXXXRRRRRQRRR

SEQ ID NO: 360 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = bAla
FCHYRWACAYGRRRRRQRRR

SEQ ID NO: 361 - protein, 18 aa, synthetic construct
  SITE >18: note = C-terminal is: NH2
  REGION 1..18: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = bAla
FCHYRWACARRRRRQRRR

SEQ ID NO: 362 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = bAla
FCHYRWACAGRRRRRQRRR

SEQ ID NO: 363 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = bAla
FCHYRWACARRRRRQRRRR

SEQ ID NO: 364 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = bAla
FCHYRWACAAYGRRRRRQRRR

SEQ ID NO: 365 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = bAla
FCHYRWACAARRRRRQRRR

SEQ ID NO: 366 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = bAla
FCHYRWACAAGRRRRRQRRR

SEQ ID NO: 367 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = bAla
FCHYRWACAARRRRRQRRRR

SEQ ID NO: 368 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = 8-amino-3,6-dioxaoctanoic acid
FCHYRWACXYGRRRRRQRRR

SEQ ID NO: 369 - protein, 18 aa, synthetic construct
  SITE >18: note = C-terminal is: NH2
  REGION 1..18: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = 8-amino-3,6-dioxaoctanoic acid
FCHYRWACXRRRRRQRRR

SEQ ID NO: 370 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = 8-amino-3,6-dioxaoctanoic acid
FCHYRWACXGRRRRRQRRR

SEQ ID NO: 371 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9: note = 8-amino-3,6-dioxaoctanoic acid
FCHYRWACXRRRRRQRRRR

SEQ ID NO: 372 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = 8-amino-3,6-dioxaoctanoic acid
FCHYRWACXXYGRRRRRQRRR

SEQ ID NO: 373 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = 8-amino-3,6-dioxaoctanoic acid
FCHYRWACXXRRRRRQRRR

SEQ ID NO: 374 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = 8-amino-3,6-dioxaoctanoic acid
FCHYRWACXXGRRRRRQRRR

SEQ ID NO: 375 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  MOD_RES 7: note = bAla
  SITE 8: note = D-Cystine
  MOD_RES 9..10: note = 8-amino-3,6-dioxaoctanoic acid
FCHYRWACXXRRRRRQRRRR

SEQ ID NO: 376 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = bAla
FCHYRWACTAYGRRRRRQRRR

SEQ ID NO: 377 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = bAla
FCHYRWACTARRRRRQRRR

SEQ ID NO: 378 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10..11: note = bAla
FCHYRWACTAAYGRRRRRQRRR

SEQ ID NO: 379 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10..11: note = bAla
FCHYRWACTAARRRRRQRRR

SEQ ID NO: 380 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = 8-amino-3,6-dioxaoctanoic acid
FCHYRWACTXYGRRRRRQRRR

SEQ ID NO: 381 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = 8-amino-3,6-dioxaoctanoic acid
FCHYRWACTXRRRRRQRRR

SEQ ID NO: 382 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10..11: note = 8-amino-3,6-dioxaoctanoic acid
FCHYRWACTXXYGRRRRRQRRR

SEQ ID NO: 383 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = bAla
FCHYRWACTAYGRRRRRQRRRR

SEQ ID NO: 384 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = bAla
FCHYRWACTARRRRRQRRRR

SEQ ID NO: 385 - protein, 23 aa, synthetic construct
  SITE >23: note = C-terminal is: NH2
  REGION 1..23: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10..11: note = bAla
FCHYRWACTAAYGRRRRRQRRRR

SEQ ID NO: 386 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10..11: note = bAla
FCHYRWACTAARRRRRQRRRR

SEQ ID NO: 387 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = 8-amino-3,6-dioxaoctanoic acid
FCHYRWACTXYGRRRRRQRRRR

SEQ ID NO: 388 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10: note = 8-amino-3,6-dioxaoctanoic acid
FCHYRWACTXRRRRRQRRRR

SEQ ID NO: 389 - protein, 23 aa, synthetic construct
  SITE >23: note = C-terminal is: NH2
  REGION 1..23: note = Description of Sequence: MC4R agonist
  SITE 1: note = D-Phenylalanine
  REGION 2..8: note = Cyclic
  MOD_RES 4: note = D-(Et)Tyr
  SITE 5: note = Homoarginine
  MOD_RES 7: note = bAla
  SITE 8: note = D-Cystine
  MOD_RES 10..11: note = 8-amino-3,6-dioxaoctanoic acid
FCHYRWACTXXYGRRRRRQRRRR

SEQ ID NO: 390 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 7: note = bAla
  SITE 8: note = D-Cystine
  MOD_RES 10..11: note = 8-amino-3,6-dioxaoctanoic acid
FCHYRWACTXXRRRRRQRRRR

SEQ ID NO: 391 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10: note = bAla
FCHYRXACTAYGRRRRRQRRR

SEQ ID NO: 392 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10: note = bAla
FCHYRXACTAYGRRRRRQRRRR

SEQ ID NO: 393 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10: note = bAla
FCHYRXACTARRRRRQRRR

SEQ ID NO: 394 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10..11: note = bAla
FCHYRXACTAAYGRRRRRQRRR

SEQ ID NO: 395 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10..11: note = bAla
FCHYRXACTAARRRRRQRRR

SEQ ID NO: 396 - protein, 21 aa, synthetic construct
  SITE >21: note = C-terminal is: NH2
  REGION 1..21: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10: note = 8-amino-3,6-dioxaoctanoic acid
FCHYRXACTXYGRRRRRQRRR

SEQ ID NO: 397 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10: note = 8-amino-3,6-dioxaoctanoic acid
FCHYRXACTXYGRRRRRQRRRR

SEQ ID NO: 398 - protein, 19 aa, synthetic construct
  SITE >19: note = C-terminal is: NH2
  REGION 1..19: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10: note = 8-amino-3,6-dioxaoctanoic acid
FCHYRXACTXRRRRRQRRR

SEQ ID NO: 399 - protein, 22 aa, synthetic construct
  SITE >22: note = C-terminal is: NH2
  REGION 1..22: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10..11: note = 8-amino-3,6-dioxaoctanoic acid
FCHYRXACTXXYGRRRRRQRRR

SEQ ID NO: 400 - protein, 20 aa, synthetic construct
  SITE >20: note = C-terminal is: NH2
  REGION 1..20: note = Description of Sequence: MC4R agonist
  SITE 1: note = D-Phenylalanine
  REGION 2..8: note = Cyclic
  SITE 4: note = D-(Et)Tyr
  SITE 5: note = Homoarginine
  MOD_RES 6: note = 4,4'-biphenylalanine
  MOD_RES 7: note = bAla
  SITE 8: note = D-Cystine
  MOD_RES 10..11: note = 8-amino-3,6-dioxaoctanoic acid
FCHYRXACTXXRRRRRQRRR

SEQ ID NO: 401 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
  MOD_RES 10: note = bAla
XCAHFRWGCAYGRRRRRQRRR

SEQ ID NO: 402 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..9: note = Cyclic
  SITE 3: note = D-Alanine
  SITE 5: note = D-Phenylalanine
  MOD_RES 10: note = bAla
XCAHFRWGCARRRRRQRRR

SEQ ID NO: 403 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = bAla
XCHFRWXCAYGRRRRRQRRR

SEQ ID NO: 404 - protein, 18 aa, synthetic construct
  SITE >18: note = C-terminal is: NH2
  REGION 1..18: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = bAla
XCHFRWXCARRRRRQRRR

SEQ ID NO: 405 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = bAla
XCHFRWXCAAYGRRRRRQRRR

SEQ ID NO: 406 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = bAla
XCHFRWXCAARRRRRQRRR

SEQ ID NO: 407 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = bAla
XCHFRWXCAYGRRRRRQRRRR

SEQ ID NO: 408 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = bAla
XCHFRWXCARRRRRQRRRR

SEQ ID NO: 409 - protein, 22 aa, synthetic construct
  SITE >22: note = C-terminal is: NH2
  REGION 1..22: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = bAla
XCHFRWXCAAYGRRRRRQRRRR

SEQ ID NO: 410 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = bAla
XCHFRWXCAARRRRRQRRRR

SEQ ID NO: 411 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = 8-amino-3,6-dioxaoctanoic acid
XCHFRWXCXYGRRRRRQRRR

SEQ ID NO: 412 - protein, 18 aa, synthetic construct
  SITE >18: note = C-terminal is: NH2
  REGION 1..18: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = 8-amino-3,6-dioxaoctanoic acid
XCHFRWXCXRRRRRQRRR

SEQ ID NO: 413 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = 8-amino-3,6-dioxaoctanoic acid
XCHFRWXCXXYGRRRRRQRRR

SEQ ID NO: 414 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = 8-amino-3,6-dioxaoctanoic acid
XCHFRWXCXXRRRRRQRRR

SEQ ID NO: 415 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = 8-amino-3,6-dioxaoctanoic acid
XCHFRWXCXYGRRRRRQRRRR

SEQ ID NO: 416 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9: note = 8-amino-3,6-dioxaoctanoic acid
XCHFRWXCXRRRRRQRRRR

SEQ ID NO: 417 - protein, 22 aa, synthetic construct
  SITE >22: note = C-terminal is: NH2
  REGION 1..22: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = 8-amino-3,6-dioxaoctanoic acid
XCHFRWXCXXYGRRRRRQRRRR

SEQ ID NO: 418 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 5-aminopentanoic acid
  MOD_RES 9..10: note = 8-amino-3,6-dioxaoctanoic acid
XCHFRWXCXXRRRRRQRRRR

SEQ ID NO: 419 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = bAla
XCLHFRWCAYGRRRRRQRRR

SEQ ID NO: 420 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = bAla
XCLHFRWCARRRRRQRRR

SEQ ID NO: 421 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = bAla
XCLHFRWCAAYGRRRRRQRRR

SEQ ID NO: 422 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = bAla
XCLHFRWCAARRRRRQRRR

SEQ ID NO: 423 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = 8-amino-3,6-dioxaoctanoic acid
XCLHFRWCXYGRRRRRQRRR

SEQ ID NO: 424 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = 8-amino-3,6-dioxaoctanoic acid
XCLHFRWCXRRRRRQRRR

SEQ ID NO: 425 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = 8-amino-3,6-dioxaoctanoic acid
XCLHFRWCXXYGRRRRRQRRR

SEQ ID NO: 426 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = 8-amino-3,6-dioxaoctanoic acid
XCLHFRWCXXRRRRRQRRR

SEQ ID NO: 427 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = bAla
XCLHFRWCAYGRRRRRQRRRR

SEQ ID NO: 428 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = bAla
XCLHFRWCARRRRRQRRRR

SEQ ID NO: 429 - protein, 22 aa, synthetic construct
  SITE >22: note = C-terminal is: NH2
  REGION 1..22: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = bAla
XCLHFRWCAAYGRRRRRQRRRR

SEQ ID NO: 430 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = bAla
XCLHFRWCAARRRRRQRRRR

SEQ ID NO: 431 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = 8-amino-3,6-dioxaoctanoic acid
XCLHFRWCXYGRRRRRQRRRR

SEQ ID NO: 432 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9: note = 8-amino-3,6-dioxaoctanoic acid
XCLHFRWCXRRRRRQRRRR

SEQ ID NO: 433 - protein, 22 aa, synthetic construct
  SITE >22: note = C-terminal is: NH2
  REGION 1..22: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = 8-amino-3,6-dioxaoctanoic acid
XCLHFRWCXXYGRRRRRQRRRR

SEQ ID NO: 434 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  SITE 3: note = D-Leu
  SITE 5: note = D-Phenylalanine
  MOD_RES 9..10: note = 8-amino-3,6-dioxaoctanoic acid
XCLHFRWCXXRRRRRQRRRR

SEQ ID NO: 435 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = bAla
XCXHFRWCAYGRRRRRQRRR

SEQ ID NO: 436 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = bAla
XCXHFRWCARRRRRQRRR

SEQ ID NO: 437 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = bAla
XCXHFRWCAAYGRRRRRQRRR

SEQ ID NO: 438 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = bAla
XCXHFRWCAARRRRRQRRR

SEQ ID NO: 439 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = 8-amino-3,6-dioxaoctanoic acid
XCXHFRWCXYGRRRRRQRRR

SEQ ID NO: 440 - protein, 18 aa, synthetic construct
  SITE >18: note = C-terminal is: NH2
  REGION 1..18: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = 8-amino-3,6-dioxaoctanoic acid
XCXHFRWCXRRRRRQRRR

SEQ ID NO: 441 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = 8-amino-3,6-dioxaoctanoic acid
XCXHFRWCXXYGRRRRRQRRR

SEQ ID NO: 442 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = 8-amino-3,6-dioxaoctanoic acid
XCXHFRWCXXRRRRRQRRR

SEQ ID NO: 443 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = bAla
XCXHFRWCAYGRRRRRQRRRR

SEQ ID NO: 444 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = bAla
XCXHFRWCARRRRRQRRRR

SEQ ID NO: 445 - protein, 22 aa, synthetic construct
  SITE >22: note = C-terminal is: NH2
  REGION 1..22: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = bAla
XCXHFRWCAAYGRRRRRQRRRR

SEQ ID NO: 446 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = bAla
XCXHFRWCAARRRRRQRRRR

SEQ ID NO: 447 - protein, 21 aa, synthetic construct
  SITE >21: note = C-terminal is: NH2
  REGION 1..21: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = 8-amino-3,6-dioxaoctanoic acid
XCXHFRWCXYGRRRRRQRRRR

SEQ ID NO: 448 - protein, 19 aa, synthetic construct
  SITE >19: note = C-terminal is: NH2
  REGION 1..19: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9: note = 8-amino-3,6-dioxaoctanoic acid
XCXHFRWCXRRRRRQRRRR

SEQ ID NO: 449 - protein, 22 aa, synthetic construct
  SITE >22: note = C-terminal is: NH2
  REGION 1..22: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = 8-amino-3,6-dioxaoctanoic acid
XCXHFRWCXXYGRRRRRQRRRR

SEQ ID NO: 450 - protein, 20 aa, synthetic construct
  SITE >20: note = C-terminal is: NH2
  REGION 1..20: note = Description of Sequence: MC4R agonist
  SITE <1: note = N-terminal is: Acetylated
  SITE 1: note = Nle
  REGION 2..8: note = Cyclic
  MOD_RES 3: note = D-Beta-cyclohexylalanine
  SITE 5: note = D-Phenylalanine
  MOD_RES 9..10: note = 8-amino-3,6-dioxaoctanoic acid
XCXHFRWCXXRRRRRQRRRR

SEQ ID NO: 451 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = bAla
XCHFRWXCAYGRRRRRQRRR

SEQ ID NO: 452 - protein, 18 aa, synthetic construct
  SITE >18: note = C-terminal is: NH2
  REGION 1..18: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = bAla
XCHFRWXCARRRRRQRRR

SEQ ID NO: 453 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = bAla
XCHFRWXCAAYGRRRRRQRRR

SEQ ID NO: 454 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = bAla
XCHFRWXCAARRRRRQRRR

SEQ ID NO: 455 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = bAla
XCHFRWXCAYGRRRRRQRRRR

SEQ ID NO: 456 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = bAla
XCHFRWXCARRRRRQRRRR

SEQ ID NO: 457 - protein, 22 aa, synthetic construct
  SITE >22: note = C-terminal is: NH2
  REGION 1..22: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = bAla
XCHFRWXCAAYGRRRRRQRRRR

SEQ ID NO: 458 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = bAla
XCHFRWXCAARRRRRQRRRR

SEQ ID NO: 459 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = 8-amino-3,6-dioxaoctanoic acid
XCHFRWXCXYGRRRRRQRRR

SEQ ID NO: 460 - protein, 18 aa, synthetic construct
  SITE >18: note = C-terminal is: NH2
  REGION 1..18: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = 8-amino-3,6-dioxaoctanoic acid
XCHFRWXCXRRRRRQRRR

SEQ ID NO: 461 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XCHFRWXCXXYGRRRRRQRRR

SEQ ID NO: 462 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XCHFRWXCXXRRRRRQRRR

SEQ ID NO: 463 - protein, 21 aa, synthetic construct
  SITE >21: note = C-terminal is: NH2
  REGION 1..21: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = 8-amino-3,6-dioxaoctanoic acid
XCHFRWXCXYGRRRRRQRRRR

SEQ ID NO: 464 - protein, 19 aa, synthetic construct
  SITE >19: note = C-terminal is: NH2
  REGION 1..19: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9: note = 8-amino-3,6-dioxaoctanoic acid
XCHFRWXCXRRRRRQRRRR

SEQ ID NO: 465 - protein, 22 aa, synthetic construct
  SITE >22: note = C-terminal is: NH2
  REGION 1..22: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XCHFRWXCXXYGRRRRRQRRRR

SEQ ID NO: 466 - protein, 20 aa, synthetic construct
  SITE >20: note = C-terminal is: NH2
  REGION 1..20: note = Description of Sequence: MC4R agonist
  SITE 1: note = Nle
  REGION 2..8: note = Cyclic
  SITE 4: note = D-Phenylalanine
  MOD_RES 7: note = 4abu
  MOD_RES 9..10: note = 8-amino-3,6-dioxaoctanoic acid
XCHFRWXCXXRRRRRQRRRR

SEQ ID NO: 467 - protein, 12 aa, synthetic construct
  SITE >12: note = C-terminal is: NH2
  REGION 1..12: note = Description of Sequence: MC4R agonist
  SITE <1: note = N-terminal is: Acetylated
  REGION 1..8: note = Cyclic
  MOD_RES 4: note = D-4-Br-Phe
CEHFRWGCPPKD

SEQ ID NO: 468 - protein, 12 aa, synthetic construct
  SITE >12: note = C-terminal is: NH2
  REGION 1..12: note = Description of Sequence: MC4R agonist
  SITE <1: note = N-terminal is: Acetylated
  REGION 1..8: note = Cyclic
  MOD_RES 4: note = D-P-(2-naphthyl)alanine
CEHXRWACPPKD

SEQ ID NO: 469 - protein, 12 aa, synthetic construct
  SITE >12: note = C-terminal is: NH2
  REGION 1..12: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
CEHXRXACPPKD

SEQ ID NO: 470 - protein, 12 aa, synthetic construct
  SITE >12: note = C-terminal is: NH2
  REGION 1..12: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 1-Nal
CEHXRXACPPKD

SEQ ID NO: 471 - protein, 12 aa, synthetic construct
  SITE >12: note = C-terminal is: NH2
  REGION 1..12: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = 3-benzothienylaaanine
CEHXRXACPPKD

SEQ ID NO: 472 - protein, 12 aa, synthetic construct
  SITE >12: note = C-terminal is: NH2
  REGION 1..12: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 7: note = bAla
CEHXRXACPPKD

SEQ ID NO: 473 - protein, 12 aa, synthetic construct
  SITE >12: note = C-terminal is: NH2
  REGION 1..12: note = Description of Sequence: MC4R agonist
  SITE <1: note = N-terminal is: Acetylated
  REGION 1..8: note = Cyclic
  MOD_RES 4: note = D-P-(2-naphthyl)alanine
  MOD_RES 6: note = P-(2-naphthyl)alanine
  MOD_RES 7: note = a-aminoisobutyric acid
CEHXRXXCPPKD

SEQ ID NO: 474 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Cys-D-Ala))-His
  SITE 2: note = D-Phenylalanine
HFRWC

SEQ ID NO: 475 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(hCys-D-Ala))-His
  SITE 2: note = D-Phenylalanine
HFRWC

SEQ ID NO: 476 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Cys-D-Ala))-His
  MOD_RES 2: note = D-P-(2-naphthyl)alanine
HXRWC

SEQ ID NO: 477 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(hCys-D-Ala))-His
  MOD_RES 2: note = D-P-(2-naphthyl)alanine
HXRWC

SEQ ID NO: 478 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Asp-D-Ala))-His
  SITE 2: note = D-Phenylalanine
HFRWK

SEQ ID NO: 479 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Asp-D-Ala))-His
  SITE 2: note = D-Phenylalanine
  MOD_RES 5: note = Orn
HFRWX

SEQ ID NO: 480 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Asp-D-Ala))-His
  SITE 2: note = D-Phenylalanine
  MOD_RES 5: note = Dab
HFRWX

SEQ ID NO: 481 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Asp-D-Ala))-His
  SITE 2: note = D-Phenylalanine
  MOD_RES 5: note = Dap
HFRWX

SEQ ID NO: 482 - skipped

SEQ ID NO: 483 - protein, 4 aa, synthetic construct
  SITE >4: note = C-terminal is: NH2
  REGION 1..4: note = Description of Sequence: MC4R agonist
  REGION 1..4: note = Cyclic
  MOD_RES 1: note = Hydantoin(C(O)-(Asp-His))-D-Phe
FRWK

SEQ ID NO: 484 - protein, 4 aa, synthetic construct
  SITE >4: note = C-terminal is: NH2
  REGION 1..4: note = Description of Sequence: MC4R agonist
  REGION 1..4: note = Cyclic
  MOD_RES 1: note = Hydantoin(C(O)-(Asp-A3c))-D-Phe
FRWK

SEQ ID NO: 485 - protein, 4 aa, synthetic construct
  SITE >4: note = C-terminal is: NH2
  REGION 1..4: note = Description of Sequence: MC4R agonist
  REGION 1..4: note = Cyclic
  MOD_RES 1: note = Hydantoin(C(O)-(Asp-A5c))-D-Phe
FRWK

SEQ ID NO: 486 - protein, 4 aa, synthetic construct
  SITE >4: note = C-terminal is: NH2
  REGION 1..4: note = Description of Sequence: MC4R agonist
  REGION 1..4: note = Cyclic
  MOD_RES 1: note = Hydantoin(C(O)-(Asp-A6c))-D-Phe
FRWK

SEQ ID NO: 487-489 - skipped (3 entries)

SEQ ID NO: 490 - protein, 4 aa, synthetic construct
  SITE >4: note = C-terminal is: NH2
  REGION 1..4: note = Description of Sequence: MC4R agonist
  REGION 1..4: note = Cyclic
  MOD_RES 1: note = Hydantoin(C(O)-(Asp-Aic))-D-Phe
FRWK

SEQ ID NO: 491 - protein, 4 aa, synthetic construct
  SITE >4: note = C-terminal is: NH2
  REGION 1..4: note = Description of Sequence: MC4R agonist
  REGION 1..4: note = Cyclic
  MOD_RES 1: note = Hydantoin(C(O)-(Asp-Apc))-D-Phe
FRWK

SEQ ID NO: 492-493 - skipped (2 entries)

SEQ ID NO: 494 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Glu-D-Ala))-His
  SITE 2: note = D-Phenylalanine
  MOD_RES 5: note = Orn
HFRWX

SEQ ID NO: 495 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Glu-D-Ala))-His
  SITE 2: note = D-Phenylalanine
  MOD_RES 5: note = Dab
HFRWX

SEQ ID NO: 496 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Glu-D-Ala))-His
  SITE 2: note = D-Phenylalanine
  MOD_RES 5: note = Dap
HFRWX

SEQ ID NO: 497 - protein, 5 aa, synthetic construct
  SITE >5: note = C-terminal is: NH2
  REGION 1..5: note = Description of Sequence: MC4R agonist
  REGION 1..5: note = Cyclic
  MOD_RES 1: note = Hydantoin(C(O)-(Glu-D-Ala))-His
  SITE 2: note = D-Phenylalanine
HFRWK

SEQ ID NO: 498 - skipped

SEQ ID NO: 499 - protein, 4 aa, synthetic construct
  SITE >4: note = C-terminal is: NH2
  REGION 1..4: note = Description of Sequence: MC4R agonist
  REGION 1..4: note = Cyclic
  MOD_RES 1: note = Hydantoin(C(O)-(Glu-His))-D-Phe
FRWK

SEQ ID NO: 500 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Arg-Gly))-Cys
  SITE 4: note = D-Phenylalanine
CEHFRWC

SEQ ID NO: 501 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Nle-Gly))-Cys
  SITE 4: note = D-Phenylalanine
CEHFRWC

SEQ ID NO: 502 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Gly))-Cys
  SITE 4: note = D-Phenylalanine
CEHFRWC

SEQ ID NO: 503 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Nle-Gly))-Cys
  SITE 2: note = D-Alanine
  SITE 4: note = D-Phenylalanine
CAHFRWC

SEQ ID NO: 504 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Gly))-Cys
  SITE 2: note = D-Alanine
  SITE 4: note = D-Phenylalanine
CAHFRWC

SEQ ID NO: 505 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Nle-Gly))-Cys
  SITE 2: note = D-Alanine
  SITE 4: note = D-Phenylalanine
  MOD_RES 7: note = Penicillamine
CAHFRWX

SEQ ID NO: 506 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Gly))-Cys
  SITE 2: note = D-Alanine
  SITE 4: note = D-Phenylalanine
  MOD_RES 7: note = Penicillamine
CAHFRWX

SEQ ID NO: 507 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Ala-Gly))-Cys
  SITE 2: note = D-Alanine
  SITE 4: note = D-Phenylalanine
CAHFRWC

SEQ ID NO: 508 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(D-Ala-Gly))-Cys
  SITE 2: note = D-Alanine
  SITE 4: note = D-Phenylalanine
CAHFRWC

SEQ ID NO: 509 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Aib-Gly))-Cys
  SITE 2: note = D-Alanine
  SITE 4: note = D-Phenylalanine
CAHFRWC

SEQ ID NO: 510 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Val-Gly))-Cys
  SITE 2: note = D-Alanine
  SITE 4: note = D-Phenylalanine
CAHFRWC

SEQ ID NO: 511 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Ile-Gly))-Cys
  SITE 2: note = D-Alanine
  SITE 4: note = D-Phenylalanine
CAHFRWC

SEQ ID NO: 512 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Leu-Gly))-Cys
  SITE 2: note = D-Alanine
  SITE 4: note = D-Phenylalanine
CAHFRWC

SEQ ID NO: 513 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Gly))-Cys
  MOD_RES 4: note = D-P-(2-naphthyl)alanine
CEHXRWC

SEQ ID NO: 514 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Nle-Gly))-Cys
  MOD_RES 4: note = D-P-(2-naphthyl)alanine
CEHXRWC

SEQ ID NO: 515 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(D-Arg-Gly))-Cys
  SITE 4: note = D-Phenylalanine
CEHFRWC

SEQ ID NO: 516 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(D-Arg-Gly))-Cys
  SITE 2: note = D-Alanine
  SITE 4: note = D-Phenylalanine
CAHFRWC

SEQ ID NO: 517 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Arg-Gly))-Cys
  SITE 2: note = D-Alanine
  SITE 4: note = D-Phenylalanine
CAHFRWC

SEQ ID NO: 518 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(D-Arg-Gly))-Cys
  SITE 2: note = D-Alanine
  MOD_RES 4: note = D-P-(2-naphthyl)alanine
CAHXRWC

SEQ ID NO: 519 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Arg-Gly))-Cys
  SITE 2: note = D-Alanine
  MOD_RES 4: note = D-P-(2-naphthyl)alanine
CAHXRWC

SEQ ID NO: 520 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Ala-Nle))-Cys
  SITE 4: note = D-Phenylalanine
CEHFRWC

SEQ ID NO: 521 - protein, 7 aa, synthetic construct
  SITE >7: note = C-terminal is: NH2
  REGION 1..7: note = Description of Artificial Sequence: Synthetic peptide
  REGION 1..7: note = Cyclic
  MOD_RES 1: note = Hydantoin(C(O)-(Val-Nle))-Cys
  SITE 4: note = D-Phenylalanine
CEHFRWC

SEQ ID NO: 522 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Nle))-Cys
  SITE 4: note = D-Phenylalanine
CEHFRWC

SEQ ID NO: 523 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(A6c-Nle))-Cys
  SITE 2: note = D-Alanine
  SITE 4: note = D-Phenylalanine
CAHFRWC

SEQ ID NO: 524 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Nle))-Cys
  SITE 2: note = D-Alanine
  SITE 4: note = D-Phenylalanine
CAHFRWC

SEQ ID NO: 525 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Ala-Nle))-Cys
  SITE 2: note = D-Alanine
  SITE 4: note = D-Phenylalanine
CAHFRWC

SEQ ID NO: 526 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(D-Ala-Nle))-Cys
  SITE 2: note = D-Alanine
  SITE 4: note = D-Phenylalanine
CAHFRWC

SEQ ID NO: 527 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Val-Nle))-Cys
  SITE 2: note = D-Alanine
  SITE 4: note = D-Phenylalanine
CAHFRWC

SEQ ID NO: 528 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Leu-Nle))-Cys
  SITE 2: note = D-Alanine
  SITE 4: note = D-Phenylalanine
CAHFRWC

SEQ ID NO: 529 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Cha-Nle))-Cys
  SITE 2: note = D-Alanine
  SITE 4: note = D-Phenylalanine
CAHFRWC

SEQ ID NO: 530 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Aib-Nle))-Cys
  SITE 2: note = D-Alanine
  SITE 4: note = D-Phenylalanine
CAHFRWC

SEQ ID NO: 531 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Arg))-Cys
  SITE 4: note = D-Phenylalanine
CEHFRWC

SEQ ID NO: 532 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Arg))-Cys
  MOD_RES 4: note = D-P-(2-naphthyl)alanine
CEHXRWC

SEQ ID NO: 533 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Arg))-Cys
  SITE 2: note = D-Alanine
  SITE 4: note = D-Phenylalanine
CAHFRWC

SEQ ID NO: 534 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-Arg))-Cys
  SITE 2: note = D-Alanine
  MOD_RES 4: note = D-P-(2-naphthyl)alanine
CAHXRWC

SEQ ID NO: 535 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-D-Arg))-Cys
  SITE 4: note = D-Phenylalanine
CEHFRWC

SEQ ID NO: 536 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-D-Arg))-Cys
  SITE 2: note = D-Alanine
  SITE 4: note = D-Phenylalanine
CAHFRWC

SEQ ID NO: 537 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Gly-D-Arg))-Cys
  SITE 2: note = D-Alanine
  MOD_RES 4: note = D-P-(2-naphthyl)alanine
CAHXRWC

SEQ ID NO: 538 - protein, 7 aa, synthetic construct
  SITE >7: note = C-terminal is: NH2
  REGION 1..7: note = Description of Sequence: MC4R agonist
  REGION 1..7: note = Cyclic
  MOD_RES 1: note = Hydantoin(C(O)-(Nle-Ala))-Cys
  SITE 4: note = D-Phenylalanine
CEHFRWC

SEQ ID NO: 539 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Nle-Cys))-D-Ala
  SITE 3: note = D-Phenylalanine
AHFRWC

SEQ ID NO: 540 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Ala-Cys))-D-Ala
  SITE 3: note = D-Phenylalanine
AHFRWC

SEQ ID NO: 541 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(D-Ala-Cys))-D-Ala
  SITE 3: note = D-Phenylalanine
AHFRWC

SEQ ID NO: 542 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Aib-Cys))-D-Ala
  SITE 3: note = D-Phenylalanine
AHFRWC

SEQ ID NO: 543 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Val-Cys))-D-Ala
  SITE 3: note = D-Phenylalanine
AHFRWC

SEQ ID NO: 544 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Abu-Cys))-D-Ala
  SITE 3: note = D-Phenylalanine
AHFRWC

SEQ ID NO: 545 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Leu-Cys))-D-Ala
  SITE 3: note = D-Phenylalanine
AHFRWC

SEQ ID NO: 546 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Ile-Cys))-D-Ala
  SITE 3: note = D-Phenylalanine
AHFRWC

SEQ ID NO: 547 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Cha-Cys))-D-Ala
  SITE 3: note = D-Phenylalanine
AHFRWC

SEQ ID NO: 548 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(A6c-Cys))-D-Ala
  SITE 3: note = D-Phenylalanine
AHFRWC

SEQ ID NO: 549 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Phe-Cys))-D-Ala
  SITE 3: note = D-Phenylalanine
AHFRWC

SEQ ID NO: 550 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Gly-Cys))-D-Ala
  SITE 3: note = D-Phenylalanine
AHFRWC

SEQ ID NO: 551 - protein, 6 aa, synthetic construct
  SITE >6: note = C-terminal is: NH2
  REGION 1..6: note = Description of Sequence: MC4R agonist
  REGION 1..6: note = Cyclic
  MOD_RES 1: note = Hydantoin(C(O)-(Gly-Cys))-Glu
  SITE 3: note = D-Phenylalanine
EHFRWC

SEQ ID NO: 552 - protein, 9 aa, synthetic construct
  SITE >9: note = C-terminal is: NH2
  REGION 1..9: note = Description of Sequence: MC4R agonist
  SITE <1: note = N-terminal is: Acetylated
  REGION 3..9: note = Cyclic
  SITE 4: note = D-Alanine
  SITE 6: note = D-Phenylalanine
YRCAHFRWC

SEQ ID NO: 553 - protein, 9 aa, synthetic construct
  SITE >9: note = C-terminal is: NH2
  REGION 1..9: note = Description of Sequence: MC4R agonist
  SITE <1: note = N-terminal is: Acetylated
  MOD_RES 1: note = P-(2-naphthyl)alanine
  REGION 3..9: note = Cyclic
  SITE 4: note = D-Alanine
  SITE 6: note = D-Phenylalanine
XRCAHFRWC

SEQ ID NO: 554 - protein, 9 aa, synthetic construct
  SITE >9: note = C-terminal is: NH2
  REGION 1..9: note = Description of Sequence: MC4R agonist
  SITE <1: note = N-terminal is: Acetylated
  MOD_RES 1: note = 1-Nal
  REGION 3..9: note = Cyclic
  SITE 4: note = D-Alanine
  SITE 6: note = D-Phenylalanine
XRCAHFRWC

SEQ ID NO: 555 - protein, 9 aa, synthetic construct
  SITE >9: note = C-terminal is: NH2
  REGION 1..9: note = Description of Sequence: MC4R agonist
  SITE <1: note = N-terminal is: Acetylated
  REGION 3..9: note = Cyclic
  SITE 4: note = D-Alanine
  SITE 6: note = D-Phenylalanine
FRCAHFRWC

SEQ ID NO: 556 - protein, 9 aa, synthetic construct
  SITE >9: note = C-terminal is: NH2
  REGION 1..9: note = Description of Sequence: MC4R agonist
  SITE <1: note = N-terminal is: Acetylated
  REGION 3..9: note = Cyclic
  SITE 4: note = D-Alanine
  SITE 6: note = D-Phenylalanine
WRCAHFRWC

SEQ ID NO: 557 - protein, 9 aa, synthetic construct
  SITE >9: note = C-terminal is: NH2
  REGION 1..9: note = Description of Sequence: MC4R agonist
  SITE <1: note = N-terminal is: Acetylated
  MOD_RES 1: note = Pff
  REGION 3..9: note = Cyclic
  SITE 4: note = D-Alanine
  SITE 6: note = D-Phenylalanine
XRCAHFRWC

SEQ ID NO: 558 - protein, 9 aa, synthetic construct
  SITE >9: note = C-terminal is: NH2
  REGION 1..9: note = Description of Sequence: MC4R agonist
  SITE <1: note = N-term is: H
  REGION 3..9: note = Cyclic
  SITE 4: note = D-Alanine
  SITE 6: note = D-Phenylalanine
HRCAHFRWC

SEQ ID NO: 559 - protein, 9 aa, synthetic construct
  SITE >9: note = C-terminal is: NH2
  REGION 1..9: note = Description of Sequence: MC4R agonist
  SITE <1: note = N-terminal is: Acetylated etylated
  REGION 3..9: note = Cyclic
  SITE 4: note = D-Alanine
  SITE 6: note = D-Phenylalanine
HRCAHFRWC